SEQUENCE LISTING (263 sequences)

SEQ ID NO: 1 - RNA, 110 nt, synthetic construct
  source 1..110: mol_type = other RNA
acctggattagagttacatctcctggatgattagttcagagatatattaaaatgccccctccctgtggatcctatagaagatttgcatcttttgtgtgatgagtgcagag

SEQ ID NO: 2 - RNA, 45 nt, synthetic construct
  source 1..45: mol_type = other RNA
gtggaatagtataacaatatgctaaatgttgttatagtatcccac

SEQ ID NO: 3 - DNA, 56 bp, synthetic construct
  source 1..56: mol_type = other DNA
gccatcagtcgccggtcccaagcccggataaaatgggagggggcgggaaaccgcct

SEQ ID NO: 4 - RNA, 56 nt, synthetic construct
  source 1..56: mol_type = other RNA
gccatcagtcgccggtcccaagcccggataaaatgggagggggcgggaaaccgcct

SEQ ID NO: 5 - DNA, 55 bp, synthetic construct
  source 1..55: mol_type = other DNA
aacactgccaatgccggtcccaagcccggataaaagtggagggtacagtccacgc

SEQ ID NO: 6 - RNA, 55 nt, synthetic construct
  source 1..55: mol_type = other RNA
aacactgccaatgccggtcccaagcccggataaaagtggagggtacagtccacgc

SEQ ID NO: 7 - DNA, 21 bp, synthetic construct
  source 1..21: mol_type = other DNA
aaccatgccgactgatggcag

SEQ ID NO: 8 - DNA, 26 bp, synthetic construct
  source 1..26: mol_type = other DNA
gatgtcaggtgcggctgactaccgtc

SEQ ID NO: 9 - RNA, 21 nt, synthetic construct
  source 1..21: mol_type = other RNA
aaccatgccgactgatggcag

SEQ ID NO: 10 - RNA, 26 nt, synthetic construct
  source 1..26: mol_type = other RNA
gatgtcaggtgcggctgactaccgtc

SEQ ID NO: 11 - DNA, 10 bp, synthetic construct
  source 1..10: mol_type = other DNA
aaaaaggggg

SEQ ID NO: 12 - DNA, 10 bp, synthetic construct
  source 1..10: mol_type = other DNA
aaaaaaaaaa

SEQ ID NO: 13 - DNA, 20 bp, synthetic construct
  source 1..20: mol_type = other DNA
cccccggggggggggccccc

SEQ ID NO: 14 - DNA, 10 bp, synthetic construct
  source 1..10: mol_type = other DNA
gggggggggg

SEQ ID NO: 15 - protein, 10 aa, synthetic construct
FFFFFYYYYY

SEQ ID NO: 16 - protein, 10 aa, synthetic construct
YYYYYYYYYY

SEQ ID NO: 17 - protein, 20 aa, synthetic construct
LLLLLFFFFFYYYYYLLLLL

SEQ ID NO: 18 - DNA, 112 bp, synthetic construct
  source 1..112: mol_type = other DNA
ctctgcactcatcacacaaaagatgcaaatcttctataggatccacagggagggggcattttaacatatctmgctgaactaatcatccaggagatgtaactctaatccaggt

SEQ ID NO: 19 - DNA, 32 bp, synthetic construct
  source 1..32: mol_type = other DNA
taggctttctggctttttaccggaaagcccct

SEQ ID NO: 20 - RNA, 32 nt, synthetic construct
  source 1..32: mol_type = other RNA
taggctttctggctttttaccggaaagcccct

SEQ ID NO: 21 - DNA, 31 bp, synthetic construct
  source 1..31: mol_type = other DNA
caggttttctgacttcggtcggaaaacccct

SEQ ID NO: 22 - RNA, 31 nt, synthetic construct
  source 1..31: mol_type = other RNA
caggttttctgacttcggtcggaaaacccct

SEQ ID NO: 23 - DNA, 11 bp, synthetic construct
  source 1..11: mol_type = other DNA
aatttttggag

SEQ ID NO: 24 - RNA, 11 nt, synthetic construct
  source 1..11: mol_type = other RNA
aatttttggag

SEQ ID NO: 25 - DNA, 249 bp, synthetic construct
  source 1..249: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 26 - DNA, 341 bp, synthetic construct
  source 1..341: mol_type = other DNA
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 27 - DNA, 263 bp, synthetic construct
  source 1..263: mol_type = other DNA
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 28 - DNA, 257 bp, synthetic construct
  source 1..257: mol_type = other DNA
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 29 - DNA, 393 bp, synthetic construct
  source 1..393: mol_type = other DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 30 - DNA, 698 bp, synthetic construct
  source 1..698: mol_type = other DNA
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 31 - DNA, 110 bp, synthetic construct
  source 1..110: mol_type = other DNA
acctggattagagttacatctcctggatgattagttcagagatatattaaaatgccccctccctgtggatcctatagaagatttgcatcttttgtgtgatgagtgcagag

SEQ ID NO: 32 - DNA, 100 bp, synthetic construct
  source 1..100: mol_type = other DNA
ctctgcactcatcacctactcgatgcaaatcttctataggatccacagggagggggcattttaacatatgcagctactaatcatccaggagatgtaactc

SEQ ID NO: 33 - DNA, 100 bp, synthetic construct
  source 1..100: mol_type = other DNA
ctctgcactcatcacacaaaagatgcaaatctgtgggcggatccacagggagggggcattttaacatatcagctccctaatcatccaggagatgtaactc

SEQ ID NO: 34 - DNA, 100 bp, synthetic construct
  source 1..100: mol_type = other DNA
cactcatcactgccgtgatgcaaatcttctataggatccacagggagggggcattttaacatatctcggtttcaatcatccaggagatgtaactctaatc

SEQ ID NO: 35 - DNA, 100 bp, synthetic construct
  source 1..100: mol_type = other DNA
atcacacaaaagatcttccccttctataggatccacagggagggggcatttcaatatatctcactcgaaatcatccaggagatgtaactctaatccaggt

SEQ ID NO: 36 - DNA, 100 bp, synthetic construct
  source 1..100: mol_type = other DNA
cactcatcacacaaaagacccctctcttctataggatccacagggagggggcattctaatattcaaacgaactaatcatccaggagatgtaactctaatc

SEQ ID NO: 37 - DNA, 100 bp, synthetic construct
  source 1..100: mol_type = other DNA
atcacactccctctgcaaatcttctataggatccacagggagggggcattctaatatatctctgaccctcgcatccaggagatgtaactctaatccaggt

SEQ ID NO: 38 - DNA, 101 bp, synthetic construct
  source 1..101: mol_type = other DNA
atcacacaaaagatgcaaatctgtggctggatccacagggagggggcattttaacgactctgtgtgaacgatcatcttactcttgtaactctaatccaggt

SEQ ID NO: 39 - RNA, 100 nt, synthetic construct
  source 1..100: mol_type = other RNA
ctctgcactcatcacctactcgatgcaaatcttctataggatccacagggagggggcattttaacatatgcagctactaatcatccaggagatgtaactc

SEQ ID NO: 40 - RNA, 100 nt, synthetic construct
  source 1..100: mol_type = other RNA
ctctgcactcatcacacaaaagatgcaaatctgtgggcggatccacagggagggggcattttaacatatcagctccctaatcatccaggagatgtaactc

SEQ ID NO: 41 - RNA, 100 nt, synthetic construct
  source 1..100: mol_type = other RNA
cactcatcactgccgtgatgcaaatcttctataggatccacagggagggggcattttaacatatctcggtttcaatcatccaggagatgtaactctaatc

SEQ ID NO: 42 - RNA, 100 nt, synthetic construct
  source 1..100: mol_type = other RNA
atcacacaaaagatcttccccttctataggatccacagggagggggcatttcaatatatctcactcgaaatcatccaggagatgtaactctaatccaggt

SEQ ID NO: 43 - RNA, 100 nt, synthetic construct
  source 1..100: mol_type = other RNA
cactcatcacacaaaagacccctctcttctataggatccacagggagggggcattctaatattcaaacgaactaatcatccaggagatgtaactctaatc

SEQ ID NO: 44 - RNA, 100 nt, synthetic construct
  source 1..100: mol_type = other RNA
atcacactccctctgcaaatcttctataggatccacagggagggggcattctaatatatctctgaccctcgcatccaggagatgtaactctaatccaggt

SEQ ID NO: 45 - RNA, 101 nt, synthetic construct
  source 1..101: mol_type = other RNA
atcacacaaaagatgcaaatctgtggctggatccacagggagggggcattttaacgactctgtgtgaacgatcatcttactcttgtaactctaatccaggt

SEQ ID NO: 46 - RNA, 101 nt, synthetic construct
  source 1..101: mol_type = other RNA
ctctgcactcatcacacaaaagacccctctcttctataggatccacagggagggggcattttaacgactctgtgtgaacgatcatcttactcttgtaactc

SEQ ID NO: 47 - DNA, 100 bp, synthetic construct
  source 1..100: mol_type = other DNA
cactcatcacctactcgatgcaaatcttctataggatccacagggagggggcattttaacatatgcagctactaatcatccaggagatgtaactctaatc

SEQ ID NO: 48 - DNA, 100 bp, synthetic construct
  source 1..100: mol_type = other DNA
catatctctgcactcatcacctactcgatgcaaatcttctataggatccacagggagggggcattttaacatatgcagctactaatcatccaggagatgt

SEQ ID NO: 49 - DNA, 100 bp, synthetic construct
  source 1..100: mol_type = other DNA
tgtgacatatctctgcactcatcacctactcgatgcaaatcttctataggatccacagggagggggcattttaacatatgcagctactaatcatccagga

SEQ ID NO: 50 - DNA, 100 bp, synthetic construct
  source 1..100: mol_type = other DNA
gatattgtgacatatctctgcactcatcacctactcgatgcaaatcttctataggatccacagggagggggcattttaacatatgcagctactaatcatc

SEQ ID NO: 51 - DNA, 100 bp, synthetic construct
  source 1..100: mol_type = other DNA
acaaaagatgcaaatctgtgggcggatccacagggagggggcattttaacatatcagctccctaatcatccaggagatgtaactctaatccaggtttgcc

SEQ ID NO: 52 - DNA, 100 bp, synthetic construct
  source 1..100: mol_type = other DNA
atcacacaaaagatgcaaatctgtgggcggatccacagggagggggcattttaacatatcagctccctaatcatccaggagatgtaactctaatccaggt

SEQ ID NO: 53 - DNA, 100 bp, synthetic construct
  source 1..100: mol_type = other DNA
cactcatcacacaaaagatgcaaatctgtgggcggatccacagggagggggcattttaacatatcagctccctaatcatccaggagatgtaactctaatc

SEQ ID NO: 54 - DNA, 100 bp, synthetic construct
  source 1..100: mol_type = other DNA
catatctctgcactcatcacacaaaagatgcaaatctgtgggcggatccacagggagggggcattttaacatatcagctccctaatcatccaggagatgt

SEQ ID NO: 55 - DNA, 100 bp, synthetic construct
  source 1..100: mol_type = other DNA
tgtgacatatctctgcactcatcacacaaaagatgcaaatctgtgggcggatccacagggagggggcattttaacatatcagctccctaatcatccagga

SEQ ID NO: 56 - DNA, 100 bp, synthetic construct
  source 1..100: mol_type = other DNA
gatattgtgacatatctctgcactcatcacacaaaagatgcaaatctgtgggcggatccacagggagggggcattttaacatatcagctccctaatcatc

SEQ ID NO: 57 - DNA, 100 bp, synthetic construct
  source 1..100: mol_type = other DNA
ctctgcactcatcactgccgtgatgcaaatcttctataggatccacagggagggggcattttaacatatctcggtttcaatcatccaggagatgtaactc

SEQ ID NO: 58 - DNA, 100 bp, synthetic construct
  source 1..100: mol_type = other DNA
catatctctgcactcatcactgccgtgatgcaaatcttctataggatccacagggagggggcattttaacatatctcggtttcaatcatccaggagatgt

SEQ ID NO: 59 - DNA, 100 bp, synthetic construct
  source 1..100: mol_type = other DNA
tgtgacatatctctgcactcatcactgccgtgatgcaaatcttctataggatccacagggagggggcattttaacatatctcggtttcaatcatccagga

SEQ ID NO: 60 - DNA, 100 bp, synthetic construct
  source 1..100: mol_type = other DNA
gatattgtgacatatctctgcactcatcactgccgtgatgcaaatcttctataggatccacagggagggggcattttaacatatctcggtttcaatcatc

SEQ ID NO: 61 - DNA, 100 bp, synthetic construct
  source 1..100: mol_type = other DNA
cactcatcacacaaaagatcttccccttctataggatccacagggagggggcatttcaatatatctcactcgaaatcatccaggagatgtaactctaatc

SEQ ID NO: 62 - DNA, 100 bp, synthetic construct
  source 1..100: mol_type = other DNA
ctctgcactcatcacacaaaagatcttccccttctataggatccacagggagggggcatttcaatatatctcactcgaaatcatccaggagatgtaactc

SEQ ID NO: 63 - DNA, 100 bp, synthetic construct
  source 1..100: mol_type = other DNA
catatctctgcactcatcacacaaaagatcttccccttctataggatccacagggagggggcatttcaatatatctcactcgaaatcatccaggagatgt

SEQ ID NO: 64 - DNA, 100 bp, synthetic construct
  source 1..100: mol_type = other DNA
tgtgacatatctctgcactcatcacacaaaagatcttccccttctataggatccacagggagggggcatttcaatatatctcactcgaaatcatccagga

SEQ ID NO: 65 - DNA, 100 bp, synthetic construct
  source 1..100: mol_type = other DNA
gatattgtgacatatctctgcactcatcacacaaaagatcttccccttctataggatccacagggagggggcatttcaatatatctcactcgaaatcatc

SEQ ID NO: 66 - DNA, 100 bp, synthetic construct
  source 1..100: mol_type = other DNA
ctctgcactcatcacacaaaagacccctctcttctataggatccacagggagggggcattctaatattcaaacgaactaatcatccaggagatgtaactc

SEQ ID NO: 67 - DNA, 100 bp, synthetic construct
  source 1..100: mol_type = other DNA
catatctctgcactcatcacacaaaagacccctctcttctataggatccacagggagggggcattctaatattcaaacgaactaatcatccaggagatgt

SEQ ID NO: 68 - DNA, 100 bp, synthetic construct
  source 1..100: mol_type = other DNA
tgtgacatatctctgcactcatcacacaaaagacccctctcttctataggatccacagggagggggcattctaatattcaaacgaactaatcatccagga

SEQ ID NO: 69 - DNA, 100 bp, synthetic construct
  source 1..100: mol_type = other DNA
gatattgtgacatatctctgcactcatcacacaaaagacccctctcttctataggatccacagggagggggcattctaatattcaaacgaactaatcatc

SEQ ID NO: 70 - DNA, 100 bp, synthetic construct
  source 1..100: mol_type = other DNA
cactcatcacactccctctgcaaatcttctataggatccacagggagggggcattctaatatatctctgaccctcgcatccaggagatgtaactctaatc

SEQ ID NO: 71 - DNA, 100 bp, synthetic construct
  source 1..100: mol_type = other DNA
ctctgcactcatcacactccctctgcaaatcttctataggatccacagggagggggcattctaatatatctctgaccctcgcatccaggagatgtaactc

SEQ ID NO: 72 - DNA, 100 bp, synthetic construct
  source 1..100: mol_type = other DNA
catatctctgcactcatcacactccctctgcaaatcttctataggatccacagggagggggcattctaatatatctctgaccctcgcatccaggagatgt

SEQ ID NO: 73 - DNA, 100 bp, synthetic construct
  source 1..100: mol_type = other DNA
tgtgacatatctctgcactcatcacactccctctgcaaatcttctataggatccacagggagggggcattctaatatatctctgaccctcgcatccagga

SEQ ID NO: 74 - DNA, 100 bp, synthetic construct
  source 1..100: mol_type = other DNA
gatattgtgacatatctctgcactcatcacactccctctgcaaatcttctataggatccacagggagggggcattctaatatatctctgaccctcgcatc

SEQ ID NO: 75 - DNA, 101 bp, synthetic construct
  source 1..101: mol_type = other DNA
cactcatcacacaaaagatgcaaatctgtgggcggatccacagggagggggcattttaacgactctgtgtgaacgatcatcttactcttgtaactctaatc

SEQ ID NO: 76 - DNA, 101 bp, synthetic construct
  source 1..101: mol_type = other DNA
cactcatcacacaaaagacccctctcttctataggatccacagggagggggcattttaacgactctgtgtgaacgatcatcttactcttgtaactctaatc

SEQ ID NO: 77 - DNA, 101 bp, synthetic construct
  source 1..101: mol_type = other DNA
cactcatcacactccctctgcaaatcttctataggatccacagggagggggcattttaacgactctgtgtgaacgatcatcttactcttgtaactctaatc

SEQ ID NO: 78 - DNA, 101 bp, synthetic construct
  source 1..101: mol_type = other DNA
ctctgcactcatcacacaaaagatgcaaatctgtgggcggatccacagggagggggcattttaacgactctgtgtgaacgatcatcttactcttgtaactc

SEQ ID NO: 79 - DNA, 101 bp, synthetic construct
  source 1..101: mol_type = other DNA
ctctgcactcatcacacaaaagacccctctcttctataggatccacagggagggggcattttaacgactctgtgtgaacgatcatcttactcttgtaactc

SEQ ID NO: 80 - DNA, 101 bp, synthetic construct
  source 1..101: mol_type = other DNA
ctctgcactcatcacactccctctgcaaatcttctataggatccacagggagggggcattttaacgactctgtgtgaacgatcatcttactcttgtaactc

SEQ ID NO: 81 - DNA, 30 bp, synthetic construct
  source 1..30: mol_type = other DNA
ggagggggcattttaatatatctctgaact

SEQ ID NO: 82 - DNA, 61 bp, synthetic construct
  source 1..61: mol_type = other DNA
ggagggggcattttaatatatctctgaactaatcatccaggagatgtaactctaatccagg

SEQ ID NO: 83 - RNA, 100 nt, synthetic construct
  source 1..100: mol_type = other RNA
cactcatcacctactcgatgcaaatcttctataggatccacagggagggggcattttaacatatgcagctactaatcatccaggagatgtaactctaatc

SEQ ID NO: 84 - RNA, 100 nt, synthetic construct
  source 1..100: mol_type = other RNA
catatctctgcactcatcacctactcgatgcaaatcttctataggatccacagggagggggcattttaacatatgcagctactaatcatccaggagatgt

SEQ ID NO: 85 - RNA, 100 nt, synthetic construct
  source 1..100: mol_type = other RNA
tgtgacatatctctgcactcatcacctactcgatgcaaatcttctataggatccacagggagggggcattttaacatatgcagctactaatcatccagga

SEQ ID NO: 86 - RNA, 100 nt, synthetic construct
  source 1..100: mol_type = other RNA
gatattgtgacatatctctgcactcatcacctactcgatgcaaatcttctataggatccacagggagggggcattttaacatatgcagctactaatcatc

SEQ ID NO: 87 - RNA, 100 nt, synthetic construct
  source 1..100: mol_type = other RNA
acaaaagatgcaaatctgtgggcggatccacagggagggggcattttaacatatcagctccctaatcatccaggagatgtaactctaatccaggtttgcc

SEQ ID NO: 88 - RNA, 100 nt, synthetic construct
  source 1..100: mol_type = other RNA
atcacacaaaagatgcaaatctgtgggcggatccacagggagggggcattttaacatatcagctccctaatcatccaggagatgtaactctaatccaggt

SEQ ID NO: 89 - RNA, 100 nt, synthetic construct
  source 1..100: mol_type = other RNA
cactcatcacacaaaagatgcaaatctgtgggcggatccacagggagggggcattttaacatatcagctccctaatcatccaggagatgtaactctaatc

SEQ ID NO: 90 - RNA, 100 nt, synthetic construct
  source 1..100: mol_type = other RNA
catatctctgcactcatcacacaaaagatgcaaatctgtgggcggatccacagggagggggcattttaacatatcagctccctaatcatccaggagatgt

SEQ ID NO: 91 - RNA, 100 nt, synthetic construct
  source 1..100: mol_type = other RNA
tgtgacatatctctgcactcatcacacaaaagatgcaaatctgtgggcggatccacagggagggggcattttaacatatcagctccctaatcatccagga

SEQ ID NO: 92 - RNA, 100 nt, synthetic construct
  source 1..100: mol_type = other RNA
gatattgtgacatatctctgcactcatcacacaaaagatgcaaatctgtgggcggatccacagggagggggcattttaacatatcagctccctaatcatc

SEQ ID NO: 93 - RNA, 100 nt, synthetic construct
  source 1..100: mol_type = other RNA
ctctgcactcatcactgccgtgatgcaaatcttctataggatccacagggagggggcattttaacatatctcggtttcaatcatccaggagatgtaactc

SEQ ID NO: 94 - RNA, 100 nt, synthetic construct
  source 1..100: mol_type = other RNA
catatctctgcactcatcactgccgtgatgcaaatcttctataggatccacagggagggggcattttaacatatctcggtttcaatcatccaggagatgt

SEQ ID NO: 95 - RNA, 100 nt, synthetic construct
  source 1..100: mol_type = other RNA
tgtgacatatctctgcactcatcactgccgtgatgcaaatcttctataggatccacagggagggggcattttaacatatctcggtttcaatcatccagga

SEQ ID NO: 96 - RNA, 100 nt, synthetic construct
  source 1..100: mol_type = other RNA
gatattgtgacatatctctgcactcatcactgccgtgatgcaaatcttctataggatccacagggagggggcattttaacatatctcggtttcaatcatc

SEQ ID NO: 97 - RNA, 100 nt, synthetic construct
  source 1..100: mol_type = other RNA
cactcatcacacaaaagatcttccccttctataggatccacagggagggggcatttcaatatatctcactcgaaatcatccaggagatgtaactctaatc

SEQ ID NO: 98 - RNA, 100 nt, synthetic construct
  source 1..100: mol_type = other RNA
ctctgcactcatcacacaaaagatcttccccttctataggatccacagggagggggcatttcaatatatctcactcgaaatcatccaggagatgtaactc

SEQ ID NO: 99 - RNA, 100 nt, synthetic construct
  source 1..100: mol_type = other RNA
catatctctgcactcatcacacaaaagatcttccccttctataggatccacagggagggggcatttcaatatatctcactcgaaatcatccaggagatgt

SEQ ID NO: 100 - RNA, 100 nt, synthetic construct
  source 1..100: mol_type = other RNA
tgtgacatatctctgcactcatcacacaaaagatcttccccttctataggatccacagggagggggcatttcaatatatctcactcgaaatcatccagga

SEQ ID NO: 101 - RNA, 100 nt, synthetic construct
  source 1..100: mol_type = other RNA
gatattgtgacatatctctgcactcatcacacaaaagatcttccccttctataggatccacagggagggggcatttcaatatatctcactcgaaatcatc

SEQ ID NO: 102 - RNA, 100 nt, synthetic construct
  source 1..100: mol_type = other RNA
ctctgcactcatcacacaaaagacccctctcttctataggatccacagggagggggcattctaatattcaaacgaactaatcatccaggagatgtaactc

SEQ ID NO: 103 - RNA, 100 nt, synthetic construct
  source 1..100: mol_type = other RNA
catatctctgcactcatcacacaaaagacccctctcttctataggatccacagggagggggcattctaatattcaaacgaactaatcatccaggagatgt

SEQ ID NO: 104 - RNA, 100 nt, synthetic construct
  source 1..100: mol_type = other RNA
tgtgacatatctctgcactcatcacacaaaagacccctctcttctataggatccacagggagggggcattctaatattcaaacgaactaatcatccagga

SEQ ID NO: 105 - RNA, 100 nt, synthetic construct
  source 1..100: mol_type = other RNA
gatattgtgacatatctctgcactcatcacacaaaagacccctctcttctataggatccacagggagggggcattctaatattcaaacgaactaatcatc

SEQ ID NO: 106 - RNA, 100 nt, synthetic construct
  source 1..100: mol_type = other RNA
cactcatcacactccctctgcaaatcttctataggatccacagggagggggcattctaatatatctctgaccctcgcatccaggagatgtaactctaatc

SEQ ID NO: 107 - RNA, 100 nt, synthetic construct
  source 1..100: mol_type = other RNA
ctctgcactcatcacactccctctgcaaatcttctataggatccacagggagggggcattctaatatatctctgaccctcgcatccaggagatgtaactc

SEQ ID NO: 108 - RNA, 100 nt, synthetic construct
  source 1..100: mol_type = other RNA
catatctctgcactcatcacactccctctgcaaatcttctataggatccacagggagggggcattctaatatatctctgaccctcgcatccaggagatgt

SEQ ID NO: 109 - RNA, 100 nt, synthetic construct
  source 1..100: mol_type = other RNA
tgtgacatatctctgcactcatcacactccctctgcaaatcttctataggatccacagggagggggcattctaatatatctctgaccctcgcatccagga

SEQ ID NO: 110 - RNA, 100 nt, synthetic construct
  source 1..100: mol_type = other RNA
gatattgtgacatatctctgcactcatcacactccctctgcaaatcttctataggatccacagggagggggcattctaatatatctctgaccctcgcatc

SEQ ID NO: 111 - RNA, 101 nt, synthetic construct
  source 1..101: mol_type = other RNA
cactcatcacacaaaagatgcaaatctgtgggcggatccacagggagggggcattttaacgactctgtgtgaacgatcatcttactcttgtaactctaatc

SEQ ID NO: 112 - RNA, 101 nt, synthetic construct
  source 1..101: mol_type = other RNA
cactcatcacacaaaagacccctctcttctataggatccacagggagggggcattttaacgactctgtgtgaacgatcatcttactcttgtaactctaatc

SEQ ID NO: 113 - RNA, 101 nt, synthetic construct
  source 1..101: mol_type = other RNA
cactcatcacactccctctgcaaatcttctataggatccacagggagggggcattttaacgactctgtgtgaacgatcatcttactcttgtaactctaatc

SEQ ID NO: 114 - RNA, 101 nt, synthetic construct
  source 1..101: mol_type = other RNA
ctctgcactcatcacacaaaagatgcaaatctgtgggcggatccacagggagggggcattttaacgactctgtgtgaacgatcatcttactcttgtaactc

SEQ ID NO: 115 - RNA, 101 nt, synthetic construct
  source 1..101: mol_type = other RNA
ctctgcactcatcacactccctctgcaaatcttctataggatccacagggagggggcattttaacgactctgtgtgaacgatcatcttactcttgtaactc

SEQ ID NO: 116 - RNA, 30 nt, synthetic construct
  source 1..30: mol_type = other RNA
ggagggggcattttaatatatctctgaact

SEQ ID NO: 117 - RNA, 61 nt, synthetic construct
  source 1..61: mol_type = other RNA
ggagggggcattttaatatatctctgaactaatcatccaggagatgtaactctaatccagg

SEQ ID NO: 118 - DNA, 100 bp, synthetic construct
  source 1..100: mol_type = other DNA
ctctgcactcatcacacaaaagatgcaaatcttctataggatccacagggagggggcattttaacatatctctgaactaatcatccaggagatgtaactc

SEQ ID NO: 119 - DNA, 95 bp, synthetic construct
  source 1..95: mol_type = other DNA
ctctgcactcatcacctactcgatgcaaatcttctataggatccacagggagggggcattttaacatatgcagctactaatcatccaggagatgt

SEQ ID NO: 120 - DNA, 95 bp, synthetic construct
  source 1..95: mol_type = other DNA
cactcatcacctactcgatgcaaatcttctataggatccacagggagggggcattttaacatatgcagctactaatcatccaggagatgtaactc

SEQ ID NO: 121 - DNA, 90 bp, synthetic construct
  source 1..90: mol_type = other DNA
ctctgcactcatcacctactcgatgcaaatcttctataggatccacagggagggggcattttaacatatgcagctactaatcatccagga

SEQ ID NO: 122 - DNA, 85 bp, synthetic construct
  source 1..85: mol_type = other DNA
ctctgcactcatcacctactcgatgcaaatcttctataggatccacagggagggggcattttaacatatgcagctactaatcatc

SEQ ID NO: 123 - skipped

SEQ ID NO: 124 - DNA, 95 bp, synthetic construct
  source 1..95: mol_type = other DNA
ctctgcactcatcacacaaaagatgcaaatctgtgggcggatccacagggagggggcattttaacatatcagctccctaatcatccaggagatgt

SEQ ID NO: 125 - DNA, 95 bp, synthetic construct
  source 1..95: mol_type = other DNA
cactcatcacacaaaagatgcaaatctgtgggcggatccacagggagggggcattttaacatatcagctccctaatcatccaggagatgtaactc

SEQ ID NO: 126 - DNA, 90 bp, synthetic construct
  source 1..90: mol_type = other DNA
ctctgcactcatcacacaaaagatgcaaatctgtgggcggatccacagggagggggcattttaacatatcagctccctaatcatccagga

SEQ ID NO: 127 - DNA, 90 bp, synthetic construct
  source 1..90: mol_type = other DNA
atcacacaaaagatgcaaatctgtgggcggatccacagggagggggcattttaacatatcagctccctaatcatccaggagatgtaactc

SEQ ID NO: 128 - DNA, 85 bp, synthetic construct
  source 1..85: mol_type = other DNA
ctctgcactcatcacacaaaagatgcaaatctgtgggcggatccacagggagggggcattttaacatatcagctccctaatcatc

SEQ ID NO: 129 - DNA, 85 bp, synthetic construct
  source 1..85: mol_type = other DNA
acaaaagatgcaaatctgtgggcggatccacagggagggggcattttaacatatcagctccctaatcatccaggagatgtaactc

SEQ ID NO: 130 - DNA, 95 bp, synthetic construct
  source 1..95: mol_type = other DNA
atcacacaaaagacccctctcttctataggatccacagggagggggcattctaatattcaaacgaactaatcatccaggagatgtaactctaatc

SEQ ID NO: 131 - DNA, 95 bp, synthetic construct
  source 1..95: mol_type = other DNA
cactcatcacacaaaagacccctctcttctataggatccacagggagggggcattctaatattcaaacgaactaatcatccaggagatgtaactc

SEQ ID NO: 132 - DNA, 90 bp, synthetic construct
  source 1..90: mol_type = other DNA
cactcatcacacaaaagacccctctcttctataggatccacagggagggggcattctaatattcaaacgaactaatcatccaggagatgt

SEQ ID NO: 133 - DNA, 85 bp, synthetic construct
  source 1..85: mol_type = other DNA
cactcatcacacaaaagacccctctcttctataggatccacagggagggggcattctaatattcaaacgaactaatcatccagga

SEQ ID NO: 134 - DNA, 80 bp, synthetic construct
  source 1..80: mol_type = other DNA
cactcatcacacaaaagacccctctcttctataggatccacagggagggggcattctaatattcaaacgaactaatcatc

SEQ ID NO: 135 - DNA, 95 bp, synthetic construct
  source 1..95: mol_type = other DNA
atcacactccctctgcaaatcttctataggatccacagggagggggcattctaatatatctctgaccctcgcatccaggagatgtaactctaatc

SEQ ID NO: 136 - DNA, 90 bp, synthetic construct
  source 1..90: mol_type = other DNA
atcacactccctctgcaaatcttctataggatccacagggagggggcattctaatatatctctgaccctcgcatccaggagatgtaactc

SEQ ID NO: 137 - DNA, 85 bp, synthetic construct
  source 1..85: mol_type = other DNA
atcacactccctctgcaaatcttctataggatccacagggagggggcattctaatatatctctgaccctcgcatccaggagatgt

SEQ ID NO: 138 - DNA, 80 bp, synthetic construct
  source 1..80: mol_type = other DNA
atcacactccctctgcaaatcttctataggatccacagggagggggcattctaatatatctctgaccctcgcatccagga

SEQ ID NO: 139 - skipped

SEQ ID NO: 140 - DNA, 100 bp, synthetic construct
  source 1..100: mol_type = other DNA
cactcatcacacaaaagatgcaaatcttctataggatccacagggagggggcattctaatatatctctgaactaatcatccaggagatgtaactctaatc

SEQ ID NO: 141 - DNA, 100 bp, synthetic construct
  source 1..100: mol_type = other DNA
cactcatcacacaaaagatgcaaatcttctataggatccacagggagggggcattttaacatatctctgaactaatcatccaggagatgtaactctaatc

SEQ ID NO: 142 - skipped

SEQ ID NO: 143 - DNA, 95 bp, synthetic construct
  source 1..95: mol_type = other DNA
cactcatcactgccgtgatgcaaatcttctataggatccacagggagggggcattttaacatatctcggtttcaatcatccaggagatgtaactc

SEQ ID NO: 144 - DNA, 90 bp, synthetic construct
  source 1..90: mol_type = other DNA
cactcatcactgccgtgatgcaaatcttctataggatccacagggagggggcattttaacatatctcggtttcaatcatccaggagatgt

SEQ ID NO: 145 - DNA, 85 bp, synthetic construct
  source 1..85: mol_type = other DNA
cactcatcactgccgtgatgcaaatcttctataggatccacagggagggggcattttaacatatctcggtttcaatcatccagga

SEQ ID NO: 146 - DNA, 95 bp, synthetic construct
  source 1..95: mol_type = other DNA
atcacacaaaagatcttccccttctataggatccacagggagggggcatttcaatatatctcactcgaaatcatccaggagatgtaactctaatc

SEQ ID NO: 147 - DNA, 85 bp, synthetic construct
  source 1..85: mol_type = other DNA
atcacacaaaagatcttccccttctataggatccacagggagggggcatttcaatatatctcactcgaaatcatccaggagatgt

SEQ ID NO: 148 - RNA, 100 nt, synthetic construct
  source 1..100: mol_type = other RNA
ctctgcactcatcacacaaaagatgcaaatcttctataggatccacagggagggggcattttaacatatctctgaactaatcatccaggagatgtaactc

SEQ ID NO: 149 - RNA, 95 nt, synthetic construct
  source 1..95: mol_type = other RNA
ctctgcactcatcacctactcgatgcaaatcttctataggatccacagggagggggcattttaacatatgcagctactaatcatccaggagatgt

SEQ ID NO: 150 - RNA, 95 nt, synthetic construct
  source 1..95: mol_type = other RNA
cactcatcacctactcgatgcaaatcttctataggatccacagggagggggcattttaacatatgcagctactaatcatccaggagatgtaactc

SEQ ID NO: 151 - RNA, 90 nt, synthetic construct
  source 1..90: mol_type = other RNA
ctctgcactcatcacctactcgatgcaaatcttctataggatccacagggagggggcattttaacatatgcagctactaatcatccagga

SEQ ID NO: 152 - RNA, 85 nt, synthetic construct
  source 1..85: mol_type = other RNA
ctctgcactcatcacctactcgatgcaaatcttctataggatccacagggagggggcattttaacatatgcagctactaatcatc

SEQ ID NO: 153 - skipped

SEQ ID NO: 154 - RNA, 95 nt, synthetic construct
  source 1..95: mol_type = other RNA
ctctgcactcatcacacaaaagatgcaaatctgtgggcggatccacagggagggggcattttaacatatcagctccctaatcatccaggagatgt

SEQ ID NO: 155 - RNA, 95 nt, synthetic construct
  source 1..95: mol_type = other RNA
cactcatcacacaaaagatgcaaatctgtgggcggatccacagggagggggcattttaacatatcagctccctaatcatccaggagatgtaactc

SEQ ID NO: 156 - RNA, 90 nt, synthetic construct
  source 1..90: mol_type = other RNA
ctctgcactcatcacacaaaagatgcaaatctgtgggcggatccacagggagggggcattttaacatatcagctccctaatcatccagga

SEQ ID NO: 157 - RNA, 90 nt, synthetic construct
  source 1..90: mol_type = other RNA
atcacacaaaagatgcaaatctgtgggcggatccacagggagggggcattttaacatatcagctccctaatcatccaggagatgtaactc

SEQ ID NO: 158 - RNA, 85 nt, synthetic construct
  source 1..85: mol_type = other RNA
ctctgcactcatcacacaaaagatgcaaatctgtgggcggatccacagggagggggcattttaacatatcagctccctaatcatc

SEQ ID NO: 159 - RNA, 85 nt, synthetic construct
  source 1..85: mol_type = other RNA
acaaaagatgcaaatctgtgggcggatccacagggagggggcattttaacatatcagctccctaatcatccaggagatgtaactc

SEQ ID NO: 160 - RNA, 95 nt, synthetic construct
  source 1..95: mol_type = other RNA
atcacacaaaagacccctctcttctataggatccacagggagggggcattctaatattcaaacgaactaatcatccaggagatgtaactctaatc

SEQ ID NO: 161 - RNA, 95 nt, synthetic construct
  source 1..95: mol_type = other RNA
cactcatcacacaaaagacccctctcttctataggatccacagggagggggcattctaatattcaaacgaactaatcatccaggagatgtaactc

SEQ ID NO: 162 - RNA, 90 nt, synthetic construct
  source 1..90: mol_type = other RNA
cactcatcacacaaaagacccctctcttctataggatccacagggagggggcattctaatattcaaacgaactaatcatccaggagatgt

SEQ ID NO: 163 - RNA, 85 nt, synthetic construct
  source 1..85: mol_type = other RNA
cactcatcacacaaaagacccctctcttctataggatccacagggagggggcattctaatattcaaacgaactaatcatccagga

SEQ ID NO: 164 - RNA, 80 nt, synthetic construct
  source 1..80: mol_type = other RNA
cactcatcacacaaaagacccctctcttctataggatccacagggagggggcattctaatattcaaacgaactaatcatc

SEQ ID NO: 165 - RNA, 95 nt, synthetic construct
  source 1..95: mol_type = other RNA
atcacactccctctgcaaatcttctataggatccacagggagggggcattctaatatatctctgaccctcgcatccaggagatgtaactctaatc

SEQ ID NO: 166 - RNA, 90 nt, synthetic construct
  source 1..90: mol_type = other RNA
atcacactccctctgcaaatcttctataggatccacagggagggggcattctaatatatctctgaccctcgcatccaggagatgtaactc

SEQ ID NO: 167 - RNA, 85 nt, synthetic construct
  source 1..85: mol_type = other RNA
atcacactccctctgcaaatcttctataggatccacagggagggggcattctaatatatctctgaccctcgcatccaggagatgt

SEQ ID NO: 168 - RNA, 80 nt, synthetic construct
  source 1..80: mol_type = other RNA
atcacactccctctgcaaatcttctataggatccacagggagggggcattctaatatatctctgaccctcgcatccagga

SEQ ID NO: 169 - skipped

SEQ ID NO: 170 - RNA, 100 nt, synthetic construct
  source 1..100: mol_type = other RNA
cactcatcacacaaaagatgcaaatcttctataggatccacagggagggggcattctaatatatctctgaactaatcatccaggagatgtaactctaatc

SEQ ID NO: 171 - RNA, 100 nt, synthetic construct
  source 1..100: mol_type = other RNA
cactcatcacacaaaagatgcaaatcttctataggatccacagggagggggcattttaacatatctctgaactaatcatccaggagatgtaactctaatc

SEQ ID NO: 172 - skipped

SEQ ID NO: 173 - RNA, 95 nt, synthetic construct
  source 1..95: mol_type = other RNA
cactcatcactgccgtgatgcaaatcttctataggatccacagggagggggcattttaacatatctcggtttcaatcatccaggagatgtaactc

SEQ ID NO: 174 - RNA, 90 nt, synthetic construct
  source 1..90: mol_type = other RNA
cactcatcactgccgtgatgcaaatcttctataggatccacagggagggggcattttaacatatctcggtttcaatcatccaggagatgt

SEQ ID NO: 175 - RNA, 85 nt, synthetic construct
  source 1..85: mol_type = other RNA
cactcatcactgccgtgatgcaaatcttctataggatccacagggagggggcattttaacatatctcggtttcaatcatccagga

SEQ ID NO: 176 - RNA, 95 nt, synthetic construct
  source 1..95: mol_type = other RNA
atcacacaaaagatcttccccttctataggatccacagggagggggcatttcaatatatctcactcgaaatcatccaggagatgtaactctaatc

SEQ ID NO: 177 - RNA, 85 nt, synthetic construct
  source 1..85: mol_type = other RNA
atcacacaaaagatcttccccttctataggatccacagggagggggcatttcaatatatctcactcgaaatcatccaggagatgt

SEQ ID NO: 178 - DNA, 100 bp, synthetic construct
  source 1..100: mol_type = other DNA
ctctgcactcatcactaatctgatgcaaatcttctataggatccacagggagggggcattttaacatatataatcactaatcatccgggaggtgtaactc

SEQ ID NO: 179 - DNA, 100 bp, synthetic construct
  source 1..100: mol_type = other DNA
ctctgcactcatcactaatctgatgcagatcttctataggatccacagggagggggcattttaacatatataatcactgatcatccgggaggtgtaactc

SEQ ID NO: 180 - DNA, 100 bp, synthetic construct
  source 1..100: mol_type = other DNA
ctctgcactcatcactaatctgatgcagatcttctatagggtctacagggagggggcattttaacatatataatcactgatcatccgggaggtgtaactc

SEQ ID NO: 181 - DNA, 100 bp, synthetic construct
  source 1..100: mol_type = other DNA
ctctgcactcatcactaatctgatgtagatcttctatagggtctacagggagggggtattttaacatatataatcactgatcatccgggaggtgtaactc

SEQ ID NO: 182 - DNA, 100 bp, synthetic construct
  source 1..100: mol_type = other DNA
ctctgcactcatcactaatctgatgtagattttctgtagggtctacagggagggggtattttaacatatataatcactgatcatccgggaggtgtaactc

SEQ ID NO: 183 - DNA, 100 bp, synthetic construct
  source 1..100: mol_type = other DNA
ctctgcactcatcactaatctgatgtagattttctgtagggtctacagggagggggtattttaacatatataatcactgattgtccgggaggtgtaactc

SEQ ID NO: 184 - DNA, 100 bp, synthetic construct
  source 1..100: mol_type = other DNA
ctctgcactcatcactaatctgatgtagattttctgtagggtctacagggagggggtattttaacatatataatcactgattgtctgggaggtgtgactc

SEQ ID NO: 185 - DNA, 100 bp, synthetic construct
  source 1..100: mol_type = other DNA
ctctgcactcatcactaatctgatgtagattttctgtagggtctacagggagggggtattttaacatatataatcactgattgtctgggaggtgtgattt

SEQ ID NO: 186 - DNA, 100 bp, synthetic construct
  source 1..100: mol_type = other DNA
ctctgcactcgttactaatctgatgtagattttctgtagggtctacagggagggggtattttaacatatataatcactgattgtctgggaggtgtgattt

SEQ ID NO: 187 - DNA, 100 bp, synthetic construct
  source 1..100: mol_type = other DNA
ctttgcgctcgttactaatctgatgtagattttctgtagggtctacagggagggggtattttaacatatataatcactgattgtctgggaggtgtgattt

SEQ ID NO: 188 - DNA, 100 bp, synthetic construct
  source 1..100: mol_type = other DNA
ctctgcactcatcactaatctgatgcagatcttctataggatccacagggagggggcattttaacatatctcttcactgatcatccgggaggtgtaactc

SEQ ID NO: 189 - DNA, 100 bp, synthetic construct
  source 1..100: mol_type = other DNA
ctctgcactcatcacacaaaagatgcaaatctaaccatggatccacagggagggggcattttaacatatcaatattctaatcatccaggagatgtagctc

SEQ ID NO: 190 - DNA, 100 bp, synthetic construct
  source 1..100: mol_type = other DNA
ctctgcactcatcacacaaaagatgcaaatctaaccatggatccacagggagggggcattttaacatatcaatattctagtcatccaggagatgtagctt

SEQ ID NO: 191 - DNA, 100 bp, synthetic construct
  source 1..100: mol_type = other DNA
ctctgcactcatcacacaaaagatgcaaatctaaccatggatccgcagggagggggtattttaacatatcaatattctagtcatccaggagatgtagctt

SEQ ID NO: 192 - DNA, 100 bp, synthetic construct
  source 1..100: mol_type = other DNA
ctctgtactcatcacacaaaagatgcagatctaaccatggatccgcagggagggggtattttaacatatcaatattctagtcatccaggagatgtagctt

SEQ ID NO: 193 - DNA, 100 bp, synthetic construct
  source 1..100: mol_type = other DNA
ctctgtactcatcacacaaaagatgcagatctaaccatggatccgcagggagggggtattttaacatatcaatattctagtcattcaggaggtgtagctt

SEQ ID NO: 194 - DNA, 100 bp, synthetic construct
  source 1..100: mol_type = other DNA
ctctgtactcatcacacaagagatgcagatctaaccatggattcgcagggagggggtattttaacatatcaatattctagtcattcaggaggtgtagctt

SEQ ID NO: 195 - DNA, 100 bp, synthetic construct
  source 1..100: mol_type = other DNA
ctctgtacttatcacacaagagatgcagatctaaccatggattcgcagggagggggtattttaacatatcaatattctagtcgttcaggaggtgtagctt

SEQ ID NO: 196 - DNA, 100 bp, synthetic construct
  source 1..100: mol_type = other DNA
ctctgtacttatcacgcaagagatgcagatctaaccatggattcgcagggagggggtattttaacatatcaatattctagttgttcaggaggtgtagctt

SEQ ID NO: 197 - DNA, 100 bp, synthetic construct
  source 1..100: mol_type = other DNA
ctctgtacttatcacgcaagagatgtagatctaaccatggattcgcagggagggggtattttaacatatcaatattctggttgttcaggaggtgtagctt

SEQ ID NO: 198 - DNA, 100 bp, synthetic construct
  source 1..100: mol_type = other DNA
ctctgtacttatcacgcgagagatgtagatttaaccatggattcgcagggagggggtattttaacatatcaatattctggttgttcaggaggtgtagctt

SEQ ID NO: 199 - DNA, 100 bp, synthetic construct
  source 1..100: mol_type = other DNA
ctctgtacttatcacgcgagagatgtagatttaaccatggattcgcagggagggggtattttaacatatctctgttctggttgttcaggaggtgtagctt

SEQ ID NO: 200 - DNA, 101 bp, synthetic construct
  source 1..101: mol_type = other DNA
ctctgcactcatcacataaaagaagattttcttctataggattcacagggagggggcattttaacgactctgtgtgaacgatcatcttttccctgtgactc

SEQ ID NO: 201 - DNA, 101 bp, synthetic construct
  source 1..101: mol_type = other DNA
ctctgcactcatcacataagagaagattttcttctataggattcacagggagggggcattttaacgactctgtgtgaacgatcatcttttccctgtgattc

SEQ ID NO: 202 - DNA, 101 bp, synthetic construct
  source 1..101: mol_type = other DNA
ctctgcacttatcacataagagaagattttcttctgtaggattcacagggagggggcattttaacgactctgtgtgaacgatcatcttttccctgtgattc

SEQ ID NO: 203 - DNA, 101 bp, synthetic construct
  source 1..101: mol_type = other DNA
ctctgcacttatcgcataagagaagattttcttttgtaggattcacagggagggggcattttaacgactctgtgtgaacgatcatcttttccctgtgattc

SEQ ID NO: 204 - DNA, 101 bp, synthetic construct
  source 1..101: mol_type = other DNA
ctctgcacttatcgcataagagaagattttcttttgtagggttcgcagggagggggcattttaacgactctgtgtgaacgatcatcttttccctgtgattc

SEQ ID NO: 205 - DNA, 101 bp, synthetic construct
  source 1..101: mol_type = other DNA
ctctgcgcttatcgcataagagaagatttttttttgtagggttcgcagggagggggcattttaacgactctgtgtgaacgatcatcttttccctgtgattc

SEQ ID NO: 206 - DNA, 101 bp, synthetic construct
  source 1..101: mol_type = other DNA
ctttgcgcttatcgcataagagaagatttttttttgtagggttcgcggggagggggcattttaacgactctgtgtgaacgatcatcttttccctgtgattc

SEQ ID NO: 207 - DNA, 101 bp, synthetic construct
  source 1..101: mol_type = other DNA
ttttgcgcttatcgcataagagaagatttttttttgtagggttcgcggggagggggcattttaacgactctgtgtgaacgatcatcttttccctgtgattt

SEQ ID NO: 208 - DNA, 101 bp, synthetic construct
  source 1..101: mol_type = other DNA
ttttgcgcttatcgcataagagaagatttttttttgtagggttcgcggggagggggtattttaacgactctgtgtgaacgatcgtcttttccctgtgattt

SEQ ID NO: 209 - DNA, 101 bp, synthetic construct
  source 1..101: mol_type = other DNA
ctctgcactcatcacataaaagatgcttttcttctataggattcacagggagggggcattttaacgactctgtgtgaacgatcatctagggcatgtgactc

SEQ ID NO: 210 - RNA, 100 nt, synthetic construct
  source 1..100: mol_type = other RNA
ctctgcactcatcactaatctgatgcaaatcttctataggatccacagggagggggcattttaacatatataatcactaatcatccgggaggtgtaactc

SEQ ID NO: 211 - RNA, 100 nt, synthetic construct
  source 1..100: mol_type = other RNA
ctctgcactcatcactaatctgatgcagatcttctataggatccacagggagggggcattttaacatatataatcactgatcatccgggaggtgtaactc

SEQ ID NO: 212 - RNA, 100 nt, synthetic construct
  source 1..100: mol_type = other RNA
ctctgcactcatcactaatctgatgcagatcttctatagggtctacagggagggggcattttaacatatataatcactgatcatccgggaggtgtaactc

SEQ ID NO: 213 - RNA, 100 nt, synthetic construct
  source 1..100: mol_type = other RNA
ctctgcactcatcactaatctgatgtagatcttctatagggtctacagggagggggtattttaacatatataatcactgatcatccgggaggtgtaactc

SEQ ID NO: 214 - RNA, 100 nt, synthetic construct
  source 1..100: mol_type = other RNA
ctctgcactcatcactaatctgatgtagattttctgtagggtctacagggagggggtattttaacatatataatcactgatcatccgggaggtgtaactc

SEQ ID NO: 215 - RNA, 100 nt, synthetic construct
  source 1..100: mol_type = other RNA
ctctgcactcatcactaatctgatgtagattttctgtagggtctacagggagggggtattttaacatatataatcactgattgtccgggaggtgtaactc

SEQ ID NO: 216 - RNA, 100 nt, synthetic construct
  source 1..100: mol_type = other RNA
ctctgcactcatcactaatctgatgtagattttctgtagggtctacagggagggggtattttaacatatataatcactgattgtctgggaggtgtgactc

SEQ ID NO: 217 - RNA, 100 nt, synthetic construct
  source 1..100: mol_type = other RNA
ctctgcactcatcactaatctgatgtagattttctgtagggtctacagggagggggtattttaacatatataatcactgattgtctgggaggtgtgattt

SEQ ID NO: 218 - RNA, 100 nt, synthetic construct
  source 1..100: mol_type = other RNA
ctctgcactcgttactaatctgatgtagattttctgtagggtctacagggagggggtattttaacatatataatcactgattgtctgggaggtgtgattt

SEQ ID NO: 219 - RNA, 100 nt, synthetic construct
  source 1..100: mol_type = other RNA
ctttgcgctcgttactaatctgatgtagattttctgtagggtctacagggagggggtattttaacatatataatcactgattgtctgggaggtgtgattt

SEQ ID NO: 220 - RNA, 100 nt, synthetic construct
  source 1..100: mol_type = other RNA
ctctgcactcatcactaatctgatgcagatcttctataggatccacagggagggggcattttaacatatctcttcactgatcatccgggaggtgtaactc

SEQ ID NO: 221 - RNA, 100 nt, synthetic construct
  source 1..100: mol_type = other RNA
ctctgcactcatcacacaaaagatgcaaatctaaccatggatccacagggagggggcattttaacatatcaatattctaatcatccaggagatgtagctc

SEQ ID NO: 222 - RNA, 100 nt, synthetic construct
  source 1..100: mol_type = other RNA
ctctgcactcatcacacaaaagatgcaaatctaaccatggatccacagggagggggcattttaacatatcaatattctagtcatccaggagatgtagctt

SEQ ID NO: 223 - RNA, 100 nt, synthetic construct
  source 1..100: mol_type = other RNA
ctctgcactcatcacacaaaagatgcaaatctaaccatggatccgcagggagggggtattttaacatatcaatattctagtcatccaggagatgtagctt

SEQ ID NO: 224 - RNA, 100 nt, synthetic construct
  source 1..100: mol_type = other RNA
ctctgtactcatcacacaaaagatgcagatctaaccatggatccgcagggagggggtattttaacatatcaatattctagtcatccaggagatgtagctt

SEQ ID NO: 225 - RNA, 100 nt, synthetic construct
  source 1..100: mol_type = other RNA
ctctgtactcatcacacaaaagatgcagatctaaccatggatccgcagggagggggtattttaacatatcaatattctagtcattcaggaggtgtagctt

SEQ ID NO: 226 - RNA, 100 nt, synthetic construct
  source 1..100: mol_type = other RNA
ctctgtactcatcacacaagagatgcagatctaaccatggattcgcagggagggggtattttaacatatcaatattctagtcattcaggaggtgtagctt

SEQ ID NO: 227 - RNA, 100 nt, synthetic construct
  source 1..100: mol_type = other RNA
ctctgtacttatcacacaagagatgcagatctaaccatggattcgcagggagggggtattttaacatatcaatattctagtcgttcaggaggtgtagctt

SEQ ID NO: 228 - RNA, 100 nt, synthetic construct
  source 1..100: mol_type = other RNA
ctctgtacttatcacgcaagagatgcagatctaaccatggattcgcagggagggggtattttaacatatcaatattctagttgttcaggaggtgtagctt

SEQ ID NO: 229 - RNA, 100 nt, synthetic construct
  source 1..100: mol_type = other RNA
ctctgtacttatcacgcaagagatgtagatctaaccatggattcgcagggagggggtattttaacatatcaatattctggttgttcaggaggtgtagctt

SEQ ID NO: 230 - RNA, 100 nt, synthetic construct
  source 1..100: mol_type = other RNA
ctctgtacttatcacgcgagagatgtagatttaaccatggattcgcagggagggggtattttaacatatcaatattctggttgttcaggaggtgtagctt

SEQ ID NO: 231 - RNA, 100 nt, synthetic construct
  source 1..100: mol_type = other RNA
ctctgtacttatcacgcgagagatgtagatttaaccatggattcgcagggagggggtattttaacatatctctgttctggttgttcaggaggtgtagctt

SEQ ID NO: 232 - RNA, 101 nt, synthetic construct
  source 1..101: mol_type = other RNA
ctctgcactcatcacataaaagaagattttcttctataggattcacagggagggggcattttaacgactctgtgtgaacgatcatcttttccctgtgactc

SEQ ID NO: 233 - RNA, 101 nt, synthetic construct
  source 1..101: mol_type = other RNA
ctctgcactcatcacataagagaagattttcttctataggattcacagggagggggcattttaacgactctgtgtgaacgatcatcttttccctgtgattc

SEQ ID NO: 234 - RNA, 101 nt, synthetic construct
  source 1..101: mol_type = other RNA
ctctgcacttatcacataagagaagattttcttctgtaggattcacagggagggggcattttaacgactctgtgtgaacgatcatcttttccctgtgattc

SEQ ID NO: 235 - RNA, 101 nt, synthetic construct
  source 1..101: mol_type = other RNA
ctctgcacttatcgcataagagaagattttcttttgtaggattcacagggagggggcattttaacgactctgtgtgaacgatcatcttttccctgtgattc

SEQ ID NO: 236 - RNA, 101 nt, synthetic construct
  source 1..101: mol_type = other RNA
ctctgcacttatcgcataagagaagattttcttttgtagggttcgcagggagggggcattttaacgactctgtgtgaacgatcatcttttccctgtgattc

SEQ ID NO: 237 - RNA, 101 nt, synthetic construct
  source 1..101: mol_type = other RNA
ctctgcgcttatcgcataagagaagatttttttttgtagggttcgcagggagggggcattttaacgactctgtgtgaacgatcatcttttccctgtgattc

SEQ ID NO: 238 - RNA, 101 nt, synthetic construct
  source 1..101: mol_type = other RNA
ctttgcgcttatcgcataagagaagatttttttttgtagggttcgcggggagggggcattttaacgactctgtgtgaacgatcatcttttccctgtgattc

SEQ ID NO: 239 - RNA, 101 nt, synthetic construct
  source 1..101: mol_type = other RNA
ttttgcgcttatcgcataagagaagatttttttttgtagggttcgcggggagggggcattttaacgactctgtgtgaacgatcatcttttccctgtgattt

SEQ ID NO: 240 - RNA, 101 nt, synthetic construct
  source 1..101: mol_type = other RNA
ttttgcgcttatcgcataagagaagatttttttttgtagggttcgcggggagggggtattttaacgactctgtgtgaacgatcgtcttttccctgtgattt

SEQ ID NO: 241 - RNA, 101 nt, synthetic construct
  source 1..101: mol_type = other RNA
ctctgcactcatcacataaaagatgcttttcttctataggattcacagggagggggcattttaacgactctgtgtgaacgatcatctagggcatgtgactc

SEQ ID NO: 242 - DNA, 100 bp, synthetic construct
  source 1..100: mol_type = other DNA
gatgattagttcagagatatattaaaatgccccctccctgtggatcctatagaagatttgcatcttttgtgtgatgagtgcagagatatgtcacaatatc

SEQ ID NO: 243 - RNA, 100 nt, synthetic construct
  source 1..100: mol_type = other RNA
gatgattagttcagagatatattaaaatgccccctccctgtggatcctatagaagatttgcatcttttgtgtgatgagtgcagagatatgtcacaatatc

SEQ ID NO: 244 - DNA, 100 bp, synthetic construct
  source 1..100: mol_type = other DNA
gagttacatctcctggatgattagttcagagatatattaaaatgccccctccctgtggatcctatagaagatttgcatcttttgtgtgatgagtgcagag

SEQ ID NO: 245 - RNA, 100 nt, synthetic construct
  source 1..100: mol_type = other RNA
gagttacatctcctggatgattagttcagagatatattaaaatgccccctccctgtggatcctatagaagatttgcatcttttgtgtgatgagtgcagag

SEQ ID NO: 246 - DNA, 100 bp, synthetic construct
  source 1..100: mol_type = other DNA
gatattgtgacatatctctgcactcatcacacaaccctcccaaatcttctataggatccacagggagggggcattttaacatatctctcgtgatatcatc

SEQ ID NO: 247 - RNA, 100 nt, synthetic construct
  source 1..100: mol_type = other RNA
gatattgtgacatatctctgcactcatcacacaaccctcccaaatcttctataggatccacagggagggggcattttaacatatctctcgtgatatcatc

SEQ ID NO: 248 - DNA, 126 bp, synthetic construct
  source 1..126: mol_type = other DNA
taggtctccaccggatattgtgacatatctctgcactcatcacacaaaagatgcaaatcttctatagacaggtcagggagggggcatttcaatatatctctgaactaatcatcgtggagagaccat

SEQ ID NO: 249 - DNA, 126 bp, synthetic construct
  source 1..126: mol_type = other DNA
taggtctccaccggatattgtgacatatctctgcactcatcacacaaccctcccaaatcttctataggatccacagggagggggcattttaacatatctctcgtgatatcatcgtggagagaccat

SEQ ID NO: 250 - DNA, 126 bp, synthetic construct
  source 1..126: mol_type = other DNA
taggtctccaccggatattgtgacatatctctgcactcatcacacaaaagatgctccctatctataggatccacagggagggggcattttaacatatccacctcctaatcatcgtggagagaccat

SEQ ID NO: 251 - DNA, 126 bp, synthetic construct
  source 1..126: mol_type = other DNA
taggtctccaccggatattgtgacatatctctgcactcatcacacctgtcctgcaaatcttctataggatccacagggagggggcatttcaatatatcagcatcctaatcatcgtggagagaccat

SEQ ID NO: 252 - DNA, 126 bp, synthetic construct
  source 1..126: mol_type = other DNA
taggtctccaccggatattgtgacatatctctgcactcatcacacaaaagatgccctggatctataggatccacagggagggggcattttaatatatgagatcactaatcatcgtggagagaccat

SEQ ID NO: 253 - DNA, 126 bp, synthetic construct
  source 1..126: mol_type = other DNA
taggtctccaccggatattgtgacatatctctgcactcatcacacaaaagacccctctcttctataggatccacagggagggggcattctaatattcaaacgaactaatcatcgtggagagaccat

SEQ ID NO: 254 - DNA, 126 bp, synthetic construct
  source 1..126: mol_type = other DNA
taggtctccaccggatattgtgacatatctctgcactcatcacacaaaagatgcacaggggctataggatccacagggagggggcattctaattccctgctgaactaatcatcgtggagagaccat

SEQ ID NO: 255 - RNA, 126 nt, synthetic construct
  source 1..126: mol_type = other RNA
taggtctccaccggatattgtgacatatctctgcactcatcacacaaaagatgcaaatcttctatagacaggtcagggagggggcatttcaatatatctctgaactaatcatcgtggagagaccat

SEQ ID NO: 256 - RNA, 126 nt, synthetic construct
  source 1..126: mol_type = other RNA
taggtctccaccggatattgtgacatatctctgcactcatcacacaaccctcccaaatcttctataggatccacagggagggggcattttaacatatctctcgtgatatcatcgtggagagaccat

SEQ ID NO: 257 - RNA, 126 nt, synthetic construct
  source 1..126: mol_type = other RNA
taggtctccaccggatattgtgacatatctctgcactcatcacacaaaagatgctccctatctataggatccacagggagggggcattttaacatatccacctcctaatcatcgtggagagaccat

SEQ ID NO: 258 - RNA, 126 nt, synthetic construct
  source 1..126: mol_type = other RNA
taggtctccaccggatattgtgacatatctctgcactcatcacacctgtcctgcaaatcttctataggatccacagggagggggcatttcaatatatcagcatcctaatcatcgtggagagaccat

SEQ ID NO: 259 - RNA, 126 nt, synthetic construct
  source 1..126: mol_type = other RNA
taggtctccaccggatattgtgacatatctctgcactcatcacacaaaagatgccctggatctataggatccacagggagggggcattttaatatatgagatcactaatcatcgtggagagaccat

SEQ ID NO: 260 - RNA, 126 nt, synthetic construct
  source 1..126: mol_type = other RNA
taggtctccaccggatattgtgacatatctctgcactcatcacacaaaagacccctctcttctataggatccacagggagggggcattctaatattcaaacgaactaatcatcgtggagagaccat

SEQ ID NO: 261 - RNA, 126 nt, synthetic construct
  source 1..126: mol_type = other RNA
taggtctccaccggatattgtgacatatctctgcactcatcacacaaaagatgcacaggggctataggatccacagggagggggcattctaattccctgctgaactaatcatcgtggagagaccat

SEQ ID NO: 262 - DNA, 105 bp, synthetic construct
  source 1..105: mol_type = other DNA
  misc_difference 1: note = a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 263 - DNA, 105 bp, synthetic construct
  source 1..105: mol_type = other DNA
  misc_difference 105: note = a, c, t, g, unknown or other
agcagccacaactccgggaacccctttgaaagtcctttcccgaatacatccacggctaaacttctcctttacaccacactgtcgtcgattaggccactcccagtn

(19) United States
(12) Patent Application Publication
(10) Pub. No.: US 2026/0209755 A1
(43) Pub. Date: Jul. 23, 2026

(54) DUX4-TARGETING ENGINEERED GUIDE RNAS AND POLYNUCLEOTIDES

(71) Applicant: Shape Therapeutics Inc., Seattle, WA (US)

(72) Inventors: Yazmin Ines ROVIRA GONZALEZ, Seattle, WA (US); Brian BOOTH, West Roxbury, MA (US); Yiannis SAVVA, Seattle, WA (US); Lina Rajili BAGEPALLI, Cambridge, MA (US); Yue JIANG, Los Angeles, CA (US); Bora BANJANIN, Seattle, WA (US); Ronald James HAUSE, JR., Seattle, WA (US)

(21) Appl. No.: 19/451,186

(22) Filed: Jan. 16, 2026

Related U.S. Application Data
(63) Continuation of application No. PCT/US2024/038652, filed on Jul. 19, 2024.
(60) Provisional application No. 63/527,906, filed on Jul. 20, 2023.
(60) Provisional application No. 63/613,133, filed on Dec. 21, 2023.
(60) Provisional application No. 63/663,748, filed on Jun. 25, 2024.

(51) Int. Cl. C12N 15/11; C12N 15/86

(52) U.S. Cl. CPC C12N 15/11; C12N 15/86; C12N 2310/20; C12N 2310/532; C12N 2750/14143

(57) ABSTRACT

Disclosed herein are engineered guide RNAs and compositions comprising the same for treatment of diseases or conditions in a subject. Also disclosed herein are methods of treating diseases or conditions in a subject by administering engineered guide RNAs or pharmaceutical compositions described herein.

### CROSS-REFERENCE TO RELATED APPLICATIONS

This application is a continuation of PCT Application No. PCT/US2024/038652 filed on Jul. 19, 2024, which claims priority under 35 U.S.C. § 119 from U.S. Provisional Application No. 63/527,906 filed on Jul. 20, 2023, U.S. Provisional Application No. 63/613,133 filed on Dec. 21, 2023, and U.S. Provisional Application No. 63/663,748 filed on Jun. 25, 2024, the disclosures of which are incorporated herein by reference in their entirety.

### SEQUENCE LISTING

The application contains a Sequence Listing which has been submitted electronically in XML format and is hereby incorporated by reference in its entirety. Said XML copy, created on Jun. 18, 2024, is named 199235-764601_SL.xml and is 249,856 bytes in size.

### BACKGROUND

Payloads that mediate RNA editing can be viable therapies for genetic diseases. However, highly efficacious payloads that can maximize on-target RNA editing while minimizing off-target RNA editing are needed. Moreover, payloads that are capable of facilitating protein knockdown are also needed.

### SUMMARY

Disclosed herein are engineered guide RNAs or polynucleotide encoding the engineered guide RNAs. In some embodiments, the engineered guide RNA can be capable of hybridizing to a DUX4 target sequence of a DUX4-FL mRNA. In some embodiments, the DUX4 target sequence can comprise a polyA signal sequence. In some embodiments, the engineered guide RNA can have at least 80%, at least 85%, at least 90%, at least 92%, at least 95%, at least 96%, at least 97%, at least 98%, at least 99% or 100% sequence identity to one of SEQ ID NO: 39, SEQ ID NO: 40, SEQ ID NO: 42, SEQ ID NO: 44, SEQ ID NO: 46, SEQ ID NO: 93, SEQ ID NO: 228, or SEQ ID NO: 239; and upon hybridization to the target sequence, can form a guide-target RNA scaffold comprising at least 3 structural features including at least one 6/6 symmetric internal loop. In some embodiments, the engineered guide can comprise 95 to 105 nucleotides or about 100 nucleotides. In some embodiments, polynucleotide encoding the engineered guide RNA can comprise a polynucleotide sequence with at least 80%, at least 85%, at least 90%, at least 92%, at least 95%, at least 96%, at least 97%, at least 98%, at least 99% or 100% sequence identity to any one of SEQ ID NO: 32, SEQ ID NO: 33, SEQ ID NO: 35, SEQ ID NO: 37, SEQ ID NO: 57, SEQ ID NO: 79, SEQ ID NO: 196, or SEQ ID NO: 207. In some embodiments, the guide-target RNA scaffold can comprise at least three wobble base pairs. In some embodiments, the engineered guide RNA can comprise SEQ ID NO: 228 or SEQ ID NO: 239. In some embodiments, the polynucleotide encoding the engineered guide RNA can comprise SEQ ID NO: 196 or SEQ ID NO: 207. In some embodiments, the guide-target RNA scaffold can comprise at least one wobble base pair. In some embodiments, the guide-target RNA scaffold can comprise at least two wobble base pairs. In some embodiments, the engineered guide RNA can comprise SEQ ID NO: 228, SEQ ID NO: 239, or SEQ ID NO: 46. In some embodiments, the polynucleotide encoding the engineered guide RNA can comprise SEQ ID NO: 196, SEQ ID NO: 207, or SEQ ID NO: 79. In some embodiments, the guide-target RNA scaffold can comprise a mismatch and at least two 6/6 symmetric internal loops. In some embodiments, the engineered guide RNA can comprise SEQ ID NO: 39, SEQ ID NO: 40, SEQ ID NO: 42, SEQ ID NO: 44, SEQ ID NO: 93, or SEQ ID NO: 228. In some embodiments, the polynucleotide encoding the engineered guide RNA can comprise SEQ ID NO: 32, SEQ ID NO: 33, SEQ ID NO: 35, SEQ ID NO: 37, SEQ ID NO: 57, or SEQ ID NO: 196. In some embodiments, the guide-target RNA scaffold can comprise at least two asymmetric bulges. In some embodiments, the engineered guide RNA can comprise SEQ ID NO: 46 or SEQ ID NO: 239. In some embodiments, the polynucleotide encoding the engineered guide RNA can comprise SEQ ID NO: 79 or SEQ ID NO: 207. In some embodiments, guide-target RNA scaffold can comprise at least one symmetric bulge. In some embodiments, the engineered guide RNA can comprise SEQ ID NO: 46 or SEQ ID NO: 239. In some embodiments, the polynucleotide encoding the engineered guide RNA can comprise SEQ ID NO: 79 or SEQ ID NO: 207. In some embodiments, the 6/6 symmetric internal loop can be at position 44 relative to a target adenosine at position 0. In some embodiments, the engineered guide RNA can comprise SEQ ID NO: 39 or SEQ ID NO: 93. In some embodiments, the polynucleotide encoding the engineered guide RNA can comprise SEQ ID NO: 32 or SEQ ID NO: 57. In some embodiments, the 6/6 symmetric internal loop can be at position −8 relative to a target adenosine at position 0. In some embodiments, the engineered guide RNA can comprise SEQ ID NO: 42 or SEQ ID NO: 93. In some embodiments, the polynucleotide encoding the engineered guide RNA can comprise SEQ ID NO: 35 or SEQ ID NO: 57. In some embodiments, the 6/6 symmetric internal loop can be at position 27 relative to a target adenosine at position 0. In some embodiments, the engineered guide RNA can comprise SEQ ID NO: 40 or SEQ ID NO: 228. In some embodiments, the polynucleotide encoding the engineered guide RNA can comprise SEQ ID NO: 33 or SEQ ID NO: 196. In some embodiments, the 6/6 symmetric internal loop can be at position −6 relative to a target adenosine at position 0. In some embodiments, the engineered guide RNA can comprise SEQ ID NO: 40 or SEQ ID NO: 228. In some embodiments, the polynucleotide encoding the engineered guide RNA can comprise SEQ ID NO: 33 or SEQ ID NO: 196. In some embodiments, the 6/6 symmetric internal loop can be at position −22 relative to a target adenosine at position 0. In some embodiments, the engineered guide RNA can comprise SEQ ID NO: 46 or SEQ ID NO: 239. In some embodiments, the polynucleotide encoding the engineered guide RNA can comprise SEQ ID NO: 79 or SEQ ID NO: 207. In some embodiments, the 6/6 symmetric internal loop can be at position −8 relative to a target adenosine at position 0. In some embodiments, the engineered guide RNA can comprise SEQ ID NO: 42 or SEQ ID NO: 93. In some embodiments, the polynucleotide encoding the engineered guide RNA can comprise SEQ ID NO: 35 or SEQ ID NO: 57. In some embodiments, the 6/6 symmetric internal loop can be at position −11 relative to a target adenosine at position 0.

In some embodiments, the engineered guide RNA can comprise SEQ ID NO: 44. In some embodiments, the polynucleotide encoding the engineered guide RNA can comprise SEQ ID NO: 37. In some embodiments, the 6/6 symmetric internal loop can be at position 36 relative to a target adenosine at position 0. In some embodiments, the engineered guide RNA can comprise SEQ ID NO: 46. In some embodiments, the polynucleotide encoding the engineered guide RNA can comprise SEQ ID NO: 79. In some embodiments, the at least 3 structural features, including the at least one 6/6 symmetric internal loop, comprise two symmetric internal loops at positions −5 and 44 relative to a target adenosine at position 0, and a mismatch at position 0 relative to the target adenosine at position 0. In some embodiments, the engineered guide RNA can comprise SEQ ID NO: 39. In some embodiments, the polynucleotide encoding the engineered guide RNA can comprise SEQ ID NO: 32. In some embodiments, the at least 3 structural features, including the at least one 6/6 symmetric internal loop, comprise two symmetric internal loops at positions −6 and 27 relative to a target adenosine at position 0, and a mismatch at position 0 relative to a target adenosine at position 0. In some embodiments, the engineered guide RNA can comprise SEQ ID NO: 40. In some embodiments, the polynucleotide encoding the engineered guide RNA can comprise SEQ ID NO: 33. In some embodiments, the at least 3 structural features, including the at least one 6/6 symmetric internal loop comprise, two symmetric internal loops at positions −6 and 27 relative to a target adenosine at position 0, a mismatch at position 0 relative to the target adenosine at position 0 and a wobble base pair at positions −35, −32, −27, −20, −18, −17, −15, 8, 20, 22, 37, 45, 49, 55, and 59 relative to the target adenosine at position 0. In some embodiments, the engineered guide RNA can comprise SEQ ID NO: 228. In some embodiments, the polynucleotide encoding the engineered guide RNA can comprise SEQ ID NO: 196. In some embodiments, the at least 3 structural features, including the at least one 6/6 symmetric internal loop, can comprise two symmetric internal loops at positions −22 and 36 relative to a target adenosine at position 0, an asymmetric bulge at positions −13 and −7 relative to the target adenosine at position 0, a symmetric bulge at position −3 relative to the target adenosine at position 0, and a wobble base pair at positions −21 and −14 relative to the target adenosine at position 0. In some embodiments, the engineered guide RNA can comprise SEQ ID NO: 46. In some embodiments, the polynucleotide encoding the engineered guide RNA can comprise SEQ ID NO: 79. In some embodiments, the at least 3 structural features, including the at least one 6/6 symmetric internal loop can comprise, a symmetric internal loop at positions −22 relative to a target adenosine at position 0, an asymmetric bulge at positions −13 and −7 relative to the target adenosine at position 0, a symmetric bulge at positions −3 and 36 relative to the target adenosine at position 0, a mismatch at position 41 relative to a target adenosine at position 0, and a wobble base pair at positions −35, −33, −31, −21, −14, 18, 20, 22, 24, 29, 31, 34, 45, 48, 51, 55, 58, 62, and 64 relative to the target adenosine at position 0. In some embodiments, the engineered guide RNA can comprise SEQ ID NO: 239. In some embodiments, the polynucleotide encoding the engineered guide RNA can comprise SEQ ID NO: 207. In some embodiments, the at least 3 structural features, including the at least one 6/6 symmetric internal loop, can comprise two symmetric internal loops at positions −8 and 35 relative to a target adenosine at position 0, and a mismatch at position 3 relative to the target adenosine at position 0. In some embodiments, the engineered guide RNA can comprise SEQ ID NO: 42. In some embodiments, the polynucleotide encoding the engineered guide RNA can comprise SEQ ID NO: 35. In some embodiments, the at least 3 structural features, including the at least one 6/6 symmetric internal loop, can comprise two symmetric internal loops at positions −8 and 44 relative to a target adenosine at position 0, and a mismatch at position 0 relative to the target adenosine at position 0. In some embodiments, the engineered guide RNA can comprise SEQ ID NO: 93. In some embodiments, the polynucleotide encoding the engineered guide RNA can comprise SEQ ID NO: 57. In some embodiments, the at least 3 structural features, including the at least one 6/6 symmetric internal loop, can comprise two symmetric internal loops at positions −11 and 42 relative to a target adenosine at position 0, and a mismatch at position 4 relative to the target adenosine at position 0. In some embodiments, the engineered guide RNA can comprise SEQ ID NO: 44. In some embodiments, the polynucleotide encoding the engineered guide RNA can comprise SEQ ID NO: 37. In some embodiments, the engineered guide RNA can hybridize to at least 80 bases of the DUX4 target sequence that has at least 80%, at least 85%, at least 90%, at least 92%, at least 95%, at least 96%, at least 97%, at least 98%, at least 99%, or 100% sequence identity to ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1). In some embodiments, the engineered guide RNA when hybridized to the DUX4 target sequence of the DUX4-FL mRNA can facilitate a knockdown of a DUX4 protein encoded by the DUX4-FL mRNA. In some embodiments, the knockdown can comprise the knockdown of at least 30%, at least 40%, at least 50%, at least 60%, at least 70%, at least 80%, or at least 90% of the DUX4-FL mRNA. In some embodiments, the engineered guide RNA when hybridized to the DUX4 target sequence can facilitate a knockdown of a gene downstream of DUX4 and wherein the gene downstream of DUX4 can comprise SLC34A2, LEUTX, ZSCAN4, PRAMEF12, TRIM43, KHDC1L, Wfdc3, Agtr2, DEFB103, or MBD3L2. In some embodiments, the knockdown can comprise the knockdown of at least 30%, at least 40%, at least 50%, at least 60%, at least 70%, at least 80%, or at least 90% of an mRNA or a protein encoded by the gene downstream of DUX4. In some embodiments, the engineered guide RNA when hybridized to the DUX4 target sequence can mask the polyA signal sequence. In some embodiments, the masking of the polyA signal sequence can facilitate a knockdown of at least about 20%, at least about 30%, at least about 40%, at least about 50%, at least about 60%, at least about 70%, at least about 80%, or at least about 90% of the DUX4-FL mRNA or a gene downstream of DUX4. In some embodiments, the gene downstream of DUX4 can comprise SLC34A2, LEUTX, ZSCAN4, PRAMEF12, TRIM43, DEFB103, KHDC1L, Wfdc3, Agtr2, or MBD3L2. In some embodiments, the engineered guide RNA when hybridized to the DUX4 target sequence can facilitate RNA editing by an RNA editing entity of one or more adenosines in the polyA signal sequence. In some embodiments, the RNA editing entity can comprise a human ADAR1, or a human ADAR2.

Also disclosed herein are AAV vectors comprising polynucleotides encoding engineered guide RNAs capable of hybridizing to a DUX4 target sequence of a DUX4-FL mRNA. In some embodiments, the DUX4 target sequence can comprise a polyA signal sequence. In some embodiments, the polynucleotide encoding the engineered guide RNA can comprise one or more sequences that have at least 80%, at least 85%, at least 90%, at least 92%, at least 95%, at least 97%, at least 99% or 100% sequence identity to SEQ ID NO: 32, SEQ ID NO: 33, SEQ ID NO: 35, SEQ ID NO: 37, SEQ ID NO: 57, SEQ ID NO: 79, SEQ ID NO: 196, or SEQ ID NO: 207. In some embodiments, the AAV vector can be AAV1, AAV2, AAV3, AAV4, AAV5, AAV6, AAV7, AAV8, AAV9, AAV10, AAV11, or a derivative, a chimera, or a variant thereof. In some embodiments, the AAV vector can be a recombinant AAV (rAAV) vector, a hybrid AAV vector, a chimeric AAV vector, a self-complementary AAV (scAAV) vector, or any combination thereof.

Also disclose herein are pharmaceutical compositions in unit dose form comprising: (a) the engineered guide RNAs or the polynucleotides encoding the engineered guide RNAs disclosed above, or the AAV vectors disclosed above; and (b) a pharmaceutically acceptable: excipient, carrier, or diluent.

Also disclosed herein are methods of treating a disease or a condition in a subject in need thereof, the method comprising administering to the subject a therapeutically effective amount of the engineered guide RNAs or the polynucleotides encoding the engineered guide RNAs disclosed above, the AAV vectors disclosed above, or the pharmaceutical compositions disclosed above. In some embodiments, the disease or condition can comprise Facioscapulohumeral muscular dystrophy (FSHD). In some embodiments, the FSHD can comprise FSHD1. In some embodiments, the subject can be a mouse, a non-human primate, or a human.

Also disclosed herein are methods of reducing DUX4 expression in a subject comprising administering to the subject an effective amount of the engineered guide RNAs or the polynucleotides encoding the engineered guide RNAs disclosed above, the AAV vectors disclosed above, or the pharmaceutical compositions disclosed above. In some embodiments, after the administering, the engineered guide RNA can hybridize to a DUX4 target sequence of a DUX4-FL mRNA, and the DUX4 target sequence can comprise a polyA signal sequence. In some embodiments, hybridization of the engineered guide RNA to the DUX4 target sequence can at least partially mask the polyA signal sequence. In some embodiments, the at least partially masking of the polyA signal sequence can facilitate destabilization of the DUX4-FL mRNA. In some embodiments, the engineered guide RNA when hybridized to the DUX4 target sequence can facilitate a knockdown of a gene downstream of DUX4 and the gene downstream of DUX4 can comprise SLC34A2, LEUTX, ZSCAN4, PRAMEF12, TRIM43, KHDC1L, Wfdc3, Agtr2, DEFB103, or MBD3L2. In some embodiments, the knockdown can comprise the knockdown of at least 30%, at least 40%, at least 50%, at least 60%, at least 70%, at least 80%, or at least 90% of an mRNA or a protein encoded by the gene downstream of DUX4. In some embodiments, the engineered guide RNA when hybridized to the DUX4 target sequence of the DUX4-FL mRNA can facilitate a knockdown of a DUX4 protein encoded by the DUX4-FL mRNA. In some embodiments, the knockdown can comprise the knockdown of at least 30%, at least 40%, at least 50%, at least 60%, at least 70%, at least 80%, or at least 90% of the DUX4-FL mRNA. In some embodiments, the subject can be a mouse, a non-human primate, or a human. In some embodiments, hybridization of the engineered guide RNA to the DUX4 target sequence can facilitate editing of the DUX4-FL mRNA by an RNA editing entity. In some embodiments, hybridization of the engineered guide RNA to the DUX4 target sequence can facilitate editing of the polyA signal sequence by the RNA editing entity. In some embodiments, the RNA editing entity can comprise an ADAR.

Also disclosed herein are in vitro methods of reducing DUX4 expression in a cell comprising administering to the cell an effective amount of the engineered guide RNAs or the polynucleotides encoding the engineered guide RNAs disclosed above, the AAV vectors disclosed above, or the pharmaceutical compositions disclosed above. In some embodiments, after the administering, the engineered guide RNA can mask a DUX 4 target sequence of a DUX4-FL RNA transcript encoding the DUX4. In some embodiments, the cell can be a muscle cell. In some embodiments, the muscle cell can comprise a myocyte, a myofibril, a myoblast, a myotube or any combination thereof. In some embodiments, the DUX 4 target sequence of the DUX4-FL RNA transcript can comprise a polyA signal sequence. In some embodiments, the masking the polyA signal sequence can facilitate a knockdown of at least about 20%, at least about 30%, at least about 40%, at least about 50%, at least about 60%, at least about 70%, at least about 80%, or at least about 90% of the DUX4 expression in the cell. In some embodiments, the masking of the polyA signal sequence facilitates a knockdown of DUX4 or a gene downstream of DUX4 and the gene downstream of DUX4 can comprise SLC34A2, LEUTX, ZSCAN4, PRAMEF12, TRIM43, DEFB103, KHDC1L, Wfdc3, Agtr2, or MBD3L2. In some embodiments, the knockdown can comprise the knockdown of at least 30%, at least 40%, at least 50%, at least 60%, at least 70%, at least 80%, or at least 90% of the mRNA encoding DUX4 or of the gene downstream of DUX4.

Also disclosed herein are kits comprising the engineered guide RNAs or the polynucleotide encoding the engineered guide RNAs disclosed above, the AAV vectors disclosed above, or the pharmaceutical compositions disclosed above and a container.

Also disclosed herein are engineered guide RNAs or polynucleotides encoding the engineered guide RNAs, comprising a polynucleotide sequence with at least 80%, at least 85%, at least 90%, at least 92%, at least 95%, at least 96%, at least 97%, at least 98%, at least 99%, or 100% sequence identity to any one of SEQ ID NO: 32, SEQ ID NO: 39, SEQ ID NO: 33, SEQ ID NO: 40, SEQ ID NO: 35, SEQ ID NO: 42, SEQ ID NO: 37, SEQ ID NO: 44, SEQ ID NO: 79, SEQ ID NO: 46, SEQ ID NO: 57, SEQ ID NO: 93, SEQ ID NO: 196, SEQ ID NO: 228, SEQ ID NO: 207, or SEQ ID NO: 239.

Also disclosed herein are engineered guide RNAs or polynucleotides encoding the engineered guide RNAs, comprising a polynucleotide sequence with at least 80%, at least 85%, at least 90%, at least 92%, at least 95%, at least 96%, at least 97%, at least 98%, at least 99%, or 100% sequence identity to any one of SEQ ID NO: 32-SEQ ID NO: 38, SEQ ID NO: 39-SEQ ID NO: 46, SEQ ID NO: 47-SEQ ID NO: 80, SEQ ID NO: 83-SEQ ID NO: 115, SEQ ID NO: 118-SEQ ID NO: 122, SEQ ID NO: 148-SEQ ID NO: 152, SEQ ID NO: 124-SEQ ID NO: 138, SEQ ID NO: 154-SEQ ID NO: 168, SEQ ID NO: 140-SEQ ID NO: 141, SEQ ID NO: 170-SEQ ID NO: 171, SEQ ID NO: 143-SEQ ID NO: 147, SEQ ID NO: 173-SEQ ID NO: 177, SEQ ID NO: 178-SEQ ID NO: 209, SEQ ID NO: 210-SEQ ID NO: 241, SEQ ID NO: 246-SEQ ID NO: 247, SEQ ID NO: 248-SEQ ID NO: 254 or SEQ ID NO: 255-SEQ ID NO: 261.

Also disclosed herein are engineered guide RNAs or a polynucleotide encoding the engineered guide RNAs, wherein the engineered guide RNAs hybridizes to at least 80 bases of a target RNA sequence with at least 80%, at least 85%, at least 90%, at least 92%, at least 95%, at least 96%, at least 97%, at least 98%, at least 99%, or 100% sequence identity to ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1) and facilitate a protein knockdown or an mRNA knockdown.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1 shows a schematic of the double homeobox 4 (DUX4) target, highlighting sites that can be targeted by engineered guide RNAs of the present disclosure.

FIG. 2 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). The figure discloses SEQ ID NOS 262-263, respectively, in order of appearance.

FIG. 3A-FIG. 3G shows shifting the macrofootprint towards the 100.55-100.65 symmetry increases efficacy of gRNAs to reduce apoptosis (cell death) as measured by caspase activity. On the graphs, the Y-axis shows the relative caspase activity, and the X-axis shows the transfection of different guide RNAs and controls. FIG. 3A shows macrofootprint adjustment in the SEQ ID NO: 86 guide. FIG. 3B shows macrofootprint adjustment in the SEQ ID NO: 92 guide. FIG. 3C shows macrofootprint adjustment in the SEQ ID NO: 96 guide. FIG. 3D shows macrofootprint adjustment in the SEQ ID NO: 101 guide. FIG. 3E shows macrofootprint adjustment in the SEQ ID NO: 105 guide. FIG. 3F shows macrofootprint adjustment in the SEQ ID NO: 110 guide. FIG. 3G shows macrofootprint adjustment in a machine learning designed guide.

FIG. 4A-FIG. 4B shows efficacy of different gRNAs to reduce apoptosis (cell death) as measured by caspase activity. On the graphs the Y-axis shows the relative caspase activity, and the X-axis shows the transfection of different guide RNAs and controls. FIG. 4A shows the results of a biologically independent replicate of the guides in a heterologous population of DUX4 inducible HEK293T cells and FIG. 4B shows the results of a biologically independent replicate of the guides in a clonal population of DUX4 inducible HEK293T cells.

FIG. 5 shows the efficacy of different gRNAs (from at least 3 biologically independent replicates) to reduce apoptosis as measured by caspase activity. On the graphs, the Y-axis shows the relative caspase activity, and the X-axis shows the transfection of different guide RNAs and controls.

FIG. 6A-FIG. 6D shows the effect of different gRNAs to reduce mRNA expression of genes downstream of DUX4. On the graphs, the Y-axis shows the relative expression of the downstream (of DUX4) gene normalized to RPL30, and the X-axis shows the stable integration of different guide RNAs and controls into immortalized affected FSHD cells. The numbers in the bars indicate the percent mRNA knockdown. FIG. 6A shows the expression change of ZSCAN4;

FIG. 6B shows the expression change of MBD3L2; FIG. 6C shows the expression change of PRAMEF12; and FIG. 6D shows the expression change of TRIM43. The data in FIGS. 6A-6D is summarized in FIG. 12.

FIG. 7 shows an alignment of different guide RNAs (shown from top to bottom as: CTCTGCACTCATCACACAAAAGATGCAAATCTTCTATAGGATCCACAGGGAGGGGG CATTCTAACATATCTMGCTCACTAATCATCCAGGAGATGTAACTCTAATCCAGGT (SEQ ID NO: 18), SEQ ID NO: 32, SEQ ID NO: 33, SEQ ID NO: 34, SEQ ID NO: 35, SEQ ID NO: 36, SEQ ID NO: 37 and SEQ ID NO: 38). The highlighted bases of each sequence show areas containing substitutions as compared to the consensus identity sequence.

FIG. 8A-FIG. 8B shows efficacy of different gRNAs to reduce apoptosis as measured by caspase activity. The guide RNAs are progeny guide RNAs of SEQ ID NO: 40. On the graphs, the Y-axis shows the relative caspase activity, and the X-axis shows the transfection of progeny guide RNAs and controls. FIG. 8A shows the first biological replicate and FIG. 8B shows the second biological replicate.

FIG. 9A-FIG. 9B shows efficacy of different gRNAs to reduce apoptosis as measured by caspase activity. The guide RNAs are progeny guide RNAs of SEQ ID NO: 39. On the graphs, the Y-axis shows the relative caspase activity, and the X-axis shows the transfection of a parental guide RNA, progeny guide RNAs, and controls. FIG. 9A shows the first biological replicate and FIG. 9B shows the second biological replicate.

FIG. 10 shows the efficacy of different gRNAs to reduce apoptosis as measured by caspase activity. The guide RNAs are progeny guide RNAs of SEQ ID NO: 46. On the graphs, the Y-axis shows the relative caspase activity, and the X-axis shows the transfection of a parental guide RNA, progeny guide RNAs, and controls.

FIG. 11 shows the efficacy of different gRNAs to reduce apoptosis as measured by caspase activity. On the graphs the Y-axis shows the relative caspase activity, and the X-axis shows the transfection of different guide RNAs and controls. FIG. 11 shows the results of 3 biologically independent replicates of selected guide RNAs and selected progeny guide RNAs in DUX4 inducible HEK293T cells.

FIG. 12 shows affected primary FSHD myotubes had decreased DUX4-activated gene expression in MBD3L2, TRIM43 and ZSCAN4. On the graphs, the Y-axis shows the relative expression of the downstream (of DUX4) gene normalized to RPL30 and the X-axis shows the stable integration of different guide RNAs into primary affected FSHD cells. The data in FIG. 12 is a summary of the data shown in FIGS. 6A-6D.

FIG. 13 shows scAAV6 transduced guides were able to reduce DUX4-activated gene expression in affected primary FSHD myotubes. The cells had decreased DUX4-activated gene expression in KHDC1L, ZSCAN4, TRIM43, MBD3L2, LEUTX and PRAMEF12. On the graphs, the Y-axis shows the relative expression of the downstream (of DUX4) gene normalized to RPL30 and the X-axis shows the transduction of guide RNAs containing different reporter constructs into affected and unaffected primary FSHD cells.

FIG. 14A-FIG. 14B shows a scAAV serotype transduction panel with immortalized and primary FSHD myotubes. On the graphs, the Y-axis shows the percentage of GFP positive cells after transduction and the X-axis shows different scAAV serotypes. FIG. 14A shows immortalized FSHD myotubes. FIG. 14B shows primary FSHD myotubes.

FIG. 15 shows RNA editing of different adenosines in the polyadenylation sequence of non-canonical DUX4 transcript with different engineered guide RNAs in DUX4 luciferase reporter LHCN muscle cells. The Y-axis shows the percentage edited and the X-axis shows the different guide RNAs and the no guide RNA transfection control (No Tfxn).

FIG. 16 shows RNA editing of the DUX4 polyadenylation sequence of non-canonical DUX4 transcripts compared to transcripts with canonical polyadenylation in DUX4 luciferase reporter HEK293T cells. The top graph shows editing of the polyadenylation sequence in transcripts with canonical polyadenylation while the bottom graph shows editing of the polyadenylation sequence in non-canonical transcripts. The Y-axis shows percent editing while the X-axis shows the target polyadenylation site—“ATTAAA”.

FIG. 17A-FIG. 17B show guide RNA expression of a RAB7A targeting guide RNA (FIG. 17A) and a DUX4 targeting guide RNA (FIG. 17B) in a DUX4 inducible mouse model. The Y-axis shows the guide RNA copies per U1snRNA while the X-axis shows the different treatment groups.

FIG. 18A-FIG. 18B shows RNA expression change in mouse DUX4-activated genes in vivo. FIG. 18A shows the expression change of Agtr2 and FIG. 18B shows the expression change in Wfdc3. The Y-axis shows the fold change of mouse DUX4-activated genes relative to relative to vehicle control, and the X-axis shows the different treatment groups and controls. The data points represent the average of technical triplicates. Dashed lines indicate no knockdown (1.0-fold change) and 50% knockdown (0.5-fold change) of mouse DUX4-activated genes relative to vehicle control.

### DETAILED DESCRIPTION

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from cytidine to give rise to uridine, or C-to-U editing or from adenosine to inosine, or A-to-I editing). RNA editing can be used to introduce mutations, correct missense mutations, or edit coding or non-coding regions of RNA to inhibit RNA translation and effect protein knockdown.

Described herein are engineered guide RNAs that facilitate RNA editing by an RNA editing entity (e.g., an adenosine Deaminase Acting on RNA (ADAR)) or biologically active fragments thereof. In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADAR1, ADAR2, ADAR3, or a combination thereof). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. The engineered guide RNAs of the present disclosure facilitate RNA editing by endogenous ADAR enzymes. In some embodiments, exogenous ADAR can be delivered alongside the engineered guide RNAs disclosed herein.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence of an RNA. The engineered guide RNAs disclosed herein can target a non-coding sequence of an RNA, for example, a polyadenylation (polyA) signal sequence in the 3′UTR.

The present disclosure, in some embodiments, provides engineered guide RNAs that facilitate edits at multiple adenosines. Hydrolytic deamination of multiple adenosines in an RNA can be referred to as hyper-editing. In some cases, hyper-editing can occur in cis (e.g. in an Alu element) or in trans (e.g. in a target RNA by an engineered guide RNA). In some cases, hyper-editing can comprise editing in the polyA signal sequence of the DUX4-FL target RNA. In some cases, hyper-editing can introduce edits in at least 2 or more nucleotides of a subject target RNA. In some cases, hyper-editing can introduce at least or at most about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or at least or at most about 100 edits in a region of a target RNA. In an embodiment, hyper-editing can occur in an untranslated region, translated region, 3′ UTR, 5′ UTR, or any combinations thereof.

PolyA Signal Sequence. In some embodiments, the engineered guide RNAs of the present disclosure target one or more adenosines in the polyA signal sequence. In some cases, the polyA signal sequence is referred to herein as the polyA signal site. In some embodiments, an engineered guide RNA facilitates ADAR-mediated RNA editing of the one or more adenosines in the polyA signal sequence, thereby resulting in disruption of RNA processing and degradation of the target mRNA and, thereby, reduction in DUX4 activity. In some embodiments, a target can have one or more polyA signal sequences. In these instances, one or more engineered guide RNAs, varying in their respective sequences, of the present disclosure can be multiplexed to target adenosines in the one or more polyA signal sequences. In both cases, the engineered guide RNAs of the present disclosure facilitated ADAR-mediated RNA editing of adenosines to inosines (read as guanosines by cellular machinery) in the polyA signal sequence, resulting in protein knockdown, mRNA knockdown, or both. In some cases, the engineered guide RNAs of the present disclosure can bind and mask the polyA signal sequence to facilitate destabilization of DUX4-FL mRNA.

Target Sequence. In some embodiments, the engineered guide RNAs of the present disclosure target one or more adenosines in the following sequence: ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1). In some embodiments, a DUX4 target sequence comprises a DUX4-FL mRNA. In some embodiments, a DUX4 target sequence comprises a polyA signal sequence.

### PolyA Signal Sequence Masking

Alternatively or in addition to RNA editing, the engineered guide RNAs of the present disclosure can facilitate knockdown of DUX4 protein expression, expression of proteins or mRNAs downstream of DUX4, or a combination thereof by hybridizing with DUX4-FL mRNA and masking the polyA signal sequence. Masking the polyA signal sequence with an engineered guide RNA can facilitate knockdown by facilitating destabilization of the target DUX4-FL mRNA. In some embodiments, an engineered guide RNA may facilitate knockdown of DUX4 protein expression, expression of proteins or mRNAs downstream of DUX4, or a combination thereof, through polyA signal sequence masking without facilitating editing of the DUX4-FL mRNA. In some embodiments, an engineered guide RNA may facilitate knockdown of DUX4 protein expression, expression of proteins or mRNAs downstream of DUX4, or a combination thereof, through polyA masking and by facilitating editing of the DUX4-FL mRNA.

### Definitions

Unless defined otherwise, all terms of art, notations and other technical and scientific terms or terminology used herein are intended to have the same meaning as commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As used herein, the term “engineered guide RNA” can be used interchangeably with “guide RNA” and refers to a designed polynucleotide that is at least partially complementary to a target RNA. An engineered guide RNA of the present disclosure can be used to facilitate modification of the target RNA. Modification of the target RNA includes alteration of RNA splicing, reduction or enhancement of protein translation, target RNA knockdown, target RNA degradation, and/or ADAR mediated RNA editing of the target RNA. In some cases, guide RNAs facilitate ADAR-mediated RNA editing for the purpose of target mRNA knockdown, downstream protein translation reduction or inhibition, downstream protein translation enhancement, correction of mutations (including correction of any G-to-A mutation, such as missense or nonsense mutations), introduction of mutations (e.g., introduction of an A-to-I (read as a G by cellular machinery) substitution), or alter the function of any adenosine containing a regulatory motif (e.g., polyadenylation signal, miRNA binding site, etc.). In some cases, a guide RNA can effect a functional outcome (e.g., target RNA modulation, downstream protein translation) via a combination of mechanisms, for example, ADAR-mediated RNA editing and binding and/or degrading target RNA. In some cases, a guide RNA can facilitate introduction of mutations at sites targeted by enzymes in order to modify the affinity of such enzymes for targeting and cleaving such sites. The guide RNAs of this disclosure can contain one or more structural features. A structural feature can be formed from latent structure in latent (unbound) guide RNA upon hybridization of the engineered latent guide RNA to a target RNA. Latent structure refers to a structural feature that forms or substantially forms only upon hybridization of a guide RNA to a target RNA. For example, upon hybridization of the guide RNA to the target RNA, the latent structural feature is formed in the resulting double stranded RNA (also referred herein as guide-target RNA scaffold). In such cases, a structural feature can include, but is not limited to, a mismatch, a wobble base pair, a symmetric internal loop, an asymmetric internal loop, a symmetric bulge, or an asymmetric bulge. In other instances, a structural feature can be a pre-formed structure (e.g., a GluR2 recruitment hairpin, or a hairpin from U7 snRNA).

As used herein, the term “targeting sequence” can be used interchangeable with “targeting domain” or “targeting region” and refers to a polynucleotide sequence within an engineered guide RNA sequence that is at least partially complementary to a target polynucleotide. The target polynucleotide (e.g., a target RNA or a target DNA) may be a region of a polynucleotide of interest, such as a gene or a messenger RNA. As used herein, a “complementary” sequence refers to a sequence that is a reverse complement relative to a second sequence.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to the ability of a polynucleotide to provide information or instructions sequence sufficient to produce a corresponding gene expression product. In a non-limiting example, mRNA can encode a polypeptide during translation, whereas DNA can encode an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows: [(Number of Identical Positions)/(Total Number of Positions in the Test Sequence)]×100%

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023 are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows: Percent Sequence Identity=(“Percent Identity” output value)×(“Query Coverage” output value)

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (SEQ ID NO: 11) (length=10 nucleotides) to reference sequence 2: AAAAAAAAAA (SEQ ID NO: 12) (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (SEQ ID NO: 13) (length=20 nucleotides) to reference sequence 4: GGGGGGGGGG (SEQ ID NO: 14) (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (SEQ ID NO: 14) (length=10 nucleotides) to reference sequence 6: CCCCCGGGGGGGGGGCCCCC (SEQ ID NO: 13) (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (SEQ ID NO: 15) (length=10 amino acids) to reference sequence 8: YYYYYYYYYY (SEQ ID NO: 16) (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (SEQ ID NO: 17) (length=20 amino acids) to reference sequence 10: FFFFFYYYYY (SEQ ID NO: 15) (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (SEQ ID NO: 15) (length=10 amino acids) to reference sequence 12: LLLLLFFFFFYYYYYLLLLL (SEQ ID NO: 17) (length=20 amino acids). The percent identity between test sequence 11 and reference sequence 12 would be [(10)/(10)]×100%=100%. Test sequence 11 has 100% sequence identity to reference sequence 12.

Latent structure refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked. The formation and structure of a latent structural feature upon binding to the target RNA depends on the guide RNA sequence. For example, formation and structure of the latent structural feature may depend on a pattern of complementary and mismatched residues in the guide RNA sequence relative to the target RNA. The guide RNA sequence may be engineered to have a latent structural feature that forms upon binding to the target RNA.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint. In some embodiments, a macro-footprint sequence can comprise a barbell macro-footprint sequence comprising latent structures that, when manifested, produce a first internal loop and a second internal loop.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids. A sequence of a polynucleotide may be provided interchangeably as an RNA sequence (containing U) or a DNA sequence (containing T). A sequence provided as an RNA sequence is intended to also cover the corresponding DNA sequence and the reverse complement RNA sequence or DNA sequence. A sequence provided as a DNA sequence is intended to also cover the corresponding RNA sequence and the reverse complement RNA sequence or DNA sequence.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes place within a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g. physiological pH, physiological temperature, physiological salt concentration, etc.).

As disclosed herein, a structured motif comprises two or more structural features in a guide-target RNA scaffold.

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### Engineered Guide RNAs

Disclosed herein are engineered guide RNAs and engineered polynucleotides encoding the same for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, tphus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off-target editing in addition to the desired editing.

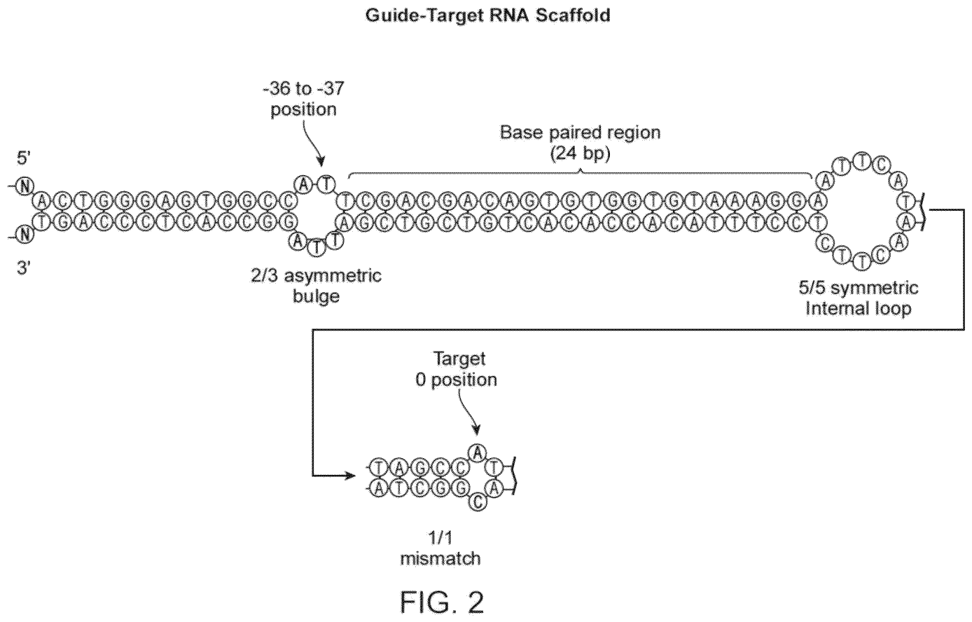

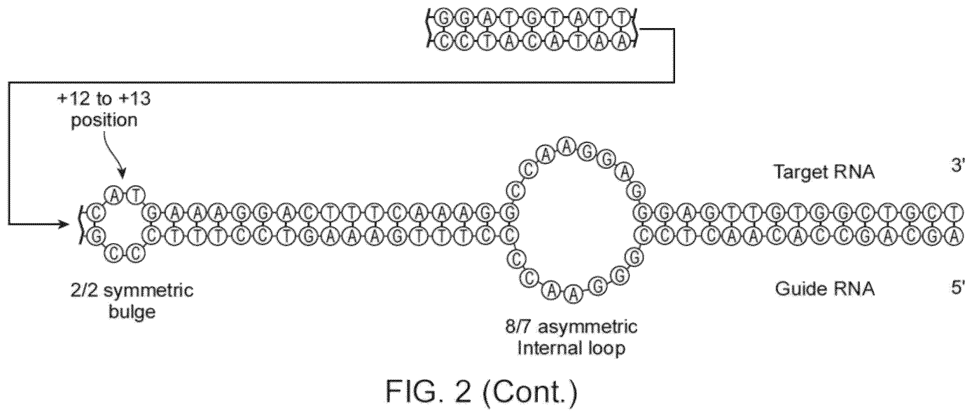

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity. FIG. 2 illustrates a target RNA scaffold with exemplary structural features. Latent structures herein can be described with respect to their position relative to a target adenosine in the target RNA transcript. The target adenosine can be indicated as position “0”. In some cases, the latent structures described herein can be described upstream (5′) or downstream (3′) of the target adenosine. As used herein, positional and directional annotation is provided with respect to the target nucleotide to be edited and on the target RNA side of the guide-target RNA scaffold.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA.

The part of the engineered guide RNA that hybridizes to the target RNA is of sufficient complementary to the sequence of the target RNA for hybridization to occur.

In some cases, an engineered guide RNA herein comprise a length of about: 20, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides. In some cases, an engineered guide RNA herein comprises a length of about: 95, 96, 97, 98, 99, 100, 101, 102, 103, 104, or 105 nucleotides. In some cases, an engineered guide RNA herein comprises a length of about: 25 to 200 nucleotides in length, 50 to 150 nucleotides in length, 75 to 100 nucleotides in length, 80 to 110 nucleotides in length, 90 to 120 nucleotides in length, 95 to 115 nucleotides in length, 60 to 200 nucleotides in length, 60 to 180 nucleotides in length, 60 to 160 nucleotides in length, 60 to 140 nucleotides in length, 70 to 200 nucleotides in length, 70 to 180 nucleotides in length, 70 to 160 nucleotides in length, 70 to 140 nucleotides in length, 80 to 200 nucleotides in length, 80 to 190 nucleotides in length, 80 to 170 nucleotides in length, 80 to 160 nucleotides in length, 80 to 150 nucleotides in length, 80 to 140 nucleotides in length, 80 to 130 nucleotides in length, 80 to 120 nucleotides in length, 90 to 200 nucleotides in length, 90 to 190 nucleotides in length, 90 to 180 nucleotides in length, 90 to 170 nucleotides in length, 90 to 160 nucleotides in length, 90 to 150 nucleotides in length, 90 to 140 nucleotides in length, 90 to 130 nucleotides in length, 90 to 120 nucleotides in length, 90 to 110 nucleotides in length, 95 to 105 nucleotides in length, 100 to 200 nucleotides in length, 100 to 190 nucleotides in length, 100 to 180 nucleotides in length, 100 to 170 nucleotides in length, 100 to 160 nucleotides in length, 100 to 150 nucleotides in length, 100 to 140 nucleotides in length, 100 to 130 nucleotides in length, 100 to 120 nucleotides in length, 110 to 200 nucleotides in length, 110 to 190 nucleotides in length, 110 to 180 nucleotides in length, 110 to 170 nucleotides in length, 110 to 160 nucleotides in length, 110 to 150 nucleotides in length, 110 to 140 nucleotides in length, 110 to 120 nucleotides in length, 120 to 200 nucleotides in length, 120 to 190 nucleotides in length, 120 to 180 nucleotides in length, 120 to 170 nucleotides in length, 120 to 160 nucleotides in length, 120 to 150 nucleotides in length, 120 to 140 nucleotides in length, 130 to 200 nucleotides in length, or about 140 to 200 nucleotides in length.

Disclosed herein are engineered guide RNAs (e.g. an engineered guide RNA that comprise a polynucleotide sequence of any one of SEQ ID NO: 39, 40, 46, 212, 228, or 239. In some cases, a polynucleotide encoding an engineered guide RNA can comprise any one of SEQ ID NO: 32, 33, 79, 180, 196, or 207. In some cases, an engineered guide RNA herein can target the DUX4 polyA signal RNA sequence. In some embodiments, the engineered guide RNA of the present disclosure targets one or more adenosines in the RNA sequence of SEQ ID NO: 1, SEQ ID NO: 243, or SEQ ID NO: 245 or a sequence that is at least 80%, at least 85%, at least 90%, at least 92%, at least 95%, at least 96%, at least 97%, at least 98%, or at least 99% identical to SEQ ID NO: 1, SEQ ID NO: 243, or SEQ ID NO: 245. In some embodiments, the engineered guide RNAs of the present disclosure can mask a region (e.g., a polyA signal) in the RNA sequence of SEQ ID NO: 1, SEQ ID NO: 243, or SEQ ID NO: 245 or a sequence that is at least 80%, at least 85%, at least 90%, at least 92%, at least 95%, at least 96%, at least 97%, at least 98%, or at least 99% identical to SEQ ID NO: 1, SEQ ID NO: 243, or SEQ ID NO: 245.

### A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region”.

The targeting sequence of an engineered guide RNA allows the engineered guide RNA to hybridize to a target polynucleotide (e.g., a target RNA) through base pairing, such as Watson Crick base pairing. A targeting sequence can be located at either the N-terminus or C-terminus of the engineered guide RNA, or both, or the targeting sequence can be within the engineered guide RNA. The targeting sequence can be of any length sufficient to hybridize with the target polynucleotide. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides in length. In an embodiment, an engineered polynucleotide comprises a targeting sequence that is about 25 to 200, 50 to 150, 75 to 100, 80 to 110, 90 to 120, 95 to 115, 60 to 200, 60 to 180, 60 to 160, 60 to 140, 70 to 200, 70 to 180, 70 to 160, 70 to 140, 80 to 200, 80 to 190, 80 to 170, 80 to 160, 80 to 150, 80 to 140, 80 to 130, 80 to 120, 90 to 200, 90 to 190, 90 to 180, 90 to 170, 90 to 160, 90 to 150, 90 to 140, 90 to 130, 90 to 120, 100 to 200, 100 to 190, 100 to 180, 100 to 170, 100 to 160, 100 to 150, 100 to 140, 100 to 130, 100 to 120, 110 to 200, 110 to 190, 110 to 180, 110 to 170, 110 to 160, 110 to 150, 110 to 140, 110 to 120, 120 to 200, 120 to 190, 120 to 180, 120 to 170, 120 to 160, 120 to 150, 120 to 140, 130 to 200, 130 to 190, 130 to 180, 130 to 170, 130 to 160, 130 to 150, 140 to 200, 140 to 190, 140 to 180, 140 to 170, 140 to 160, 150 to 200, 150 to 190, 150 to 180, 150 to 170, 160 to 200, 160 to 190 or 160 to 180 nucleotides in length.

A targeting sequence comprises at least partial sequence complementarity to a target polynucleotide. The targeting sequence may have a degree of sequence complementarity to the target polynucleotide sufficient to hybridize with the target polynucleotide. In some cases, the targeting sequence comprises 95%, 96%, 97%, 98%, 99%, or 100% sequence complementarity to the target polynucleotide. In some cases, the targeting sequence comprises less than 100% complementarity to the target polynucleotide sequence. For example, the targeting sequence may have a single base mismatch relative to the target polynucleotide when bound to the target polynucleotide. In other cases, the targeting sequence comprises at least about 1, 2, 3, 4, 5, 6, 7, 8, 9, 10, 11, 12, 13, 14, 15, 16, 20, 30, 40 or up to about 50 base mismatches relative to the target polynucleotide when bound to the target polynucleotide. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype polynucleotide of a subject target polynucleotide.

A targeting sequence comprises nucleotide residues having complementarity to a target polynucleotide. The targeting sequence may have a number of residues with complementarity to the target polynucleotide sufficient to hybridize with the target polynucleotide. The complementary residues may be contiguous or non-contiguous. In some cases, the targeting sequence comprises at least 50 nucleotides having complementarity to the target polynucleotide. In some cases, the targeting sequence comprises from 50 to 150 nucleotides having complementarity to the target polynucleotide. In some cases, the targeting sequence comprises from 50 to 200 nucleotides having complementarity to the target polynucleotide. In some cases, the targeting sequence comprises from 50 to 250 nucleotides having complementarity to the target polynucleotide. In some cases, the targeting sequence comprises from 50 to 300 nucleotides having complementarity to the target polynucleotide. In some cases, the targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to the target polynucleotide. In some cases, the targeting sequence comprises more than 50 nucleotides total and has at least 50 nucleotides having complementarity to the target polynucleotide. In some cases, the targeting sequence comprises from 50 to 400 nucleotides total and has from 50 to 150 nucleotides having complementarity to the target polynucleotide. In some cases, the targeting sequence comprises from 50 to 400 nucleotides total and has from 50 to 200 nucleotides having complementarity to the target polynucleotide. In some cases, the targeting sequence comprises from 50 to 400 nucleotides total and has from 50 to 250 nucleotides having complementarity to the target polynucleotide. In some cases, the targeting sequence comprises from 50 to 400 nucleotides total and has from 50 to 300 nucleotides having complementarity to the target polynucleotide. In some cases, the at least 50 nucleotides having complementarity to the target polynucleotide are separated by one or more mismatches, one or more bulges, or one or more loops, or any combination thereof. In some cases, the from 50 to 150 nucleotides having complementarity to the target polynucleotide are separated by one or more mismatches, one or more bulges, or one or more loops, or any combination thereof. In some cases, the from 50 to 200 nucleotides having complementarity to the target polynucleotide are separated by one or more mismatches, one or more bulges, or one or more loops, or any combination thereof. In some cases, the from 50 to 250 nucleotides having complementarity to the target polynucleotide are separated by one or more mismatches, one or more bulges, or one or more loops, or any combination thereof. In some cases, the from 50 to 300 nucleotides having complementarity to the target polynucleotide are separated by one or more mismatches, one or more bulges, or one or more loops, or any combination thereof. For example, a targeting sequence comprises a total of 54 nucleotides wherein, sequentially, 25 nucleotides are complementarity to the target polynucleotide, 4 nucleotides form a bulge, and 25 nucleotides are complementarity to the target polynucleotide. As another example, a targeting sequence comprises a total of 118 nucleotides wherein, sequentially, 25 nucleotides are complementarity to the target polynucleotide, 4 nucleotides form a bulge, 25 nucleotides are complementarity to the target polynucleotide, 14 nucleotides form a loop, and 50 nucleotides are complementary to the target polynucleotide.

In some embodiments, a guide RNA or a polynucleotide encoding a guide RNA disclosed herein can comprise a targeting sequence disclosed in Table 1, Table 3, Table 4, Table 5, Table 6, Table 8, Table 11, Table 12, Table 13, or Table 14. In some embodiments, an engineered guide RNA or a polynucleotide encoding an engineered guide RNA can comprise a sequence with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to any sequence in in Table 1, Table 3, Table 4, Table 5, Table 6, Table 8, Table 11, Table 12, Table 13, or Table 14.

In some embodiments, a composition can comprise an engineered guide RNA comprising SEQ ID NO: 39, SEQ ID NO: 40, SEQ ID NO: 42, SEQ ID NO: 44, SEQ ID NO: 46, SEQ ID NO: 93, SEQ ID NO: 228, and/or SEQ ID NO: 239. In some embodiments, a composition can comprise an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO: 39, SEQ ID NO: 40, SEQ ID NO: 42, SEQ ID NO: 44, SEQ ID NO: 46, SEQ ID NO: 93, SEQ ID NO: 228, and/or SEQ ID NO: 239. In some embodiments, a composition can comprise a polynucleotide encoding an engineered guide RNA comprising SEQ ID SEQ ID NO: 32, SEQ ID NO: 33, SEQ ID NO: 35, SEQ ID NO: 37, SEQ ID NO: 57, SEQ ID NO: 79, SEQ ID NO: 196, and/or SEQ ID NO: 207. In some embodiments, a composition can comprise a polynucleotide encoding an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 32, SEQ ID NO: 33, SEQ ID NO: 35, SEQ ID NO: 37, SEQ ID NO: 57, SEQ ID NO: 79, SEQ ID NO: 196, and/or SEQ ID NO: 207.

In some embodiments, a composition can comprise an engineered guide RNA comprising SEQ ID NO: 39-SEQ ID NO: 46, SEQ ID NO: 83-SEQ ID NO: 117, SEQ ID NO: 148-SEQ ID NO: 152, SEQ ID NO: 154-SEQ ID NO: 168, SEQ ID NO: 170-SEQ ID NO: 171, SEQ ID NO: 173-SEQ ID NO: 177, SEQ ID NO: 210-SEQ ID NO: 241, SEQ ID NO: 255-SEQ ID NO: 261 and/or SEQ ID NO: 247. In some embodiments, a composition can comprise an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to SEQ ID NO: 39-SEQ ID NO: 46, SEQ ID NO: 83-SEQ ID NO: 117, SEQ ID NO: 148-SEQ ID NO: 152, SEQ ID NO: 154-SEQ ID NO: 168, SEQ ID NO: 170-SEQ ID NO: 171, SEQ ID NO: 173-SEQ ID NO: 177, SEQ ID NO: 210-SEQ ID NO: 241, SEQ ID NO: 255-SEQ ID NO: 261 and/or SEQ ID NO: 247. In some embodiments, a composition can comprise a polynucleotide encoding an engineered guide RNA comprising SEQ ID NO: 32-SEQ ID NO: 38, SEQ ID NO: 47-SEQ ID NO: 82, SEQ ID NO: 118-SEQ ID NO: 122, SEQ ID NO: 124-SEQ ID NO: 138, SEQ ID NO: 140-SEQ ID NO: 141, SEQ ID NO: 143-SEQ ID NO: 147, SEQ ID NO: 178-SEQ ID NO: 209, SEQ ID NO: 248-SEQ ID NO: 254, and/or SEQ ID NO: 246. In some embodiments, a composition can comprise a polynucleotide encoding an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 32-SEQ ID NO: 38, SEQ ID NO: 47-SEQ ID NO: 82, SEQ ID NO: 118-SEQ ID NO: 122, SEQ ID NO: 124-SEQ ID NO: 138, SEQ ID NO: 140-SEQ ID NO: 141, SEQ ID NO: 143-SEQ ID NO: 147, SEQ ID NO: 178-SEQ ID NO: 209, SEQ ID NO: 248-SEQ ID NO: 254, and/or SEQ ID NO: 246. In some embodiments, a composition can comprise an engineered guide RNA comprising any one of SEQ ID NO: 39-SEQ ID NO: 46 or SEQ ID NO: 212, 228 and/or 239. In some embodiments, a composition can comprise an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to any one of SEQ ID NO: 39-SEQ ID NO: 46 or SEQ ID NO: 212, 228 and/or 239. In some embodiments, a composition can comprise a polynucleotide encoding an engineered guide RNA comprising any one of SEQ ID NO: 32-SEQ ID NO: 38 or SEQ ID NO: 79, 180, 196, and/or 207. In some embodiments, a composition can comprise a polynucleotide encoding an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99% sequence identity to any one of any one of SEQ ID NO: 32-SEQ ID NO: 38 or SEQ ID NO: 79, 180, 196, and/or 207.

Table 1 provide different guide RNA sequences (DNA and RNA sequences) and the latent structural features associated with guide RNA sequences. For each sequence, the structural features formed in the double stranded RNA substrate upon hybridization of the guide RNA to the target DUX4-FL RNA, are shown in the last column of Table 1. For reference, each structural feature formed within a guide-target RNA scaffold (target RNA sequence hybridized to an engineered guide RNA) is annotated as follows:
a) the position of the structural feature with respect to the target A (position 0—the first A (bolded) in the polyA signal sequence AUUAAA) of the target RNA sequence, with a negative value indicating upstream (5′) of the target A and a positive value indicating downstream (3′) of the target A;
b) the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c) the name of the structural feature (e.g., symmetric bulge, symmetric internal loop, asymmetric bulge, asymmetric internal loop, mismatch, or wobble base pair), and
d) the sequences of bases on the target RNA side and the engineered guide RNA side that participate in forming the structural feature.

For example, with reference to SEQ ID NO: 39, “−5_6-6_internal_loop-symmetric_UCAGAG-GCAGCU, 0_1-1_mismatch_A-C, 44_6-6_internal loop-symmetric UUUUGU-CUACUC” is read as a structural feature formed in a guide-target RNA scaffold (target DUX4 RNA sequence hybridized to an engineered guide RNA of SEQ ID NO: 39), where a structural feature starts 5 nucleotides upstream (5′) (the −5 position) from the target A (0 position) of the target RNA sequence; six contiguous bases from the target RNA sequence and six contiguous bases from the engineered guide RNA form the structural feature; the structural feature is an internal symmetric loop; and a sequence of UCAGAG from the target RNA side and a sequence of GCAGCU from the engineered guide RNA side participate in forming the internal symmetric loop. A structural feature starts at the target A (0 position) of the target RNA sequence; 1 base from the target RNA and 1 base from the engineered guide RNA form the structural feature; the structural feature is a mismatch; and the sequence of A from the target RNA side and a sequence of C from the engineered guide RNA side participate in forming the mismatch. A structural feature starts 44 nucleotides downstream (3′) (the +44 position) from the target A (0 position) of the target RNA sequence; six contiguous bases from the target RNA sequence and six contiguous bases from the engineered guide RNA form the structural feature; the structural feature is an internal symmetric loop; and a sequence of UUUUGU from the target RNA side and a sequence of CUACUC from the engineered guide RNA side participate in forming the internal symmetric loop.

TABLE 1
DUX4 targeting guide RNA sequences

| | Corresponding Guide | |
|---|---|---|
| Guide DNA Sequence | RNA sequence | Structural Features |
| CTCTGCACTCATCACC | CUCUGCACUCAUCA | −5_6-6_internal_loop- |
| TACTCGATGCAAATCT | CCUACUCGAUGCAA | symmetric_UCAGAG-GCAGCU |
| TCTATAGGATCCACAG | AUCUUCUAUAGGA | 0_1-1_mismatch_A-C |
| GGAGGGGGCATTTTAA | UCCACAGGGAGGGG | 44_6-6_internal_loop- |
| CATATGCAGCTACTAA | GCAUUUUAACAUA | symmetric_UUUUGU-CUACUC |
| TCATCCAGGAGATGTA | UGCAGCUACUAAUC | |
| ACTC (SEQ ID NO: 32) | AUCCAGGAGAUGU | |
| | AACUC (SEQ ID NO: 39) | |
| CTCTGCACTCATCACA | CUCUGCACUCAUCA | −6_6-6_internal_loop- |
| CAAAAGATGCAAATCT | CACAAAAGAUGCAA | symmetric_UUCAGA-AGCUCC |
| GTGGGCGGATCCACA | AUCUGUGGGCGGA | 0_1-1_mismatch_A-C |
| GGGAGGGGGCATTTTA | UCCACAGGGAGGGG | 27_6-6_internal_loop- |
| ACATATCAGCTCCCTA | GCAUUUUAACAUA | symmetric_UAUAGA-GUGGGC |
| ATCATCCAGGAGATGT | UCAGCUCCCUAAUC | |
| AACTC (SEQ ID NO: 33) | AUCCAGGAGAUGU | |
| | AACUC (SEQ ID NO: 40) | |
| CACTCATCACTGCCGT | CACUCAUCACUGCC | −8_6-6_internal_loop- |
| GATGCAAATCTTCTAT | GUGAUGCAAAUCU | symmetric_AGUUCA-GGUUUC |
| AGGATCCACAGGGAG | UCUAUAGGAUCCAC | 0_1-1_mismatch_A-C |
| GGGGCATTTTAACATA | AGGGAGGGGGCAU | 44_6-6_internal_loop- |
| TCTCGGTTTCAATCAT | UUUAACAUAUCUCG | symmetric_UUUUGU-UGCCGU |
| CCAGGAGATGTAACTC | GUUUCAAUCAUCCA | |
| TAATC (SEQ ID NO: 34) | GGAGAUGUAACUC | |
| | UAAUC (SEQ ID NO: 41) | |
| ATCACACAAAAGATCT | AUCACACAAAAGAU | −8_6-6_internal_loop- |
| TCCCCTTCTATAGGAT | CUUCCCCUUCUAUA | symmetric_AGUUCA-ACUCGA |
| CCACAGGGAGGGGGC | GGAUCCACAGGGAG | 3_1-1_mismatch_A-C |
| ATTTCAATATATCTCA | GGGGCAUUUCAAU | 35_6-6_internal_loop- |
| CTCGAAATCATCCAGG | AUAUCUCACUCGAA | symmetric_AUUUGC-CUUCCC |
| AGATGTAACTCTAATC | AUCAUCCAGGAGAU | |
| CAGGT (SEQ ID NO: 35) | GUAACUCUAAUCCA | |
| | GGU (SEQ ID NO: 42) | |
| CACTCATCACACAAAA | CACUCAUCACACAA | −3_6-6_internal_loop- |
| GACCCCTCTCTTCTAT | AAGACCCCUCUCUU | symmetric_AGAGAU-UCAAAC |
| AGGATCCACAGGGAG | CUAUAGGAUCCACA | 4_1-1_mismatch_A-C |
| GGGGCATTCTAATATT | GGGAGGGGGCAUU | 36_6-6_internal_loop- |
| CAAACGAACTAATCAT | CUAAUAUUCAAACG | symmetric_UUUGCA-CCCCUC |
| CCAGGAGATGTAACTC | AACUAAUCAUCCAG | |
| TAATC (SEQ ID NO: 36) | GAGAUGUAACUCU | |
| | AAUC (SEQ ID NO: 43) | |
| ATCACACTCCCTCTGC | AUCACACUCCCUCU | −11_6-6_internal_loop- |
| AAATCTTCTATAGGAT | GCAAAUCUUCUAUA | symmetric_AUUAGU-CCCUCG |
| CCACAGGGAGGGGGC | GGAUCCACAGGGAG | 4_1-1_mismatch_A-C |
| ATTCTAATATATCTCT | GGGGCAUUCUAAU | 42_6-6_internal_loop- |
| GACCCTCGCATCCAGG | AUAUCUCUGACCCU | symmetric_UCUUUU-UCCCUC |
| AGATGTAACTCTAATC | CGCAUCCAGGAGAU | |
| CAGGT (SEQ ID NO: 37) | GUAACUCUAAUCCA | |
| | GGU (SEQ ID NO: 44) | |
| ATCACACAAAAGATG | AUCACACAAAAGAU | −22_6-6_internal_loop- |
| CAAATCTGTGGCTGGA | GCAAAUCUGUGGCU | symmetric_UCUCCU-UACUCU |
| TCCACAGGGAGGGGG | GGAUCCACAGGGAG | −21_1-1_wobble_G-U |
| CATTTTAACGACTCTG | GGGGCAUUUUAAC | −14_1-1_wobble_U-G |
| TGTGAACGATCATCTT | GACUCUGUGUGAAC | −13_1-0_bulge-asymmetric_A- |
| ACTCTTGTAACTCTAA | GAUCAUCUUACUCU | −7_1-3_bulge-asymmetric_G-GUG |
| TCCAGGT (SEQ ID NO: | UGUAACUCUAAUCC | −3−>0_4-4_bulge-symmetric_UAUA- |
| 38) | AGGU (SEQ ID NO: 45) | CGAC |
| | | 27_6-6_internal_loop- |
| | | symmetric_UAUAGA-GUGGCU |
| CTCTGCACTCATCACA | CUCUGCACUCAUCA | −22_6-6_internal_loop- |
| CAAAAGACCCCTCTCT | CACAAAAGACCCCU | symmetric_UCUCCU-UACUCU |
| TCTATAGGATCCACAG | CUCUUCUAUAGGAU | −21_1-1_wobble_G-U |
| GGAGGGGGCATTTTAA | CCACAGGGAGGGGG | −14_1-1_wobble_U-G |
| CGACTCTGTGTGAACG | CAUUUUAACGACUC | −13_1-0_bulge-asymmetric_A- |
| ATCATCTTACTCTTGT | UGUGUGAACGAUC | −7_1-3_bulge-asymmetric_G-GUG |
| AACTC (SEQ ID NO: 79) | AUCUUACUCUUGUA | −3−>0_4-4_bulge-symmetric_UAUA- |
| | ACUC (SEQ ID NO: 46) | CGAC |
| | | 36_6-6_internal_loop- |
| | | symmetric_UUUGCA-CCCCUC |
| CTCTGCACTCATCACT | CUCUGCACUCAUCA | −8_6-6_internal_loop- |
| GCCGTGATGCAAATCT | CUGCCGUGAUGCAA | symmetric_AGUUCA-GGUUUC |
| TCTATAGGATCCACAG | AUCUUCUAUAGGA | 0_1-1_mismatch_A-C |
| GGAGGGGGCATTTTAA | UCCACAGGGAGGGG | 44_6-6_internal_loop- |
| CATATCTCGGTTTCAA | GCAUUUUAACAUA | symmetric_UUUUGU-UGCCGU |
| TCATCCAGGAGATGTA | UCUCGGUUUCAAUC | |
| ACTC (SEQ ID NO: 57) | AUCCAGGAGAUGU | |
| | AACUC (SEQ ID NO: 93) | |
| CTCTGCACTCATCACT | CUCUGCACUCAUCA | −27_1-1_wobble_U-G |
| AATCTGATGCAGATCT | CUAAUCUGAUGCAG | −22_1-1_wobble_U-G |
| TCTATAGGGTCTACAG | AUCUUCUAUAGGG | −14_1-1_wobble_U-G |
| GGAGGGGGCATTTTAA | UCUACAGGGAGGG | −5_6-6_internal_loop- |
| CATATATAATCACTGA | GGCAUUUUAACAU | symmetric_UCAGAG-AUAAUC |
| TCATCCGGGAGGTGTA | AUAUAAUCACUGA | 0_1-1_mismatch_A-C |
| ACTC (SEQ ID NO: 180) | UCAUCCGGGAGGUG | 21_1-1_wobble_G-U |
| | UAACUC (SEQ ID NO: | 24_1-1_wobble_U-G |
| | 212) | 37_1-1_wobble_U-G |
| | | 44_6-6_internal_loop- |
| | | symmetric_UUUUGU-UAAUCU |
| CTCTGTACTTATCACG | CUCUGUACUUAUCA | −35_1-1_wobble_G-U |
| CAAGAGATGCAGATCT | CGCAAGAGAUGCAG | −32_1-1_wobble_U-G |
| AACCATGGATTCGCAG | AUCUAACCAUGGAU | −27_1-1_wobble_U-G |
| GGAGGGGGTATTTTAA | UCGCAGGGAGGGG | −20_1-1_wobble_G-U |
| CATATCAATATTCTAG | GUAUUUUAACAUA | −18_1-1_wobble_U-G |
| TTGTTCAGGAGGTGTA | UCAAUAUUCUAGU | −17_1-1_wobble_G-U |
| GCTT (SEQ ID NO: 196) | UGUUCAGGAGGUG | −15_1-1_wobble_U-G |
| | UAGCUU (SEQ ID | −6_6-6_internal_loop- |
| | NO: 228) | symmetric_UUCAGA-AAUAUU |
| | | 0_1-1_mismatch_A-C |
| | | 8_1-1_wobble_G-U |
| | | 20_1-1_wobble_U-G |
| | | 22_1-1_wobble_G-U |
| | | 27_6-6_internal_loop- |
| | | symmetric_UAUAGA-AACCAU |
| | | 37_1-1_wobble_U-G |
| | | 45_1-1_wobble_U-G |
| | | 49_1-1_wobble_U-G |
| | | 55_1-1_wobble_G-U |
| | | 59_1-1_wobble_G-U |
| TTTTGCGCTTATCGCA | UUUUGCGCUUAUCG | −35_1-1__wobble_G-U |
| TAAGAGAAGATTTTTT | CAUAAGAGAAGAU | −33_1-1_wobble_G-U |
| TTTGTAGGGTTCGCGG | UUUUUUUUGUAGG | −31_1-1_wobble_U-G |
| GGAGGGGGCATTTTAA | GUUCGCGGGGAGG | −22_6-6_internal_loop- |
| CGACTCTGTGTGAACG | GGGCAUUUUAACG | symmetric_UCUCCU-UUUCCC |
| ATCATCTTTTCCCTGT | ACUCUGUGUGAACG | −21_1-1_wobble_G-U |
| GATTT (SEQ ID NO: | AUCAUCUUUUCCCU | −14_1-1_wobble_U-G |
| 207) | GUGAUUU (SEQ ID | −13_1-0_bulge-asymmetric_A- |
| | NO: 239) | −7_1-3_bulge-asymmetric_G-GUG |
| | | −3−>0_4-4_bulge-symmetric_UAUA- |
| | | CGAC |
| | | 18_1-1_wobble_U-G |
| | | 20_1-1_wobble_U-G |
| | | 22_1-1_wobble_G-U |
| | | 24_1-1_wobble_U-G |
| | | 29_1-1_wobble_U-G |
| | | 31_1-1_wobble_G-U |
| | | 34_1-1_wobble_G-U |
| | | 36_4-4_bulge-symmetric_UUUG- |
| | | AUUU |
| | | 41_1-1_mismatch_A-A |
| | | 45_1-1_wobble_U-G |
| | | 48_1-1_wobble_G-U |
| | | 51_1-1_wobble_U-G |
| | | 55_wobble_G-U |
| | | 58_1-1_wobble_U-G |
| | | 62_1-1_wobble_G-U |
| | | 64_1-1_wobble_G-U |

The present disclosure provides engineered guide RNAs (e.g., SEQ ID NO: 39, SEQ ID NO: 40, SEQ ID NO: 42, SEQ ID NO: 44, SEQ ID NO: 46, SEQ ID NO: 93, SEQ ID NO: 228, or SEQ ID NO: 239) that target a sequence of an DUX4 target RNA (for example, the polyA signal sequence of DUX4-FL mRNA).

In some embodiments, an engineered guide RNA of the present disclosure that targets the polyA signal sequence of DUX4 comprises one or more structural features.

In some embodiments, a first 6/6 symmetric internal loop is at position −5, relative to the target adenosine at position 0. In some embodiments, the one or more structural features further comprises at least one structural feature selected from the group consisting of: a 1-1 mismatch at position 0 relative to the target adenosine at position 0, a second 6/6 symmetric internal loop at position 44 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 39. In some embodiments, a polynucleotide encoding the engineered guide RNA comprises at least about: 80%, 85%, 90%, 92%, 95%, 97%, 99%, or 100% sequence identity to SEQ ID NO: 32.

In some embodiments, a first 6/6 symmetric internal loop is at position −6, relative to the target adenosine at position 0. In some embodiments, the one or more structural features further comprises at least one structural feature selected from the group consisting of: a 1-1 mismatch at position 0 relative to the target adenosine at position 0, a second 6/6 symmetric internal loop at position 27 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 40. In some embodiments, a polynucleotide encoding the engineered guide RNA comprises at least about: 80%, 85%, 90%, 92%, 95%, 97%, 99%, or 100% sequence identity to SEQ ID NO: 33.

In some embodiments, a first 6/6 symmetric internal loop is at position −6, relative to the target adenosine at position 0. In some embodiments, the one or more structural features further comprises at least one structural feature selected from the group consisting of: a wobble base at position −35 relative to the target adenosine at position 0, a wobble base at position −32 relative to the target adenosine at position 0, a wobble base at position −27 relative to the target adenosine at position 0, a wobble base at position −20 relative to the target adenosine at position 0, a wobble base at position −18 relative to the target adenosine at position 0, a wobble base at position −17 relative to the target adenosine at position 0, a wobble base at position −15 relative to the target adenosine at position 0, a wobble base at position 8 relative to the target adenosine at position 0, a wobble base at position 20 relative to the target adenosine at position 0, a wobble base at position 22 relative to the target adenosine at position 0, a wobble base at position 37 relative to the target adenosine at position 0, a wobble base at position 45 relative to the target adenosine at position 0, a wobble base at position 49 relative to the target adenosine at position 0, a wobble base at position 55 relative to the target adenosine at position 0, a wobble base at position 59 relative to the target adenosine at position 0, a 1-1 mismatch at position 0 relative to the target adenosine at position 0, a second 6/6 symmetric internal loop at position 27 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 228. In some embodiments, a polynucleotide encoding the engineered guide RNA comprises at least about: 80%, 85%, 90%, 92%, 95%, 97%, 99%, or 100% sequence identity to SEQ ID NO: 196.

In some embodiments, a first 6/6 symmetric internal loop is at position −22, relative to the target adenosine at position 0. In some embodiments, the one or more structural features further comprises at least one structural feature selected from the group consisting of a wobble base at position −21 relative to the target adenosine at position 0, a wobble base at position −14 relative to the target adenosine at position 0, a 1-0 asymmetric bulge at position −13 relative to the target adenosine at position 0, a 1-3 asymmetric bulge at position −7 relative to the target adenosine at position 0, a 4-4 symmetric bulge at position −3 relative to the target adenosine at position 0, a second 6/6 symmetric internal loop at position 36 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 46. In some embodiments, a polynucleotide encoding the engineered guide RNA comprises at least about: 80%, 85%, 90%, 92%, 95%, 97%, 99%, or 100% sequence identity to SEQ ID NO: 79.

In some embodiments, a 6/6 symmetric internal loop is at position −22, relative to the target adenosine at position 0. In some embodiments, the one or more structural features further comprises at least one structural feature selected from the group consisting of a wobble base at position −35 relative to the target adenosine at position 0, a wobble base at position −33 relative to the target adenosine at position 0, a wobble base at position −31 relative to the target adenosine at position 0, a wobble base at position −21 relative to the target adenosine at position 0, a wobble base at position −14 relative to the target adenosine at position 0, a wobble base at position 18 relative to the target adenosine at position 0, a wobble base at position 20 relative to the target adenosine at position 0, a wobble base at position 22 relative to the target adenosine at position 0, a wobble base at position 24 relative to the target adenosine at position 0, a wobble base at position 29 relative to the target adenosine at position 0, a wobble base at position 31 relative to the target adenosine at position 0, a wobble base at position 34 relative to the target adenosine at position 0, a 1-1 mismatch at position 41 relative to the target adenosine at position 0, a wobble base at position 45 relative to the target adenosine at position 0, a wobble base at position 48 relative to the target adenosine at position 0, a wobble base at position 51 relative to the target adenosine at position 0, a wobble base at position 55 relative to the target adenosine at position 0, a wobble base at position 58 relative to the target adenosine at position 0, a wobble base at position 62 relative to the target adenosine at position 0, a wobble base at position 64 relative to the target adenosine at position 0, a 1-0 asymmetric bulge at position −13 relative to the target adenosine at position 0, a 1-3 asymmetric bulge at position −7 relative to the target adenosine at position 0, a 4/4 symmetric bulge at position −3 relative to the target adenosine at position 0, a 4/4 symmetric internal loop at position 36 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 239. In some embodiments, a polynucleotide encoding the engineered guide RNA comprises at least about: 80%, 85%, 90%, 92%, 95%, 97%, 99%, or 100% sequence identity to SEQ ID NO: 207.

In some embodiments, a first 6/6 symmetric internal loop is at position −8, relative to the target adenosine at position 0. In some embodiments, the one or more structural features further comprises at least one structural feature selected from the group consisting of a 1-1 mismatch at position 3 relative to the target adenosine at position 0, a second 6/6 symmetric internal loop at position 35 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 42. In some embodiments, a polynucleotide encoding the engineered guide RNA comprises at least about: 80%, 85%, 90%, 92%, 95%, 97%, 99%, or 100% sequence identity to SEQ ID NO: 35.

In some embodiments, a first 6/6 symmetric internal loop is at position −8, relative to the target adenosine at position 0. In some embodiments, the one or more structural features further comprises at least one structural feature selected from the group consisting of a 1-1 mismatch at position 0 relative to the target adenosine at position 0, a second 6/6 symmetric internal loop at position 44 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 93. In some embodiments, a polynucleotide encoding the engineered guide RNA comprises at least about: 80%, 85%, 90%, 92%, 95%, 97%, 99%, or 100% sequence identity to SEQ ID NO: 57.

In some embodiments, a first 6/6 symmetric internal loop is at position −11, relative to the target adenosine at position 0. In some embodiments, the one or more structural features further comprises at least one structural feature selected from the group consisting of: a 1-1 mismatch at position 4 relative to the target adenosine at position 0, a second 6/6 symmetric internal loop at position 42 relative to the target adenosine at position 0, and any combination thereof. In some embodiments, the engineered guide RNA comprises at least about: 80%, 85%, 90%, 92%, 95%, 97%, 99%, or 100% sequence identity to SEQ ID NO: 44. In some embodiments, a polynucleotide encoding the engineered guide RNA comprises at least about: 80%, 85%, 90%, 92%, 95%, 97%, 99%, or 100% sequence identity to SEQ ID NO: 37.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 32. In some cases, an engineered guide RNA herein when hybridized to the target RNA can comprise a 6/6 symmetric internal loop at position −5 relative to the target adenosine at position 0, an A/C mismatch at the target adenosine (position 0), and a 6/6 symmetric internal loop at position 44 relative to the target adenosine at position 0.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 33. In some cases, an engineered guide RNA when hybridized to the target RNA herein can comprise a 6/6 symmetric internal loop at position −6 relative to the target adenosine at position 0, an A/C mismatch at the target adenosine (position 0), and a 6/6 symmetric internal loop at position 27 relative to the target adenosine at position 0.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 46. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 79. In some cases, an engineered guide RNA when hybridized to the target RNA herein can comprise a 6/6 symmetric internal loop at position −22 relative to the target adenosine at position 0, a wobble base pair at the −21 and −14 positions relative to the target adenosine at position 0, a I/O asymmetric bulge at position −13 relative to the target adenosine at position 0, a 1/3 asymmetric bulge at position −7 relative to the target adenosine at position 0, a 4/4 symmetric bulge at position −3 relative to the target adenosine at position 0, and a 6/6 symmetric internal loop at position 36 relative to the target adenosine at position 0.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 212. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 180. In some cases, an engineered guide RNA when hybridized to the target RNA herein can comprise a 6/6 symmetric internal loop at position −5 relative to the target adenosine at position 0, a wobble base pair at the −27, −22, −14, 21, 24, and 37 positions relative to the target adenosine at position 0, an A/C mismatch at the target adenosine (position 0), and a 6/6 symmetric internal loop at position 44 relative to the target adenosine at position 0.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 228. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 196. In some cases, an engineered guide RNA when hybridized to the target RNA herein can comprise a 6/6 symmetric internal loop at position −6 relative to the target adenosine at position 0, a wobble base pair at the −35, −32, −27, −20, −18, −17, −15, 8, 20, 22, 37, 45, 49, 55, and 59 positions relative to the target adenosine at position 0, an A/C mismatch at the target adenosine (position 0), and a 6/6 symmetric internal loop at position 27 relative to the target adenosine at position 0.

In some cases, an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 239. In some cases, polynucleotide encoding an engineered guide RNA can comprise a sequence with at least: 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% sequence identity to SEQ ID NO: 207. In some cases, an engineered guide RNA when hybridized to the target RNA herein can comprise a 6/6 symmetric internal loop at position −22 relative to the target adenosine at position 0, a wobble base pair at the −35, −33, −31, −21, −14, 18, 20, 22, 24, 29, 31, 34, 45, 48, 51, 55, 58, 62 and 64 positions relative to the target adenosine at position 0, a 1-1 mismatch at position 41 relative to the target adenosine at position 0, 1/0 asymmetric bulge at position −13 relative to the target adenosine at position 0, a 1/3 asymmetric bulge at position −7 relative to the target adenosine at position 0, a 4/4 symmetric bulge at position −3 relative to the target adenosine at position 0, and a 4/4 symmetric bulge at position 36 relative to the target adenosine at position 0.

In some embodiments, hybridization of a targeting domain of an engineered guide RNA to a target DUX4 mRNA results in mRNA knockdown, protein knockdown, or both. For example, hybridization of a targeting domain of an engineered guide RNA to a sequence of a target DUX4 mRNA containing a polyA signal sequence can result in degradation and/or silencing of the target DUX4 mRNA. This degradation and/or silencing can occur due to, for example, where the hybridization of the targeting domain to the target DUX4 mRNA results in ADAR-mediated editing of an adenosine of the polyA signal sequence, thus converting tan “A” in the polyA signal sequence to a “G” and silencing expression of DUX4 mRNA. Alternatively or in addition, degradation and/or silencing can occur due to masking of a polyA signal sequence of a target DUX4 mRNA by the engineered guide RNA. In some cases, masking of the polyA signal sequence can facilitate destabilization of DUX4-FL mRNA. In some embodiments, hybridization of a targeting domain of an engineered guide RNA to a sequence of a target DUX4 mRNA containing a polyA signal sequence can result in expression knockdown of a gene downstream of DUX4 (e.g., SLC34A2, LEUTX, ZSCAN4, PRAMEF12, TRIM43, KHDC1L, DEFB103, Wfdc3, Agtr2, MBD3L2, or combinations thereof). For example, hybridization of a targeting domain of an engineered guide RNA to a sequence of a target DUX4 mRNA containing a polyA signal sequence can result in knockdown of a mRNA, a protein, or both encoded by SLC34A2, LEUTX, ZSCAN4, PRAMEF12, TRIM43, KHDC1L, DEFB103, Wfdc3, Agtr2, or MBD3L2

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can be configured to facilitate editing of a base of a nucleotide of a polynucleotide of a region of a subject target RNA, modulation expression of a polypeptide encoded by the subject target RNA, or both. In some cases, an engineered guide can be configured to facilitate an editing of a base of a nucleotide or polynucleotide of a region of an RNA by a subject RNA editing entity. In order to facilitate editing, an engineered guide RNA of the disclosure can recruit an RNA editing entity. Various RNA editing entity recruiting domains can be utilized. In some examples, a recruiting domain comprises: Glutamate ionotropic receptor AMPA type subunit 2 (GluR2), APOBEC, or Alu.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence, for example an antisense sequence, hybridizes to a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In an embodiment, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 2). In some cases, a recruiting domain can comprise at least about 80% sequence homology to at least about 10, 15, 20, 25, or 30 nucleotides of SEQ ID NO: 2. In some examples, a recruiting domain can comprise at least about 90%, 95%, 96%, 97%, 98%, or 99% sequence homology and/or length to SEQ ID NO: 2.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of subject guides. In some cases, a recruiting domain can be on an N-terminus, middle, or C-terminus of a polynucleotide. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribonucleotides and deoxyribonucleotides can in some cases not be excluded.

C. Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked.

A double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are structural features that can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

FIG. 2 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence (e.g., recruiting region) can be absent, an engineered guide RNA can be still capable of associating with a subject RNA editing entity (e.g., ADAR) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A double stranded RNA (dsRNA) substrate (i.e., a guide-target RNA scaffold) is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch in a guide RNA is to a G, a C, or a U in the DUX4 target RNA. For example, a G in the DUX4 target RNA can mismatch with a G, an A or a U in the guide RNA. In another example, a C in the DUX4 target RNA can mismatch with a C, an A, or a U in the guide RNA. In another example, a U in the DUX4 target RNA can mismatch with a U, a G, or a C in the guide RNA. In some embodiments, a mismatch in a guide RNA is to an A in the DUX4 target RNA. For example, an A in the DUX4 target RNA can mismatch with an A, a G, or a C in the guide RNA. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a guide RNA of the present disclosure may not have an A/C mismatch and each A of the target RNA is base paired to a U in the engineered guide RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

A recruitment hairpin, as disclosed herein, can recruit at least in part an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A double stranded RNA (dsRNA) substrate (i.e., a guide-target RNA scaffold) is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. The nucleotides in a bulge of the guide RNA can comprise any nucleotide, in any order so long as they are not complementary to their positional counterparts on the target RNA. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A double stranded RNA (dsRNA) substrate (i.e., a guide-target RNA scaffold) is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. The nucleotides in an internal loop of the guide RNA can comprise any nucleotide, in any order so long as they are not complementary to their positional counterparts on the target RNA. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 11 nucleotides on the engineered guide RNA side of the guide-target RNA scaffold target and 11 nucleotides on the target RNA side of the guide-target RNA scaffold. A symmetrical internal loop of the present disclosure can be formed by 12 nucleotides on the engineered guide RNA side of the guide-target RNA scaffold target and 12 nucleotides on the target RNA side of the guide-target RNA scaffold. A symmetrical internal loop of the present disclosure can be formed by 13 nucleotides on the engineered guide RNA side of the guide-target RNA scaffold target and 13 nucleotides on the target RNA side of the guide-target RNA scaffold. A symmetrical internal loop of the present disclosure can be formed by 14 nucleotides on the engineered guide RNA side of the guide-target RNA scaffold target and 14 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

In some embodiments, a symmetrical internal loop can be positioned upstream (5′) of the target A (0 position), downstream (3′) of the target A, or both. In some embodiments, when referring to a location of a structural feature a “−” or negative integer indicates a nucleotide upstream (5′) of the target A or of a specified position (e.g., position 0 ATTAAA), while a positive integer indicates a nucleotide downstream (3′) of the target A, or of a specified position. In some instances, a first symmetrical internal loop can be downstream of the target A and a second symmetrical internal loop can be upstream of the target A. In some cases, a symmetric internal loop can be from position: −1 to −25, −2 to −10, −4 to −8, −5 to −7, −2 to −15, −4 to −20, −8 to −15, or −10 to −22 relative to the target A. In some cases, a symmetric internal loop can be located at position: −25, −24, −23, −22, −21, −20, −19, −18, −17, −16, −15, −14, −13, −12, −11, −10, −9, −8, −7, −6, −5, −4, −3, −2, or −1 relative to the target A. In some cases, a symmetric internal loop can be from position: +1 to +60, +10 to +50, +10 to +40, +20 to +50, +20 to +40, +25 to +45, +31 to +35, +10 to +20, +15 to +30, +25 to +45, or +45 to +60 relative to the target A. In some cases, a symmetric internal loop can be located at position: 1, +2, +3, +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, or +60 relative to the target A. In some cases, a first symmetric internal loop within about: 80 bp, 70 bp, 60 bp, 50 bp, 40 bp, 30 bp, 25 bp, 20 bp, 15 bp, 10 bp, or 5 bp of the 5′ end of the guide RNA, and a second symmetric internal loop within about: 80 bp, 70 bp, 60 bp, 50 bp, 40 bp, 30 bp, 25 bp, 20 bp, 15 bp, 10 bp, or 5 bp of the 3′ end of the guide RNA.

A double stranded RNA (dsRNA) substrate (i.e., a guide-target RNA scaffold) is formed upon hybridization of an engineered guide RNA of the present disclosure to a target RNA. An internal loop can be a symmetrical internal loop or an asymmetrical internal loop. An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a base paired (bp) region refers to a region of the guide-target RNA scaffold in which bases in the guide RNA (e.g., the bases in the targeting sequence of the guide RNA) are paired with opposing bases in the target polynucleotide. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 to 50, 1 to 75, 1 to 100, 1 to 125, 1 to 150, 1 to 175, 1 to 200, 1 to 225, 1 to 250, 1 to 275, 1 to 300, 50 to 75, 50 to 100, 50 to 125, 50 to 150, 50 to 175, 50 to 200, 50 to 225, 50 to 250, 50 to 275, 50 to 300, 60 to 75, 60 to 100, 60 to 125, 60 to 150, 60 to 175, 60 to 200, 60 to 225, 60 to 250, 60 to 275, 60 to 300, 70 to 100, 70 to 125, 70 to 150, 70 to 175, 70 to 200, 70 to 225, 70 to 250, 70 to 275, 70 to 300, 80 to 100, 80 to 125, 80 to 150, 80 to 175, 80 to 200, 80 to 225, 80 to 250, 80 to 275, 80 to 300, 90 to 125, 90 to 150, 90 to 175, 90 to 200, 90 to 225, 90 to 250, 90 to 275, 90 to 300, 100 to 125, 100 to 150, 100 to 175, 100 to 200, 100 to 225, 100 to 250, 100 to 275, 100 to 300, 150 to 200, 150 to 225, 150 to 250, 150 to 275, or 150 to 300 base pairs. In some embodiments, a base paired region has at least 1, 2, 3, 4, 5, 6, 7, 8, 9, 10, 12, 14, 16, 18, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 base pairs.

D. Guides with Macro-Footprints

Guide RNAs of the present disclosure can further comprise a macro-footprint. In some embodiments, the macro-footprint comprises a barbell macro-footprint. A macro-footprint can serve to guide an RNA editing enzyme and direct its activity towards the target adenosine to be edited. A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint. In some cases, barbell internal loops can comprise at least one structural feature that facilitates editing of a specific target RNA.

As described herein, a “micro-footprint” sequence refers to a sequence with latent structures that, when manifested, facilitate editing of the adenosine of a target RNA via an adenosine deaminase enzyme. A macro-footprint can serve to guide an or focus RNA editing entity (e.g., ADAR) and direct its activity towards a micro-footprint. In some embodiments, included within the micro-footprint sequence is a nucleotide that is positioned such that, when the guide RNA is hybridized to the target RNA, said nucleotide is opposite the adenosine to be edited by the ADAR enzyme and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

In some embodiments, the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the increase in the one or more aspects of editing provided by the barbell macro-footprint structures can be independent of the particular target RNA. For example, macro-footprints (e.g., barbell macro-footprints) and micro-footprints can provide an increased amount of on target adenosine editing relative to an otherwise comparable guide RNA lacking the barbells. In other embodiments, the presence of the barbell macro-footprint in addition to the micro-footprint described here can result in a lower amount of local off-target adenosine editing, relative to an otherwise comparable guide RNA, upon hybridization of the guide RNA and target RNA to form a guide-target RNA scaffold lacking the barbells.

A dumbbell design in an engineered guide RNA comprises two symmetrical internal loops, wherein the target A to be edited is positioned between the two symmetrical loops for selective editing of the target A. The two symmetrical internal loops are each formed by 6 nucleotides on the guide RNA side of the guide-target RNA scaffold and 6 nucleotides on the target RNA side of the guide-target RNA scaffold. Thus, a dumbbell can be a structural feature formed from latent structure provided by an engineered latent guide RNA.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, a first internal loop or a second internal loop independently comprises a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, provided herein are engineered guide RNAs comprising a barbell macro-footprint. In some embodiments, provided herein are engineered guide RNAs comprising a micro-footprint. In some embodiments, provided herein are engineered guide RNAs comprising a macro-footprint and a micro-footprint. In some cases, an engineered guide RNA disclosed herein can comprise a micro-footprint in the absence of a macro-footprint. In some cases, an engineered guide RNA disclosed herein can comprise a macro-footprint in the absence of a micro-footprint.

In some embodiments, a macro-footprint sequence can comprise a barbell macro-footprint sequence comprising latent structures that, when manifested, produce a first internal loop and a second internal loop.

In some examples, a first internal loop is positioned near the 5′ end of the guide-target RNA scaffold and a second internal loop is positioned near the 3′ end of the guide-target RNA scaffold. The length of the dsRNA comprises a 5′ end and a 3′ end, where up to half of the length of the guide-target RNA scaffold at the 5′ end can be considered to be “near the 5′ end” while up to half of the length of the guide-target RNA scaffold at the 3′ end can be considered “near the 3′ end.” Non-limiting examples of the 5′ end can include about 50% or less of the total length of the dsRNA at the 5′ end, about 45%, about 40%, about 35%, about 30%, about 25%, about 20%, about 15%, about 10%, or about 5%. Non-limiting examples of the 3′ end can include about 50% or less of the total length of the dsRNA at the 3′ end about 45%, about 40%, about 35%, about 30%, about 25%, about 20%, about 15%, about 10%, or about 5%.

In some embodiments, the engineered guide RNAs of the disclosure comprising a barbell macro-footprint sequence (that manifests as a first internal loop and a second internal loop) can improve RNA editing efficiency, increase the amount or percentage of RNA editing generally, as well as for on-target nucleotide editing, such as on-target adenosine. In some embodiments, the engineered guide RNAs of the disclosure comprising a first internal loop and a second internal loop can also facilitate a decrease in the amount of or reduce off-target nucleotide editing, such as off-target adenosine or unintended adenosine editing. The decrease or reduction in some examples can be of the number of off-target edits or the percentage of off-target edits.

Each of the first and second internal loops of the barbell macro-footprint can independently be symmetrical or asymmetrical, where symmetry is determined by the number of bases or nucleotides of the engineered guide RNA and the number of bases or nucleotides of the target RNA, that together form each of the first and second internal loops.

### E. Engineered Polynucleotides Encoding Engineered Guide RNAs

An engineered polynucleotide as described herein can comprise one or more polynucleotide sequence(s) that encode one or more engineered guide RNA(s). For example, an engineered polynucleotide can comprise 1, 2, 3, 4, or more than 4 polynucleotide sequence(s) that encode 1, 2, 3, 4, or more than 4 engineered guide RNAs.

In some instances, the engineered polynucleotide can comprise one or more polynucleotide sequence(s) encoding one or more engineered guide RNA(s) that independently hybridize to (target): (1) different target sequences of the same target RNA, or (2) different target sequences of different target RNAs. For example, a first engineered guide RNA encoded by a first polynucleotide sequence can hybridize to a target sequence of a first target RNA while a second engineered guide RNA encoded by a second polynucleotide sequence can hybridize to a target sequence of a second target RNA, in some instances resulting in ADAR-mediated editing of an adenosine in the target sequence of the first target RNA and an adenosine in the target sequence of the second target RNA.

In some instances, the engineered polynucleotide can comprise one or more polynucleotide sequence(s) encoding one or more engineered guide RNA(s) that independently hybridize to (target) the same target sequence of a target RNA. For example, the one or more engineered guide RNA(s) encoded by the one or more polynucleotide sequence(s) can each independently hybridize to a target sequence of a target RNA and/or facilitate editing of the same adenosine in the target sequence of the target RNA via ADAR. In some cases, the one or more engineered guide RNA(s) that hybridize to (target) the same target sequence of a target RNA have identical sequences (i.e., the one or more engineered guide RNAs are copies of each other).

Alternatively, two or more engineered guide RNA(s) that hybridize to (target) the same target sequence of a target RNA can comprise different sequences. For example, a first engineered guide RNA encoded by an engineered polynucleotide can have a sequence identity of less than, greater than, or equal to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a second engineered guide RNA encoded by the engineered polynucleotide, where the second engineered guide RNA hybridizes to (targets) the same target sequence of a target RNA as the first engineered guide RNA. In some instances, a first engineered guide RNA encoded by an engineered polynucleotide can have at least about 70% to about 99% sequence identity, at least about 60% to about 99% sequence identity, at least about 80% to about 99% sequence identity, at least about 60% to about 70% sequence identity, at least about 70% to about 80% sequence identity, at least about 75% to about 85% sequence identity, at least about 85% to about 99% sequence identity, at least about 85% to about 90% sequence identity, at least about 88% to about 93% sequence identity, at least about 90% to about 95% sequence identity, at least about 92% to about 99% sequence identity, or at least about 95% to about 99% sequence identity to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 60% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 61% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 62% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 63% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 64% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 65% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 66% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 67% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 68% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 69% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 70% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 71% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 72% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 73% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 74% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 75% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 76% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 77% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 78% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 79% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 80% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 81% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 82% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 83% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 84% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 85% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 86% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 87%, to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 88% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 89% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 90% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 91% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 92% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 93% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 94% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 95% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 96% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 97% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 98% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 99% to a second engineered guide RNA encoded by the engineered polynucleotide, where the second engineered guide RNA hybridizes to (targets) the same target sequence of a target RNA as the first engineered guide RNA. In some embodiments, polynucleotides encoding a first engineered guide RNA, a second engineered guide RNA, or both can be delivered via an AAV. In some instances, the AAV can be formulated in a composition, such as any of the pharmaceutical compositions disclosed herein.

### F. Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. For example, PCT/US2021/034301 provides a description of circular guide RNAs and their structures, sequences of circular guide RNAs, and methods of engineering circularized polynucleotide domains, and each of these descriptions in PCT/US2021/034301 is herein incorporated by reference.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g. increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g., a Schistosoma mansoni ribozyme), a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g., from a precursor engineered polypeptide) after transcription in a cell. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme. A ribozyme can comprise 5′ GCCATCAGTCGCCGGTCCCAAGCCCGGATAAAATGGGAGGGGGCGGGAAACCGCCT 3′ (SEQ ID NO: 3). A ribozyme can comprise 5′ GCCAUCAGUCGCCGGUCCCAAGCCCGGAUAAAAUGGGAGGGGGCGGGAAACCGCC U 3′ (SEQ ID NO: 4). A ribozyme can comprise at least about: 70%, 75%, 80%, 85%, 90%, 95%, or 100% sequence homology to 5′ GCCATCAGTCGCCGGTCCCAAGCCCGGATAAAATGGGAGGGGGCGGGAAACCGCCT 3′ (SEQ ID NO: 3). A ribozyme can comprise at least about: 70%, 75%, 80%, 85%, 90%, 95%, or 100% sequence homology to 5′ GCCAUCAGUCGCCGGUCCCAAGCCCGGAUAAAAUGGGAGGGGGCGGGAAACCGCC U 3′ (SEQ ID NO: 4). A ribozyme can include a P1 Twister Ribozyme. A ribozyme can include 5′ AACACTGCCAATGCCGGTCCCAAGCCCGGATAAAAGTGGAGGGTACAGTCCACGC 3′ (SEQ ID NO: 5). A ribozyme can include 5′ AACACUGCCAAUGCCGGUCCCAAGCCCGGAUAAAAGUGGAGGGUACAGUCCACGC 3′ (SEQ ID NO: 6). A ribozyme can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACGC 3′ (SEQ ID NO: 5). A ribozyme can comprise at least about: 70%, 75%, 80%, 85%, 90%, 95%, or 100% sequence homology to 5′ AACACUGCCAAUGCCGGUCCCAAGCCCGGAUAAAAGUGGAGGGUACAGUCCACGC 3′ (SEQ ID NO: 6).

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain. In some cases, a ligation domain can comprise 5′ AACCATGCCGACTGATGGCAG 3′ (SEQ ID NO: 7). In some embodiments, a ligation domain can comprise 5′ GATGTCAGGTGCGGCTGACTACCGTC 3′ (SEQ ID NO: 8). In some cases, a ligation domain can comprise 5′ AACCAUGCCGACUGAUGGCAG 3′ (SEQ ID NO: 9). In some cases, a ligation domain can comprise 5′ GAUGUCAGGUGCGGCUGACUACCGUC 3′ (SEQ ID NO: 10). In some cases, a ligation domain can comprise at least about: 70%, 75%, 80%, 85%, 90%, 95%, or 100% sequence homology to 5′ AACCATGCCGACTGATGGCAG 3′ (SEQ ID NO: 7). In some cases, a ligation domain can comprise at least about: 70%, 75%, 80%, 85%, 90%, 95%, or 100% sequence homology to 5′ GATGTCAGGTGCGGCTGACTACCGTC 3′ (SEQ ID NO: 8). In some cases, a ligation domain can comprise at least about: 70%, 75%, 80%, 85%, 90%, 95%, or 100% sequence homology to 5′ AACCAUGCCGACUGAUGGCAG 3′ (SEQ ID NO: 9). In some cases, a ligation domain can comprise at least about: 70%, 75%, 80%, 85%, 90%, 95%, or 100% sequence homology to 5′ GAUGUCAGGUGCGGCUGACUACCGUC 3′ (SEQ ID NO: 10).

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features. Promoters and snRNA components are described in PCT/US2021/028618 and PCT/US2022/078801, and each of these descriptions in PCT/US2021/028618 and PCT/US2022/078801 are herein incorporated by reference.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 19) or RNA: UAGGCUUUCUGGCUUUUUACCGGAAAGCCCCU (SEQ ID NO: 20). In some cases, a mouse U7 hairpin sequence comprises CAGGTTTTCTGACTTCGGTCGGAAAACCCCT (SEQ ID NO: 21) or RNA: CAGGUUUUCUGACUUCGGUCGGAAAACCCCU (SEQ ID NO: 22). In some embodiments, the SmOPT sequence has a sequence of AATTTTTGGAG (SEQ ID NO: 23) or RNA: AAUUUUUGGAG (SEQ ID NO: 24). In some embodiments, a guide RNA can comprise a guide RNA comprising a U7 hairpin sequence (e.g., a human or a mouse U7 hairpin sequence), an SmOPT sequence, or a combination thereof. In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

Also disclosed herein are promoters for driving the expression of a guide RNA disclosed herein. In some cases, the promoters for driving expression can be 5′ to the guide RNA sequence disclosed herein. In some cases, a promoter can comprise a U1 promoter, a U7 promoter, a U6 promoter or any combination thereof. In some cases, a promoter can comprise a CMV promoter. In some cases, a U7 promoter, or a U6 promoter can be a mouse U7 promoter, or a mouse U6 promoter. In some cases, a U1 promoter, a U7 promoter, or a U6 promoter can be a human U1 promoter, a human U7 promoter, or a human U6 promoter. In some cases, a human U6 promoter can comprise a sequence with at least about: 70%, 75%, 80%, 90%, 91%, 92%, 93%, 94%, 95%, 96%, 97%, 98%, 99% or 100% sequence identity to: GAGGGCCTATTTCCCATGATTCCTTCATATTTGCATATACGATACAAGGCTGTTAGA GAGATAATTAGAATTAATTTGACTGTAAACACAAAGATATTAGTACAAAATACGTG ACGTAGAAAGTAATAATTTCTTGGGTAGTTTGCAGTTTTAAAATTATGTTTTAAAATG GACTATCATATGCTTACCGTAACTTGAAAGTATTTCGATTTCTTGGCTTTATATATCT TGTGGAAAGGACGAAACACC (SEQ ID NO: 25). In some cases, a mouse U6 promoter can comprise a sequence with at least about: 70%, 75%, 80%, 85%, 90%, 91%, 92%, 93%, 94%, 95%, 96%, 97%, 98%, 99% or 100% sequence identity to: GTACTGAGTCGCCCAGTCTCAGATAGATCCGACGCCGCCATCTCTAGGCCCGCGCCG GCCCCCTCGCACAGACTTGTGGGAGAAGCTCGGCTACTCCCCTGCCCCGGTTAATTT GCATATAATATTTCCTAGTAACTATAGAGGCTTAATGTGCGATAAAAGACAGATAAT CTGTTCTTTTTAATACTAGCTACATTTTACATGATAGGCTTGGATTTCTATAAGAGAT ACAAATACTAAATTATTATTTTAAAAAACAGCACAAAAGGAAACTCACCCTAACTGT AAAGTAATTGTGTGTTTTGAGACTATAAATATCCCTTGGAGAAAAGCCTTGTTTG (SEQ ID NO: 26). In some cases, a human U7 promoter can comprise a sequence with at least about: 70%, 75%, 80%, 85%, 90%, 91%, 92%, 93%, 94%, 95%, 96%, 97%, 98%, 99% or 100% sequence identity to: TTAACAACAACGAAGGGGCTGTGACTGGCTGCTTTCTCAACCAATCAGCACCGAACT CATTTGCATGGGCTGAGAACAAATGTTCGCGAACTCTAGAAATGAATGACTTAAGTA AGTTCCTTAGAATATTATTTTTCCTACTGAAAGTTACCACATGCGTCGTTGTTTATAC AGTAATAGGAACAAGAAAAAAGTCACCTAAGCTCACCCTCATCAATTGTGGAGTTC CTTTATATCCCATCTTCTCTCCAAACACATACGCA (SEQ ID NO: 27). In some cases, a mouse U7 promoter can comprise a sequence with at least about: 70%, 75%, 80%, 85%, 90%, 91%, 92%, 93%, 94%, 95%, 96%, 97%, 98%, 99% or 100% sequence identity to: TTAACAACATAGGAGCTGTGATTGGCTGTTTTCAGCCAATCAGCACTGACTCATTTG CATAGCCTTTACAAGCGGTCACAAACTCAAGAAACGAGCGGTTTTAATAGTCTTTTA GAATATTGTTTATCGAACCGAATAAGGAACTGTGCTTTGTGATTCACATATCAGTGG AGGGGTGTGGAAATGGCACCTTGATCTCACCCTCATCGAAAGTGGAGTTGATGTCCT TCCCTGGCTCGCTACAGACGCACTTCCGC (SEQ ID NO: 28). In some cases, a human U1 promoter can comprise a sequence with at least about: 70%, 75%, 80%, 90%, 91%, 92%, 93%, 94%, 95%, 96%, 97%, 98%, 99% or 100% sequence identity to: TAAGGACCAGCTTCTTTGGGAGAGAACAGACGCAGGGGCGGGAGGGAAAAAGGGA GAGGCAGACGTCACTTCCTCTTGGCGACTCTGGCAGCAGATTGGTCGGTTGAGTGGC AGAAAGGCAGACGGGGACTGGGCAAGGCACTGTCGGTGACATCACGGACAGGGCG ACTTCTATGTAGATGAGGCAGCGCAGAGGCTGCTGCTTCGCCACTTGCTGCTTCGCC ACGAAGGGAGTTCCCGTGCCCTGGGAGCGGGTTCAGGACCGCTGATCGGAAGTGAG AATCCCAGCTGTGTGTCAGGGCTGGAAAGGGCTCGGGAGTGCGCGGGGCAAGTGAC CGTGTGTGTAAAGAGTGAGGCGTATGAGGCTGTGTCGGGGCAGAGCCCGAAGATCT C (SEQ ID NO: 29). In some cases, a CMV promoter can comprise a sequence with at least about: 70%, 75%, 80%, 90%, 91%, 92%, 93%, 94%, 95%, 96%, 97%, 98%, 99% or 100% sequence identity to:

(SEQ ID NO: 30)
ATACGCGTTGACATTGATTATTGACTAGTTATTAATAGTAATCAATTACG
GGGTCATTAGTTCATAGCCCATATATGGAGTTCCGCGTTACATAACTTAC
GGTAAATGGCCCGCCTGGCTGACCGCCCAACGACCCCCGCCCATTGACGT
CAATAATGACGTATGTTCCCATAGTAACGCCAATAGGGACTTTCCATTGA
CGTCAATGGGTGGAGTATTTACGGTAAACTGCCCACTTGGCAGTACATCA
AGTGTATCATATGCCAAGTACGCCCCCTATTGACGTCAATGACGGTAAAT
GGCCCGCCTGGCATTATGCCCAGTACATGACCTTATGGGACTTTCCTACT
TGGCAGTACATCTACGTATTAGTCATCGCTATTACCATGGTGATGCGGTT
TTGGCAGTACATCAATGGGCGTGGATAGCGGTTTGACTCACGGGGATTTC
CAAGTCTCCACCCCATTGACGTCAATGGGAGTTTGTTTTGGCACCAAAAT
CAACGGGACTTTCCAAAATGTCGTAACAACTCCGCCCCATTGACGCAAAT
GGGCGGTAGGCGTGTACGGTGGGAGGTCTATATAAGCAGAGCTCGTTTAG
TGAACCGTCAGATCGCCTGGAGACGCCATCCACGCTGTTTTGACCTCCAT
AGAAGACACCGGGACCGATCCAGCCTCCGGACTCTAGAGGATCGAACC.

### G. Chemically Modified Guide RNAs

An engineered guide RNA as described herein for use in treating a disease or condition in a subject can comprise at least one chemical modification. In some embodiments, the engineered guide RNA can comprise at least one, two, three, four, five, six, seven, eight, nine, ten, 11, 12, 13, 14, 15, 16, 17, 18, 19, 20, 30, 50, 100, or more chemical modifications. In some embodiments, the engineered guide RNA described herein may not comprise a chemical modification. In some cases, the engineered guide RNAs disclosed herein with barbell macro-footprints can be manufactured, chemically modified, and delivered directly to a subject in need thereof as RNA (without a vector, such as an AAV).

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-×, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3 phosphorothioate or any combinations thereof.

A chemical modification can be made at any location of the engineered guide RNA. In some cases, a modification may be located in a 5′ or 3′ end, or both. In some cases, a polynucleotide can comprise a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. In some cases, more than one modification can be made to the engineered guide RNA. In some cases, a modification can be permanent. In other cases, a modification can be transient. In some cases, multiple modifications may be made to the engineered guide RNA. The engineered guide RNA modification can alter physio-chemical properties of a nucleotide, such as their conformation, polarity, hydrophobicity, chemical reactivity, base-pairing interactions, or any combination thereof.

In some embodiments, a chemical modification can also be a phosphorothioate substitute. In some cases, a natural phosphodiester bond can be susceptible to rapid degradation by cellular nucleases and a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, a chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, a chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, an engineered guide RNA can comprise 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, a chemical modification of the engineered guide RNA can comprise a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in Table 2.

TABLE 2
Exemplary Chemical Modification

| Modification of | |
|---|---|
| engineered guide RNA | Examples |
| Modification of one or | sulfur (S), selenium (Se), BR3 (wherein R can be, |
| both of the non-linking | e.g., hydrogen, alkyl, or aryl), C (e.g., an |
| phosphate oxygens in | alkyl group, an aryl group, and the like), H, |
| the phosphodiester | NR2, wherein R can be, e.g., hydrogen, alkyl, or |
| backbone linkage | aryl, or wherein R can be, e.g., alkyl or aryl |
| Modification of one or | sulfur (S), selenium (Se), BR3 (wherein R can be, |
| more of the linking | e.g., hydrogen, alkyl, or aryl), C (e.g., an |
| phosphate oxygens in | alkyl group, an aryl group, and the like), H, |
| the phosphodiester | NR2, wherein R can be, e.g., hydrogen, alkyl, or |
| backbone linkage | aryl, or wherein R can be, e.g., alkyl or aryl |
| Replacement of the | methyl phosphonate, hydroxylamino, siloxane, |
| phosphate moiety with | carbonate, carboxymethyl, carbamate, amide, |
| “dephospho” linkers | thioether, ethylene oxide linker, sulfonate, |
| | sulfonamide, thioformacetal, formacetal, oxime, |
| | methyleneimino, methylenemethylimino, |
| | methylenehydrazo, methylenedimethylhydrazo, or |
| | methyleneoxymethylimino |
| Modification or | Nucleic acid analog (examples of nucleotide |
| replacement of a | analogs can be found in PCT/US2015/025175, |
| naturally occurring | PCT/US2014/050423, PCT/US2016/067353, |
| nucleobase | PCT/US2018/041503, PCT/US18/041509, |
| | PCT/US2004/011786, or PCT/US2004/011833, |
| | all of which are expressly incorporated by |
| | reference in their entireties |
| Modification of the | phosphorothioate, phosphonothioacetate, |
| ribose-phosphate | phosphoroselenates, boranophosphates, borano |
| backbone | phosphate esters, hydrogen phosphonates, |
| | phosphonocarboxylate, phosphoroamidates, alkyl |
| | or aryl phosphonates, phosphonoacetate, or |
| | phosphotriesters |
| Modification of 5′ end | 5′ cap or modification of 5′ cap -OH |
| of polynucleotide | |
| Modification of 3′ end | 3′ tail or modification of 3′ end -OH |
| of polynucleotide | |
| Modification of the | phosphorothioate, phosphonothioacetate, |
| deoxyribose phosphate | phosphoroselenates, borano phosphates, borano |
| backbone | phosphate esters, hydrogen phosphonates, |
| | phosphoroamidates, alkyl or aryl phosphonates, or |
| | phosphotriesters |
| Substitution of the | methyl phosphonate, hydroxylamino, siloxane, |
| phosphate group | carbonate, carboxymethyl, carbamate, amide, |
| | thioether, ethylene oxide linker, sulfonate, |
| | sulfonamide, thioformacetal, formacetal, oxime, |
| | methyleneimino, methylenemethylimino, |
| | methylenehydrazo, methylenedimethylhydrazo, or |
| | methyleneoxymethylimino. |
| Modification of the | morpholino, cyclobutyl, pyrrolidine, or peptide |
| ribophosphate | nucleic acid (PNA) nucleoside surrogates |
| backbone | |
| Modifications to the | Locked nucleic acid (LNA), unlocked nucleic |
| sugar of a nucleotide | acid (UNA), or bridged nucleic acid (BNA) |
| Modification of a | 2′-O-methyl, 2′-O-methoxy-ethyl (2′-MOE), |
| constituent of the | 2′-fluoro, 2′-aminoethyl, 2′-deoxy-2′-fuloarabinou- |
| ribose sugar | cleic acid, 2′-deoxy, 2′-O-methyl, 3′- |
| | phosphorothioate, 3′-phosphonoacetate (PACE), |
| | or 3′-phosphonothioacetate (thioPACE) |
| Modifications to the | Modification of A, T, C, G, or U |
| base of a nucleotide | |
| Stereopure of | S conformation of phosphorothioate or R |
| nucleotide | conformation of phosphorothioate |

### Modification of Phosphate Backbone

In some embodiments, the chemical modification can comprise modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the engineered guide RNA can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). In some cases, the replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

In some cases, backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g., a combination of phosphate linkages such as a combination of phosphodiester and phosphorothioate linkages.

In some cases, substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. In some cases, conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, a chemical modification described herein can comprise modification of a phosphate backbone. In some embodiments, the engineered guide RNA described herein can comprise at least one chemically modified phosphate backbone. Exemplary chemically modification of the phosphate group or backbone can include replacing one or more of the oxygens with a different substituent. Furthermore, the modified nucleotide present in the engineered guide RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified engineered guide RNA can be stereopure (e.g., S or R confirmation). In some cases, a chemically modified engineered guide RNA comprises stereopure phosphate modification. For example, the chemically modified engineered guide RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

In some cases, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered guide RNA can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. In some cases, the phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, a chemical modification described herein can comprise modification by replacement of a phosphate group. In some embodiments, the engineered guide RNA described herein can comprise at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein can comprise modifying ribophosphate backbone of the engineered guide RNA. In some embodiments, the engineered guide RNA described herein can comprise at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein can comprise modifying of sugar. In some embodiments, the engineered guide RNA described herein can comprise at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH2CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Ci-₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the I position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g., L-nucleosides. In some aspects, the engineered guide RNA described herein includes the sugar group ribose, which may be a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered guide RNA comprises modifying the engineered guide RNA to include locked nucleic acid (LNA), unlocked nucleic acid (UNA), or bridged nucleic acid (BNA).

### Modification of a Constituent of the Ribose Sugar

In some embodiments, the engineered guide RNA described herein can comprise at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinoucleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers that can be prepared for incorporation into oligonucleotides. In some cases, the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids can contain one or more nucleosides wherein the sugar group has been modified. Such sugar modified nucleosides may impart enhanced nuclease stability, increased binding affinity, or some other beneficial biological property. In certain embodiments, nucleic acids can comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R1)(R2) (R═H, C1-C12 alkyl or a protecting group); and combinations thereof.

In some instances, the engineered guide RNA described herein can comprise modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta]anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

In some cases, modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]2, where n and m may be from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C_{1O} alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein can include one or more bicyclic nucleic acids. In certain such embodiments, the bicyclic nucleic acid comprises a bridge between the 4′ and the 2′ ribosyl ring atoms. In certain embodiments, nucleic acids provided herein can include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′—(CH₂)2-O-2′ (ENA); 4′-CH(CH₃)—O-2′ and 4′-CH(CH₂OCH₃)—O-2′, and analogs thereof, 4′-C(CH₃)(CH₃)—O-2′ and analogs thereof.

### Modifications on the Base of Nucleotide

In some embodiments, the chemical modification described herein can comprise modification of the base of nucleotide (e.g., the nucleobase). Exemplary nucleobases can include adenine (A), thymine (T), guanine (G), cytosine (C), and uracil (U). These nucleobases can be modified or replaced to in the engineered guide RNA described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein can comprise modifying an uracil. In some embodiments, the engineered guide RNA described herein can comprise at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, l-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein can comprise modifying a cytosine. In some embodiments, the engineered guide RNA described herein can comprise at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein can comprise modifying an adenine. In some embodiments, the engineered guide RNA described herein can comprise at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′—O-dimethyl-adenosine, N6-Methyl-2′-deoxyadenosine, N6, N6, 2′—O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein can comprise modifying a guanine. In some embodiments, the engineered guide RNA described herein can comprise at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′—O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some cases, the chemical modification of the engineered guide RNA can include introducing or substituting a nucleic acid analog or an unnatural nucleic acid into the engineered guide RNA. In some embodiments, nucleic acid analog can be any one of the chemically modified nucleic acid described herein. Exemplary nucleic acid analog can be found in PCT/US2021/034272, PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. In some cases, the chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′—O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein can comprise at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-th io-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein can comprise at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein can comprise at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

In some embodiments, a modified base of a unnatural nucleic acid includes, but may be not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered guide RNA. In some embodiments, the engineered guide RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Targets and Methods of Treatment

The present disclosure provides for compositions of engineered guide RNAs or engineered polynucleotides encoding guide RNAs and methods of use thereof, such as methods of treatment. The present disclosure also provides for compositions of engineered guide RNAs or engineered polynucleotides encoding guide RNAs for use in methods, such as methods of treatment, as described herein. In some embodiments, the engineered polynucleotides of the present disclosure encode guide RNAs targeting a non-coding sequence of RNA (e.g., a polyA signal sequence). In some embodiments, the present disclosure provides compositions of engineered polynucleotides targeting the polyA signal sequence. In some embodiments, the engineered guide RNAs disclosed herein facilitate ADAR-mediated RNA editing of adenosines in the polyA signal sequence. Alternatively, or in addition, the engineered guide RNAs disclosed herein facilitate knockdown by masking the polyA signal sequence and destabilization of the DUX4-FL mRNA. In some embodiments, engineered guide RNAs disclosed herein can be screened by in vitro and in vivo methods to determine their ability to facilitate ADAR mediated RNA editing of adenosines in a target RNA. In some instances, a therapeutic effect for a disease associated with DUX4 (e.g., FSHD) can be due to reduced levels of mRNA and/or protein of DUX4. In some instances, a therapeutic effect for a disease associated with DUX4 (e.g., FSHD) can be due to changes (e.g., reduced levels) of mRNA and/or protein of genes downstream DUX4. In some cases, a gene downstream of DUX4 can comprise SLC34A2, LEUTX, ZSCAN4, PRAMEF12, TRIM43, KHDC1L, Wfdc3, Agtr2, DEFB103, or MBD3L2. In some cases, a screening method can comprise a cell-based reporter. In some cases, a screening method can comprise a cell-based inducible model and caspase activity assay as described herein.

DUX4. The present disclosure provides for engineered guide RNAs that knockdown expression of DUX4-FL mRNA, and hence DUX4 activity. In some cases, an engineered guide RNA described herein can hybridize to a DUX4 target sequence (e.g., DUX4-FL mRNA). In some instances, the DUX4 target sequence can comprise a polyA signal sequence. as Alternatively or in addition, the engineered guide RNAs of the present disclosure facilitate knockdown of DUX4-activated genes. Expression knockdown (e.g., knockdown of DUX4-FL mRNA, DUX4 activated genes, or a combination thereof) may result from RNA editing of DUX4-FL mRNA (e.g., editing of the polyA signal sequence) facilitated by an engineered guide RNA, destabilization of the DUX4-FL mRNA (e.g., by masking the polyA signal sequence) facilitated by an engineered guide RNA, or a combination thereof. Facioscapulohumeral muscular dystrophy (FSHD) is a rare neuromuscular disease characterized by progressive skeletal muscle weakness and wasting with significant heterogeneity in phenotypic severity and age of onset. FSHD affects mostly the face (facio), shoulder girdle (scapula), and upper arm (humeral) regions of the body. As the disease progresses, muscles of the upper arms, the legs, and the postural muscles in the back loose mass and strength. Patients often first present with weakness of the face and periscapular muscles, eventually resulting in the inability to raise their arms above shoulder height, make facial expressions, or even close their eyes. In about 20% of the patients with FSHD, paraspinal muscle weakness is debilitating enough to result in patients becoming wheelchair-bound. FSHD is one of the most prevalent adult muscular dystrophies caused by an epigenetic derepression of the subtelomeric D4Z4 microsatellite array on chromosome 4q. This epigenetic derepression leads to hypomethylation in the distal-most D4Z4 unit and misexpression of the DUX4 gene in skeletal muscle. There are two subtypes of FSHD—FSHD1 and FSHD2. FSHD1 accounts for 95% of FSHD cases and is associated with the pathogenic contraction of D4Z4 microsatellite repeats, while FSHD2 accounts for 5% of the FSHD cases and is contraction-independent but associated with mutations in the chromatin regulator gene SMCHD1. The mutations for both FSHD1 and FSHD2 result in derepression of D4Z4 array and DUX4 mRNA misexpression. Said DUX4 mutations are autosomal dominant in 2/3 of FSHD1 patients and is prevalent in 1:8,000-12,000 (~16,000-38,000 patients in the US). DUX4 (double homeobox 4) is a germline transcription factor and its misexpression in muscle activates the expression of a broad set of genes (DUX4-activated genes), many involved in stem and germ cell biology. Some known DUX4-activated genes include MBD3L2, TRIM43, PRAMEF12, ZSCAN4, and LEUTX. In some cases, treatment of a DUX4-associated disease such as FSHD can be at least in part due to the knockdown of DUX4. In some cases, treatment of a DUX4-associated disease such as FSHD can be at least in part due to the knockdown of protein, mRNA, or both of a gene downstream of DUX4. In some cases, treatment of a DUX4-associated disease such as FSHD can be at least in part due to the knockdown of protein, mRNA, or both of a DUX4-activated gene. Although physical therapy, pain management, and surgery can alleviate some of the disabilities associated with FSHD, these treatments are not curative, and none of them address the underlying cause of the disease pathology. While healthy subjects generate a non-toxic splice form of DUX4 mRNA that lacks the C-term transactivation domain of DUX4 (referred to as DUX4-S for short), affected subjects produce a toxic splice form of DUX4 mRNA (referred to as DUX4-FL for full length) leading to expression of a toxic form of the DUX4 protein in muscle. Although various pharmaceutical and cell-based intervention approaches are being explored to treat FSHD, these generally offer little to no therapeutic benefit based on results from clinical trials. To develop a more targeted form of treatment, approaches that reduce muscle-specific DUX4-FL expression and DUX4-mediated toxicity have become attractive goals of FSHD therapy. Indeed, genetic treatments that target the root cause of the disease (e.g., DUX4) are expected to lead to a more effective or far-reaching therapeutic effect. The exact amount of DUX4 inhibition required for effective therapy is currently unknown, but data from clinically affected and asymptomatic FSHD patients support the idea that any reduction in DUX4-FL mRNA expression will have a therapeutic benefit. In some embodiments, the present disclosure provides compositions of engineered guide RNAs that target DUX4 and facilitate ADAR-mediated RNA editing of DUX4, specifically, DUX4-FL to mediate DUX4-FL knockdown. In some embodiments, the engineered guide RNAs of the present disclosure target a coding sequence in DUX4-FL mRNA. In some embodiments, the engineered guide RNAs of the present disclosure target a non-coding sequence in DUX4. The non-coding sequence can be a polyA signal sequence (ATTAAA) in the pLAM region. The engineered guide RNA can facilitate ADAR-mediated RNA editing of one or more adenosines in the polyA signal sequence of DUX4. RNA editing of this polyA signal sequence reduces mRNA cleavage and polyadenylation, and genetic excision of the DUX4-FL polyA sequence can result in DUX4-FL mRNA knockdown, DUX4-FL protein knockdown, or both. In some embodiments, engineered guide RNAs of the present disclosure can be designed to target more than one adenosine within the polyA signal sequence in DUX4. In some embodiments, engineered guide RNAs of the present disclosure can be multiplexed to target more than one polyA signal sequence. In some instances, the guide RNAs disclosed herein can bind and mask the polyA signal site and result in destabilization of the DUX4-FL mRNA. In some cases, masking the polyA signal site can comprise binding to and inhibiting translation of DUX4-FL mRNA.

In some embodiments, a target tissue for a guide RNA targeting DUX4 can comprise a muscle. In some cases, a muscle can comprise a muscle of the face, an arm muscle, a neck muscle, a shoulder muscle, a thigh muscle, a hip muscle, an abdominal muscle, a back muscle, a foot muscle, a hand muscle, or any combination thereof. In some cases, a muscle can comprise an orbicularis oculi, an orbicularis oris, a risorius, a zygomaticus major and minor, a biceps brachii, a triceps brichii, a trapezius, a rhomboids, a levator scapulae, a latissimus dorsi, a pectorals major, a pelvic girdle muscles, an abdominal muscles, a tibialis anterior, or any combination thereof. In some cases, a muscle of the face can comprise an occipitofrontalis muscle, a orbicularis oculi muscle, a temporalis muscle, a buccinator muscle, a masseter muscle, a mentalis muscle, a depressor labii inferioris muscle, a orbicularis oris muscle, a levator anguli oris muscle, a levator labii superioris muscle, a depressor anguli oris muscle, a levator labii superioris alaeque nasi muscle, zygomaticus major and minor muscle, a orbicularis oculi muscle, a corrugator supercilii muscle, or a risorius muscle. In some cases, a neck muscle can comprise an omohyoid muscle, a platysma muscle, a sternohyoid muscle, a stemocleidomastoid muscle, a levator scapulae muscle, a scalene muscle, a trapezius muscle, a semispinalis capitis muscle, a serratus posterior superior muscle, or any combination thereof. In some cases, shoulder muscle can comprise a deltoid muscle, a supraspinatus muscle, a rhomboids muscle, an infraspinatus muscle, a teres minor muscle, a teres major muscle, a pectoralis major muscle, a pectoralis minor, a serratus anterior muscle, or any combination thereof. In some cases, an arm muscle can comprise a triceps brachii muscle, a biceps brachii muscle, a brachialis muscle, a brachioradialis muscle, a carpal muscle, an extensor digitorum muscle, a extensor indicis muscle, an extensor digiti minimi muscle, a flexor digitorum superficialis muscle, a flexor digitorum profundus muscle, flexor pollicis longus muscle, extensor pollicis longus muscle, extensor pollicis brevis muscle, abductor pollicis longus muscle, a thenar muscles muscle, an adductor pollicis muscle, a hypothenar muscles muscle, a lumbricales muscle, a dorsal interossei muscle, a palmar interossei muscle, or any combination thereof. In some cases, a hip muscle can comprise a tensor fasciae muscle, a gluteus minimus muscle, a gluteus maximus muscle, a gluteus medius muscle, a piriformis muscle, an obturator intermus muscle, or any combination thereof. In some cases, an abdominal muscle can comprise a pyramidalis muscle, a rectus abdominus muscle, an external oblique muscle, an internal oblique muscle, a transversus abdominis muscle, or any combination thereof. In some cases, a back muscle can comprise a trapezius muscle, a rhomboids muscle, a latissimus dorsi muscle, an erector spinae muscle, a multifidus muscle, a quadratus lumborum muscle, or any combination thereof. In some cases, a leg muscle can comprise a vastus lateralis muscle, a vastus medialis muscle, a vastus intermedius muscle, a rectus femoris muscle, a biceps femoris muscle, a semimembranosus muscle, a semitendinosus muscle, a gastrocnemius muscle, a soleus muscle, a plantaris muscle, or any combination thereof. In some cases, a foot muscle can comprise an abductor hallucis muscle, a tibialis anterior muscle, an extensor digitorum longus muscle, a flexor digitorum longus muscle, a fibularis longus muscle, a fibularis tertius muscle, a fibularis brevis muscle, or any combination thereof.

In some embodiments, a target cell for a guide RNA targeting DUX4 can comprise a somatic (e.g., a muscle cell) or a gamete cell. For example, a somatic cell can comprise a cell of an internal organ, the skin, a muscle, a bone, a blood cell, a connective tissue cell, or any combination thereof. In some cases, a somatic cell can comprise a muscle cell. In some cases, a muscle cell can comprise a skeletal muscle cell, a cardiac muscle cell, a smooth muscle cell, or a combination thereof. In some cases, a muscle cell can comprise a myocyte, a myofibril, a myoblast, a myotube, a cardiomyocyte, or any combination thereof.

The engineered guide RNAs of the present disclosure facilitated ADAR-mediated RNA editing of DUX4 polyA signal sequence thereby, affecting mRNA knockdown, protein knockdown, or both. In some embodiments, the engineered guide RNAs of the present disclosure facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine. In some cases, off-target adenosine editing comprises global off target editing or local off target editing.

In some embodiments, the DUX4 RNA comprises a pre-mRNA transcript of DUX4. In some embodiments, an engineered guide RNA of the present disclosure can facilitate editing of at least one edit in the polyA signal sequence the pre-mRNA transcript of DUX4. In some cases, at least 40%, at least 50%, at least 55%, at least 60%, at least 65%, at least 70%, at least 75%, at least 80%, at least 85%, at least 90%, at least 95%, or at least 99% of the pre-mRNA transcripts of DUX4 have at least one edit in the polyA signal sequence. In some cases, at least 80%, of the pre-mRNA transcripts of DUX4 have at least one edit in the polyA signal sequence. In some cases, 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of the pre-mRNA transcripts of DUX4 have at least one edit in the polyA signal sequence.

In some embodiments, a mutation in the polyA signal sequence (ATTAAA) in the pLAM region of DUX4-FL results in a DUX4-FL mRNA knockdown, a DUX4 protein knockdown, or both. As RNA, the polyA signal sequence corresponds to the sequence AUUAAA. In some cases, the polyA signal sequence (AUUAAA) can be mutated to AUUAAG; AUUAGA; AUUGAA; GUUAAA; or GUUGGG. In some cases, an engineered guide RNA disclosed herein can facilitate ADAR-mediated RNA editing of the unmodified polyA signal sequence (AUUAAA) to AUUAAG; AUUAGA; AUUGAA; GUUAAA; or GUUGGG. In some instances, ADAR-mediated RNA editing of the unmodified polyA signal sequence to AUUAAG; AUUAGA; AUUGAA; GUUAAA; or GUUGGG results in a DUX4-FL mRNA knockdown, a DUX4 protein knockdown, or both. Any adenosine in the ATTAAA sequence can be a target adenosine.

In some embodiments, an engineered guide disclosed herein can facilitate ADAR-mediated RNA editing of one or more adenosines in the non-coding polyA signal sequence (ATTAAA) in the pLAM region of DUX4. In some cases, a method of editing DUX4 RNA can comprise contacting the DUX4 RNA with an engineered guide disclosed herein and an RNA editing entity. In some cases, the method can comprise editing the non-coding polyA signal sequence. As RNA, the polyA signal sequence corresponds to the sequence AUUAAA. The corresponding positions for each “A” in the polyA signal sequence (AUUAAA) are denoted as 0, 3, 4, and 5 from left to right. In some cases, the first A in the polyA signal sequence is designated as the target adenosine at position 0. In some cases, editing the polyA signal sequence can comprise editing the polyA signal sequence at any A. In some cases, editing can comprise editing from about: 20% to about 95%, 30% to about 95%, 40% to about 95%, 44% to about 91%, 60% to about 95%, 50% to about 99%, or 80% to about 91% of any A position in the polyA signal sequence.

In some embodiments, an engineered guide disclosed herein is at least partially complementary to a target RNA. In some cases, the target RNA is a DUX4 RNA. In some cases, the target RNA comprises the sequence of ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1). In some cases, the target DNA sequence encoding the target RNA sequence comprises the sequence of ACCTGGATTAGAGTTACATCTCCTGGATGATTAGTTCAGAGATATATTAAAATGCCC CCTCCCTGTGGATCCTATAGAAGATTTGCATCTTTTGTGTGATGAGTGCAGAG (SEQ ID NO: 31). In some cases, the target RNA can comprise a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, SEQ ID NO: 243, or SEQ ID NO: 245. In some cases, the target DNA sequence encoding the target RNA sequence comprises a sequence with at least 80%, at least 810%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31, SEQ ID NO: 242, or SEQ ID NO: 244. An engineered guide RNA of the present disclosure can be used to facilitate modification of the target RNA. In some embodiments, an engineered guide disclosed herein can facilitate ADAR-mediated RNA editing of one or more adenosines in the target RNA sequence of SEQ ID NO: 1, SEQ ID NO: 243, or SEQ ID NO: 245.

Assays for Measuring Efficacious Engineered gRNAs Targeting DUX4

In some embodiments, the engineered guide RNAs of the present disclosure facilitate ADAR-mediated RNA editing of DUX4. In some cases, the engineered guide RNAs of the present disclosure can bind and mask the DUX4 polyA signal site to facilitate destabilization of DUX4-FL mRNA. In some cases, a guide RNA herein can facilitate ADAR-mediated RNA editing of DUX4 and mask the DUX4 polyA signal site. In some embodiments, ADAR-mediated RNA editing of DUX4 can result in a knockdown (e.g., a reduction) of protein levels, a knockdown in mRNA levels, or both. In some embodiments, masking of the DUX4 polyA signal site can result in a knockdown (e.g., a reduction) of protein levels, a knockdown in mRNA levels, or both. In some cases, a knockdown of protein levels can be of DUX4 or of a protein downstream of DUX4. In some cases, a knockdown of mRNA levels can be of DUX4 or an mRNA downstream of DUX4. In some cases, a knockdown of protein levels, mRNA levels, or both of DUX4 or a gene downstream of DUX4 can be facilitated by an engineered guide that masks the polyA signal sequence of DUX4-FL, facilitated by an engineered guide RNA that edits a polyA signal sequence of DUX4-FL, or both. In some instances, the knockdown of protein levels and/or mRNA levels is at least partially an ADAR dependent knockdown. In some instances, the knockdown of protein levels and/or mRNA levels is at least partially an ADAR independent knockdown.

In some embodiments, an assay is used to determine the efficacy of a guide RNA disclosed herein. In some cases, an assay can comprise measuring RNA editing, mRNA levels, or protein levels in a cell. In some cases, an assay can comprise measuring RNA editing, mRNA levels, or protein levels in a cell before and after a treatment with a guide RNA disclosed herein. In some cases, cells can be sampled in a time course assay. In some cases, a cell can comprise a cell with a functional ADAR gene. In some cases, a cell can comprise a cell with a nonfunctional ADAR gene. For example, a cell can comprise a truncated or mutated ADAR gene or a cell can comprise a deleted ADAR gene. In some cases, an assay can be used to compare editing levels, levels of mRNA, or levels of protein, in a cell with a functional copy of an ADAR gene and in a cell without a functional ADAR gene. In some cases, the reduction of mRNA or protein levels in the cell can be identified as ADAR dependent reduction in mRNA or protein levels. Protein levels in a cell can be measured by any standard technique, for example a Western Blot immunofluorescence, immunohistochemistry, or an enzyme-linked immunosorbent assay (ELISA). mRNA levels in a cell can be measured by any standard technique, for example by Real-Time Quantitative Reverse Transcription PCR, RNA sequencing, or droplet digital PCR. In some cases, protein levels can be determined by a functional assay specific to a protein of interest. For example, an assay can be used to determine the amount of a protein by an enzymatic assay measuring the enzyme kinetics of the protein. In some cases, the efficacy of a guide RNA can be determined by physiological improvements. In some cases, a physiological improvement can comprise treadmill performance (increase in distance traveled) and grip strength improvement.

In some embodiments, a guide RNA disclosed herein can facilitate ADAR dependent knockdown of mRNA levels or protein levels of 1 to 100%. In some cases, a guide RNA disclosed herein can facilitate ADAR-dependent knockdown of mRNA levels or protein levels from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as compared to a cell before treatment with the guide RNA. In some cases, ADAR dependent knockdown of mRNA levels or protein levels can be compared between a cell comprising a functional copy of ADAR and a cell comprising a nonfunctional copy of ADAR.

In some embodiments, the engineered guide RNAs of the present disclosure facilitate ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5% editing of a target adenosine. In some embodiments, the engineered guide RNAs of the present disclosure can facilitate at least 10% editing of a target adenosine. 15% editing of a target adenosine. In some embodiments, the engineered guide RNAs of the present disclosure can facilitate at least 20% editing of a target adenosine. In some embodiments, the engineered guide RNAs of the present disclosure can facilitate at least 25% editing of a target adenosine. In some embodiments, the engineered guide RNAs of the present disclosure can facilitate at least 30% editing of a target adenosine. In some embodiments, the engineered guide RNAs of the present disclosure can facilitate at least 35% editing of a target adenosine. In some embodiments, the engineered guide RNAs of the present disclosure can facilitate at least 40% editing of a target adenosine. In some embodiments, the engineered guide RNAs of the present disclosure can facilitate at least 45% editing of a target adenosine. In some embodiments, the engineered guide RNAs of the present disclosure can facilitate at least 50% editing of a target adenosine. In some embodiments, the engineered guide RNAs of the present disclosure can facilitate at least 55% editing of a target adenosine. In some embodiments, the engineered guide RNAs of the present disclosure can facilitate at least 60% editing of a target adenosine. In some embodiments, the engineered guide RNAs of the present disclosure can facilitate at least 65% editing of a target adenosine. In some embodiments, the engineered guide RNAs of the present disclosure can facilitate at least 70% editing of a target adenosine. In some embodiments, the engineered guide RNAs of the present disclosure can facilitate at least 75% editing of a target adenosine. In some embodiments, the engineered guide RNAs of the present disclosure can facilitate at least 80% editing of a target adenosine. In some embodiments, the engineered guide RNAs of the present disclosure can facilitate at least 85% editing of a target adenosine. In some embodiments, the engineered guide RNAs of the present disclosure can facilitate at least 90% editing of a target adenosine. In some embodiments, the engineered guide RNAs of the present disclosure can facilitate at least 95% editing of a target adenosine. In some embodiments, the engineered guide RNAs of the present disclosure can facilitate 100% editing of a target adenosine. In some embodiments, the engineered guide RNAs of the present disclosure can facilitate from 5 to 20% editing of a target adenosine. In some embodiments, the engineered guide RNAs of the present disclosure can facilitate from 20 to 40% editing of a target adenosine. In some embodiments, the engineered guide RNAs of the present disclosure can facilitate from 40 to 60% editing of a target adenosine. In some embodiments, the engineered guide RNAs of the present disclosure can facilitate from 50 to 85% editing of a target adenosine. In some embodiments, the engineered guide RNAs of the present disclosure can facilitate from 60 to 80% editing of a target adenosine. In some embodiments, the engineered guide RNAs of the present disclosure can facilitate from 80 to 100% editing of a target adenosine. In some embodiments, the engineered guide RNAs of the present disclosure can facilitate from 60 to 80% editing of a target adenosine. In some embodiments, the engineered guide RNAs of the present disclosure can facilitate from 70 to 90% editing of a target adenosine. In some embodiments, the engineered guide RNAs of the present disclosure can facilitate these levels of on-target RNA editing while maintaining less than 30% editing of an off-target adenosine. In some embodiments, the engineered guide RNAs of the present disclosure can facilitate these levels of on-target RNA editing while maintaining less than 25% editing of an off-target adenosine. In some embodiments, the engineered guide RNAs of the present disclosure can facilitate these levels of on-target RNA editing while maintaining less than 20% editing of an off-target adenosine. In some embodiments, the engineered guide RNAs of the present disclosure can facilitate these levels of on-target RNA editing while maintaining less than 15% editing of an off-target adenosine. In some embodiments, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 9% editing of an off-target adenosine. In some embodiments, the engineered guide RNAs of the present disclosure can facilitate these levels of on-target RNA editing while maintaining less than 8% editing of an off-target adenosine. In some embodiments, the engineered guide RNAs of the present disclosure can facilitate these levels of on-target RNA editing while maintaining less than 7% editing of an off-target adenosine. In some embodiments, the engineered guide RNAs of the present disclosure can facilitate these levels of on-target RNA editing while maintaining less than 6% editing of an off-target adenosine. In some embodiments, the engineered guide RNAs of the present disclosure can facilitate these levels of on-target RNA editing while maintaining less than 5% editing of an off-target adenosine. In some embodiments, the engineered guide RNAs of the present disclosure can facilitate these levels of on-target RNA editing while maintaining less than 4% editing of an off-target adenosine. In some embodiments, the engineered guide RNAs of the present disclosure can facilitate these levels of on-target RNA editing while maintaining less than 3% editing of an off-target adenosine. In some embodiments, the engineered guide RNAs of the present disclosure can facilitate these levels of on-target RNA editing while maintaining less than 2% editing of an off-target adenosine. In some embodiments, the engineered guide RNAs of the present disclosure can facilitate these levels of on-target RNA editing while maintaining less than 1% editing of an off-target adenosine. In some embodiments, the engineered guide RNAs of the present disclosure can facilitate these levels of on-target RNA editing while maintaining 0% editing of an off-target adenosine.

In some cases, an off-target edit comprises an edit of a polynucleotide (e.g., a RNA) that is not a target RNA species, for example a different mRNA other than a mRNA encoding DUX4. This can be referred to as global off-target editing. The engineered guide RNAs disclosed herein facilitate little to no global off-target editing. In some cases, the engineered guide RNAs disclosed herein facilitate less about: 3%, 2%, 1%, 0.1%, 0.01% global off-target editing. In comparison, in some instances, an off-target edit comprises an edit of an adenosine in a target RNA that is not the target adenosine. This can be referred to as local off-target editing.

In some embodiments, the engineered guide RNAs of the present disclosure facilitate ADAR-mediated RNA editing of DUX4-FL mRNA, which results in knockdown of protein levels. In some embodiments, the engineered guide RNAs of the present disclosure can bind and mask the polyA signal site of DUX4-FL mRNA, which results in knockdown of protein levels. The knockdown in protein levels can be quantitated as a reduction in expression of the DUX4-FL protein. The engineered guide RNAs of the present disclosure can facilitate from 1% to 100% DUX4-FL protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% DUX4-FL protein knockdown. In some embodiments, the engineered guide RNAs of the present disclosure facilitate from 30% to 60% DUX4-FL protein knockdown. Protein knockdown (e.g., DUX4-FL knockdown) can be measured by an assay comparing a sample or subject treated with the engineered guide RNA to a control sample or subject not treated with the engineered guide RNA. In some cases, protein knockdown can be measured by comparing the amount of the protein present in a sample or subject before a treatment with a guide RNA disclosed herein and comparing it to the amount of the protein after the treatment.

In some embodiments, ADAR-mediated RNA editing of DUX4-FL mRNA, results in knockdown of downstream protein levels of one or more proteins downstream of DUX4. In some embodiments, the engineered guide RNAs of the present disclosure can bind and mask the polyA signal site of DUX4-FL mRNA, which results in knockdown of downstream protein levels of one or more proteins downstream of DUX4. In some instances, a knockdown of a protein downstream of DUX4 can be used to determine the reduction of DUX4 protein levels. In some cases, a downstream protein of DUX4 is encoded by a gene comprising SLC34A2, LEUTX, ZSCAN4, PRAMEF12, KHDC1L, Wfdc3, Agtr2, TRIM43, DEFB103, or MBD3L2. The knockdown in protein or mRNA levels of a downstream of DUX4 can be quantitated as a reduction in expression of the SLC34A2 gene, the LEUTX gene, the ZSCAN4 gene, the PRAMEF12 gene, the TRIM43 gene, the DEFB103 gene, the Wfdc3 gene, the Agtr2 gene, the MBD3L2 gene, or combinations thereof. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% knockdown of a mRNA, a protein, or both encoded by SLC34A2, LEUTX, ZSCAN4, PRAMEF12, TRIM43, DEFB103, Wfdc3, Agtr2, KHDC1L, MBD3L2, or another gene downstream of DUX4, or combinations thereof. In some embodiments, increased knockdown of the DUX4 activity by the guide RNA is measured in an assay. In some cases, the increased knockdown comprises an increase in a protein knockdown of DUX4-FL and/or of a protein downstream of DUX4. In some cases, the assay can comprise measuring the level of a protein in a sample before and after treatment with a guide RNA described herein. In some cases, the assay can comprise measuring the level of a protein in a sample that is not treated with a guide RNA and measuring the protein in a sample that is treated with a guide RNA described herein.

In some embodiments, the engineered guide RNAs of the present disclosure facilitate ADAR-mediated RNA editing of DUX4, which results in knockdown of mRNA levels. In some embodiments, the engineered guide RNAs of the present disclosure can bind and mask the polyA signal site of DUX4-FL mRNA, which results in knockdown mRNA levels. The knockdown in mRNA levels can be quantitated as a reduction in expression of the DUX4 mRNA transcript protein. In some cases, the knockdown in DUX4-FL mRNA levels can be quantitated as a reduction in expression of the DUX4-activated genes, knockdown of cell death (e.g., caspase 3/7 activity), increase in cell viability, and/or increase in myotube fusion and diameter. The engineered guide RNAs of the present disclosure can facilitate a 1% to 100% decrease of DUX4 mRNA. The engineered guide RNAs of the present disclosure can facilitate a decrease of: 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of DUX4 mRNA. The engineered guide RNAs of the present disclosure can facilitate a decrease of: at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of DUX4 mRNA. The engineered guide RNAs of the present disclosure can facilitate a decrease of at least 50%, or at least 70% of DUX4 mRNA. In some embodiments, the engineered guide RNAs of the present disclosure facilitate a decrease of 50% to 75% of DUX4 mRNA. DUX4 (e.g., DUX4-FL) mRNA levels can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of DUX4, which results in expression knockdown of mRNA, protein, or both encoded by a gene downstream of DUX4. In some cases, the expression knockdown of a gene downstream of DUX4 can result in the treatment of a disease or condition. For example, the knockdown of SLC34A2, LEUTX, ZSCAN4, PRAMEF12, TRIM43, KHDC1L, DEFB103, Wfdc3, Agtr2, or MBD3L2 can result in a treatment for FSHD. In some embodiments, the engineered guide RNAs of the present disclosure can bind and mask the DUX4 polyA signal sequence, which results in expression knockdown of mRNA, protein, or both encoded by a gene downstream of DUX4. In some cases, a gene downstream of DUX4 can comprise SLC34A2, LEUTX, ZSCAN4, PRAMEF12, TRIM43, KHDC1L, DEFB103, Wfdc3, Agtr2, or MBD3L2. In some cases, a reduction in the expression of the mRNA of SLC34A2, LEUTX, ZSCAN4, PRAMEF12, TRIM43, KHDC1L, DEFB103, Wfdc3, Agtr2, MBD3L2, or combinations thereof can indicate a reduction in the expression of DUX4. The engineered guide RNAs of the present disclosure can facilitate a 1% to 100% decrease of SLC34A2, LEUTX, ZSCAN4, PRAMEF12, TRIM43, KHDC1L, DEFB103, Wfdc3, Agtr2, or MBD3L2 mRNA, or combinations thereof. The engineered guide RNAs of the present disclosure can facilitate a decrease of: 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of SLC34A2, LEUTX, ZSCAN4, PRAMEF12, TRIM43, KHDC1L, DEFB103, Wfdc3, Agtr2, or MBD3L2 mRNA, or combinations thereof. The engineered guide RNAs of the present disclosure can facilitate a decrease of: at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of SLC34A2, LEUTX, ZSCAN4, PRAMEF12, TRIM43, KHDC1L, DEFB103, Wfdc3, Agtr2, or MBD3L2 mRNA, or combinations thereof. SLC34A2, LEUTX, ZSCAN4, PRAMEF12, TRIM43, KHDC1L, DEFB103, Wfdc3, Agtr2, or MBD3L2 mRNA levels can be measured by an assay comparing a sample or subject treated with the engineered guide RNA to a control sample or subject not treated with the engineered guide RNA.

In some embodiments, a guide RNA disclosed herein can facilitate knockdown of mRNA levels or protein levels of 1 to 100%. In some cases, a guide RNA disclosed herein can facilitate knockdown of mRNA levels or protein levels in a muscle tissue or muscle cells from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as compared to a muscle tissue or muscle cells before treatment with the guide RNA or as compared to treatment with a control (e.g., a control scrambled guide RNA).

An engineered guide RNA of the present disclosure can be used in a method of treating a disorder in a subject in need thereof. For example, an engineered guide RNA disclosed herein can be used to treat facioscapulohumeral muscular dystrophy. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., FSHD). In some embodiments, an engineered guide RNA disclosed herein can be used to treat FSHD. In some cases, FSHD can comprise FSHD type 1 (FSHD1) or FSHD type 2 (FSHD2). In some embodiments, an engineered guide RNA disclosed herein can be used to treat FSHD 1. In some embodiments, an engineered guide RNA disclosed herein can be used to treat FSHD 2. In some embodiments, an engineered guide RNA of the present disclosure can be used to treat a condition associated with one or more mutations. For example, disclosed herein are methods of treating FSHD with engineered guide RNAs targeting DUX4.

In some embodiments, treatment of FSHD comprises treatment of the symptoms associated with FSHD. A symptom of FSHD can comprise a weakness or atrophy of muscle, such as a muscle of the face, an arm muscle, a neck muscle, a shoulder muscle, a thigh muscle, a hip muscle, an abdominal muscle, a back muscle, a foot muscle, a hand muscle, or any combination thereof. In some cases, a symptom of FSHD can comprise a vision loss, a respiratory insufficiency, a dysphagia, a lordosis, a scoliosis, a hearing loss, a pain, an inflammation (e.g., inflammation of muscles), shoulder weakness, unequal (nonsymmetrical weakness) of the body, or any combination thereof.

### Pharmaceutical Compositions

The compositions described herein (e.g., compositions comprising an engineered guide RNA or an engineered polynucleotide encoding an engineered guide RNA) can be formulated with a pharmaceutically acceptable carrier for administration to a subject (e.g., a human or a non-human animal). The compositions herein can be pharmaceutical compositions. The compositions described herein (e.g., compositions comprising an engineered guide RNA or an engineered polynucleotide encoding an engineered guide RNA) can be formulated with a pharmaceutically acceptable: excipient, carrier, diluent or any combination thereof for administration to a subject (e.g., a human or a non-human animal). A pharmaceutically acceptable carrier and/or diluent can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. A composition herein, such as a pharmaceutical composition can be in unit dose form. The compositions also can include stabilizers and preservatives. Additional examples of carriers, stabilizers, and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

### Delivery

An engineered guide RNA of the present disclosure or an engineered polynucleotide of the present disclosure (e.g., an engineered polynucleotide encoding an engineered guide RNA) can be delivered via a delivery vehicle. In some cases, a delivery vehicle can be a pharmaceutical composition in unit dose form. In some embodiments, the delivery vehicle is a vector. A vector can facilitate delivery of the engineered guide RNA or the engineered polynucleotide into a cell to genetically modify the cell. Target tissues and cells include but are not limited to satellite cells, myoblasts, myocytes, and myotubes of the face, shoulders, and upper limbs. In some cases, a target cell can be an HEK293T cell. In some cases, a target cell can be a patient-derived immortal cell. In some cases, a target cell can be a patient-derived primary cell. In some cases, a target cell can comprise an unaffected (U or V) or affected (A) muscle cell, such as 12Ubic cell, 12Abic cell, 15Vbic cell, a 15Abic cell, a 18Ubic cell, or a 18Abic cell. In some cases, a target cell can be a modified LHCN immortalized muscle cell. For example, the LHCN cell can be integrated with a reporter (e.g., luciferase) DUX4 expression system. In some cases, a target cell can be a modified HEK293T cell. For example, the HEK293T cell can be integrated with an inducible (e.g., by tetracycline) DUX4 expression system. In some cases, a vector can facilitate delivery of the engineered guide RNA or the engineered polynucleotide into target cell or an animal model, such as a mouse model. In some cases, a mouse model can comprise a transgenic mouse, a bi-transgenic mouse, a C57BL/6J mouse, a DUX4 inducible mouse model, or a human xenograft mouse model. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some cases, a delivery vehicle can comprise a non-viral delivery vehicle. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some cases, the engineered guide RNA of the present disclosure can be an in vitro transcribed (IVT) RNA. In some cases, an engineered guide RNA can be delivered as a formulation comprising the engineered guide RNA. In some cases, the engineered guide RNA may not be comprised in a vector. In some examples, the engineered guide RNA (e.g., as an oligonucleotide) can be formulated for delivery through direct injection. In some examples, the engineered guide RNA, as an oligo nucleotide can be formulated for delivery through intravenous administration, intramuscular administration, or oral administration.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the AAV can comprise an AAV5 serotype, an AAV6 serotype, an AAV8 serotype, or an AAV9 serotype. In some embodiments, the viral vector can be of a specific serotype. In some embodiments, the viral vector can be an AAV1 serotype, an AAV2 serotype, an AAV3 serotype, an AAV4 serotype, an AAV5 serotype, an AAV6 serotype, an AAV7 serotype, an AAV8 serotype, an AAV9 serotype, an AAV10 serotype, an AAV11 serotype, an AAV12 serotype, an AAV13 serotype, an AAV14 serotype, an AAV15 serotype, an AAV16 serotype, an AAV.rh8 serotype, an AAV.rh10 serotype, an AAV.rh20 serotype, an AAV.rh39 serotype, an AAV.Rh74 serotype, an AAV.RHM4-1 serotype, an AAV.hu37 serotype, an AAV.Anc80 serotype, an AAV.Anc80L65 serotype, an AAV.7m8 serotype, an AAV.PHP.B serotype, an AAV2.5 serotype, an AAV2tYF serotype, an AAV3B serotype, an AAV.LK03 serotype, an AAV.HSC1 serotype, an AAV.HSC2 serotype, an AAV.HSC3 serotype, an AAV.HSC4 serotype, an AAV.HSC5 serotype, an AAV.HSC6 serotype, an AAV.HSC7 serotype, an AAV.HSC8 serotype, an AAV.HSC9 serotype, an AAV.HSC10 serotype, an AAV.HSC11 serotype, an AAV.HSC12 serotype, an AAV.HSC13 serotype, an AAV.HSC14 serotype, an AAV.HSC15 serotype, an AAV.HSC16 serotype, and an AAVhu68 serotype, a derivative of any of these serotypes, a chimera of any of these serotypes, a variant of any of these serotypes or any combination thereof.

In some embodiments, the AAV vector can be a recombinant vector, a hybrid AAV vector, a chimeric AAV vector, a self-complementary AAV (scAAV) vector, a single-stranded AAV, or any combination thereof. In some cases, an scAAV vector can comprise scAAV6. In some cases, an scAAV vector can comprise scAAV1, scAAV2, scAAV5, scAAV8, or scAAV9.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding an engineered guide RNA) in an AAV vector. In some examples, methods of producing the delivery vectors described herein comprise, (a) introducing into a cell: (i) a polynucleotide comprising a promoter and an engineered guide RNA payload disclosed herein; and (ii) a viral genome comprising a Replication (Rep) gene and Capsid (Cap) gene that encodes a wild-type AAV capsid protein or modified version thereof, (b) expressing in the cell the wild-type AAV capsid protein or modified version thereof, (c) assembling an AAV particle; and (d) packaging the payload disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some cases, the delivery vehicle can be a DNA encoding the engineered guide RNA. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system can be a liposome or polymeric nanoparticle. In some cases, a non-viral vector system can be a lipid nanoparticle (LNP) or a polymer nanoparticle. In some embodiments, the engineered polynucleotide or a non-viral vector comprising the engineered guide RNA or the engineered polynucleotide is delivered to a cell by hydrodynamic injection or ultrasound.

### Administration

Administration can refer to methods that can be used to enable the delivery of a composition described herein (e.g. comprising an engineered guide RNA or an engineered polynucleotide encoding the same) to the desired site of biological action. For example, an engineered guide RNA can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some cases, administration disclosed herein can be a systemic administration. In some instances, administration can be systemic administration by an injection (e.g., intravenous administration or any administration by an injection) or oral delivery. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracomeal, intracoronal, intracoronary, intracorpous cavemaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof.

In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, an engineered guide RNA disclosed herein or a polynucleotide encoding the engineered guide RNA can be administered with a second therapeutic. In some cases, the second therapeutic can be administered in an amount sufficient to treat a disease or condition. In some cases, administration of the second therapeutic can be concurrent administration or consecutive administration to administration of the engineered guide RNA disclosed herein or the polynucleotide encoding the engineered guide RNA. In some cases, the second therapeutic can comprise losmapimod or a salt thereof. In some cases, losmapimod or a salt thereof can be administered in an amount of about: 0.0001 gram to about 100 grams or about 1 mg to about 100 mg.

In some embodiments, disclosed herein can be a method, comprising administering a composition disclosed herein to a subject (e.g., a human) in need thereof. In some instances, the method can treat or prevent a disease in the subject.

### NUMBERED EMBODIMENTS

A number of compositions, methods, and kits are disclosed herein. Specific exemplary embodiments of these compositions, methods, and kits are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

### Embodiments Section 1

Embodiment 1. An engineered guide RNA or a polynucleotide encoding the engineered guide RNA, wherein the engineered guide RNA is capable of hybridizing to a target DUX4 polyA signal RNA sequence and wherein the engineered guide RNA: a) comprises from 95 to 105 nucleotides; b) has at least 80%, at least 85%, at least 90%, at least 92%, at least 95%, at least 96%, at least 97%, at least 98%, at least 99% or 100% sequence identity to SEQ ID NO: 39, SEQ ID NO: 212, SEQ ID NO: 40, SEQ ID NO: 228, SEQ ID NO: 46, or SEQ ID NO: 239; and c) upon hybridization to the target DUX4 polyA signal RNA sequence, forms a guide-target RNA scaffold comprising at least 3 structural features including at least one 6/6 symmetric internal loop.
Embodiment 2. The engineered guide RNA or the polynucleotide encoding the engineered guide RNA of embodiment 1, wherein the engineered guide comprises 100 nucleotides.
Embodiment 3. The engineered guide RNA or the polynucleotide encoding the engineered guide RNA of embodiment 1, wherein polynucleotide encoding the engineered guide RNA comprises SEQ ID NO: 32, SEQ ID NO: 180, SEQ ID NO: 33, SEQ ID NO: 196, SEQ ID NO: 79, or SEQ ID NO: 207.
Embodiment 4. The engineered guide RNA or the polynucleotide encoding the engineered guide RNA of embodiment 1, the guide-target RNA scaffold comprises at least three or more wobble base pairs.
Embodiment 5. The engineered guide RNA or the polynucleotide encoding the engineered guide RNA of embodiment 4, wherein the engineered guide RNA comprises SEQ ID NO: 212, 228, or 239.
Embodiment 6. The engineered guide RNA or the polynucleotide encoding the engineered guide RNA of embodiment 4, wherein the polynucleotide encoding the engineered guide RNA comprises SEQ ID NO: 180, 196, or 207.
Embodiment 7. The engineered guide RNA or the polynucleotide encoding the engineered guide RNA of embodiment 1, wherein the guide-target RNA scaffold comprises at least one wobble base pair.
Embodiment 8. The engineered guide RNA or the polynucleotide encoding the engineered guide RNA of embodiment 7, wherein the guide-target RNA scaffold comprises at least two wobble base pairs.
Embodiment 9. The engineered guide RNA or the polynucleotide encoding the engineered guide RNA of embodiment 8, wherein the engineered guide RNA comprises SEQ ID NO: 212, 228, 239, or 46.
Embodiment 10. The engineered guide RNA or the polynucleotide encoding the engineered guide RNA of embodiment 8, wherein the polynucleotide encoding the engineered guide RNA comprises SEQ ID NO: 180, 196, 207, or 79.
Embodiment 11. The engineered guide RNA or the polynucleotide encoding the engineered guide RNA of embodiment 1, wherein the guide-target RNA scaffold comprises a mismatch and at least two 6/6 symmetric internal loops.
Embodiment 12. The engineered guide RNA or the polynucleotide encoding the engineered guide RNA of embodiment 11, wherein the engineered guide RNA comprises SEQ ID NO: 39, 40, 212, or 228.
Embodiment 13. The engineered guide RNA or the polynucleotide encoding the engineered guide RNA of embodiment 11, wherein the polynucleotide encoding the engineered guide RNA comprises SEQ ID NO: 32, 33, 180, or 196.
Embodiment 14. The engineered guide RNA or the polynucleotide encoding the engineered guide RNA of embodiment 1, wherein the guide-target RNA scaffold comprises at least two asymmetric bulges.
Embodiment 15. The engineered guide RNA or the polynucleotide encoding the engineered guide RNA of embodiment 14, wherein the engineered guide RNA comprises SEQ ID NO: 46 or 239.
Embodiment 16. The engineered guide RNA or the polynucleotide encoding the engineered guide RNA of embodiment 14, wherein the polynucleotide encoding the engineered guide RNA comprises SEQ ID NO: 79 or 207.
Embodiment 17. The engineered guide RNA or the polynucleotide encoding the engineered guide RNA of embodiment 1, wherein the guide-target RNA scaffold comprises at least one symmetric bulge.
Embodiment 18. The engineered guide RNA or the polynucleotide encoding the engineered guide RNA of embodiment 17, wherein the engineered guide RNA comprises SEQ ID NO: 46 or 239.
Embodiment 19. The engineered guide RNA or the polynucleotide encoding the engineered guide RNA of embodiment 17, wherein the polynucleotide encoding the engineered guide RNA comprises SEQ ID NO: 79 or 207.
Embodiment 20. The engineered guide RNA or the polynucleotide encoding the engineered guide RNA of embodiment 1, wherein the 6/6 symmetric internal loop is at position 44 relative to a target adenosine at position 0.
Embodiment 21. The engineered guide RNA or the polynucleotide encoding the engineered guide RNA of embodiment 20, wherein the engineered guide RNA comprises SEQ ID NO: 39 or 212.
Embodiment 22. The engineered guide RNA or the polynucleotide encoding the engineered guide RNA of embodiment 20, wherein the polynucleotide encoding the engineered guide RNA comprises SEQ ID NO: 32 or 180.
Embodiment 23. The engineered guide RNA or the polynucleotide encoding the engineered guide RNA of embodiment 1, wherein the 6/6 symmetric internal loop is at position −5 relative to a target adenosine at position 0.
Embodiment 24. The engineered guide RNA or the polynucleotide encoding the engineered guide RNA of embodiment 23, wherein the engineered guide RNA comprises SEQ ID NO: 39 or 212.
Embodiment 25. The engineered guide RNA or the polynucleotide encoding the engineered guide RNA of embodiment 23, wherein the polynucleotide encoding the engineered guide RNA comprises SEQ ID NO: 32 or 180.
Embodiment 26. The engineered guide RNA or the polynucleotide encoding the engineered guide RNA of embodiment 1, wherein the 6/6 symmetric internal loop is at position 27 relative to a target adenosine at position 0.
Embodiment 27. The engineered guide RNA or the polynucleotide encoding the engineered guide RNA of embodiment 26, wherein the engineered guide RNA comprises SEQ ID NO: 40 or 228.
Embodiment 28. The engineered guide RNA or the polynucleotide encoding the engineered guide RNA of embodiment 26, wherein the polynucleotide encoding the engineered guide RNA comprises SEQ ID NO: 33 or 196.
Embodiment 29. The engineered guide RNA or the polynucleotide encoding the engineered guide RNA of embodiment 1, wherein the 6/6 symmetric internal loop is at position −6 relative to a target adenosine at position 0.
Embodiment 30. The engineered guide RNA or the polynucleotide encoding the engineered guide RNA of embodiment 29, wherein the engineered guide RNA comprises SEQ ID NO: 40 or 228.
Embodiment 31. The engineered guide RNA or the polynucleotide encoding the engineered guide RNA of embodiment 29, wherein the polynucleotide encoding the engineered guide RNA comprises SEQ ID NO: 33 or 196.
Embodiment 32. The engineered guide RNA or the polynucleotide encoding the engineered guide RNA of embodiment 1, wherein the 6/6 symmetric internal loop is at position −22 relative to a target adenosine at position 0.
Embodiment 33. The engineered guide RNA or the polynucleotide encoding the engineered guide RNA of embodiment 32, wherein the engineered guide RNA comprises SEQ ID NO: 46 or 239.
Embodiment 34. The engineered guide RNA or the polynucleotide encoding the engineered guide RNA of embodiment 32, wherein the polynucleotide encoding the engineered guide RNA comprises SEQ ID NO: 79 or 207.
Embodiment 35. The engineered guide RNA or the polynucleotide encoding the engineered guide RNA of embodiment 1, wherein the 6/6 symmetric internal loop is at position 36 relative to a target adenosine at position 0.
Embodiment 36. The engineered guide RNA or the polynucleotide encoding the engineered guide RNA of embodiment 35, wherein the engineered guide RNA comprises SEQ ID NO: 46.
Embodiment 37. The engineered guide RNA or the polynucleotide encoding the engineered guide RNA of embodiment 35, wherein the polynucleotide encoding the engineered guide RNA comprises SEQ ID NO: 79.
Embodiment 38. An AAV vector encoding an engineered guide RNA capable of hybridizing to a target DUX4 polyA signal RNA sequence, wherein the AAV vector comprises one or more sequences that have at least 80%, at least 85%, at least 90%, at least 92%, at least 95%, at least 97%, at least 99% or 100% sequence identity to SEQ ID NO: 32, SEQ ID NO: 180, SEQ ID NO: 33, SEQ ID NO: 196, SEQ ID NO: 79, or SEQ ID NO: 207.
Embodiment 39. A pharmaceutical composition comprising: a) the engineered guide RNA or the polynucleotide encoding the engineered guide RNA of any one of embodiments 1-37, or the AAV vector of embodiment 38; and b) a pharmaceutically acceptable: excipient, carrier, or diluent.
Embodiment 40. A method of treating a disease or a condition in a subject in need thereof, the method comprising administering to the subject a therapeutically effective amount of the engineered guide RNA or the polynucleotide encoding the engineered guide RNA of any one of embodiments 1-37, the AAV vector of embodiment 38, or the pharmaceutical composition of embodiment 39.
Embodiment 41. The method of embodiment 40, wherein the disease or condition comprises Facioscapulohumeral muscular dystrophy (FSHD).
Embodiment 42. An engineered guide RNA or a polynucleotide encoding the engineered guide RNA, wherein the engineered guide RNA hybridizes to at least 80 bases of a target RNA sequence with at least 80%, at least 85%, at least 90%, at least 92%, at least 95%, at least 96%, at least 97%, at least 98%, at least 99%, or 100% sequence identity to

(SEQ ID NO: 1)
ACCUGGAUUAGAGUUACAUCUCCUGGAUGAUUAGUUCAGAGAUAUAUUAA
AAUGCCCCCUCCCUGUGGAUCCUAUAGAAGAUUUGCAUCUUUUGUGUGAU
GAGUGCAGAG.

Embodiment 43. The engineered guide RNA or the polynucleotide encoding the engineered guide RNA of embodiment 42, wherein upon hybridization of the engineered guide RNA to the target RNA sequence, the engineered guide RNA facilitates RNA editing of one or more adenosines in the target RNA sequence by an RNA editing entity.
Embodiment 44. The engineered guide RNA or the polynucleotide encoding the engineered guide RNA of embodiment 43, wherein the one or more adenosines is in a polyA signal site, which comprises AUUAAA.
Embodiment 45. The engineered guide RNA or the polynucleotide encoding the engineered guide RNA of embodiment 43, wherein the engineered guide RNA upon hybridization to the target RNA sequence forms a guide-target RNA scaffold that comprises one or more structural features.
Embodiment 46. The engineered guide RNA or the polynucleotide encoding the engineered guide RNA of embodiment 45, wherein the one or more structural features comprises a bulge, wherein the bulge is a symmetric bulge.
Embodiment 47. The engineered guide RNA or the polynucleotide encoding the engineered guide RNA of embodiment 45, wherein the one or more structural features comprises a bulge, wherein the bulge is an asymmetric bulge.
Embodiment 48. The engineered guide RNA or the polynucleotide encoding the engineered guide RNA of embodiment 45, wherein the one or more structural features comprises an internal loop, wherein the internal loop is a symmetric internal loop.
Embodiment 49. The engineered guide RNA or the polynucleotide encoding the engineered guide RNA of embodiment 45, wherein the one or more structural features comprises an internal loop, wherein the internal loop is an asymmetric internal loop.
Embodiment 50. The engineered guide RNA or the polynucleotide encoding the engineered guide RNA of embodiment 45, wherein the one or more structural features comprises a Wobble base pair.
Embodiment 51. The engineered guide RNA or the polynucleotide encoding the engineered guide RNA of embodiment 45, wherein the one or more structural features comprises a hairpin, wherein the hairpin is a recruitment hairpin or a non-recruitment hairpin.
Embodiment 52. The engineered guide RNA or the polynucleotide encoding the engineered guide RNA of any one of embodiments 43-51, wherein the RNA editing entity comprises ADAR1, ADAR2, ADAR3, or any combination thereof.
Embodiment 53. The engineered guide RNA or the polynucleotide encoding the engineered guide RNA of embodiment 42, wherein the polynucleotide encoding the engineered guide RNA is comprised in or on a vector.
Embodiment 54. The engineered guide RNA or the polynucleotide encoding the engineered guide RNA of embodiment 53, wherein the vector is a viral vector, and wherein the engineered guide RNA or the polynucleotide encoding the engineered guide RNA is encapsidated in the viral vector.
Embodiment 55. The engineered guide RNA or the polynucleotide encoding the engineered guide RNA of embodiment 54, wherein the viral vector is an adeno-associated viral (AAV) vector or a derivative thereof.
Embodiment 56. The engineered guide RNA or the polynucleotide encoding the engineered guide RNA of embodiment 55, wherein the AAV vector is AAV1, AAV2, AAV3, AAV4, AAV5, AAV6, AAV7, AAV8, AAV9, AAV10, AAV11, or a derivative, a chimera, or a variant thereof.
Embodiment 57. The engineered guide RNA or the polynucleotide encoding the engineered guide RNA of embodiment 55 or 56, wherein the AAV vector is a recombinant AAV (rAAV) vector, a hybrid AAV vector, a chimeric AAV vector, a self-complementary AAV (scAAV) vector, or any combination thereof.
Embodiment 58. A pharmaceutical composition comprising: a) the engineered guide RNA or the polynucleotide encoding the engineered guide RNA of any one of embodiments 42-57; and b) a pharmaceutically acceptable: excipient, carrier, or diluent.
Embodiment 59. A method of treating a disease or a condition in a subject in need thereof, the method comprising administering to the subject a therapeutically effective amount of the engineered guide RNA or the polynucleotide encoding the engineered guide RNA of any one of embodiments 42-57 or the pharmaceutical composition of embodiment 58.
Embodiment 60. The method of embodiment 59, wherein the disease or condition comprises Facioscapulohumeral muscular dystrophy (FSHD).
Embodiment 61. The method of any one of embodiments 59-60, wherein the subject is human or a non-human animal.
Embodiment 62. An engineered guide RNA or a polynucleotide encoding the engineered guide RNA, comprising a polynucleotide sequence with at least 80%, at least 85%, at least 90%, at least 92%, at least 95%, at least 96%, at least 97%, at least 98%, at least 99%, or 100% sequence identity to any one of SEQ ID NO: 32-SEQ ID NO: 38, SEQ ID NO: 39-SEQ ID NO: 46; or SEQ ID NO: 79, 180, 196, 207, 212, 228, and 239.
Embodiment 63. An engineered guide RNA or a polynucleotide encoding the engineered guide RNA, wherein the engineered guide RNA hybridizes to at least 80 bases of a target RNA sequence with at least 80%, at least 85%, at least 90%, at least 92%, at least 95%, at least 96%, at least 97%, at least 98%, at least 99%, or 100% sequence identity to ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1) and facilitates a protein knockdown.

### Embodiments Section 2

Embodiment 1. An engineered guide RNA or a polynucleotide encoding the engineered guide RNA, wherein the engineered guide RNA is capable of hybridizing to a DUX4 target sequence of a DUX4-FL mRNA, wherein the DUX4 target sequence comprises a polyA signal sequence, and wherein the engineered guide RNA: a) has at least 80%, at least 85%, at least 90%, at least 92%, at least 95%, at least 96%, at least 97%, at least 98%, at least 99% or 100% sequence identity to one of SEQ ID NO: 39, SEQ ID NO: 40, SEQ ID NO: 42, SEQ ID NO: 44, SEQ ID NO: 46, SEQ ID NO: 93, SEQ ID NO: 228, or SEQ ID NO: 239; and b) upon hybridization to the target sequence, forms a guide-target RNA scaffold comprising at least 3 structural features including at least one 6/6 symmetric internal loop.
Embodiment 2. The engineered guide RNA or the polynucleotide encoding the engineered guide RNA of embodiment 1, wherein the engineered guide comprises 95 to 105 nucleotides or about 100 nucleotides.
Embodiment 3. The engineered guide RNA or the polynucleotide encoding the engineered guide RNA of embodiment 1, wherein polynucleotide encoding the engineered guide RNA comprises SEQ ID NO: 32, SEQ ID NO: 33, SEQ ID NO: 35, SEQ ID NO: 37, SEQ ID NO: 57, SEQ ID NO: 79, SEQ ID NO: 196, or SEQ ID NO: 207.
Embodiment 4. The engineered guide RNA or the polynucleotide encoding the engineered guide RNA of embodiment 1, the guide-target RNA scaffold comprises at least three wobble base pairs.
Embodiment 5. The engineered guide RNA or the polynucleotide encoding the engineered guide RNA of embodiment 4, wherein the engineered guide RNA comprises SEQ ID NO: 228 or SEQ ID NO: 239.
Embodiment 6. The engineered guide RNA or the polynucleotide encoding the engineered guide RNA of embodiment 4, wherein the polynucleotide encoding the engineered guide RNA comprises SEQ ID NO: 196 or SEQ ID NO: 207.
Embodiment 7. The engineered guide RNA or the polynucleotide encoding the engineered guide RNA of embodiment 1, wherein the guide-target RNA scaffold comprises at least one wobble base pair.
Embodiment 8. The engineered guide RNA or the polynucleotide encoding the engineered guide RNA of embodiment 7, wherein the guide-target RNA scaffold comprises at least two wobble base pairs.
Embodiment 9. The engineered guide RNA or the polynucleotide encoding the engineered guide RNA of embodiment 8, wherein the engineered guide RNA comprises SEQ ID NO: 228, SEQ ID NO: 239, or SEQ ID NO: 46.
Embodiment 10. The engineered guide RNA or the polynucleotide encoding the engineered guide RNA of embodiment 8, wherein the polynucleotide encoding the engineered guide RNA comprises SEQ ID NO: 196, SEQ ID NO: 207, or SEQ ID NO: 79.
Embodiment 11. The engineered guide RNA or the polynucleotide encoding the engineered guide RNA of embodiment 1, wherein the guide-target RNA scaffold comprises a mismatch and at least two 6/6 symmetric internal loops.
Embodiment 12. The engineered guide RNA or the polynucleotide encoding the engineered guide RNA of embodiment 11, wherein the engineered guide RNA comprises SEQ ID NO: 39, SEQ ID NO: 40, SEQ ID NO: 42, SEQ ID NO: 44, SEQ ID NO: 93, or SEQ ID NO: 228.
Embodiment 13. The engineered guide RNA or the polynucleotide encoding the engineered guide RNA of embodiment 11, wherein the polynucleotide encoding the engineered guide RNA comprises SEQ ID NO: 32, SEQ ID NO: 33, SEQ ID NO: 35, SEQ ID NO: 37, SEQ ID NO: 57, or SEQ ID NO: 196.
Embodiment 14. The engineered guide RNA or the polynucleotide encoding the engineered guide RNA of embodiment 1, wherein the guide-target RNA scaffold comprises at least two asymmetric bulges.
Embodiment 15. The engineered guide RNA or the polynucleotide encoding the engineered guide RNA of embodiment 14, wherein the engineered guide RNA comprises SEQ ID NO: 46 or SEQ ID NO: 239.
Embodiment 16. The engineered guide RNA or the polynucleotide encoding the engineered guide RNA of embodiment 14, wherein the polynucleotide encoding the engineered guide RNA comprises SEQ ID NO: 79 or SEQ ID NO: 207.
Embodiment 17. The engineered guide RNA or the polynucleotide encoding the engineered guide RNA of embodiment 1, wherein the guide-target RNA scaffold comprises at least one symmetric bulge.
Embodiment 18. The engineered guide RNA or the polynucleotide encoding the engineered guide RNA of embodiment 17, wherein the engineered guide RNA comprises SEQ ID NO: 46 or SEQ ID NO: 239.
Embodiment 19. The engineered guide RNA or the polynucleotide encoding the engineered guide RNA of embodiment 17, wherein the polynucleotide encoding the engineered guide RNA comprises SEQ ID NO: 79 or SEQ ID NO: 207.
Embodiment 20. The engineered guide RNA or the polynucleotide encoding the engineered guide RNA of embodiment 1, wherein the 6/6 symmetric internal loop is at position 44 relative to a target adenosine at position 0.
Embodiment 21. The engineered guide RNA or the polynucleotide encoding the engineered guide RNA of embodiment 20, wherein the engineered guide RNA comprises SEQ ID NO: 39 or SEQ ID NO: 93.
Embodiment 22. The engineered guide RNA or the polynucleotide encoding the engineered guide RNA of embodiment 20, wherein the polynucleotide encoding the engineered guide RNA comprises SEQ ID NO: 32 or SEQ ID NO: 57.
Embodiment 23. The engineered guide RNA or the polynucleotide encoding the engineered guide RNA of embodiment 1, wherein the 6/6 symmetric internal loop is at position −8 relative to a target adenosine at position 0.
Embodiment 24. The engineered guide RNA or the polynucleotide encoding the engineered guide RNA of embodiment 23, wherein the engineered guide RNA comprises SEQ ID NO: 42 or SEQ ID NO: 93.
Embodiment 25. The engineered guide RNA or the polynucleotide encoding the engineered guide RNA of embodiment 23, wherein the polynucleotide encoding the engineered guide RNA comprises SEQ ID NO: 35 or SEQ ID NO: 57.
Embodiment 26. The engineered guide RNA or the polynucleotide encoding the engineered guide RNA of embodiment 1, wherein the 6/6 symmetric internal loop is at position 27 relative to a target adenosine at position 0.
Embodiment 27. The engineered guide RNA or the polynucleotide encoding the engineered guide RNA of embodiment 26, wherein the engineered guide RNA comprises SEQ ID NO: 40 or SEQ ID NO: 228.
Embodiment 28. The engineered guide RNA or the polynucleotide encoding the engineered guide RNA of embodiment 26, wherein the polynucleotide encoding the engineered guide RNA comprises SEQ ID NO: 33 or SEQ ID NO: 196.
Embodiment 29. The engineered guide RNA or the polynucleotide encoding the engineered guide RNA of embodiment 1, wherein the 6/6 symmetric internal loop is at position −6 relative to a target adenosine at position 0.
Embodiment 30. The engineered guide RNA or the polynucleotide encoding the engineered guide RNA of embodiment 29, wherein the engineered guide RNA comprises SEQ ID NO: 40 or SEQ ID NO: 228.
Embodiment 31. The engineered guide RNA or the polynucleotide encoding the engineered guide RNA of embodiment 29, wherein the polynucleotide encoding the engineered guide RNA comprises SEQ ID NO: 33 or SEQ ID NO: 196.
Embodiment 32. The engineered guide RNA or the polynucleotide encoding the engineered guide RNA of embodiment 1, wherein the 6/6 symmetric internal loop is at position −22 relative to a target adenosine at position 0.
Embodiment 33. The engineered guide RNA or the polynucleotide encoding the engineered guide RNA of embodiment 32, wherein the engineered guide RNA comprises SEQ ID NO: 46 or SEQ ID NO: 239.
Embodiment 34. The engineered guide RNA or the polynucleotide encoding the engineered guide RNA of embodiment 32, wherein the polynucleotide encoding the engineered guide RNA comprises SEQ ID NO: 79 or SEQ ID NO: 207.
Embodiment 35. The engineered guide RNA or the polynucleotide encoding the engineered guide RNA of embodiment 1, wherein the 6/6 symmetric internal loop is at position −8 relative to a target adenosine at position 0.
Embodiment 36. The engineered guide RNA or the polynucleotide encoding the engineered guide RNA of embodiment 35, wherein the engineered guide RNA comprises SEQ ID NO: 42 or SEQ ID NO: 93.
Embodiment 37. The engineered guide RNA or the polynucleotide encoding the engineered guide RNA of embodiment 35, wherein the polynucleotide encoding the engineered guide RNA comprises SEQ ID NO: 35 or SEQ ID NO: 57.
Embodiment 38. The engineered guide RNA or the polynucleotide encoding the engineered guide RNA of embodiment 1, wherein the 6/6 symmetric internal loop is at position −11 relative to a target adenosine at position 0.
Embodiment 39. The engineered guide RNA or the polynucleotide encoding the engineered guide RNA of embodiment 38, wherein the engineered guide RNA comprises SEQ ID NO: 44.
Embodiment 40. The engineered guide RNA or the polynucleotide encoding the engineered guide RNA of embodiment 38, wherein the polynucleotide encoding the engineered guide RNA comprises SEQ ID NO: 37.
Embodiment 41. The engineered guide RNA or the polynucleotide encoding the engineered guide RNA of embodiment 1, wherein the 6/6 symmetric internal loop is at position 36 relative to a target adenosine at position 0.
Embodiment 42. The engineered guide RNA or the polynucleotide encoding the engineered guide RNA of embodiment 41, wherein the engineered guide RNA comprises SEQ ID NO: 46.
Embodiment 43. The engineered guide RNA or the polynucleotide encoding the engineered guide RNA of embodiment 41, wherein the polynucleotide encoding the engineered guide RNA comprises SEQ ID NO: 79.
Embodiment 44. The engineered guide RNA or the polynucleotide encoding the engineered guide RNA of embodiment 1, wherein the at least 3 structural features, including the at least one 6/6 symmetric internal loop, comprise two symmetric internal loops at positions −5 and 44 relative to a target adenosine at position 0, and a mismatch at position 0 relative to the target adenosine at position 0.
Embodiment 45. The engineered guide RNA or the polynucleotide encoding the engineered guide RNA of embodiment 44, wherein the engineered guide RNA comprises SEQ ID NO: 39.
Embodiment 46. The engineered guide RNA or the polynucleotide encoding the engineered guide RNA of embodiment 44, wherein the polynucleotide encoding the engineered guide RNA comprises SEQ ID NO: 32.
Embodiment 47. The engineered guide RNA or the polynucleotide encoding the engineered guide RNA of embodiment 1, wherein the at least 3 structural features, including the at least one 6/6 symmetric internal loop, comprise two symmetric internal loops at positions −6 and 27 relative to a target adenosine at position 0, and a mismatch at position 0 relative to a target adenosine at position 0.
Embodiment 48. The engineered guide RNA or the polynucleotide encoding the engineered guide RNA of embodiment 47, wherein the engineered guide RNA comprises SEQ ID NO: 40.
Embodiment 49. The engineered guide RNA or the polynucleotide encoding the engineered guide RNA of embodiment 47, wherein the polynucleotide encoding the engineered guide RNA comprises SEQ ID NO: 33.
Embodiment 50. The engineered guide RNA or the polynucleotide encoding the engineered guide RNA of embodiment 1, wherein the at least 3 structural features, including the at least one 6/6 symmetric internal loop comprise, two symmetric internal loops at positions −6 and 27 relative to a target adenosine at position 0, a mismatch at position 0 relative to the target adenosine at position 0 and a wobble base pair at positions −35, −32, −27, −20, −18, −17, −15, 8, 20, 22, 37, 45, 49, 55, and 59 relative to the target adenosine at position 0.
Embodiment 51. The engineered guide RNA or the polynucleotide encoding the engineered guide RNA of embodiment 50, wherein the engineered guide RNA comprises SEQ ID NO: 228.
Embodiment 52. The engineered guide RNA or the polynucleotide encoding the engineered guide RNA of embodiment 50, wherein the polynucleotide encoding the engineered guide RNA comprises SEQ ID NO: 196.
Embodiment 53. The engineered guide RNA or the polynucleotide encoding the engineered guide RNA of embodiment 1, wherein the at least 3 structural features, including the at least one 6/6 symmetric internal loop, comprise two symmetric internal loops at positions −22 and 36 relative to a target adenosine at position 0, an asymmetric bulge at positions −13 and −7 relative to the target adenosine at position 0, a symmetric bulge at position −3 relative to the target adenosine at position 0, and a wobble base pair at positions −21 and −14 relative to the target adenosine at position 0.
Embodiment 54. The engineered guide RNA or the polynucleotide encoding the engineered guide RNA of embodiment 53, wherein the engineered guide RNA comprises SEQ ID NO: 46.
Embodiment 55. The engineered guide RNA or the polynucleotide encoding the engineered guide RNA of embodiment 53, wherein the polynucleotide encoding the engineered guide RNA comprises SEQ ID NO: 79.
Embodiment 56. The engineered guide RNA or the polynucleotide encoding the engineered guide RNA of embodiment 1, wherein the at least 3 structural features, including the at least one 6/6 symmetric internal loop comprise, a symmetric internal loop at positions −22 relative to a target adenosine at position 0, an asymmetric bulge at positions −13 and −7 relative to the target adenosine at position 0, a symmetric bulge at positions −3 and 36 relative to the target adenosine at position 0, a mismatch at position 41 relative to a target adenosine at position 0, and a wobble base pair at positions −35, −33, −31, −21, −14, 18, 20, 22, 24, 29, 31, 34, 45, 48, 51, 55, 58, 62, and 64 relative to the target adenosine at position 0.
Embodiment 57. The engineered guide RNA or the polynucleotide encoding the engineered guide RNA of embodiment 56, wherein the engineered guide RNA comprises SEQ ID NO: 239.
Embodiment 58. The engineered guide RNA or the polynucleotide encoding the engineered guide RNA of embodiment 56, wherein the polynucleotide encoding the engineered guide RNA comprises SEQ ID NO: 207.
Embodiment 59. The engineered guide RNA or the polynucleotide encoding the engineered guide RNA of embodiment 1, wherein the at least 3 structural features, including the at least one 6/6 symmetric internal loop, comprise two symmetric internal loops at positions −8 and 35 relative to a target adenosine at position 0, and a mismatch at position 3 relative to the target adenosine at position 0.
Embodiment 60. The engineered guide RNA or the polynucleotide encoding the engineered guide RNA of embodiment 59, wherein the engineered guide RNA comprises SEQ ID NO: 42.
Embodiment 61. The engineered guide RNA or the polynucleotide encoding the engineered guide RNA of embodiment 59, wherein the polynucleotide encoding the engineered guide RNA comprises SEQ ID NO: 35.
Embodiment 62. The engineered guide RNA or the polynucleotide encoding the engineered guide RNA of embodiment 1, wherein the at least 3 structural features, including the at least one 6/6 symmetric internal loop, comprise two symmetric internal loops at positions −8 and 44 relative to a target adenosine at position 0, and a mismatch at position 0 relative to the target adenosine at position 0.
Embodiment 63. The engineered guide RNA or the polynucleotide encoding the engineered guide RNA of embodiment 62, wherein the engineered guide RNA comprises SEQ ID NO: 93.
Embodiment 64. The engineered guide RNA or the polynucleotide encoding the engineered guide RNA of embodiment 62, wherein the polynucleotide encoding the engineered guide RNA comprises SEQ ID NO: 57.
Embodiment 65. The engineered guide RNA or the polynucleotide encoding the engineered guide RNA of embodiment 1, wherein the at least 3 structural features, including the at least one 6/6 symmetric internal loop, comprise two symmetric internal loops at positions −11 and 42 relative to a target adenosine at position 0, and a mismatch at position 4 relative to the target adenosine at position 0.
Embodiment 66. The engineered guide RNA or the polynucleotide encoding the engineered guide RNA of embodiment 65, wherein the engineered guide RNA comprises SEQ ID NO: 44.
Embodiment 67. The engineered guide RNA or the polynucleotide encoding the engineered guide RNA of embodiment 65, wherein the polynucleotide encoding the engineered guide RNA comprises SEQ ID NO: 37.
Embodiment 68. The engineered guide RNA or the polynucleotide encoding the engineered guide RNA of embodiment 1, wherein the engineered guide RNA hybridizes to at least 80 bases of the DUX4 target sequence that has at least 80%, at least 85%, at least 90%, at least 92%, at least 95%, at least 96%, at least 97%, at least 98%, at least 99%, or 100% sequence identity to

(SEQ ID NO: 1)
ACCUGGAUUAGAGUUACAUCUCCUGGAUGAUUAGUUCAGAGAUAUAUUAA
AAUGCCCCCUCCCUGUGGAUCCUAUAGAAGAUUUGCAUCUUUUGUGUGAU
GAGUGCAGAG.

Embodiment 69. The engineered guide RNA or the polynucleotide encoding the engineered guide RNA of embodiment 1, wherein the engineered guide RNA when hybridized to the DUX4 target sequence of the DUX4-FL mRNA facilitates a knockdown of a DUX4 protein encoded by the DUX4-FL mRNA.
Embodiment 70. The engineered guide RNA or the polynucleotide encoding the engineered guide RNA of embodiment 69, wherein the knockdown comprises the knockdown of at least 30%, at least 40%, at least 50%, at least 60%, at least 70%, at least 80%, or at least 90% of the DUX4-FL mRNA.
Embodiment 71. The engineered guide RNA or the polynucleotide encoding the engineered guide RNA of embodiment 1, wherein the engineered guide RNA when hybridized to the DUX4 target sequence facilitates a knockdown of a gene downstream of DUX4 and wherein the gene downstream of DUX4 comprises SLC34A2, LEUTX, ZSCAN4, PRAMEF12, TRIM43, KHDC1L, Wfdc3, Agtr2, DEFB103, or MBD3L2.
Embodiment 72. The engineered guide RNA or the polynucleotide encoding the engineered guide RNA of embodiment 71, wherein the knockdown comprises the knockdown of at least 30%, at least 40%, at least 50%, at least 60%, at least 70%, at least 80%, or at least 90% of an mRNA or a protein encoded by the gene downstream of DUX4.
Embodiment 73. The engineered guide RNA or the polynucleotide encoding the engineered guide RNA of embodiment 1, wherein the engineered guide RNA when hybridized to the DUX4 target sequence masks the polyA signal sequence.
Embodiment 74. The engineered guide RNA or the polynucleotide encoding the engineered guide RNA of embodiment 73, wherein the masking of the polyA signal sequence facilitates knockdown of at least about 20%, at least about 30%, at least about 40%, at least about 50%, at least about 60%, at least about 70%, at least about 80%, or at least about 90% of the DUX4-FL mRNA or a gene downstream of DUX4.
Embodiment 75. The engineered guide RNA or the polynucleotide encoding the engineered guide RNA of embodiment 1, wherein the engineered guide RNA when hybridized to the DUX4 target sequence facilitates RNA editing by an RNA editing entity of one or more adenosines in the polyA signal sequence.
Embodiment 76. The engineered guide RNA or the polynucleotide encoding the engineered guide RNA of embodiment 75, wherein the RNA editing entity comprises a human ADAR1, or a human ADAR2.
Embodiment 77. An AAV vector comprising a polynucleotide encoding an engineered guide RNA capable of hybridizing to a DUX4 target sequence of a DUX4-FL mRNA, wherein the DUX4 target sequence comprises a polyA signal sequence, wherein the polynucleotide encoding the engineered guide RNA comprises one or more sequences that have at least 80%, at least 85%, at least 90%, at least 92%, at least 95%, at least 97%, at least 99% or 100% sequence identity to SEQ ID NO: 32, SEQ ID NO: 33, SEQ ID NO: 35, SEQ ID NO: 37, SEQ ID NO: 57, SEQ ID NO: 79, SEQ ID NO: 196, or SEQ ID NO: 207.
Embodiment 78. The AAV vector of embodiment 77, wherein the AAV vector is AAV1, AAV2, AAV3, AAV4, AAV5, AAV6, AAV7, AAV8, AAV9, AAV10, AAV11, or a derivative, a chimera, or a variant thereof.
Embodiment 79. The AAV vector of embodiment 77, wherein the AAV vector is a recombinant AAV (rAAV) vector, a hybrid AAV vector, a chimeric AAV vector, a self-complementary AAV (scAAV) vector, or any combination thereof.
Embodiment 80. A pharmaceutical composition in unit dose form comprising: (a) the engineered guide RNA or the polynucleotide encoding the engineered guide RNA of any one of embodiments 1-76, or the AAV vector of any one of embodiments 77-79; and (b) a pharmaceutically acceptable: excipient, carrier, or diluent.
Embodiment 81. An in vitro method of editing an DUX4-FL RNA transcript in a cell comprising administering to the cell an effective amount of the engineered guide RNA or the polynucleotide encoding the engineered guide RNA of any one of embodiments 1-76, the AAV vector of any one of embodiments 77-79, or the pharmaceutical composition of embodiment 80, wherein after the administering the DUX4-FL RNA transcript is edited in the cell.
Embodiment 82. The method of embodiment 81, wherein the cell is a muscle cell.
Embodiment 83. The method of embodiment 82, wherein the muscle cell comprises a myocyte, a myofibril, a myoblast, a myotube or any combination thereof.
Embodiment 84. The method of embodiment 81, wherein the editing of the DUX4-FL RNA transcript comprises editing of a polyA signal sequence.
Embodiment 85. The method of embodiment 84, wherein the editing of polyA signal sequence comprises editing of at least about 20%, at least about 30%, at least about 40%, at least about 50%, at least about 60%, at least about 70%, at least about 80%, or at least about 90% of the total DUX4-FL transcripts in the cell.
Embodiment 86. The method of embodiment 84, wherein the editing of polyA signal sequence facilitates a knockdown of DUX4 or a gene downstream of DUX4 and wherein the gene downstream of DUX4 comprises SLC34A2, LEUTX, ZSCAN4, PRAMEF12, TRIM43, DEFB103, KHDC1L, Wfdc3, Agtr2, or MBD3L2.
Embodiment 87. The method of embodiment 86, wherein the knockdown comprises the knockdown of at least 30%, at least 40%, at least 50%, at least 60%, at least 70%, at least 80%, or at least 90% of the mRNA encoding DUX4 or of the gene downstream of DUX4.

### EXAMPLES

The following illustrative examples are representative of embodiments of the stimulation, systems, and methods described herein and are not meant to be limiting in any way.

### Example 1

Engineered Guide RNAs for Editing DUX4 polyA Signal Sequence

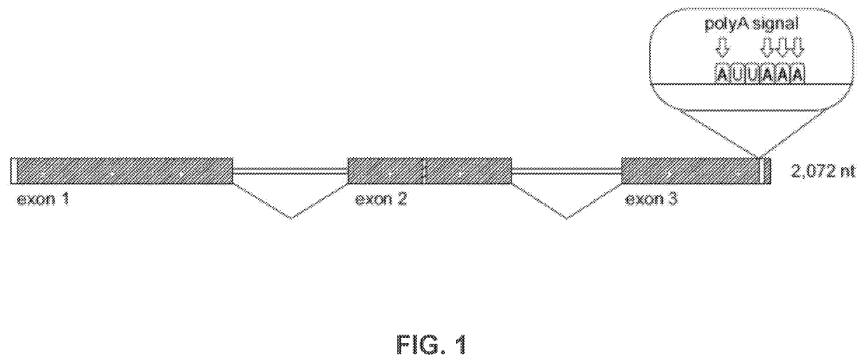

This example describes engineered guide RNAs for editing DUX4 (DUX4-FL) RNA to knockdown expression of the corresponding DUX4 protein. A schematic of the DUX4 target is shown in FIG. 1, highlighting sites that can be targeted by engineered guide RNAs of the present disclosure. Engineered guide RNAs of the present disclosure are designed to target one or more adenosines in the single polyA signal sequence (ATTAAA) of DUX4-FL RNA (e.g., can be directed to hybridize to a region of the target DUX4 mRNA having a sequence of or within SEQ ID NO: 1) and facilitate ADAR-mediated RNA editing of said one or more adenosines, thus, leading to disruption of RNA processing and inducement of degradation of the mRNA. This in turn leads to knockdown of the toxic DUX4-FL protein. Upon administration of the engineered guide RNAs, in vitro or in vivo, the engineered guide RNAs edit the DUX4 polyA signal sequence, thereby reducing expression of DUX4 target genes. Upon administration to a subject having facioscapulohumeral muscular dystrophy (FSHD), the engineered guide RNAs are therapeutically effective and restore proper muscle function.

### Example 2

### Compositions for the Treatment of Facioscapulohumeral Muscular Dystrophy (FSHD)

This example describes a vector for treatment of FSHD. A subject is diagnosed with FSHD, which causes misexpression of the DUX4-FL gene in muscle cells. The subject is prescribed a dosing regimen of a pharmaceutical composition. The pharmaceutical composition comprises a vector comprising an engineered guide RNA described herein that is directed to target DUX4-FL mRNA sequence of or within ACCUGGAUUAGAGUUACAUCUCCUGGAUGAUUAGUUCAGAGAUAUAUUAAAAUG CCCCCUCCCUGUGGAUCCUAUAGAAGAUUUGCAUCUUUUGUGUGAUGAGUGCAGA G (SEQ ID NO: 1). The pharmaceutical composition is administered systemically to the subject by intravenous administration in an effective amount to treat the FSHD disease.

### Example 3

### Identification of Guide RNAs for Targeting DUX4 RNA

Engineered guide RNAs were identified from a cell-free high throughput screen (HTS) targeting the polyA signal sequence (AUUAAA) of DUX4 mRNA. Guide RNAs were further validated in a Hek-Luc-Dux4 reporter screen for reduced expression in luciferase as a proxy for reduced DUX4. Guide RNAs were tested in a 100.80 format. Guide RNAs described as ###.## (e.g., 100.55) indicate the length of the guide RNA and the location of the mismatch. The first number indicates the length of the target RNA region to which the guide RNA hybridizes, and the second number indicates the position of the target adenosine within this region of the target RNA, counting from the 3′ end. Select guide RNAs were further identified by leveraging machine learning-based algorithms from guide RNAs trained on the cell-free high throughput screen (e.g., SEQ ID NO: 45-46 and SEQ ID NO: 111-115).

To further validate the selected guide RNAs, an integrated tetracycline (tet) inducible DUX4 expression system was developed in HEK293T cells to test the effect of DUX4 expression on cell survival. Induced expression of DUX4 mRNA and protein by doxycycline in the inducible system leads to cell death by overexpression of DUX4. Relative caspase activity (caspase 3 and 7) is determined by a fluorometric readout. Increased caspase activity indicates apoptosis, which is represented by an increase in luminescence units. Using the inducible DUX4 cell line, select guide RNAs were further modified by macro-footprint positioning and guide RNA shortening.

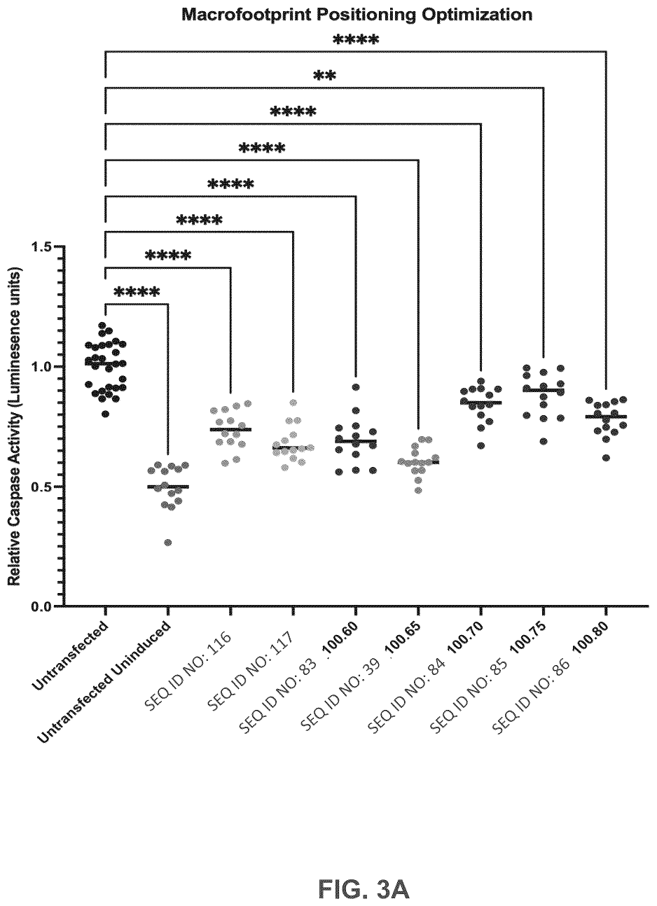

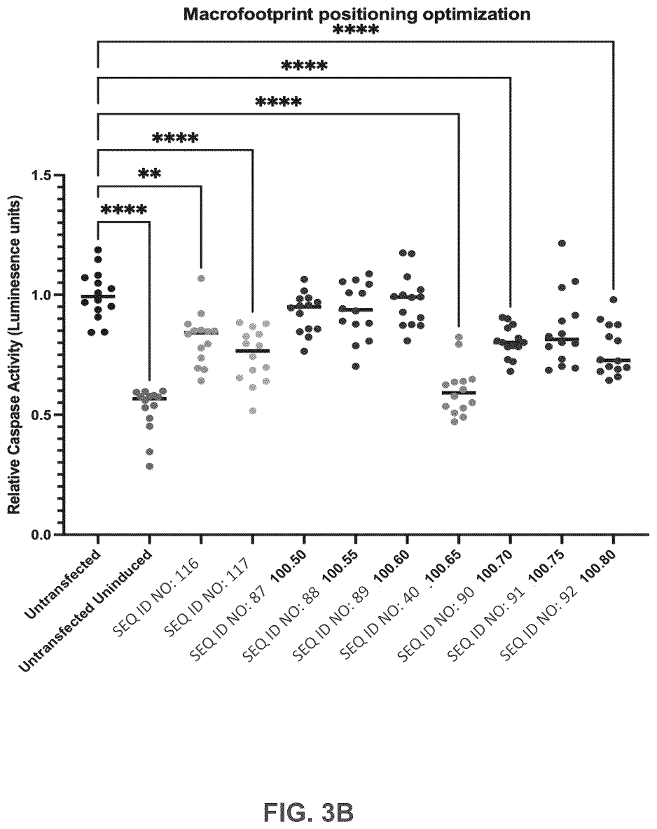

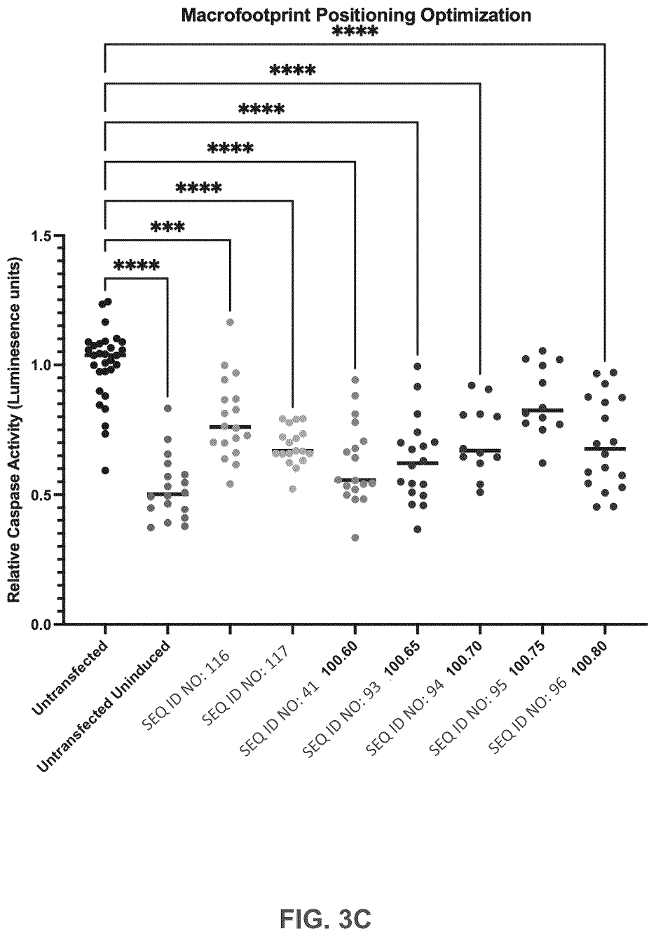

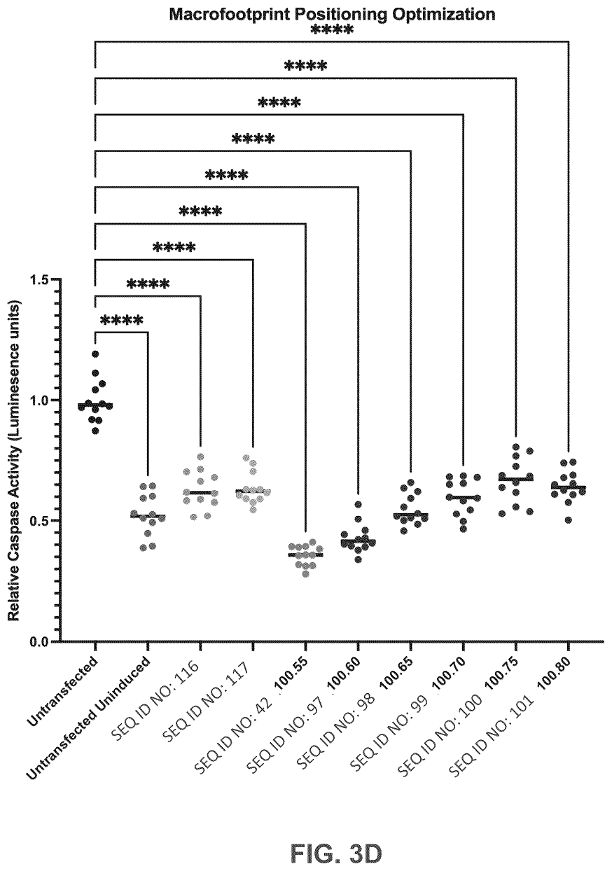

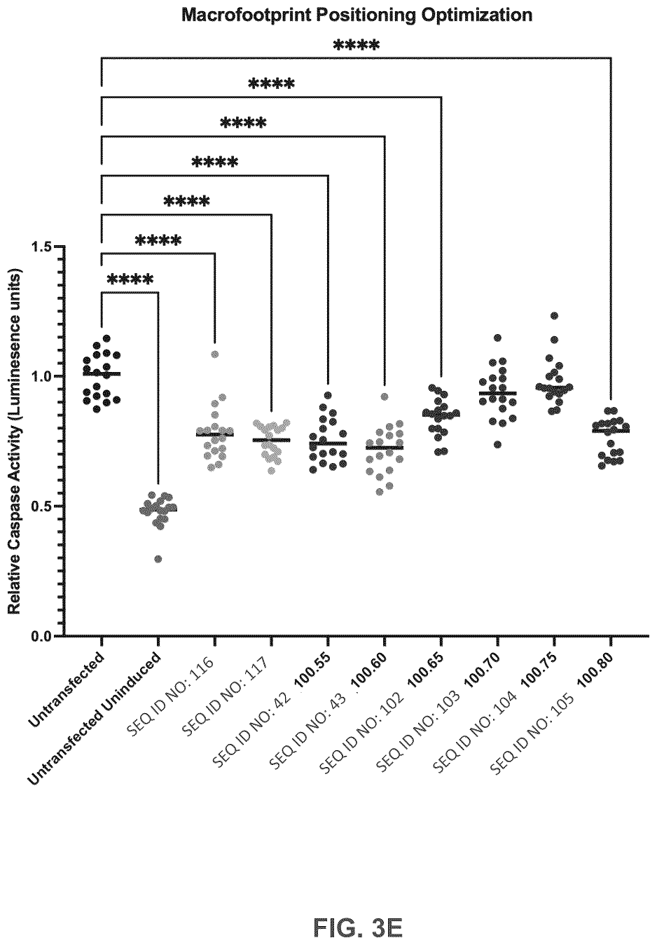

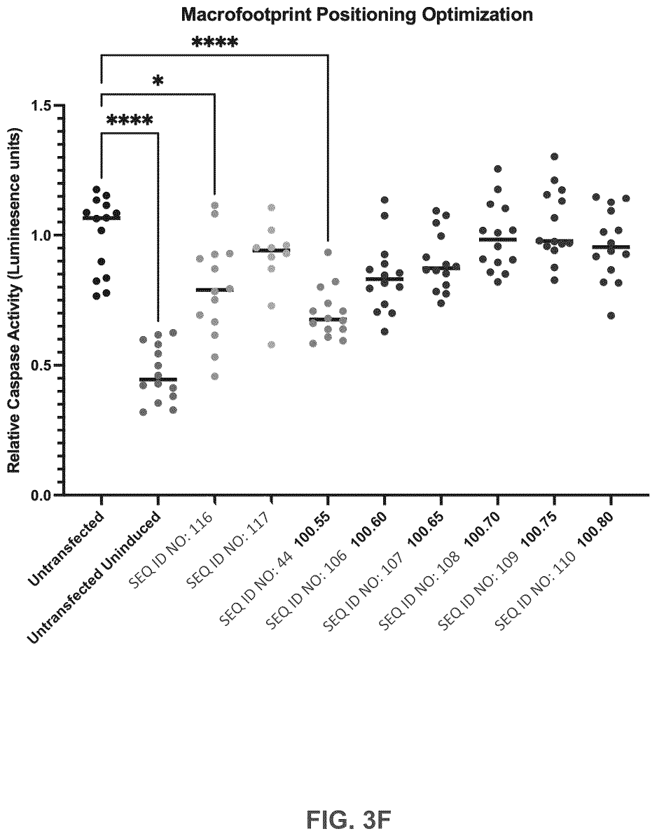

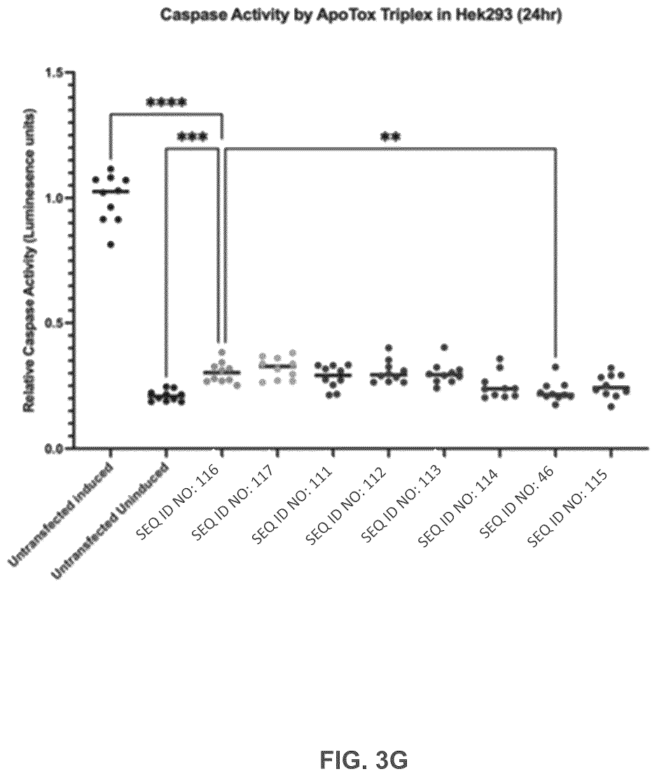

FIG. 3A-3G show the relative caspase activity in cells using the HEK293T Tet-inducible DUX4 expression system. Guide RNAs were tested for reduced caspase activity by shifting the micro-footprint in select guide RNA templates. FIG. 3A-3F show different guide RNAs with the same macro-footprint, but which have changes in the location of the mismatch relative to the target adenosine. FIG. 3G shows guide RNAs with different locations of internal loops and/or changes in the location of the mismatch relative to the target adenosine. FIG. 3A shows macro-footprint adjustment of the SEQ ID NO: 86 template guide RNA. The guide RNA with a mismatch to the target adenosine at nucleotide 65 (SEQ ID NO: 39), numbered according to the 3′ end of the target RNA region to which the guide RNA hybridizes, had the largest reduction in caspase activity. FIG. 3B shows macro-footprint adjustment in the SEQ ID NO: 92 template guide RNA. The guide RNA with a mismatch to the target adenosine at nucleotide 65 (SEQ ID NO: 40), numbered according to the 3′ end of the target RNA region to which the guide RNA hybridizes, had the largest reduction in caspase activity. FIG. 3C shows macro-footprint adjustment in the SEQ ID NO: 96 guide RNA. The guide RNA with a mismatch to the target adenosine at nucleotide 60 (SEQ ID NO: 41), numbered according to the numbered according to the 3′ end of the target RNA region to which the guide RNA hybridizes, had the largest reduction in caspase activity. FIG. 3D shows macro-footprint adjustments in the SEQ ID NO: 101 template guide RNA. The guide RNA with the mismatch to the target adenosine at nucleotide 55 (SEQ ID NO: 42), numbered according to the numbered according to the 3′ end of the target RNA region to which the guide RNA hybridizes, had the largest reduction in caspase activity. FIG. 3E shows macro-footprint adjustment in the SEQ ID NO: 105 template guide RNA. The guide RNA with a mismatch to the target adenosine at nucleotide 60 (SEQ ID NO: 43), numbered according to the numbered according to the 3′ end of the target RNA region to which the guide RNA hybridizes, had the largest reduction in caspase activity. FIG. 3F shows macro-footprint adjustment in the SEQ ID NO: 110 template guide RNA. The guide RNA with the mismatch to the target adenosine at nucleotide 55 (SEQ ID NO: 44), numbered according to the numbered according to the 3′ end of the target RNA region to which the guide RNA hybridizes, had the largest reduction in caspase activity. FIG. 3G shows macro-footprint adjustment in a machine learning template guide RNA. The guide RNA with the mismatch to the target adenosine at nucleotide 65 numbered according to the numbered according to the 3′ end of the target RNA region to which the guide RNA hybridizes, (SEQ ID NO: 46) and internal loops at −22 and +36 nucleotide positions, had the largest reduction in caspase activity. The sequences of the guide RNAs in FIG. 3A-3G are shown in Table 3, along with several control guide RNAs used to confirm activity of the guide RNAs. Table 3 also shows the relative caspase activity based on luminescence units from each guide RNA as compared to the untransfected induced cells. Overall, shifting the macro-footprint towards having the target mismatch at nucleotide positions 55 to 65, numbered according to the numbered according to the 3′ end of the target RNA region to which the guide RNA hybridizes, increased the efficacy of guide RNAs to reduce apoptosis as measured by caspase activity.

For each sequence, the structural features formed in the double stranded RNA substrate upon hybridization of the guide RNA to the target DUX4 RNA, are shown in the last column of Table 3. For reference, each structural feature formed within a guide-target RNA scaffold (target RNA sequence hybridized to an engineered guide RNA) is annotated as follows:
a) the position of the structural feature with respect to the target A (position 0—the first A (bolded) in the polyA signal sequence AUUAAA) of the target RNA sequence, with a negative value indicating upstream (5′) of the target A and a positive value indicating downstream (3′) of the target A;
b) the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c) the name of the structural feature (e.g., symmetric bulge, symmetric internal loop, asymmetric bulge, asymmetric internal loop, mismatch, or wobble base pair), and
d) the sequences of bases on the target RNA side and the engineered guide RNA side that participate in forming the structural feature.

For example, with reference to SEQ ID NO: 83, ‘-5_6-6_internal_loop-symmetric_UCAGAG-GCAGCU’ ‘0_1-1_mismatch_A-C’ ‘44_6-6_internal_loop-symmetric UUUUGU-CUACUC” is read as a structural feature formed in a guide-target RNA scaffold (target DUX4 RNA sequence hybridized to an engineered guide RNA of SEQ ID NO: 83), where a structural feature starts 5 nucleotides upstream (5′) (the −5 position) from the target A (0 position) of the target RNA sequence; six contiguous bases from the target RNA sequence and six contiguous bases from the engineered guide RNA form the structural feature; the structural feature is an internal symmetric loop; and a sequence of UCAGAG from the target RNA side and a sequence of GCAGCU from the engineered guide RNA side participate in forming the internal symmetric loop. A structural feature is located at the target A (0 position) of the target RNA sequence; 1 base from the target RNA and 1 base from the engineered guide RNA form the structural feature; the structural feature is a mismatch; and a sequence of A from the target RNA side and a sequence of C from the engineered guide RNA side participate in forming the mismatch. A structural feature starts 44 nucleotides downstream (3′) (the +44 position) from the target A (0 position) of the target RNA sequence; six contiguous bases from the target RNA sequence and six contiguous bases from the engineered guide RNA form the structural feature; the structural feature is an internal symmetric loop; and a sequence of UUUUGU from the target RNA side and a sequence of CUACUC from the engineered guide RNA side participate in forming the internal symmetric loop.

TABLE 3
Guide RNAs for targeting the polyA signal sequence of DUX4

| | | | Relative caspase | |
|---|---|---|---|---|
| | | | activity to | Structural Features of |
| | | | untransfected | the guide-target RNA |
| FIG. | Guide DNA sequence | Guide RNA sequence | induced cells | scaffold |
| FIG. 3A | CACTCATCACCTACTC | CACUCAUCACCUACUCG | 0.68 | −5_6-6_internal_loop- |
| | GATGCAAATCTTCTAT | AUGCAAAUCUUCUAUAG | | symmetric_UCAGAG- |
| | AGGATCCACAGGGAG | GAUCCACAGGGAGGGGG | | GCAGCU |
| | GGGGCATTTTAACAT | CAUUUUAACAUAUGCAG | | 0_1-1_mismatch_A-C |
| | ATGCAGCTACTAATC | CUACUAAUCAUCCAGGA | | 44_6-6_internal_loop- |
| | ATCCAGGAGATGTAA | GAUGUAACUCUAAUC | | symmetric_UUUUGU- |
| | CTCTAATC (SEQ ID | (SEQ ID NO: 83) | | CUACUC |
| | NO: 47) | | | |
| FIG. 3A | CTCTGCACTCATCACC | CUCUGCACUCAUCACCU | 0.601 | −5_6-6_internal_loop- |
| | TACTCGATGCAAATC | ACUCGAUGCAAAUCUUC | | symmetric_UCAGAG- |
| | TTCTATAGGATCCAC | UAUAGGAUCCACAGGGA | | GCAGCU |
| | AGGGAGGGGGCATTT | GGGGGCAUUUUAACAUA | | 0_1-1_mismatch_A-C |
| | TAACATATGCAGCTA | UGCAGCUACUAAUCAUC | | 44_6-6_internal_loop- |
| | CTAATCATCCAGGAG | CAGGAGAUGUAACUC | | symmetric_UUUUGU- |
| | ATGTAACTC (SEQ ID | (SEQ ID NO: 39) | | CUACUC |
| | NO: 32) | | | |
| FIG. 3A | CATATCTCTGCACTCA | CAUAUCUCUGCACUCAU | 0.849 | −5_6-6_internal_loop- |
| | TCACCTACTCGATGC | CACCUACUCGAUGCAAA | | symmetric_UCAGAG- |
| | AAATCTTCTATAGGA | UCUUCUAUAGGAUCCAC | | GCAGCU |
| | TCCACAGGGAGGGGG | AGGGAGGGGGCAUUUUA | | 0_1-1_mismatch_A-C |
| | CATTTTAACATATGCA | ACAUAUGCAGCUACUAA | | 44_6-6_internal_loop- |
| | GCTACTAATCATCCA | UCAUCCAGGAGAUGU | | symmetric_UUUUGU- |
| | GGAGATGT (SEQ ID | (SEQ ID NO: 84) | | CUACUC |
| | NO: 48) | | | |
| FIG. 3A | TGTGACATATCTCTGC | UGUGACAUAUCUCUGCA | 0.90 | −5_6-6_internal_loop- |
| | ACTCATCACCTACTCG | CUCAUCACCUACUCGAU | | symmetric_UCAGAG- |
| | ATGCAAATCTTCTATA | GCAAAUCUUCUAUAGGA | | GCAGCU |
| | GGATCCACAGGGAGG | UCCACAGGGAGGGGGCA | | 0_1-1_mismatch_A-C |
| | GGGCATTTTAACATA | UUUUAACAUAUGCAGCU | | 44_6-6_internal_loop- |
| | TGCAGCTACTAATCA | ACUAAUCAUCCAGGA | | symmetric_UUUUGU- |
| | TCCAGGA (SEQ ID NO: | (SEQ ID NO: 85) | | CUACUC |
| | 49) | | | |
| FIG. 3A | GATATTGTGACATAT | GAUAUUGUGACAUAUCU | 0.79 | −5_6-6_internal_loop- |
| | CTCTGCACTCATCACC | CUGCACUCAUCACCUAC | | symmetric_UCAGAG- |
| | TACTCGATGCAAATC | UCGAUGCAAAUCUUCUA | | GCAGCU |
| | TTCTATAGGATCCAC | UAGGAUCCACAGGGAGG | | 0_1-1_mismatch_A-C |
| | AGGGAGGGGGCATTT | GGGCAUUUUAACAUAUG | | 44_6-6_internal_loop- |
| | TAACATATGCAGCTA | CAGCUACUAAUCAUC | | symmetric_UUUUGU- |
| | CTAATCATC (SEQ ID | (SEQ ID NO: 86) | | CUACUC |
| | NO: 50) | | | |
| FIG. 3A | ACAAAAGATGCAAAT | ACAAAAGAUGCAAAUCU | 0.949 | −6_6-6_internal_loop- |
| | CTGTGGGCGGATCCA | GUGGGCGGAUCCACAGG | | symmetric_UUCAGA- |
| | CAGGGAGGGGGCATT | GAGGGGGCAUUUUAACA | | AGCUCC |
| | TTAACATATCAGCTCC | UAUCAGCUCCCUAAUCA | | 0_1-1_mismatch_A-C |
| | CTAATCATCCAGGAG | UCCAGGAGAUGUAACUC | | 27_6-6_internal_loop- |
| | ATGTAACTCTAATCC | UAAUCCAGGUUUGCC | | symmetric_UAUAGA- |
| | AGGTTTGCC (SEQ ID | (SEQ ID NO: 87) | | GUGGGC |
| | NO: 51) | | | |
| FIG. 3B | ATCACACAAAAGATG | AUCACACAAAAGAUGCA | 0.937 | −6_6-6_internal_loop- |
| | CAAATCTGTGGGCGG | AAUCUGUGGGCGGAUCC | | symmetric_UUCAGA- |
| | ATCCACAGGGAGGGG | ACAGGGAGGGGGCAUUU | | AGCUCC |
| | GCATTTTAACATATCA | UAACAUAUCAGCUCCCU | | 0_1-1_mismatch_A-C |
| | GCTCCCTAATCATCCA | AAUCAUCCAGGAGAUGU | | 27_6-6_internal_loop- |
| | GGAGATGTAACTCTA | AACUCUAAUCCAGGU | | symmetric_UAUAGA- |
| | ATCCAGGT (SEQ ID | (SEQ ID NO: 88) | | GUGGGC |
| | NO: 52) | | | |
| FIG. 3B | CACTCATCACACAAA | CACUCAUCACACAAAAG | 0.98 | −6_6-6_internal_loop- |
| | AGATGCAAATCTGTG | AUGCAAAUCUGUGGGCG | | symmetric_UUCAGA- |
| | GGCGGATCCACAGGG | GAUCCACAGGGAGGGGG | | AGCUCC |
| | AGGGGGCATTTTAAC | CAUUUUAACAUAUCAGC | | 0_1-1_mismatch_A-C |
| | ATATCAGCTCCCTAAT | UCCCUAAUCAUCCAGGA | | 27_6-6_internal_loop- |
| | CATCCAGGAGATGTA | GAUGUAACUCUAAUC | | symmetric_UAUAGA- |
| | ACTCTAATC (SEQ ID | (SEQ ID NO: 89) | | GUGGGC |
| | NO: 53) | | | |
| FIG. 3B | CTCTGCACTCATCACA | CUCUGCACUCAUCACAC | 0.59 | −6_6-6_internal_loop- |
| | CAAAAGATGCAAATC | AAAAGAUGCAAAUCUGU | | symmetric_UUCAGA- |
| | TGTGGGCGGATCCAC | GGGCGGAUCCACAGGGA | | AGCUCC |
| | AGGGAGGGGGCATTT | GGGGGCAUUUUAACAUA | | 0_1-1_mismatch_A-C |
| | TAACATATCAGCTCC | UCAGCUCCCUAAUCAUC | | 27_6-6_internal_loop- |
| | CTAATCATCCAGGAG | CAGGAGAUGUAACUC | | symmetric_UAUAGA- |
| | ATGTAACTC (SEQ ID | (SEQ ID NO: 40) | | GUGGGC |
| | NO: 33) | | | |
| FIG. 3B | CATATCTCTGCACTCA | CAUAUCUCUGCACUCAU | 0.80 | −6_6-6_internal_loop- |
| | TCACACAAAAGATGC | CACACAAAAGAUGCAAA | | symmetric_UUCAGA- |
| | AAATCTGTGGGCGGA | UCUGUGGGCGGAUCCAC | | AGCUCC |
| | TCCACAGGGAGGGGG | AGGGAGGGGGCAUUUUA | | 0_1-1_mismatch_A-C |
| | CATTTTAACATATCAG | ACAUAUCAGCUCCCUAA | | 27_6-6_internal_loop- |
| | CTCCCTAATCATCCAG | UCAUCCAGGAGAUGU | | symmetric_UAUAGA- |
| | GAGATGT (SEQ ID NO: | (SEQ ID NO: 90) | | GUGGGC |
| | 54) | | | |
| FIG. 3B | TGTGACATATCTCTGC | UGUGACAUAUCUCUGCA | 0.91 | −6_6-6_internal_loop- |
| | ACTCATCACACAAAA | CUCAUCACACAAAAGAU | | symmetric_UUCAGA- |
| | GATGCAAATCTGTGG | GCAAAUCUGUGGGCGGA | | AGCUCC |
| | GCGGATCCACAGGGA | UCCACAGGGAGGGGGCA | | 0_1-1_mismatch_A-C |
| | GGGGGCATTTTAACA | UUUUAACAUAUCAGCUC | | 27_6-6_internal_loop- |
| | TATCAGCTCCCTAATC | CCUAAUCAUCCAGGA | | symmetric_UAUAGA- |
| | ATCCAGGA (SEQ ID | (SEQ ID NO: 91) | | GUGGGC |
| | NO: 55) | | | |
| FIG. 3B | GATATTGTGACATAT | GAUAUUGUGACAUAUCU | 0.72 | −6_6-6_internal_loop- |
| | CTCTGCACTCATCACA | CUGCACUCAUCACACAA | | symmetric_UUCAGA- |
| | CAAAAGATGCAAATC | AAGAUGCAAAUCUGUGG | | AGCUCC |
| | TGTGGGCGGATCCAC | GCGGAUCCACAGGGAGG | | 0_1-1_mismatch_A-C |
| | AGGGAGGGGGCATTT | GGGCAUUUUAACAUAUC | | 27_6-6_internal_loop- |
| | TAACATATCAGCTCC | AGCUCCCUAAUCAUC | | symmetric_UAUAGA- |
| | CTAATCATC (SEQ ID | (SEQ ID NO: 92) | | GUGGGC |
| | NO: 56) | | | |
| FIG. 3C | CACTCATCACTGCCGT | CACUCAUCACUGCCGUG | 0.56 | −8_6-6_internal_loop- |
| | GATGCAAATCTTCTAT | AUGCAAAUCUUCUAUAG | | symmetric_AGUUCA- |
| | AGGATCCACAGGGAG | GAUCCACAGGGAGGGGG | | GGUUUC |
| | GGGGCATTTTAACAT | CAUUUUAACAUAUCUCG | | 0_1-1_mismatch_A-C |
| | ATCTCGGTTTCAATCA | GUUUCAAUCAUCCAGGA | | 44_6-6_internal_loop- |
| | TCCAGGAGATGTAAC | GAUGUAACUCUAAUC | | symmetric_UUUUGU- |
| | TCTAATC (SEQ ID NO: | (SEQ ID NO: 41) | | UGCCGU |
| | 34 | | | |
| FIG. 3C | CTCTGCACTCATCACT | CUCUGCACUCAUCACUG | 0.62 | −8_6-6_internal_loop- |
| | GCCGTGATGCAAATC | CCGUGAUGCAAAUCUUC | | symmetric_AGUUCA- |
| | TTCTATAGGATCCAC | UAUAGGAUCCACAGGGA | | GGUUUC |
| | AGGGAGGGGGCATTT | GGGGGCAUUUUAACAUA | | 0_1-1_mismatch_A-C |
| | TAACATATCTCGGTTT | UCUCGGUUUCAAUCAUC | | 44_6-6_internal_loop- |
| | CAATCATCCAGGAGA | CAGGAGAUGUAACUC | | symmetric_UUUUGU- |
| | TGTAACTC (SEQ ID | (SEQ ID NO: 93) | | UGCCGU |
| | NO: 57) | | | |
| FIG. 3C | CATATCTCTGCACTCA | CAUAUCUCUGCACUCAU | 0.66 | −8_6-6_internal_loop- |
| | TCACTGCCGTGATGC | CACUGCCGUGAUGCAAA | | symmetric_AGUUCA- |
| | AAATCTTCTATAGGA | UCUUCUAUAGGAUCCAC | | GGUUUC |
| | TCCACAGGGAGGGGG | AGGGAGGGGGCAUUUUA | | 0_1-1_mismatch_A-C |
| | CATTTTAACATATCTC | ACAUAUCUCGGUUUCAA | | 44_6-6_internal_loop- |
| | GGTTTCAATCATCCA | UCAUCCAGGAGAUGU | | symmetric_UUUUGU- |
| | GGAGATGT (SEQ ID | (SEQ ID NO: 94) | | UGCCGU |
| | NO: 58) | | | |
| FIG. 3C | TGTGACATATCTCTGC | UGUGACAUAUCUCUGCA | 0.82 | −8_6-6_internal_loop- |
| | ACTCATCACTGCCGT | CUCAUCACUGCCGUGAU | | symmetric_AGUUCA- |
| | GATGCAAATCTTCTAT | GCAAAUCUUCUAUAGGA | | GGUUUC |
| | AGGATCCACAGGGAG | UCCACAGGGAGGGGGCA | | 0_1-1_mismatch_A-C |
| | GGGGCATTTTAACAT | UUUUAACAUAUCUCGGU | | 44_6-6_internal_loop- |
| | ATCTCGGTTTCAATCA | UUCAAUCAUCCAGGA | | symmetric_UUUUGU- |
| | TCCAGGA (SEQ ID NO: | (SEQ ID NO: 95) | | UGCCGU |
| | 59) | | | |
| FIG. 3C | GATATTGTGACATAT | GAUAUUGUGACAUAUCU | 0.67 | −8_6-6_internal_loop- |
| | CTCTGCACTCATCACT | CUGCACUCAUCACUGCC | | symmetric_AGUUCA- |
| | GCCGTGATGCAAATC | GUGAUGCAAAUCUUCUA | | GGUUUC |
| | TTCTATAGGATCCAC | UAGGAUCCACAGGGAGG | | 0_1-1_mismatch A-C |
| | AGGGAGGGGGCATTT | GGGCAUUUUAACAUAUC | | 44_6-6_internal_loop- |
| | TAACATATCTCGGTTT | UCGGUUUCAAUCAUC | | symmetric_UUUUGU- |
| | CAATCATC (SEQ ID | (SEQ ID NO: 96) | | UGCCGU |
| | NO: 60) | | | |
| FIG. 3D | ATCACACAAAAGATC | AUCACACAAAAGAUCUU | 0.36 | −8_6-6_internal_loop- |
| | TTCCCCTTCTATAGGA | CCCCUUCUAUAGGAUCC | | symmetric_AGUUCA- |
| | TCCACAGGGAGGGGG | ACAGGGAGGGGGCAUUU | | ACUCGA |
| | CATTTCAATATATCTC | CAAUAUAUCUCACUCGA | | 3_1-1_mismatch_A-C |
| | ACTCGAAATCATCCA | AAUCAUCCAGGAGAUGU | | 35_6-6_internal_loop- |
| | GGAGATGTAACTCTA | AACUCUAAUCCAGGU | | symmetric_AUUUGC- |
| | ATCCAGGT (SEQ ID | (SEQ ID NO: 42) | | CUUCCC |
| | NO: 35) | | | |
| FIG. 3D | CACTCATCACACAAA | CACUCAUCACACAAAAG | 0.414 | −8_6-6_internal_loop- |
| | AGATCTTCCCCTTCTA | AUCUUCCCCUUCUAUAG | | symmetric_AGUUCA- |
| | TAGGATCCACAGGGA | GAUCCACAGGGAGGGGG | | ACUCGA |
| | GGGGGCATTTCAATA | CAUUUCAAUAUAUCUCA | | 3_1-1_mismatch_A-C |
| | TATCTCACTCGAAATC | CUCGAAAUCAUCCAGGA | | 35_6-6_internal_loop- |
| | ATCCAGGAGATGTAA | GAUGUAACUCUAAUC | | symmetric_AUUUGC- |
| | CTCTAATC (SEQ ID | (SEQ ID NO: 97) | | CUUCCC |
| | NO: 61) | | | |
| FIG. 3D | CTCTGCACTCATCACA | CUCUGCACUCAUCACAC | 0.52 | −8_6-6_internal_loop- |
| | CAAAAGATCTTCCCC | AAAAGAUCUUCCCCUUC | | symmetric_AGUUCA- |
| | TTCTATAGGATCCAC | UAUAGGAUCCACAGGGA | | ACUCGA |
| | AGGGAGGGGGCATTT | GGGGGCAUUUCAAUAUA | | 3_1-1_mismatch_A-C |
| | CAATATATCTCACTCG | UCUCACUCGAAAUCAUC | | 35_6-6_internal_loop- |
| | AAATCATCCAGGAGA | CAGGAGAUGUAACUC | | symmetric_AUUUGC- |
| | TGTAACTC (SEQ ID | (SEQ ID NO: 98) | | CUUCCC |
| | NO: 62) | | | |
| FIG. 3D | CATATCTCTGCACTCA | CAUAUCUCUGCACUCAU | 0.59 | −8_6-6_internal_loop- |
| | TCACACAAAAGATCT | CACACAAAAGAUCUUCC | | symmetric_AGUUCA- |
| | TCCCCTTCTATAGGAT | CCUUCUAUAGGAUCCAC | | ACUCGA |
| | CCACAGGGAGGGGGC | AGGGAGGGGGCAUUUCA | | 3_1-1_mismatch_A-C |
| | ATTTCAATATATCTCA | AUAUAUCUCACUCGAAA | | 35_6-6_internal_loop- |
| | CTCGAAATCATCCAG | UCAUCCAGGAGAUGU | | symmetric_AUUUGC- |
| | GAGATGT (SEQ ID NO: | (SEQ ID NO: 99) | | CUUCCC |
| | 63) | | | |
| FIG. 3D | TGTGACATATCTCTGC | UGUGACAUAUCUCUGCA | 0.67 | −8_6-6_internal_loop- |
| | ACTCATCACACAAAA | CUCAUCACACAAAAGAU | | symmetric_AGUUCA- |
| | GATCTTCCCCTTCTAT | CUUCCCCUUCUAUAGGA | | ACUCGA |
| | AGGATCCACAGGGAG | UCCACAGGGAGGGGGCA | | 3_1-1_mismatch_A-C |
| | GGGGCATTTCAATAT | UUUCAAUAUAUCUCACU | | 35_6-6_internal_loop- |
| | ATCTCACTCGAAATC | CGAAAUCAUCCAGGA | | symmetric_AUUUGC- |
| | ATCCAGGA (SEQ ID | (SEQ ID NO: 100) | | CUUCCC |
| | NO: 64) | | | |
| FIG. 3D | GATATTGTGACATAT | GAUAUUGUGACAUAUCU | 0.63 | −8_6-6_internal_loop- |
| | CTCTGCACTCATCACA | CUGCACUCAUCACACAA | | symmetric_AGUUCA- |
| | CAAAAGATCTTCCCC | AAGAUCUUCCCCUUCUA | | ACUCGA |
| | TTCTATAGGATCCAC | UAGGAUCCACAGGGAGG | | 3_1-1_mismatch_A-C |
| | AGGGAGGGGGCATTT | GGGCAUUUCAAUAUAUC | | 35_6-6_internal_loop- |
| | CAATATATCTCACTCG | UCACUCGAAAUCAUC | | symmetric_AUUUGC- |
| | AAATCATC (SEQ ID | (SEQ ID NO: 101) | | CUUCCC |
| | NO: 65) | | | |
| FIG. 3E | ATCACACAAAAGATC | AUCACACAAAAGAUCUU | 0.74 | −8_6-6_internal_loop- |
| | TTCCCCTTCTATAGGA | CCCCUUCUAUAGGAUCC | | symmetric_AGUUCA- |
| | TCCACAGGGAGGGGG | ACAGGGAGGGGGCAUUU | | ACUCGA |
| | CATTTCAATATATCTC | CAAUAUAUCUCACUCGA | | 3_1-1_mismatch_A-C |
| | ACTCGAAATCATCCA | AAUCAUCCAGGAGAUGU | | 35_6-6_internal_loop- |
| | GGAGATGTAACTCTA | AACUCUAAUCCAGGU | | symmetric_AUUUGC- |
| | ATCCAGGT (SEQ ID | (SEQ ID NO: 42) | | CUUCCC |
| | NO: 35) | | | |
| FIG. 3E | CACTCATCACACAAA | CACUCAUCACACAAAAG | 0.72 | −3_6-6_internal_loop- |
| | AGACCCCTCTCTTCTA | ACCCCUCUCUUCUAUAG | | symmetric_AGAGAU- |
| | TAGGATCCACAGGGA | GAUCCACAGGGAGGGGG | | UCAAAC |
| | GGGGGCATTCTAATA | CAUUCUAAUAUUCAAAC | | 4_1-1_mismatch_A-C |
| | TTCAAACGAACTAAT | GAACUAAUCAUCCAGGA | | 36_6-6_internal_loop- |
| | CATCCAGGAGATGTA | GAUGUAACUCUAAUC | | symmetric_UUUGCA- |
| | ACTCTAATC (SEQ ID | (SEQ ID NO: 43) | | CCCCUC |
| | NO: 36) | | | |
| FIG. 3E | CTCTGCACTCATCACA | CUCUGCACUCAUCACAC | 0.85 | −3_6-6_internal_loop- |
| | CAAAAGACCCCTCTC | AAAAGACCCCUCUCUUC | | symmetric_AGAGAU- |
| | TTCTATAGGATCCAC | UAUAGGAUCCACAGGGA | | UCAAAC |
| | AGGGAGGGGGCATTC | GGGGGCAUUCUAAUAUU | | 4_1-1_mismatch_A-C |
| | TAATATTCAAACGAA | CAAACGAACUAAUCAUC | | 36_6-6_internal_loop- |
| | CTAATCATCCAGGAG | CAGGAGAUGUAACUC | | symmetric_UUUGCA- |
| | ATGTAACTC (SEQ ID | (SEQ ID NO: 102) | | CCCCUC |
| | NO: 66) | | | |
| FIG. 3E | CATATCTCTGCACTCA | CAUAUCUCUGCACUCAU | 0.93 | −3_6-6_internal_loop- |
| | TCACACAAAAGACCC | CACACAAAAGACCCCUC | | symmetric_AGAGAU- |
| | CTCTCTTCTATAGGAT | UCUUCUAUAGGAUCCAC | | UCAAAC |
| | CCACAGGGAGGGGGC | AGGGAGGGGGCAUUCUA | | 4_1-1_mismatch_A-C |
| | ATTCTAATATTCAAAC | AUAUUCAAACGAACUAA | | 36_6-6_internal_loop- |
| | GAACTAATCATCCAG | UCAUCCAGGAGAUGU | | symmetric_UUUGCA- |
| | GAGATGT (SEQ ID NO: | (SEQ ID NO: 103) | | CCCCUC |
| | 67) | | | |
| FIG. 3E | TGTGACATATCTCTGC | UGUGACAUAUCUCUGCA | 0.95 | −3_6-6_internal_loop- |
| | ACTCATCACACAAAA | CUCAUCACACAAAAGAC | | symmetric_AGAGAU- |
| | GACCCCTCTCTTCTAT | CCCUCUCUUCUAUAGGA | | UCAAAC |
| | AGGATCCACAGGGAG | UCCACAGGGAGGGGGCA | | 4_1-1_mismatch_A-C |
| | GGGGCATTCTAATAT | UUCUAAUAUUCAAACGA | | 36_6-6_internal_loop- |
| | TCAAACGAACTAATC | ACUAAUCAUCCAGGA | | symmetric_UUUGCA- |
| | ATCCAGGA (SEQ ID | (SEQ ID NO: 104) | | CCCCUC |
| | NO: 68) | | | |
| FIG. 3E | GATATTGTGACATAT | GAUAUUGUGACAUAUCU | 0.78 | −3_6-6_internal_loop- |
| | CTCTGCACTCATCACA | CUGCACUCAUCACACAA | | symmetric_AGAGAU- |
| | CAAAAGACCCCTCTC | AAGACCCCUCUCUUCUA | | UCAAAC |
| | TTCTATAGGATCCAC | UAGGAUCCACAGGGAGG | | 4_1-1_mismatch_A-C |
| | AGGGAGGGGGCATTC | GGGCAUUCUAAUAUUCA | | 36_6-6_internal_loop- |
| | TAATATTCAAACGAA | AACGAACUAAUCAUC | | symmetric_UUUGCA- |
| | CTAATCATC (SEQ ID | (SEQ ID NO: 105) | | CCCCUC |
| | NO: 69) | | | |
| FIG. 3F | ATCACACTCCCTCTGC | AUCACACUCCCUCUGCA | 0.67 | −11_6-6_internal_loop- |
| | AAATCTTCTATAGGA | AAUCUUCUAUAGGAUCC | | symmetric_AUUAGU- |
| | TCCACAGGGAGGGGG | ACAGGGAGGGGGCAUUC | | CCCUCG |
| | CATTCTAATATATCTC | UAAUAUAUCUCUGACCC | | 4_1-1_mismatch_A-C |
| | TGACCCTCGCATCCA | UCGCAUCCAGGAGAUGU | | 42_6-6_internal_loop- |
| | GGAGATGTAACTCTA | AACUCUAAUCCAGGU | | symmetric_UCUUUU- |
| | ATCCAGGT (SEQ ID | (SEQ ID NO: 44) | | UCCCUC |
| | NO: 37) | | | |
| FIG. 3F | CACTCATCACACTCCC | CACUCAUCACACUCCCU | 0.81 | −11_6-6_internal_loop- |
| | TCTGCAAATCTTCTAT | CUGCAAAUCUUCUAUAG | | symmetric_AUUAGU- |
| | AGGATCCACAGGGAG | GAUCCACAGGGAGGGGG | | CCCUCG |
| | GGGGCATTCTAATAT | CAUUCUAAUAUAUCUCU | | 4_1-1_mismatch_A-C |
| | ATCTCTGACCCTCGCA | GACCCUCGCAUCCAGGA | | 42_6-6_internal_loop- |
| | TCCAGGAGATGTAAC | GAUGUAACUCUAAUC | | symmetric_UCUUUU- |
| | TCTAATC (SEQ ID NO: | (SEQ ID NO: 106) | | UCCCUC |
| | 70) | | | |
| FIG. 3F | CTCTGCACTCATCACA | CUCUGCACUCAUCACAC | 0.87 | −11_6-6_internal_loop- |
| | CTCCCTCTGCAAATCT | UCCCUCUGCAAAUCUUC | | symmetric_AUUAGU- |
| | TCTATAGGATCCACA | UAUAGGAUCCACAGGGA | | CCCUCG |
| | GGGAGGGGGCATTCT | GGGGGCAUUCUAAUAUA | | 4_1-1_mismatch_A-C |
| | AATATATCTCTGACCC | UCUCUGACCCUCGCAUC | | 42_6-6_internal_loop- |
| | TCGCATCCAGGAGAT | CAGGAGAUGUAACUC | | symmetric_UCUUUU- |
| | GTAACTC (SEQ ID NO: | (SEQ ID NO: 107) | | UCCCUC |
| | 71) | | | |
| FIG. 3F | CATATCTCTGCACTCA | CAUAUCUCUGCACUCAU | 0.98 | −11_6-6_internal_loop- |
| | TCACACTCCCTCTGCA | CACACUCCCUCUGCAAA | | symmetric_AUUAGU- |
| | AATCTTCTATAGGATC | UCUUCUAUAGGAUCCAC | | CCCUCG |
| | CACAGGGAGGGGGCA | AGGGAGGGGGCAUUCUA | | 4_1-1_mismatch_A-C |
| | TTCTAATATATCTCTG | AUAUAUCUCUGACCCUC | | 42_6-6_internal_loop- |
| | ACCCTCGCATCCAGG | GCAUCCAGGAGAUGU | | symmetric_UCUUUU- |
| | AGATGT (SEQ ID NO: | (SEQ ID NO: 108) | | UCCCUC |
| | 72) | | | |
| FIG. 3F | TGTGACATATCTCTGC | UGUGACAUAUCUCUGCA | 0.96 | −11_6-6_internal_loop- |
| | ACTCATCACACTCCCT | CUCAUCACACUCCCUCU | | symmetric_AUUAGU- |
| | CTGCAAATCTTCTATA | GCAAAUCUUCUAUAGGA | | CCCUCG |
| | GGATCCACAGGGAGG | UCCACAGGGAGGGGGCA | | 4_1-1_mismatch_A-C |
| | GGGCATTCTAATATA | UUCUAAUAUAUCUCUGA | | 42_6-6_internal_loop- |
| | TCTCTGACCCTCGCAT | CCCUCGCAUCCAGGA | | symmetric_UCUUUU- |
| | CCAGGA (SEQ ID NO: | (SEQ ID NO: 109) | | UCCCUC |
| | 73) | | | |
| FIG. 3F | GATATTGTGACATAT | GAUAUUGUGACAUAUCU | 0.95 | −11_6-6_internal_loop- |
| | CTCTGCACTCATCACA | CUGCACUCAUCACACUC | | symmetric_AUUAGU- |
| | CTCCCTCTGCAAATCT | CCUCUGCAAAUCUUCUA | | CCCUCG |
| | TCTATAGGATCCACA | UAGGAUCCACAGGGAGG | | 4_1-1_mismatch_A-C |
| | GGGAGGGGGCATTCT | GGGCAUUCUAAUAUAUC | | 42_6-6_internal_loop- |
| | AATATATCTCTGACCC | UCUGACCCUCGCAUC | | symmetric_UCUUUU- |
| | TCGCATC (SEQ ID NO: | (SEQ ID NO: 110) | | UCCCUC |
| | 74) | | | |
| FIG. 3G | CACTCATCACACAAA | CACUCAUCACACAAAAG | 0.29 | −22_6-6_internal_loop- |
| | AGATGCAAATCTGTG | AUGCAAAUCUGUGGGCG | | symmetric_UCUCCU- |
| | GGCGGATCCACAGGG | GAUCCACAGGGAGGGGG | | UACUCU |
| | AGGGGGCATTTTAAC | CAUUUUAACGACUCUGU | | −21_1-1_wobble_G-U |
| | GACTCTGTGTGAACG | GUGAACGAUCAUCUUAC | | −14_1-1_wobble_U-G |
| | ATCATCTTACTCTTGT | UCUUGUAACUCUAAUC | | −13_1-0_bulge- |
| | AACTCTAATC (SEQ ID | (SEQ ID NO: 111) | | asymmetric_A- |
| | NO: 75) | | | −7_1-3_bulge- |
| | | | | asymmetric_G-GUG |
| | | | | −3−>0_4-4_bulge- |
| | | | | symmetric_UAUA-CGAC |
| | | | | 27_6-6_internal_loop- |
| | | | | symmetric_UAUAGA- |
| | | | | GUGGGC |
| FIG. 3G | CACTCATCACACAAA | CACUCAUCACACAAAAG | 0.28 | −22_6-6_internal_loop- |
| | AGACCCCTCTCTTCTA | ACCCCUCUCUUCUAUAG | | symmetric_UCUCCU- |
| | TAGGATCCACAGGGA | GAUCCACAGGGAGGGGG | | UACUCU |
| | GGGGGCATTTTAACG | CAUUUUAACGACUCUGU | | −21_1-1_wobble_G-U |
| | ACTCTGTGTGAACGA | GUGAACGAUCAUCUUAC | | −14_1-1_wobble_U-G |
| | TCATCTTACTCTTGTA | UCUUGUAACUCUAAUC | | −13_1-0_bulge- |
| | ACTCTAATC (SEQ ID | (SEQ ID NO: 112) | | asymmetric_A- |
| | NO: 76) | | | −7_1-3_bulge- |
| | | | | asymmetric_G-GUG |
| | | | | −3−>0_4-4_bulge- |
| | | | | symmetric_UAUA-CGAC |
| | | | | 36_6-6_internal_loop- |
| | | | | symmetric_UUUGCA- |
| | | | | CCCCUC |
| FIG. 3G | CACTCATCACACTCCC | CACUCAUCACACUCCCU | 0.29 | −22_6-6_internal_loop- |
| | TCTGCAAATCTTCTAT | CUGCAAAUCUUCUAUAG | | symmetric_UCUCCU- |
| | AGGATCCACAGGGAG | GAUCCACAGGGAGGGGG | | UACUCU |
| | GGGGCATTTTAACGA | CAUUUUAACGACUCUGU | | −21_1-1_wobble_G-U |
| | CTCTGTGTGAACGAT | GUGAACGAUCAUCUUAC | | −14_1-1_wobble_U-G |
| | CATCTTACTCTTGTAA | UCUUGUAACUCUAAUC | | −13_1-0_bulge- |
| | CTCTAATC (SEQ ID | (SEQ ID NO: 113) | | asymmetric_A- |
| | NO: 77) | | | −7_1-3_bulge- |
| | | | | asymmetric_G-GUG |
| | | | | −3−>0_4-4_bulge- |
| | | | | symmetric_UAUA-CGAC |
| | | | | 42_6-6_internal_loop- |
| | | | | symmetric_UCUUUU- |
| | | | | UCCCUC |
| FIG. 3G | CTCTGCACTCATCACA | CUCUGCACUCAUCACAC | 0.23 | −22_6-6_internal_loop- |
| | CAAAAGATGCAAATC | AAAAGAUGCAAAUCUGU | | symmetric_UCUCCU- |
| | TGTGGGCGGATCCAC | GGGCGGAUCCACAGGGA | | UACUCU |
| | AGGGAGGGGGCATTT | GGGGGCAUUUUAACGAC | | −21_1-1_wobble_G-U |
| | TAACGACTCTGTGTG | UCUGUGUGAACGAUCAU | | −14_1-1_wobble_U-G |
| | AACGATCATCTTACTC | CUUACUCUUGUAACUC | | −13_1-0_bulge- |
| | TTGTAACTC (SEQ ID | (SEQ ID NO: 114) | | asymmetric_A- |
| | NO: 78) | | | −7_1-3_bulge- |
| | | | | asymmetric_G-GUG |
| | | | | −3−>0_4-4_bulge- |
| | | | | symmetric_UAUA-CGAC |
| | | | | 27_6-6_internal_loop- |
| | | | | symmetric_UAUAGA- |
| | | | | GUGGGC |
| FIG. 3G | CTCTGCACTCATCACA | CUCUGCACUCAUCACAC | 0.20 | −22_6-6_internal_loop- |
| | CAAAAGACCCCTCTC | AAAAGACCCCUCUCUUC | | symmetric_UCUCCU- |
| | TTCTATAGGATCCAC | UAUAGGAUCCACAGGGA | | UACUCU |
| | AGGGAGGGGGCATTT | GGGGGCAUUUUAACGAC | | −21_1-1_wobble_G-U |
| | TAACGACTCTGTGTG | UCUGUGUGAACGAUCAU | | −14_1-1_wobble_U-G |
| | AACGATCATCTTACTC | CUUACUCUUGUAACUC | | −13_1-0_bulge- |
| | TTGTAACTC (SEQ ID | (SEQ ID NO: 46) | | asymmetric_A- |
| | NO: 79) | | | −7_1-3_bulge- |
| | | | | asymmetric_G-GUG |
| | | | | −3−>0_4-4_bulge- |
| | | | | symmetric_UAUA-CGAC |
| | | | | 36_6-6_internal_loop- |
| | | | | symmetric_UUUGCA- |
| | | | | CCCCUC |
| FIG. 3G | CTCTGCACTCATCACA | CUCUGCACUCAUCACAC | 0.24 | −22_6-6_internal_loop- |
| | CTCCCTCTGCAAATCT | UCCCUCUGCAAAUCUUC | | symmetric_UCUCCU- |
| | TCTATAGGATCCACA | UAUAGGAUCCACAGGGA | | UACUCU |
| | GGGAGGGGGCATTTT | GGGGGCAUUUUAACGAC | | −21_1-1_wobble_G-U |
| | AACGACTCTGTGTGA | UCUGUGUGAACGAUCAU | | −14_1-1_wobble_U-G |
| | ACGATCATCTTACTCT | CUUACUCUUGUAACUC | | −13_1-0_bulge- |
| | TGTAACTC (SEQ ID | (SEQ ID NO: 115) | | asymmetric_A- |
| | NO: 80) | | | −7_1-3_bulge- |
| | | | | asymmetric_G-GUG |
| | | | | −3−>0_4-4_bulge- |
| | | | | symmetric_UAUA-CGAC |
| | | | | 42_6-6_internal_loop- |
| | | | | symmetric_UCUUUU- |
| | | | | UCCCUC |
| FIG. 3A- | GGAGGGGGCATTTTA | GGAGGGGGCAUUUUAAU | 0.28-0.84 | |
| 3G | ATATATCTCTGAACT | AUAUCUCUGAACU (SEQ | | |
| | (SEQ ID NO: 81) | ID NO: 116) | | |
| FIG. 3A- | GGAGGGGGCATTTTA | GGAGGGGGCAUUUUAAU | 0.25-0.76 | |
| 3G | ATATATCTCTGAACTA | AUAUCUCUGAACUAAUC | | |
| | ATCATCCAGGAGATG | AUCCAGGAGAUGUAACU | | |
| | TAACTCTAATCCAGG | CUAAUCCAGG (SEQ ID | | |
| | (SEQ ID NO: 82) | NO: 117) | | |

Additionally, several guide RNAs were shortened to various lengths to test for editing efficiency. The shortened guide RNAs showed reduction in caspase levels that were comparable to guide RNAs 100 nt in length. The shortened and original length guide RNAs are shown in Table 4.

For each sequence, the structural features formed in the double stranded RNA substrate upon hybridization of the guide RNA to the target DUX4 RNA, are shown in the last column of Table 4. For reference, each structural feature formed within a guide-target RNA scaffold (target RNA sequence hybridized to an engineered guide RNA) is annotated as follows:
a) the position of the structural feature with respect to the target A (position 0—the first A (bolded) in the polyA signal sequence AUUAAA) of the target RNA sequence, with a negative value indicating upstream (5′) of the target A and a positive value indicating downstream (3′) of the target A;
b) the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c) the name of the structural feature (e.g., symmetric bulge, symmetric internal loop, asymmetric bulge, asymmetric internal loop, mismatch, or wobble base pair), and
d) the sequences of bases on the target RNA side and the engineered guide RNA side that participate in forming the structural feature.

TABLE 4
Shortened Guide RNAs for targeting the polyA signal sequence of DUX4

| | | Structural Features of the |
|---|---|---|
| Guide DNA sequence | Guide RNA sequence | guide-target RNA scaffold |
| CTCTGCACTCATCAC | CUCUGCACUCAUCACACA | 0_1-1_mismatch_A-C |
| ACAAAAGATGCAAA | AAAGAUGCAAAUCUUCU | |
| TCTTCTATAGGATCC | AUAGGAUCCACAGGGAG | |
| ACAGGGAGGGGGCA | GGGGCAUUUUAACAUAU | |
| TTTTAACATATCTCT | CUCUGAACUAAUCAUCC | |
| GAACTAATCATCCAG | AGGAGAUGUAACUC (SEQ | |
| GAGATGTAACTC | ID NO: 148) | |
| (SEQ ID NO: 118) | | |
| CTCTGCACTCATCAC | CUCUGCACUCAUCACCUA | −5_6-6_internal_loop- |
| CTACTCGATGCAAAT | CUCGAUGCAAAUCUUCU | symmetric_UCAGAG-GCAGCU |
| CTTCTATAGGATCCA | AUAGGAUCCACAGGGAG | 0_1-1_mismatch_A-C |
| CAGGGAGGGGGCAT | GGGGCAUUUUAACAUAU | 44_6-6_internal_loop- |
| TTTAACATATGCAGC | GCAGCUACUAAUCAUCC | symmetric_UUUUGU-CUACUC |
| TACTAATCATCCAGG | AGGAGAUGUAACUC (SEQ | |
| AGATGTAACTC (SEQ | ID NO: 39) | |
| ID NO: 32) | | |
| CTCTGCACTCATCAC | CUCUGCACUCAUCACCUA | −5_6-6_internal_loop- |
| CTACTCGATGCAAAT | CUCGAUGCAAAUCUUCU | symmetric_UCAGAG-GCAGCU |
| CTTCTATAGGATCCA | AUAGGAUCCACAGGGAG | 0_1-1_mismatch_A-C |
| CAGGGAGGGGGCAT | GGGGCAUUUUAACAUAU | 44_6-6_internal_loop- |
| TTTAACATATGCAGC | GCAGCUACUAAUCAUCC | symmetric_UUUUGU-CUACUC |
| TACTAATCATCCAGG | AGGAGAUGU (SEQ ID NO: | |
| AGATGT (SEQ ID NO: | 149) | |
| 119) | | |
| CACTCATCACCTACT | CACUCAUCACCUACUCGA | −5_6-6_internal_loop- |
| CGATGCAAATCTTCT | UGCAAAUCUUCUAUAGG | symmetric_UCAGAG-GCAGCU |
| ATAGGATCCACAGG | AUCCACAGGGAGGGGGC | 0_1-1_mismatch_A-C |
| GAGGGGGCATTTTA | AUUUUAACAUAUGCAGC | 44_6-6_internal_loop- |
| ACATATGCAGCTACT | UACUAAUCAUCCAGGAG | symmetric_UUUUGU-CUACUC |
| AATCATCCAGGAGA | AUGUAACUC (SEQ ID NO: | |
| TGTAACTC (SEQ ID | 150) | |
| NO: 120) | | |
| CTCTGCACTCATCAC | CUCUGCACUCAUCACCUA | −5_6-6_internal_loop- |
| CTACTCGATGCAAAT | CUCGAUGCAAAUCUUCU | symmetric_UCAGAG-GCAGCU |
| CTTCTATAGGATCCA | AUAGGAUCCACAGGGAG | 0_1-1_mismatch_A-C |
| CAGGGAGGGGGCAT | GGGGCAUUUUAACAUAU | 44_6-6_internal_loop- |
| TTTAACATATGCAGC | GCAGCUACUAAUCAUCC | symmetric_UUUUGU-CUACUC |
| TACTAATCATCCAGG | AGGA (SEQ ID NO: 151) | |
| A (SEQ ID NO: 121) | | |
| CTCTGCACTCATCAC | CUCUGCACUCAUCACCUA | −5_6-6_internal_loop- |
| CTACTCGATGCAAAT | CUCGAUGCAAAUCUUCU | symmetric_UCAGAG-GCAGCU |
| CTTCTATAGGATCCA | AUAGGAUCCACAGGGAG | 0_1-1_mismatch_A-C |
| CAGGGAGGGGGCAT | GGGGCAUUUUAACAUAU | 44_6-6_internal_loop- |
| TTTAACATATGCAGC | GCAGCUACUAAUCAUC | symmetric_UUUUGU-CUACUC |
| TACTAATCATC (SEQ | (SEQ ID NO: 152) | |
| ID NO: 122) | | |
| CTCTGCACTCATCAC | CUCUGCACUCAUCACACA | −6_6-6_internal_loop- |
| ACAAAAGATGCAAA | AAAGAUGCAAAUCUGUG | symmetric_UUCAGA-AGCUCC |
| TCTGTGGGCGGATCC | GGCGGAUCCACAGGGAG | 0_1-1_mismatch_A-C |
| ACAGGGAGGGGGCA | GGGGCAUUUUAACAUAU | 27_6-6_internal_loop- |
| TTTTAACATATCAGC | CAGCUCCCUAAUCAUCCA | symmetric_UAUAGA-GUGGGC |
| TCCCTAATCATCCAG | GGAGAUGUAACUC (SEQ | |
| GAGATGTAACTC | ID NO: 40) | |
| (SEQ ID NO: 33) | | |
| CTCTGCACTCATCAC | CUCUGCACUCAUCACACA | −6_6-6_internal_loop- |
| ACAAAAGATGCAAA | AAAGAUGCAAAUCUGUG | symmetric_UUCAGA-AGCUCC |
| TCTGTGGGCGGATCC | GGCGGAUCCACAGGGAG | 0_1-1_mismatch_A-C |
| ACAGGGAGGGGGCA | GGGGCAUUUUAACAUAU | 27_6-6_internal_loop- |
| TTTTAACATATCAGC | CAGCUCCCUAAUCAUCCA | symmetric_UAUAGA-GUGGGC |
| TCCCTAATCATCCAG | GGAGAUGU (SEQ ID NO: | |
| GAGATGT (SEQ ID | 154) | |
| NO: 124) | | |
| CACTCATCACACAAA | CACUCAUCACACAAAAG | −6_6-6_internal_loop- |
| AGATGCAAATCTGTG | AUGCAAAUCUGUGGGCG | symmetric_UUCAGA-AGCUCC |
| GGCGGATCCACAGG | GAUCCACAGGGAGGGGG | 0_1-1_mismatch_A-C |
| GAGGGGGCATTTTA | CAUUUUAACAUAUCAGC | 27_6-6_internal_loop- |
| ACATATCAGCTCCCT | UCCCUAAUCAUCCAGGA | symmetric_UAUAGA-GUGGGC |
| AATCATCCAGGAGA | GAUGUAACUC (SEQ ID | |
| TGTAACTC (SEQ ID | NO: 155) | |
| NO: 125) | | |
| CTCTGCACTCATCAC | CUCUGCACUCAUCACACA | −6_6-6_internal_loop- |
| ACAAAAGATGCAAA | AAAGAUGCAAAUCUGUG | symmetric_UUCAGA-AGCUCC |
| TCTGTGGGCGGATCC | GGCGGAUCCACAGGGAG | 0_1-1_mismatch_A-C |
| ACAGGGAGGGGGCA | GGGGCAUUUUAACAUAU | 27_6-6_internal_loop- |
| TTTTAACATATCAGC | CAGCUCCCUAAUCAUCCA | symmetric_UAUAGA-GUGGGC |
| TCCCTAATCATCCAG | GGA (SEQ ID NO: 156) | |
| GA (SEQ ID NO: 126) | | |
| ATCACACAAAAGAT | AUCACACAAAAGAUGCA | −6_6-6_internal_loop- |
| GCAAATCTGTGGGC | AAUCUGUGGGCGGAUCC | symmetric_UUCAGA-AGCUCC |
| GGATCCACAGGGAG | ACAGGGAGGGGGCAUUU | 0_1-1_mismatch_A-C |
| GGGGCATTTTAACAT | UAACAUAUCAGCUCCCU | 27_6-6_internal_loop- |
| ATCAGCTCCCTAATC | AAUCAUCCAGGAGAUGU | symmetric_UAUAGA-GUGGGC |
| ATCCAGGAGATGTA | AACUC (SEQ ID NO: 157) | |
| ACTC (SEQ ID NO: 127) | | |
| CTCTGCACTCATCAC | CUCUGCACUCAUCACACA | −6_6-6_internal_loop- |
| ACAAAAGATGCAAA | AAAGAUGCAAAUCUGUG | symmetric_UUCAGA-AGCUCC |
| TCTGTGGGCGGATCC | GGCGGAUCCACAGGGAG | 0_1-1_mismatch_A-C |
| ACAGGGAGGGGGCA | GGGGCAUUUUAACAUAU | 27_6-6_internal_loop- |
| TTTTAACATATCAGC | CAGCUCCCUAAUCAUC | symmetric_UAUAGA-GUGGGC |
| TCCCTAATCATC | (SEQ ID NO: 158) | |
| (SEQ ID NO: 128) | | |
| ACAAAAGATGCAAA | ACAAAAGAUGCAAAUCU | −6_6-6_internal_loop- |
| TCTGTGGGCGGATCC | GUGGGCGGAUCCACAGG | symmetric_UUCAGA-AGCUCC |
| ACAGGGAGGGGGCA | GAGGGGGCAUUUUAACA | 0_1-1_mismatch_A-C |
| TTTTAACATATCAGC | UAUCAGCUCCCUAAUCA | 27_6-6_internal_loop- |
| TCCCTAATCATCCAG | UCCAGGAGAUGUAACUC | symmetric_UAUAGA-GUGGGC |
| GAGATGTAACTC | (SEQ ID NO: 159) | |
| (SEQ ID NO: 129) | | |
| CACTCATCACACAAA | CACUCAUCACACAAAAG | −3_6-6_internal_loop- |
| AGACCCCTCTCTTCT | ACCCCUCUCUUCUAUAG | symmetric_AGAGAU-UCAAAC |
| ATAGGATCCACAGG | GAUCCACAGGGAGGGGG | 4_1-1_mismatch_A-C |
| GAGGGGGCATTCTA | CAUUCUAAUAUUCAAAC | 36_6-6_internal_loop- |
| ATATTCAAACGAACT | GAACUAAUCAUCCAGGA | symmetric_UUUGCA-CCCCUC |
| AATCATCCAGGAGA | GAUGUAACUCUAAUC | |
| TGTAACTCTAATC | (SEQ ID NO: 43) | |
| (SEQ ID NO: 36) | | |
| ATCACACAAAAGAC | AUCACACAAAAGACCCC | −3_6-6_internal_loop- |
| CCCTCTCTTCTATAG | UCUCUUCUAUAGGAUCC | symmetric_AGAGAU-UCAAAC |
| GATCCACAGGGAGG | ACAGGGAGGGGGCAUUC | 4_1-1_mismatch_A-C |
| GGGCATTCTAATATT | UAAUAUUCAAACGAACU | 36_6-6_internal_loop- |
| CAAACGAACTAATC | AAUCAUCCAGGAGAUGU | symmetric_UUUGCA-CCCCUC |
| ATCCAGGAGATGTA | AACUCUAAUC (SEQ ID | |
| ACTCTAATC (SEQ ID | NO: 160) | |
| NO: 130) | | |
| CACTCATCACACAA | CACUCAUCACACAAAAG | −3_6-6_internal_loop- |
| AAGACCCCTCTCTTC | ACCCCUCUCUUCUAUAG | symmetric_AGAGAU-UCAAAC |
| TATAGGATCCACAG | GAUCCACAGGGAGGGGG | 4_1-1_mismatch_A-C |
| GGAGGGGGCATTCT | CAUUCUAAUAUUCAAAC | 36_6-6_internal_loop- |
| AATATTCAAACGAA | GAACUAAUCAUCCAGGA | symmetric_UUUGCA-CCCCUC |
| CTAATCATCCAGGAG | GAUGUAACUC (SEQ ID | |
| ATGTAACTC (SEQ ID | NO: 161) | |
| NO: 131) | | |
| CACTCATCACACAA | CACUCAUCACACAAAAG | −3_6-6_internal_loop- |
| AAGACCCCTCTCTTC | ACCCCUCUCUUCUAUAG | symmetric_AGAGAU-UCAAAC |
| TATAGGATCCACAG | GAUCCACAGGGAGGGGG | 4_1-1_mismatch_A-C |
| GGAGGGGGCATTCT | CAUUCUAAUAUUCAAAC | 36_6-6_internal_loop- |
| AATATTCAAACGAA | GAACUAAUCAUCCAGGA | symmetric_UUUGCA-CCCCUC |
| CTAATCATCCAGGAG | GAUGU (SEQ ID NO: 162) | |
| ATGT (SEQ ID NO: 132) | | |
| CACTCATCACACAA | CACUCAUCACACAAAAG | −3_6-6_internal_loop- |
| AAGACCCCTCTCTTC | ACCCCUCUCUUCUAUAG | symmetric_AGAGAU-UCAAAC |
| TATAGGATCCACAG | GAUCCACAGGGAGGGGG | 4_1-1_mismatch_A-C |
| GGAGGGGGCATTCT | CAUUCUAAUAUUCAAAC | 36_6-6_internal_loop- |
| AATATTCAAACGAA | GAACUAAUCAUCCAGGA | symmetric_UUUGCA-CCCCUC |
| CTAATCATCCAGGA | (SEQ ID NO: 163) | |
| (SEQ ID NO: 133) | | |
| CACTCATCACACAA | CACUCAUCACACAAAAG | −3_6-6_internal_loop- |
| AAGACCCCTCTCTTC | ACCCCUCUCUUCUAUAG | symmetric_AGAGAU-UCAAAC |
| TATAGGATCCACAG | GAUCCACAGGGAGGGGG | 4_1-1_mismatch_A-C |
| GGAGGGGGCATTCT | CAUUCUAAUAUUCAAAC | 36_6-6_internal_loop- |
| AATATTCAAACGAA | GAACUAAUCAUC (SEQ ID | symmetric_UUUGCA-CCCCUC |
| CTAATCATC (SEQ ID | NO: 164) | |
| NO: 134) | | |
| ATCACACTCCCTCTG | AUCACACUCCCUCUGCAA | −11_6-6_internal_loop- |
| CAAATCTTCTATAGG | AUCUUCUAUAGGAUCCA | symmetric_AUUAGU-CCCUCG |
| ATCCACAGGGAGGG | CAGGGAGGGGGCAUUCU | 4_1-1_mismatch_A-C |
| GGCATTCTAATATAT | AAUAUAUCUCUGACCCU | 42_6-6_internal_loop- |
| CTCTGACCCTCGCAT | CGCAUCCAGGAGAUGUA | symmetric_UCUUUU-UCCCUC |
| CCAGGAGATGTAAC | ACUCUAAUCCAGGU (SEQ | |
| TCTAATCCAGGT | ID NO: 44) | |
| (SEQ ID NO: 37) | | |
| ATCACACTCCCTCTG | AUCACACUCCCUCUGCA | −11_6-6_internal_loop- |
| CAAATCTTCTATAGG | AAUCUUCUAUAGGAUCC | symmetric_AUUAGU-CCCUCG |
| ATCCACAGGGAGGG | ACAGGGAGGGGGCAUUC | 4_1-1_mismatch_A-C |
| GGCATTCTAATATAT | UAAUAUAUCUCUGACCC | 42_6-6_internal_loop- |
| CTCTGACCCTCGCAT | UCGCAUCCAGGAGAUGU | symmetric_UCUUUU-UCCCUC |
| CCAGGAGATGTAAC | AACUCUAAUC (SEQ ID | |
| TCTAATC (SEQ ID | NO: 165) | |
| NO: 135) | | |
| ATCACACTCCCTCTG | AUCACACUCCCUCUGCA | −11_6-6_internal_loop- |
| CAAATCTTCTATAGG | AAUCUUCUAUAGGAUCC | symmetric_AUUAGU-CCCUCG |
| ATCCACAGGGAGGG | ACAGGGAGGGGGCAUUC | 4_1-1_mismatch_A-C |
| GGCATTCTAATATAT | UAAUAUAUCUCUGACCC | 42_6-6_internal_loop- |
| CTCTGACCCTCGCAT | UCGCAUCCAGGAGAUGU | symmetric_UCUUUU-UCCCUC |
| CCAGGAGATGTAAC | AACUC (SEQ ID NO: 166) | |
| TC (SEQ ID NO: 136) | | |
| ATCACACTCCCTCTG | AUCACACUCCCUCUGCA | −11_6-6_internal_loop- |
| CAAATCTTCTATAGG | AAUCUUCUAUAGGAUCC | symmetric_AUUAGU-CCCUCG |
| ATCCACAGGGAGGG | ACAGGGAGGGGGCAUUC | 4_1-1_mismatch_A-C |
| GGCATTCTAATATAT | UAAUAUAUCUCUGACCC | 42_6-6_internal_loop- |
| CTCTGACCCTCGCAT | UCGCAUCCAGGAGAUGU | symmetric_UCUUUU-UCCCUC |
| CCAGGAGATGT (SEQ | (SEQ ID NO: 167) | |
| ID NO: 137) | | |
| ATCACACTCCCTCTG | AUCACACUCCCUCUGCA | −11_6-6_internal_loop- |
| CAAATCTTCTATAGG | AAUCUUCUAUAGGAUCC | symmetric_AUUAGU-CCCUCG |
| ATCCACAGGGAGGG | ACAGGGAGGGGGCAUUC | 4_1-1_mismatch_A-C |
| GGCATTCTAATATAT | UAAUAUAUCUCUGACCC | 42_6-6_internal_loop- |
| CTCTGACCCTCGCAT | UCGCAUCCAGGA (SEQ ID | symmetric_UCUUUU-UCCCUC |
| CCAGGA (SEQ ID NO: | NO: 168) | |
| 138) | | |
| CACTCATCACACAAA | CACUCAUCACACAAAAG | 4_1-1_mismatch_A-C |
| AGATGCAAATCTTCT | AUGCAAAUCUUCUAUAG | |
| ATAGGATCCACAGG | GAUCCACAGGGAGGGGG | |
| GAGGGGGCATTCTA | CAUUCUAAUAUAUCUCU | |
| ATATATCTCTGAACT | GAACUAAUCAUCCAGGA | |
| AATCATCCAGGAGA | GAUGUAACUCUAAUC | |
| TGTAACTCTAATC | (SEQ ID NO: 170) | |
| (SEQ ID NO: 140) | | |
| CACTCATCACACAAA | CACUCAUCACACAAAAG | 0_1-1_mismatch_A-C |
| AGATGCAAATCTTCT | AUGCAAAUCUUCUAUAG | |
| ATAGGATCCACAGG | GAUCCACAGGGAGGGGG | |
| GAGGGGGCATTTTA | CAUUUUAACAUAUCUCU | |
| ACATATCTCTGAACT | GAACUAAUCAUCCAGGA | |
| AATCATCCAGGAGA | GAUGUAACUCUAAUC | |
| TGTAACTCTAATC | (SEQ ID NO: 171) | |
| (SEQ ID NO: 141) | | |
| CACTCATCACTGCCG | CACUCAUCACUGCCGUG | −8_6-6_internal_loop- |
| TGATGCAAATCTTCT | AUGCAAAUCUUCUAUAG | symmetric_AGUUCA-GGUUUC |
| ATAGGATCCACAGG | GAUCCACAGGGAGGGGG | 0_1-1_mismatch_A-C |
| GAGGGGGCATTTTA | CAUUUUAACAUAUCUCG | 44_6-6_internal_loop- |
| ACATATCTCGGTTTC | GUUUCAAUCAUCCAGGA | symmetric_UUUUGU-UGCCGU |
| AATCATCCAGGAGA | GAUGUAACUCUAAUC | |
| TGTAACTCTAATC | (SEQ ID NO: 41) | |
| (SEQ ID NO: 34) | | |
| CACTCATCACTGCCG | CACUCAUCACUGCCGUG | −8_6-6_internal_loop- |
| TGATGCAAATCTTCT | AUGCAAAUCUUCUAUAG | symmetric_AGUUCA-GGUUUC |
| ATAGGATCCACAGG | GAUCCACAGGGAGGGGG | 0_1-1_mismatch_A-C |
| GAGGGGGCATTTTA | CAUUUUAACAUAUCUCG | 44_6-6_internal_loop- |
| ACATATCTCGGTTTC | GUUUCAAUCAUCCAGGA | symmetric_UUUUGU-UGCCGU |
| AATCATCCAGGAGA | GAUGUAACUC (SEQ ID | |
| TGTAACTC (SEQ ID | NO: 173) | |
| NO: 143) | | |
| CACTCATCACTGCCG | CACUCAUCACUGCCGUG | −8_6-6_internal_loop- |
| TGATGCAAATCTTCT | AUGCAAAUCUUCUAUAG | symmetric_AGUUCA-GGUUUC |
| ATAGGATCCACAGG | GAUCCACAGGGAGGGGG | 0_1-1_mismatch_A-C |
| GAGGGGGCATTTTA | CAUUUUAACAUAUCUCG | 44_6-6_internal_loop- |
| ACATATCTCGGTTTC | GUUUCAAUCAUCCAGGA | symmetric_UUUUGU-UGCCGU |
| AATCATCCAGGAGA | GAUGU (SEQ ID NO: 174) | |
| TGT (SEQ ID NO: 144) | | |
| CACTCATCACTGCCG | CACUCAUCACUGCCGUG | −8_6-6_internal_loop- |
| TGATGCAAATCTTCT | AUGCAAAUCUUCUAUAG | symmetric_AGUUCA-GGUUUC |
| ATAGGATCCACAGG | GAUCCACAGGGAGGGGG | 0_1-1_mismatch_A-C |
| GAGGGGGCATTTTA | CAUUUUAACAUAUCUCG | 44_6-6_internal_loop- |
| ACATATCTCGGTTTC | GUUUCAAUCAUCCAGGA | symmetric_UUUUGU-UGCCGU |
| AATCATCCAGGA | (SEQ ID NO: 175) | |
| (SEQ ID NO: 145) | | |
| ATCACACAAAAGAT | AUCACACAAAAGAUCUU | −8_6-6_internal_loop- |
| CTTCCCCTTCTATAG | CCCCUUCUAUAGGAUCC | symmetric_AGUUCA-ACUCGA |
| GATCCACAGGGAGG | ACAGGGAGGGGGCAUUU | 3_1-1_mismatch_A-C |
| GGGCATTTCAATATA | CAAUAUAUCUCACUCGA | 35_6-6_internal_loop- |
| TCTCACTCGAAATCA | AAUCAUCCAGGAGAUGU | symmetric_AUUUGC-CUUCCC |
| TCCAGGAGATGTAA | AACUCUAAUCCAGGU | |
| CTCTAATCCAGGT | (SEQ ID NO: 42) | |
| (SEQ ID NO: 35) | | |
| ATCACACAAAAGAT | AUCACACAAAAGAUCUU | −8_6-6_internal_loop- |
| CTTCCCCTTCTATAG | CCCCUUCUAUAGGAUCC | symmetric_AGUUCA-ACUCGA |
| GATCCACAGGGAGG | ACAGGGAGGGGGCAUUU | 3_1-1_mismatch_A-C |
| GGGCATTTCAATATA | CAAUAUAUCUCACUCGA | 35_6-6_internal_loop- |
| TCTCACTCGAAATCA | AAUCAUCCAGGAGAUGU | symmetric_AUUUGC-CUUCCC |
| TCCAGGAGATGTAA | AACUCUAAUC (SEQ ID | |
| CTCTAATC (SEQ ID | NO: 176) | |
| NO: 146) | | |
| ATCACACAAAAGAT | AUCACACAAAAGAUCUU | −8_6-6_internal_loop- |
| CTTCCCCTTCTATAG | CCCCUUCUAUAGGAUCC | symmetric_AGUUCA-ACUCGA |
| GATCCACAGGGAGG | ACAGGGAGGGGGCAUUU | 3_1-1_mismatch_A-C |
| GGGCATTTCAATATA | CAAUAUAUCUCACUCGA | 35_6-6_internal_loop- |
| TCTCACTCGAAATCA | AAUCAUCCAGGAGAUGU | symmetric_AUUUGC-CUUCCC |
| TCCAGGAGATGT | (SEQ ID NO: 177) | |
| (SEQ ID NO: 147) | | |

### Example 4

### Guide RNAs for Targeting DUX4 RNA

Guide RNAs were selected from the macrofootprint and guide RNA length assays described in Example 3 and which are shown below in Table 5.

For each sequence, the structural features formed in the double stranded RNA substrate upon hybridization of the guide RNA to the target DUX4 RNA, are shown in the last column of Table 5. For reference, each structural feature formed within a guide-target RNA scaffold (target RNA sequence hybridized to an engineered guide RNA) is annotated as follows:
a) the position of the structural feature with respect to the target A (position 0—the first A (bolded) in the polyA signal sequence AUUAAA) of the target RNA sequence, with a negative value indicating upstream (5′) of the target A and a positive value indicating downstream (3′) of the target A;
b) the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c) the name of the structural feature (e.g., symmetric bulge, symmetric internal loop, asymmetric bulge, asymmetric internal loop, mismatch, or wobble base pair), and
d) the sequences of bases on the target RNA side and the engineered guide RNA side that participate in forming the structural feature.

For example, with reference to SEQ ID NO: 39, “−5_6-6_internal_loop-symmetric_UCAGAG-GCAGCU, 0_1-1_mismatch_A-C, 44_6-6_internal_loop-symmetric_UUUUGU-CUACUC” is read as a structural feature formed in a guide-target RNA scaffold (target DUX4 RNA sequence hybridized to an engineered guide RNA of SEQ ID NO: 39), where a structural feature starts 5 nucleotides upstream (5′) (the −5 position) from the target A (0 position) of the target RNA sequence; six contiguous bases from the target RNA sequence and six contiguous bases from the engineered guide RNA form the structural feature; the structural feature is an internal symmetric loop; and a sequence of UCAGAG from the target RNA side and a sequence of GCAGCU from the engineered guide RNA side participate in forming the internal symmetric loop. A structural feature is located at the target A (0 position) of the target RNA sequence; 1 base from the target RNA and 1 base from the engineered guide RNA form the structural feature; the structural feature is a mismatch; and a sequence of A from the target RNA side and a sequence of C from the engineered guide RNA side participate in forming the mismatch. A structural feature starts 44 nucleotides downstream (3′) (the +44 position) from the target A (0 position) of the target RNA sequence; six contiguous bases from the target RNA sequence and six contiguous bases from the engineered guide RNA form the structural feature; the structural feature is an internal symmetric loop; and a sequence of UUUUGU from the target RNA side and a sequence of CUACUC from the engineered guide RNA side participate in forming the internal symmetric loop.

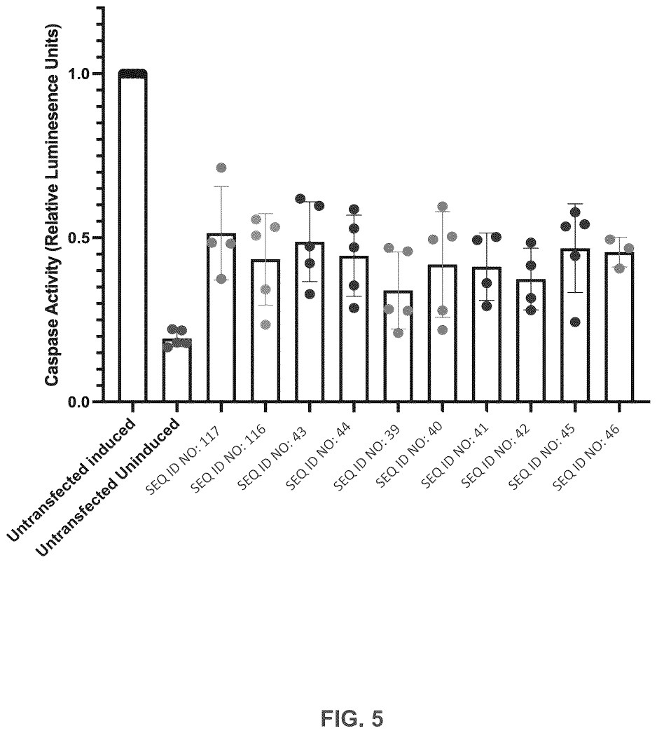

TABLE 5
Guide RNAs for targeting the polyA signal sequence of DUX4

| | | Relative | |
|---|---|---|---|
| | | caspase | |
| | | activity to | |
| | | untransfected | |
| | | induced cells | Structural Features of the |
| Guide DNA sequence | Guide RNA sequence | from FIG. 5 | guide-target RNA scaffold |
| CTCTGCACTCATCACCT | CUCUGCACUCAUCACC | 0.34 | −5_6-6_internal_loop- |
| ACTCGATGCAAATCTTC | UACUCGAUGCAAAUCU | | symmetric_UCAGAG-GCAGCU |
| TATAGGATCCACAGGGA | UCUAUAGGAUCCACAG | | 0_1-1_mismatch_A-C |
| GGGGGCATTTTAACATA | GGAGGGGGCAUUUUA | | 44_6-6_internal_loop- |
| TGCAGCTACTAATCATC | ACAUAUGCAGCUACUA | | symmetric_UUUUGU-CUACUC |
| CAGGAGATGTAACTC | AUCAUCCAGGAGAUGU | | |
| (SEQ ID NO: 32) | AACUC (SEQ ID NO: 39) | | |
| CTCTGCACTCATCACAC | CUCUGCACUCAUCACA | 0.41 | −6_6-6_internal_loop- |
| AAAAGATGCAAATCTGT | CAAAAGAUGCAAAUCU | | symmetric_UUCAGA-AGCUCC |
| GGGCGGATCCACAGGG | GUGGGCGGAUCCACAG | | 0_1-1_mismatch_A-C |
| AGGGGGCATTTTAACAT | GGAGGGGGCAUUUUA | | 27_6-6_internal_loop- |
| ATCAGCTCCCTAATCAT | ACAUAUCAGCUCCCUA | | symmetric_UAUAGA-GUGGGC |
| CCAGGAGATGTAACTC | AUCAUCCAGGAGAUGU | | |
| (SEQ ID NO: 33) | AACUC (SEQ ID NO: 40) | | |
| CACTCATCACTGCCGTG | CACUCAUCACUGCCGU | 0.41 | −8_6-6_internal_loop- |
| ATGCAAATCTTCTATAG | GAUGCAAAUCUUCUAU | | symmetric_AGUUCA-GGUUUC |
| GATCCACAGGGAGGGG | AGGAUCCACAGGGAGG | | 0_1-1_mismatch_A-C |
| GCATTTTAACATATCTC | GGGCAUUUUAACAUAU | | 44_6-6_internal_loop- |
| GGTTTCAATCATCCAGG | CUCGGUUUCAAUCAUC | | symmetric_UUUUGU-UGCCGU |
| AGATGTAACTCTAATC | CAGGAGAUGUAACUCU | | |
| (SEQ ID NO: 34) | AAUC (SEQ ID NO: 41) | | |
| ATCACACAAAAGATCTT | AUCACACAAAAGAUCU | 0.37 | −8_6-6_internal_loop- |
| CCCCTTCTATAGGATCC | UCCCCUUCUAUAGGAU | | symmetric_AGUUCA-ACUCGA |
| ACAGGGAGGGGGCATTT | CCACAGGGAGGGGGCA | | 3_1-1_mismatch_A-C |
| CAATATATCTCACTCGA | UUUCAAUAUAUCUCAC | | 35_6-6_internal_loop- |
| AATCATCCAGGAGATGT | UCGAAAUCAUCCAGGA | | symmetric_AUUUGC-CUUCCC |
| AACTCTAATCCAGGT | GAUGUAACUCUAAUCC | | |
| (SEQ ID NO: 35) | AGGU (SEQ ID NO: 42) | | |
| CACTCATCACACAAAAG | CACUCAUCACACAAAA | 0.48 | −3_6-6_internal_loop- |
| ACCCCTCTCTTCTATAG | GACCCCUCUCUUCUAU | | symmetric_AGAGAU-UCAAAC |
| GATCCACAGGGAGGGG | AGGAUCCACAGGGAGG | | 4_1-1_mismatch_A-C |
| GCATTCTAATATTCAAA | GGGCAUUCUAAUAUUC | | 36_6-6_internal_loop- |
| CGAACTAATCATCCAGG | AAACGAACUAAUCAUC | | symmetric_UUUGCA-CCCCUC |
| AGATGTAACTCTAATC | CAGGAGAUGUAACUCU | | |
| (SEQ ID NO: 36) | AAUC (SEQ ID NO: 43) | | |
| ATCACACTCCCTCTGCA | AUCACACUCCCUCUGC | 0.44 | −11_6-6_internal_loop- |
| AATCTTCTATAGGATCC | AAAUCUUCUAUAGGAU | | symmetric_AUUAGU-CCCUCG |
| ACAGGGAGGGGGCATT | CCACAGGGAGGGGGCA | | 4_1-1_mismatch_A-C |
| CTAATATATCTCTGACC | UUCUAAUAUAUCUCUG | | 42_6-6_internal_loop- |
| CTCGCATCCAGGAGATG | ACCCUCGCAUCCAGGA | | symmetric_UCUUUU-UCCCUC |
| TAACTCTAATCCAGGT | GAUGUAACUCUAAUCC | | |
| (SEQ ID NO: 37) | AGGU (SEQ ID NO: 44) | | |
| ATCACACAAAAGATGCA | AUCACACAAAAGAUGC | 0.46 | −22_6-6_internal_loop- |
| AATCTGTGGCTGGATCC | AAAUCUGUGGCUGGAU | | symmetric_UCUCCU-UACUCU |
| ACAGGGAGGGGGCATTT | CCACAGGGAGGGGGCA | | -21_1-1_wobble_G-U |
| TAACGACTCTGTGTGAA | UUUUAACGACUCUGUG | | -14_1-1_wobble_U-G |
| CGATCATCTTACTCTTGT | UGAACGAUCAUCUUAC | | -13_1-0_bulge-asymmetric_A- |
| AACTCTAATCCAGGT | UCUUGUAACUCUAAUC | | −7_1-3_bulge-asymmetric_G-GUG |
| (SEQ ID NO: 38) | CAGGU (SEQ ID NO: 45) | | −3−>0 4-4_bulge-symmetric_UAUA-CGAC |
| | | | 27_6-6_internal_loop- |
| | | | symmetric_UAUAGA-GUGGCU |
| CTCTGCACTCATCACAC | CUCUGCACUCAUCACA | 0.45 | −22_6-6_internal_loop- |
| AAAAGACCCCTCTCTTC | CAAAAGACCCCUCUCU | | symmetric_UCUCCU-UACUCU |
| TATAGGATCCACAGGGA | UCUAUAGGAUCCACAG | | −21_1-1_wobble_G-U |
| GGGGGCATTTTAACGAC | GGAGGGGGCAUUUUA | | −14_1-1_wobble_U-G |
| TCTGTGTGAACGATCAT | ACGACUCUGUGUGAAC | | −13_1-0_bulge-asymmetric_A- |
| CTTACTCTTGTAACTC | GAUCAUCUUACUCUUG | | −7_1-3_bulge-asymmetric_G-GUG |
| (SEQ ID NO: 79) | UAACUC (SEQ ID NO: | | −3−>0_4-4_bulge-symmetric_UAUA-CGAC |
| | 46) | | 36_6-6_internal_loop- |
| | | | symmetric_UUUGCA-CCCCUC |

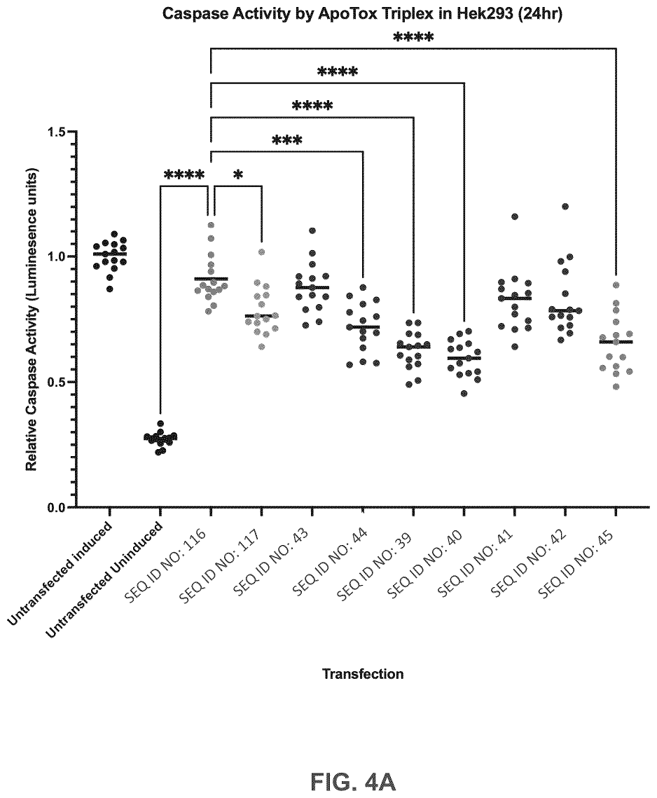

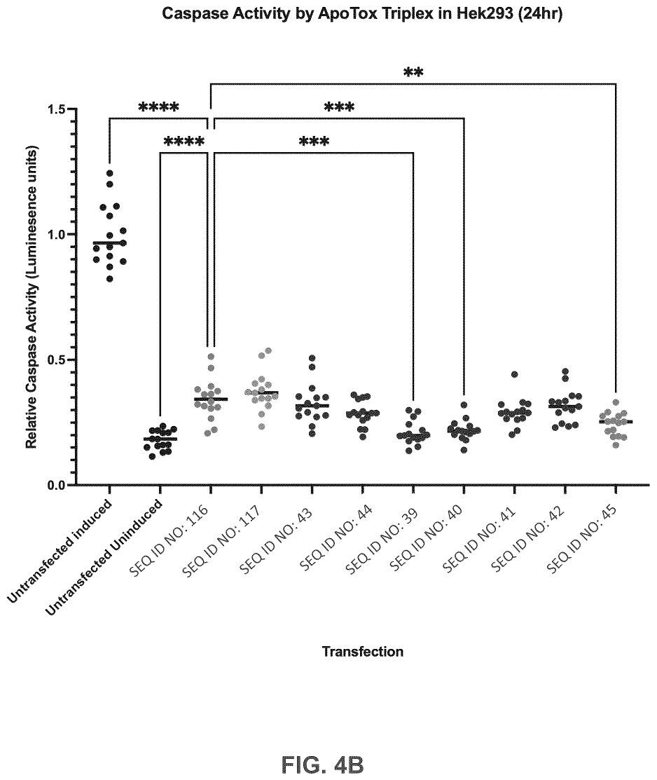

The guide RNAs of Table 5 were tested in a heterologous population of HEK293T cells integrated with a tetracycline (tet) inducible DUX4 expression system as described in Example 3. Additionally, the guide RNAs were further tested in a clonal population of cells isolated from the heterologous population of inducible DUX4 HEK293T cells. The clonal population was isolated from the heterologous population of inducible DUX4 HEK293T cells because the heterologous population showed variable expression of the cloned selection reporter gene (red fluorescent protein). The results from the heterologous population of cells from a single biological replicate are shown in FIG. 4A, and the results from the clonal population of cells of a single biological replicate are shown in FIG. 4B. Similar results were seen for two additional independent replicates of the heterologous population of cells, and at least three addition independent replicates of the clonal population of cells. FIG. 5 shows the combined results for the reduction in caspase activity in the clonal inducible DUX4 HEK293T cell line from at least 3 biologically independent experiments. FIG. 5 shows that guide RNAs having a sequence of SEQ ID NO: 39-SEQ ID NO: 45 were able to reduce caspase activity. Table 5 also shows the relative caspase activity based on luminescence units from each guide RNA as compared to the untransfected induced cells as shown in FIG. 5. These results are comparable to the biologically independent replicates shown in FIG. 4A-4B and show the guide RNAs produced reduced caspase activity in DUX4 induced cells, indicating a reduction in DUX4-induced toxicity levels.

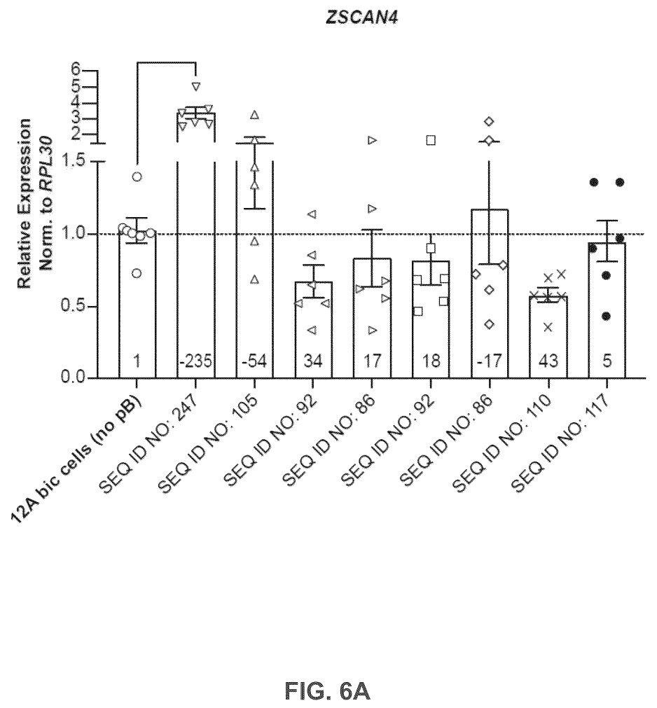

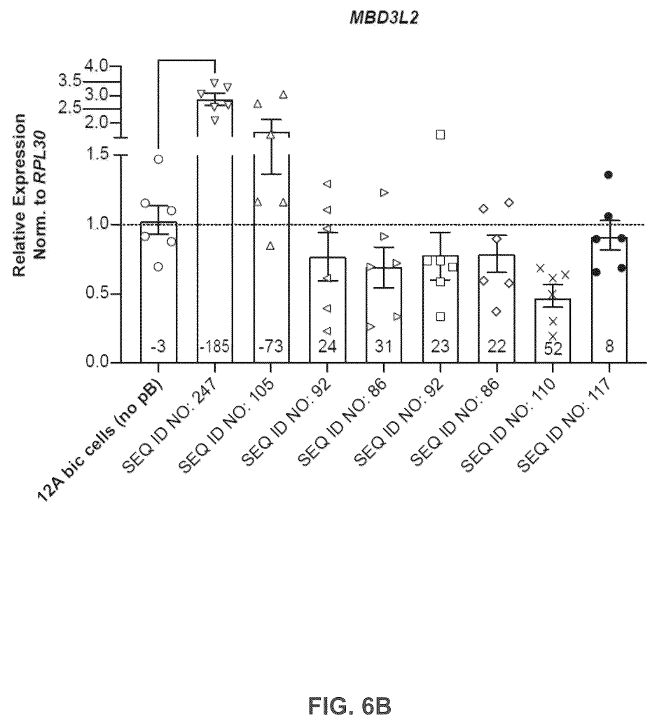

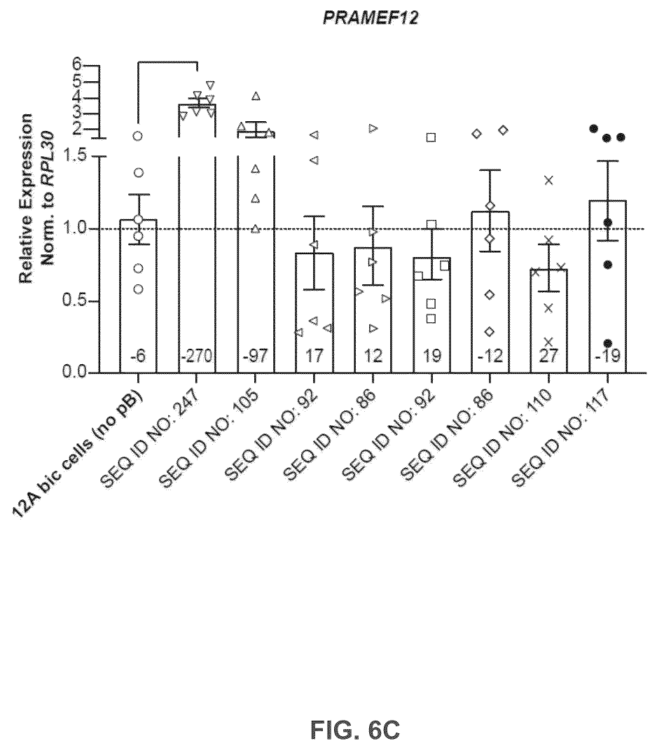

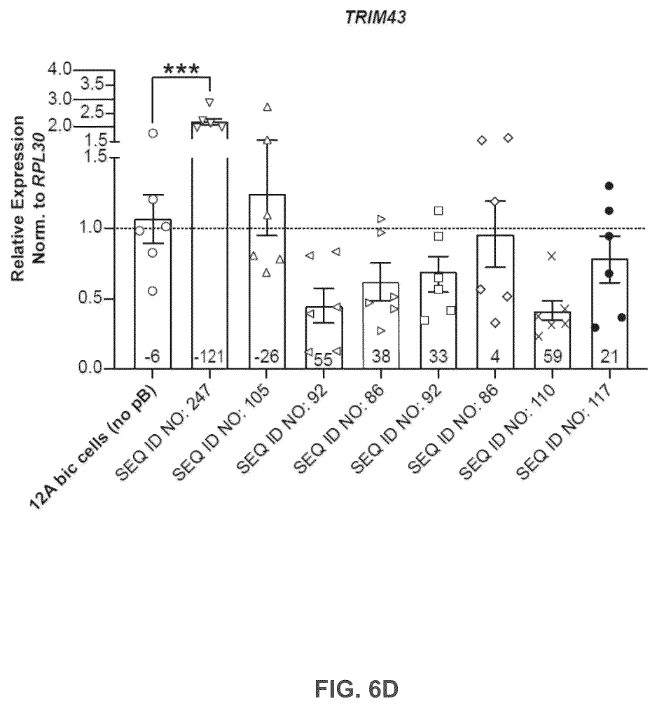

Although DUX4 misexpression causes FSHD, it is expressed in sporadic bursts throughout the lifetime of a patient and it is lowly abundant in FSHD biopsies, making it difficult to detect reliably. On the other hand, the target genes activated by the misexpression of DUX4 are more readily detected than DUX4-FL in FSHD biopsies and are therefore used as a proxy for DUX4 activity in FSHD muscle. These DUX4 target genes are aberrantly expressed in affected FSHD skeletal muscle but not in control/unaffected muscle biopsies. A validated readout for detecting reduction in DUX4 levels is the reduction in expression of genes downstream of DUX4. FIGS. 6A-6D show reduced expression of ZSCAN4, MBD3L2, PRAMEF12 and TRIM43 in immortalized patient-derived affected FSHD cells stably transfected with guide RNAs targeting DUX4-FL mRNA. The guide RNAs were transfected with different promoters and/or SmOPT variants. The target RNA sequence for DUX4-FL was: DNA—GATGATTAGTTCAGAGATATATTAAAATGCCCCCTCCCTGTGGATCCTATAGAAGATTT GCATCTTTTGTGTGATGAGTGCAGAGATATGTCACAATATC (SEQ ID NO: 242) or RNA—GAUGAUUAGUUCAGAGAUAUAUAAAAUGCCCCCUCCCUGUGGAUCCUAUAGAAGA UUUGCAUCUUUUGUGUGAUGAGUGCAGAGAUAUGUCACAAUAUC (SEQ ID NO: 243).

FIG. 6A shows reduced expression in ZSCAN4 by SEQ ID NOS: 92, 86, and 110. FIG. 6B shows reduced expression in MBD3L2 by SEQ ID NOS: 92, 86, and 110. FIG. 6C shows reduced expression in PRAMEF12 by SEQ ID NOS: 92, 86, and 110. FIG. 6D shows reduced expression in TRIM43 by SEQ ID NOS: 92, 86, and 110. These results show guide RNAs can reduce expression of genes downstream of DUX4, such as ZSCAN4, MBD3L2, PRAMEF12 and TRIM43.

The guide RNAs from FIGS. 6A-6D) are shown in Table 6. The guide-target scaffold structural features for SEQ ID NO: 247 are: −9_6-6internal_loop-symmetric_UAGUUC-CGUGAU; 0_1-1_mismatch_A-C; and 40_6-6_internal_loop-symmetric_CAUCUU-CCCUCC.

TABLE 6
Guide RNA sequences for targeting the polyA signal sequence of DUX4-FL

| Guide DNA sequence | Guide RNA sequence |
|---|---|
| GATATTGTGACATATCTCTGCACT | GAUAUUGUGACAUAUCUCUGCACUCAU |
| CATCACACAACCCTCCCAAATCTT | CACACAACCCUCCCAAAUCUUCUAUAG |
| CTATAGGATCCACAGGGAGGGGG | GAUCCACAGGGAGGGGGCAUUUUAACA |
| CATTTTAACATATCTCTCGTGATAT | UAUCUCUCGUGAUAUCAUC (SEQ ID NO: 247) |
| CATC (SEQ ID NO: 246) | |
| GATATTGTGACATATCTCTGCACT | GAUAUUGUGACAUAUCUCUGCACUCAU |
| CATCACACAAAAGACCCCTCTCTT | CACACAAAAGACCCCUCUCUUCUAUAG |
| CTATAGGATCCACAGGGAGGGGG | GAUCCACAGGGAGGGGGCAUUCUAAUA |
| CATTCTAATATTCAAACGAACTAA | UUCAAACGAACUAAUCAUC (SEQ ID NO: 105) |
| TCATC (SEQ ID NO: 69) | |
| GATATTGTGACATATCTCTGCACT | GAUAUUGUGACAUAUCUCUGCACUCAU |
| CATCACACAAAAGATGCAAATCTG | CACACAAAAGAUGCAAAUCUGUGGGCG |
| TGGGCGGATCCACAGGGAGGGGG | GAUCCACAGGGAGGGGGCAUUUUAACA |
| CATTTTAACATATCAGCTCCCTAAT | UAUCAGCUCCCUAAUCAUC (SEQ ID NO: 92 |
| CATC (SEQ ID NO: 56) | |
| GATATTGTGACATATCTCTGCACT | GAUAUUGUGACAUAUCUCUGCACUCAU |
| CATCACCTACTCGATGCAAATCTT | CACCUACUCGAUGCAAAUCUUCUAUAG |
| CTATAGGATCCACAGGGAGGGGG | GAUCCACAGGGAGGGGGCAUUUUAACA |
| CATTTTAACATATGCAGCTACTAA | UAUGCAGCUACUAAUCAUC (SEQ ID NO: 86) |
| TCATC (SEQ ID NO: 50) | |
| GATATTGTGACATATCTCTGCACT | GAUAUUGUGACAUAUCUCUGCACUCAU |
| CATCACACAAAAGATGCAAATCTG | CACACAAAAGAUGCAAAUCUGUGGGCG |
| TGGGCGGATCCACAGGGAGGGGG | GAUCCACAGGGAGGGGGCAUUUUAACA |
| CATTTTAACATATCAGCTCCCTAAT | UAUCAGCUCCCUAAUCAUC (SEQ ID NO: 92) |
| CATC (SEQ ID NO: 56) | |
| GATATTGTGACATATCTCTGCACT | GAUAUUGUGACAUAUCUCUGCACUCAU |
| CATCACCTACTCGATGCAAATCTT | CACCUACUCGAUGCAAAUCUUCUAUAG |
| CTATAGGATCCACAGGGAGGGGG | GAUCCACAGGGAGGGGGCAUUUUAACA |
| CATTTTAACATATGCAGCTACTAA | UAUGCAGCUACUAAUCAUC (SEQ ID NO: 86) |
| TCATC (SEQ ID NO: 50) | |
| GATATTGTGACATATCTCTGCACT | GAUAUUGUGACAUAUCUCUGCACUCAU |
| CATCACACTCCCTCTGCAAATCTTC | CACACUCCCUCUGCAAAUCUUCUAUAG |
| TATAGGATCCACAGGGAGGGGGC | GAUCCACAGGGAGGGGGCAUUCUAAUA |
| ATTCTAATATATCTCTGACCCTCGC | UAUCUCUGACCCUCGCAUC (SEQ ID NO: 110) |
| ATC (SEQ ID NO: 74) | |
| GGAGGGGGCATTTTAATATATCTC | GGAGGGGGCAUUUUAAUAUAUCUCUGA |
| TGAACTAATCATCCAGGAGATGTA | ACUAAUCAUCCAGGAGAUGUAACUCUA |
| ACTCTAATCCAGG (SEQ ID NO: 82) | AUCCAGG (SEQ ID NO: 117) |

### Example 5

### Progeny Guide RNAs Targeting DUX4

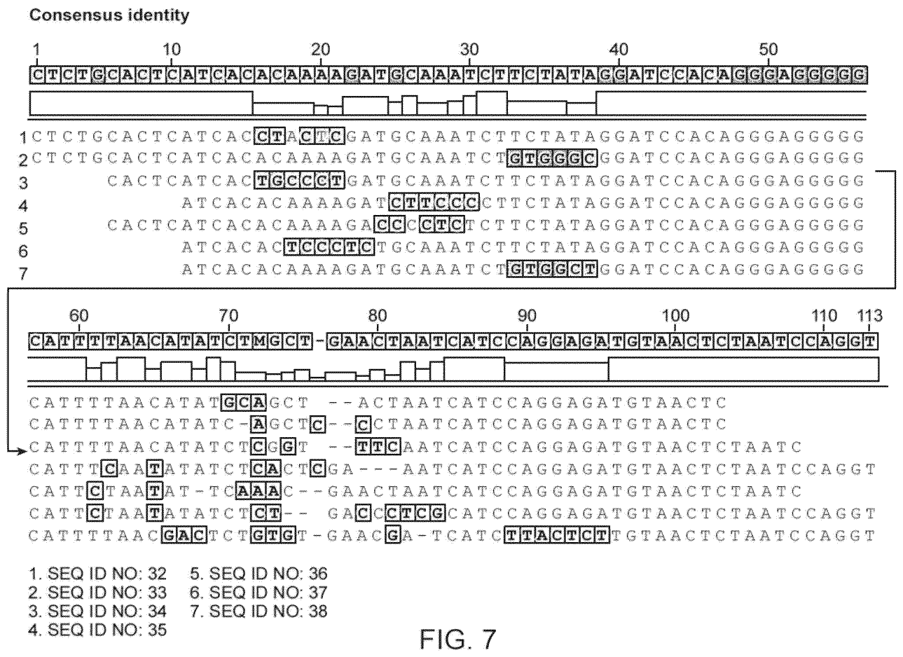

Guides RNAs for targeting DUX4 have shared regions of homology. FIG. 7 shows a sequence alignment of the DNA sequence (SEQ ID NOS: 32-38) encoding guide RNAs (SEQ ID NOS: 39-45 that target the polyA signal sequence of DUX4. The identity information is also shown in Table 7 as the percent identity between different guide RNA sequences. The different guide RNAs have sequence identity levels of greater than about 6700.

TABLE 7
Percent Distance between guide RNA sequences

| | SEQ ID | SEQ ID | SEQ ID | SEQ ID | SEQ ID | SEQ ID | SEQ ID |
|---|---|---|---|---|---|---|---|
| Guide Sequence | NO: 39 | NO: 40 | NO: 41 | NO: 42 | NO: 43 | NO: 44 | NO: 45 |
| SEQ ID NO: 39 | | 85.15% | 87.37% | 75.27% | 80.41% | 77.17% | 67.39% |
| SEQ ID NO: 40 | 85.15% | | 80.21% | 76.34% | 78.57% | 72.04% | 76.34% |
| SEQ ID NO: 41 | 87.37% | 80.21% | | 77.55% | 76.47% | 80.41% | 69.07% |
| SEQ ID NO: 42 | 75.27% | 76.34% | 77.55% | | 84.69% | 76.70% | 68.93% |
| SEQ ID NO: 43 | 80.41% | 78.57% | 76.47% | 84.69% | | 78.13% | 69.07% |
| SEQ ID NO: 44 | 77.17% | 72.04% | 80.41% | 76.70% | 78.13% | | 68.63% |
| SEQ ID NO: 45 | 67.39% | 76.34% | 69.07% | 68.93% | 69.07% | 68.63% | |

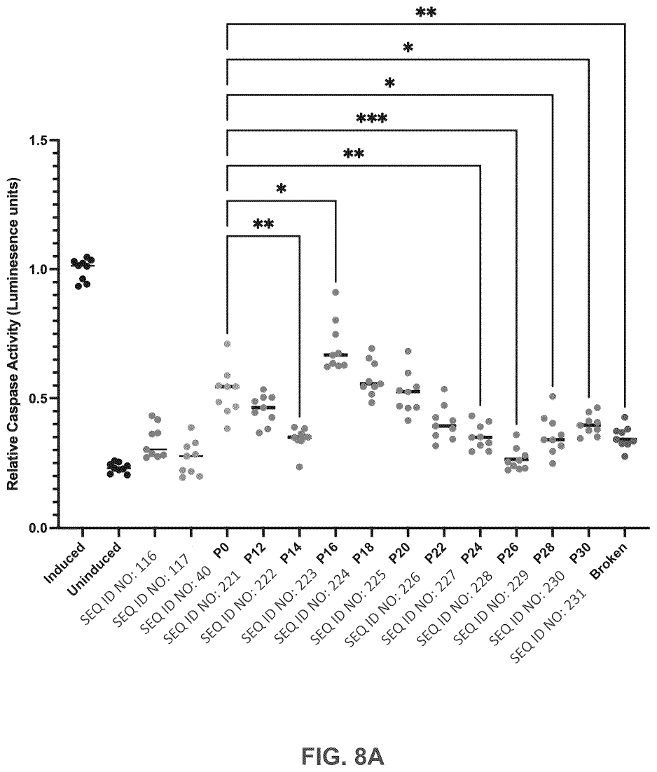

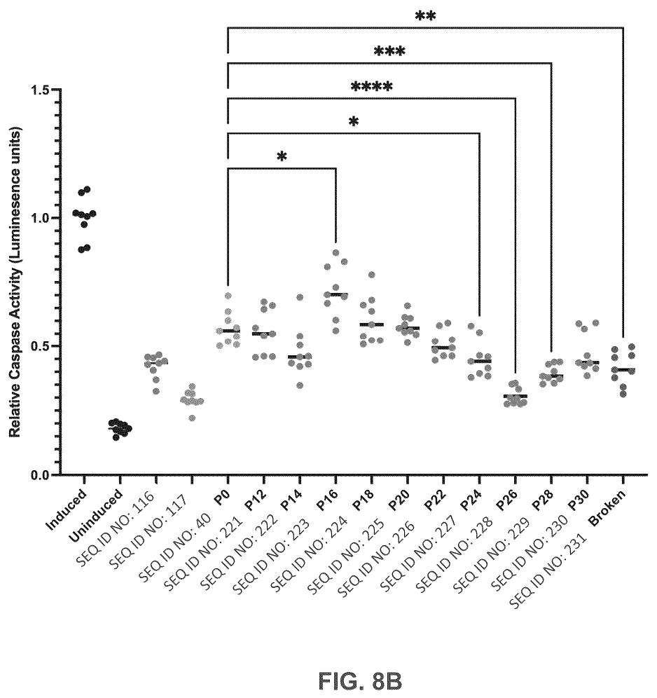

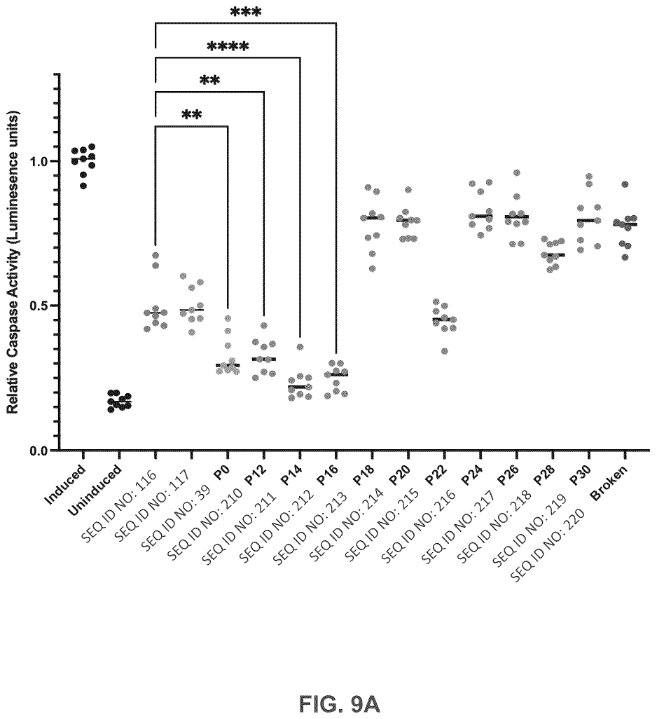

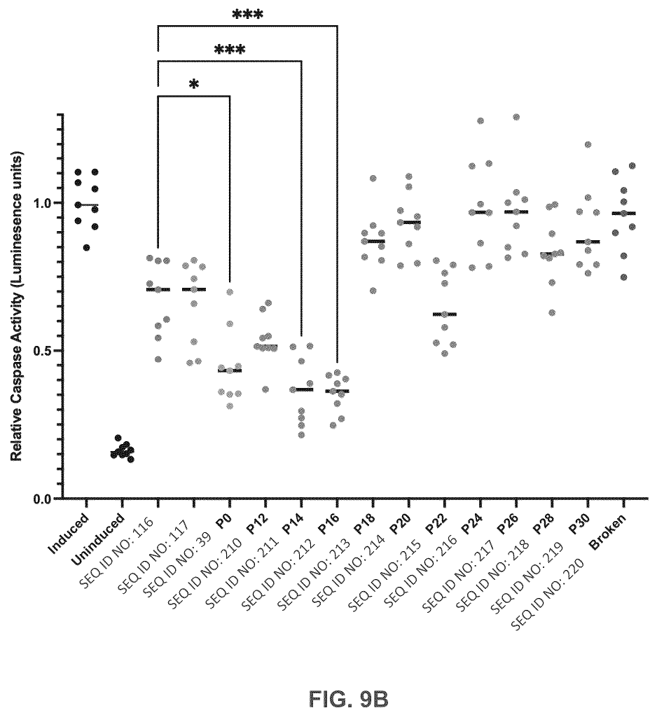

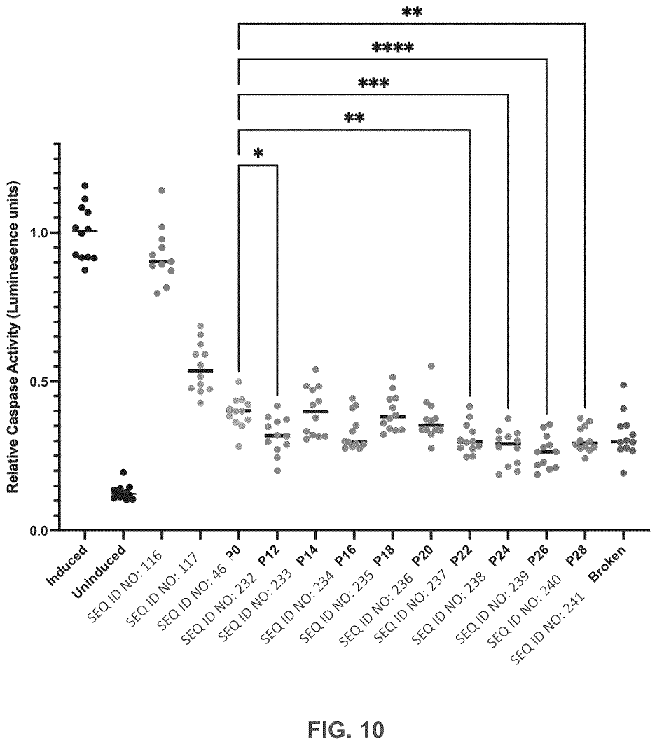

Parental Guide RNAs were tested compared with different progeny guide RNAs in a plasmid construct as shown FIG. 8A-8B, FIG. 9A-9B, and FIG. 10. FIG. 8A-8B show two biological replicates of progeny guide RNAs of SEQ ID NO: 40. All progeny guides were able to reduce caspase active in the inducible DUX4 HEK293T cells, however the P24 (SEQ ID NO: 227); P26 (SEQ ID NO: 228); P28 (SEQ ID NO: 229); and P30 (SEQ ID NO: 230) significantly reduced caspase activity as compared to the parental guide (SEQ ID NO: 40). FIG. 9A-9B show two biological replicates of progeny guide RNAs of SEQ ID NO: 39. P12 (SEQ ID NO: 210); P14 (SEQ ID NO: 211); and P16 (SEQ ID NO: 212) had comparable reduced caspase activity in the inducible DUX4 HEK293T cells as compared to the positive controls from literature (SEQ ID NO: 39). FIG. 10 shows progeny guide RNAs of SEQ ID NO: 46. All progeny guides were able to reduce caspase active in inducible DUX4 HEK293T cells, however the P22 (SEQ ID NO: 237); P24 (SEQ ID NO: 238); P26 (SEQ ID NO: 239); and P28 (SEQ ID NO: 240) had significantly reduced caspase activity as compared to the parental guide (SEQ ID NO: 46). The results show progeny guide RNAs of SEQ ID NO: 40, SEQ ID NO: 39 and SEQ ID NO: 46 were able to reduce caspase activity in inducible DUX4 HEK293T cells.

The sequences for the parental and progeny guides are shown in Table 8.

For each sequence, the structural features formed in the double stranded RNA substrate upon hybridization of the guide RNA to the target DUX4 RNA, are shown in the last column of Table 8. For reference, each structural feature formed within a guide-target RNA scaffold (target RNA sequence hybridized to an engineered guide RNA) is annotated as follows:
a) the position of the structural feature with respect to the target A (position 0—the first A (bolded) in the polyA signal sequence AUUAAA) of the target RNA sequence, with a negative value indicating upstream (5′) of the target A and a positive value indicating downstream (3′) of the target A;
b) the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c) the name of the structural feature (e.g., symmetric bulge, symmetric internal loop, asymmetric bulge, asymmetric internal loop, mismatch, or wobble base pair), and
d) the sequences of bases on the target RNA side and the engineered guide RNA side that participate in forming the structural feature.

For example, with reference to SEQ ID NO: 39, “−5_6-6_internal_loop-symmetric_UCAGAG-GCAGCU’ ‘0_1-1_mismatch_A-C’ ‘44_6-6_internal_loop-symmetric_UUUUGU-CUACUC” is read as a structural feature formed in a guide-target RNA scaffold (target DUX4 RNA sequence hybridized to an engineered guide RNA of SEQ ID NO: 39), where a structural feature starts 5 nucleotides upstream (5′) (the −5 position) from the target A (0 position) of the target RNA sequence; six contiguous bases from the target RNA sequence and six contiguous bases from the engineered guide RNA form the structural feature; the structural feature is an internal symmetric loop; and a sequence of UCAGAG from the target RNA side and a sequence of GCAGCU from the engineered guide RNA side participate in forming the internal symmetric loop. A structural feature is located at the target A (0 position) of the target RNA sequence; 1 base from the target RNA and 1 base from the engineered guide RNA form the structural feature; the structural feature is a mismatch; and a sequence of A from the target RNA side and a sequence of C from the engineered guide RNA side participate in forming the mismatch. A structural feature starts 44 nucleotides downstream (3′) (the +44 position) from the target A (0 position) of the target RNA sequence; six contiguous bases from the target RNA sequence and six contiguous bases from the engineered guide RNA form the structural feature; the structural feature is an internal symmetric loop; and a sequence of UUUUGU from the target RNA side and a sequence of CUACUC from the engineered guide RNA side participate in forming the internal symmetric loop.

TABLE 8
Parental and Progeny Guide Sequences

| Sequence | Guide DNA | Guide RNA | Structural Features of the |
|---|---|---|---|
| progeny | sequence | sequence | guide-target RNA scaffold |
| P0 | CTCTGCACTCA | CUCUGCACUCA | −5_6-6_internal_loop- |
| | TCACCTACTCG | UCACCUACUCG | symmetric_UCAGAG-GCAGCU |
| | ATGCAAATCT | AUGCAAAUCU | 0_1-1 mismatch_A-C |
| | TCTATAGGAT | UCUAUAGGAU | 44_6-6_internal_loop- |
| | CCACAGGGAG | CCACAGGGAG | symmetric_UUUUGU-CUACUC |
| | GGGGCATTTT | GGGGCAUUUU | |
| | AACATATGCA | AACAUAUGCA | |
| | GCTACTAATC | GCUACUAAUC | |
| | ATCCAGGAGA | AUCCAGGAGA | |
| | TGTAACTC | UGUAACUC | |
| | (SEQ ID | (SEQ ID | |
| | NO: 32) | NO: 39) | |
| P12 | CTCTGCACTCA | CUCUGCACUCA | −27_1-1_wobble_U-G |
| | TCACTAATCTG | UCACUAAUCU | −22_1-1_wobble_U-G |
| | ATGCAAATCT | GAUGCAAAUC | −5_6-6_internal_loop- |
| | TCTATAGGAT | UUCUAUAGGA | symmetric_UCAGAG-AUAAUC |
| | CCACAGGGAG | UCCACAGGGA | 0_1-1_mismatch_A-C |
| | GGGGCATTTT | GGGGGCAUUU | 44_6-6_internal_loop- |
| | AACATATATA | UAACAUAUAU | symmetric_UUUUGU-UAAUCU |
| | ATCACTAATC | AAUCACUAAU | |
| | ATCCGGGAGG | CAUCCGGGAG | |
| | TGTAACTC | GUGUAACUC | |
| | (SEQ ID | (SEQ ID | |
| | NO: 178) | NO: 210) | |
| P14 | CTCTGCACTCA | CUCUGCACUCA | −27_1-1_wobble_U-G |
| | TCACTAATCTG | UCACUAAUCU | −22_1-1_wobble_U-G |
| | ATGCAGATCT | GAUGCAGAUC | −14_1-1_wobble_U-G |
| | TCTATAGGAT | UUCUAUAGGA | −5_6-6_internal_loop- |
| | CCACAGGGAG | UCCACAGGGA | symmetric_UCAGAG-AUAAUC |
| | GGGGCATTTT | GGGGGCAUUU | 0_1-1_mismatch_A-C |
| | AACATATATA | UAACAUAUAU | 37_1-1_wobble_U-G |
| | ATCACTGATC | AAUCACUGAU | 44_6-6_internal_loop- |
| | ATCCGGGAGG | CAUCCGGGAG | symmetric_UUUUGU-UAAUCU |
| | TGTAACTC | GUGUAACUC | |
| | (SEQ ID | (SEQ ID | |
| | NO: 179) | NO: 211) | |
| P16 | CTCTGCACTCA | CUCUGCACUCA | −27_1-1_wobble_U-G |
| | TCACTAATCTG | UCACUAAUCU | −22_1-1_wobble_U-G |
| | ATGCAGATCT | GAUGCAGAUC | −14_1-1_wobble_U-G |
| | TCTATAGGGT | UUCUAUAGGG | −5_6-6_internal_loop- |
| | CTACAGGGAG | UCUACAGGGA | symmetric_UCAGAG-AUAAUC |
| | GGGGCATTTT | GGGGGCAUUU | 0_1-1_mismatch_A-C |
| | AACATATATA | UAACAUAUAU | 21_1-1_wobble_G-U |
| | ATCACTGATC | AAUCACUGAU | 24_1-1_wobble_U-G |
| | ATCCGGGAGG | CAUCCGGGAG | 37_1-1_wobble_U-G |
| | TGTAACTC | GUGUAACUC | 44_6-6_internal_loop- |
| | (SEQ ID | (SEQ ID | symmetric_UUUUGU-UAAUCU |
| | NO: 180) | NO: 212) | |
| P18 | CTCTGCACTCA | CUCUGCACUCA | −27_1-1_wobble_U-G |
| | TCACTAATCTG | UCACUAAUCU | −22_1-1_wobble_U-G |
| | ATGTAGATCTT | GAUGUAGAUC | −14_1-1_wobble_U-G |
| | CTATAGGGTC | UUCUAUAGGG | −5_6-6_internal_loop- |
| | TACAGGGAGG | UCUACAGGGA | symmetric_UCAGAG-AUAAUC |
| | GGGTATTTTA | GGGGGUAUUU | 0_1-1_mismatch_A-C |
| | ACATATATAA | UAACAUAUAU | 8_1-1_wobble_G-U |
| | TCACTGATCAT | AAUCACUGAU | 21_1-1_wobble_G-U |
| | CCGGGAGGTG | CAUCCGGGAG | 24_1-1_wobble_U-G |
| | TAACTC | GUGUAACUC | 37_1-1_wobble_U-G |
| | (SEQ ID | (SEQ ID | 39_1-1_wobble_G-U |
| | NO: 181) | NO: 213) | 44_6-6_internal_loop- |
| | | | symmetric_UUUUGU-UAAUCU |
| P20 | CTCTGCACTCA | CUCUGCACUCA | −27_1-1_wobble_U-G |
| | TCACTAATCTG | UCACUAAUCU | −22_1-1_wobble_U-G |
| | ATGTAGATTTT | GAUGUAGAUU | −14_1-1_wobble_U-G |
| | CTGTAGGGTC | UUCUGUAGGG | −5_6-6_internal_loop- |
| | TACAGGGAGG | UCUACAGGGA | symmetric_UCAGAG-AUAAUC |
| | GGGTATTTTA | GGGGGUAUUU | 0_1-1_mismatch_A-C |
| | ACATATATAA | UAACAUAUAU | 8_1-1_wobble_G-U |
| | TCACTGATCAT | AAUCACUGAU | 21_1-1_wobble_G-U |
| | CCGGGAGGTG | CAUCCGGGAG | 24_1-1_wobble_U-G |
| | TAACTC | GUGUAACUC | 29_1-1_wobble_U-G |
| | (SEQ ID | (SEQ ID | 34_1-1_wobble_G-U |
| | NO: 182) | NO: 214) | 37_1-1_wobble_U-G |
| | | | 39_1-1_wobble_G-U |
| | | | 44_6-6_internal_loop- |
| | | | symmetric_UUUUGU-UAAUCU |
| P22 | CTCTGCACTCA | CUCUGCACUCA | −27_1-1_wobble_U-G |
| | TCACTAATCTG | UCACUAAUCU | −22_1-1_wobble_U-G |
| | ATGTAGATTTT | GAUGUAGAUU | −18_1-1_wobble_U-G |
| | CTGTAGGGTC | UUCUGUAGGG | −17_1-1_wobble_G-U |
| | TACAGGGAGG | UCUACAGGGA | −14_1-1_wobble_U-G |
| | GGGTATTTTA | GGGGGUAUUU | −5_6-6_internal_loop- |
| | ACATATATAA | UAACAUAUAU | symmetric_UCAGAG-AUAAUC |
| | TCACTGATTGT | AAUCACUGAU | 0_1-1_mismatch_A-C |
| | CCGGGAGGTG | UGUCCGGGAG | 8_1-1_wobble_G-U |
| | TAACTC | GUGUAACUC | 21_1-1_wobble_G-U |
| | (SEQ ID | (SEQ ID | 24_1-1_wobble_U-G |
| | NO: 183) | NO: 215) | 29_1-1_wobble_U-G |
| | | | 34_1-1_wobble_G-U |
| | | | 37_1-1_wobble_U-G |
| | | | 39_1-1_wobble_G-U |
| | | | 44_6-6_internal_loop- |
| | | | symmetric_UUUUGU-UAAUCU |
| P24 | CTCTGCACTCA | CUCUGCACUCA | −31_1-1_wobble_U-G |
| | TCACTAATCTG | UCACUAAUCU | −27_1-1_wobble_U-G |
| | ATGTAGATTTT | GAUGUAGAUU | −22_1-1_wobble_U-G |
| | CTGTAGGGTC | UUCUGUAGGG | −21_1-1_wobble_G-U |
| | TACAGGGAGG | UCUACAGGGA | −18_1-1_wobble_U-G |
| | GGGTATTTTA | GGGGGUAUUU | −17_1-1_wobble_G-U |
| | ACATATATAA | UAACAUAUAU | −14_1-1_wobble_U-G |
| | TCACTGATTGT | AAUCACUGAU | −5_6-6_internal_loop- |
| | CTGGGAGGTG | UGUCUGGGAG | symmetric_UCAGAG-AUAAUC |
| | TGACTC | GUGUGACUC | 0_1-1_mismatch_A-C |
| | (SEQ ID | (SEQ ID | 8_1-1_wobble_G-U |
| | NO: 184) | NO: 216) | 21_1-1_wobble_G-U |
| | | | 24_1-1_wobble_U-G |
| | | | 29_1-1_wobble_U-G |
| | | | 34_1-1_wobble_G-U |
| | | | 37_1-1_wobble_U-G |
| | | | 39_1-1_wobble_G-U |
| | | | 44_6-6_internal_loop- |
| | | | symmetric_UUUUGU-UAAUCU |
| P26 | CTCTGCACTCA | CUCUGCACUCA | −35_1-1_wobble_G-U |
| | TCACTAATCTG | UCACUAAUCU | −33_1-1_wobble_G-U |
| | ATGTAGATTTT | GAUGUAGAUU | −31_1-1_wobble_U-G |
| | CTGTAGGGTC | UUCUGUAGGG | −27_1-1_wobble_U-G |
| | TACAGGGAGG | UCUACAGGGA | −22_1-1_wobble_U-G |
| | GGGTATTTTA | GGGGGUAUUU | −21_1-1_wobble_G-U |
| | ACATATATAA | UAACAUAUAU | −18_1-1_wobble_U-G |
| | TCACTGATTGT | AAUCACUGAU | −17_1-1_wobble_G-U |
| | CTGGGAGGTG | UGUCUGGGAG | −14_1-1_wobble_U-G |
| | TGATTT | GUGUGAUUU | −5_6-6_internal_loop- |
| | (SEQ ID | (SEQ ID | symmetric_UCAGAG-AUAAUC |
| | NO: 185) | NO: 217) | 0_1-1_mismatch_A-C |
| | | | 8_1-1_wobble_G-U |
| | | | 21_1-1_wobble_G-U |
| | | | 24_1-1_wobble_U-G |
| | | | 29_1-1_wobble_U-G |
| | | | 34_1-1_wobble_G-U |
| | | | 37_1-1_wobble_U-G |
| | | | 39_1-1_wobble_G-U |
| | | | 44_6-6_internal_loop- |
| | | | symmetric_UUUUGU-UAAUCU |
| P28 | CTCTGCACTCG | CUCUGCACUCG | −35_1-1_wobble_G-U |
| | TTACTAATCTG | UUACUAAUCU | −33_1-1_wobble_G-U |
| | ATGTAGATTTT | GAUGUAGAUU | −31_1-1_wobble_U-G |
| | CTGTAGGGTC | UUCUGUAGGG | −27_1-1_wobble_U-G |
| | TACAGGGAGG | UCUACAGGGA | −22_1-1_wobble_U-G |
| | GGGTATTTTA | GGGGGUAUUU | −21_1-1_wobble_G-U |
| | ACATATATAA | UAACAUAUAU | −18_1-1_wobble_U-G |
| | TCACTGATTGT | AAUCACUGAU | −17_1-1_wobble_G-U |
| | CTGGGAGGTG | UGUCUGGGAG | −14_1-1_wobble_U-G |
| | TGATTT | GUGUGAUUU | −5_6-6_internal_loop- |
| | (SEQ ID | (SEQ ID | symmetric_UCAGAG-AUAAUC |
| | NO: 186) | NO: 218) | 0_1-1_mismatch_A-C |
| | | | 8_1-1_wobble_G-U |
| | | | 21_1-1_wobble_G-U |
| | | | 24_1-1_wobble_U-G |
| | | | 29_1-1_wobble_U-G |
| | | | 34_1-1_wobble_G-U |
| | | | 37_1-1_wobble_U-G |
| | | | 39_1-1_wobble_G-U |
| | | | 44_6-6_internal_loop- |
| | | | symmetric_UUUUGU-UAAUCU |
| | | | 52_1-1_wobble_G-U |
| | | | 54_1-1_wobble_U-G |
| P30 | CTTTGCGCTCG | CUUUGCGCUCG | −35_1-1_wobble_G-U |
| | TTACTAATCTG | UUACUAAUCU | −33_1-1_wobble_G-U |
| | ATGTAGATTTT | GAUGUAGAUU | −31_1-1_wobble_U-G |
| | CTGTAGGGTC | UUCUGUAGGG | −27_1-1_wobble_U-G |
| | TACAGGGAGG | UCUACAGGGA | −22_1-1_wobble_U-G |
| | GGGTATTTTA | GGGGGUAUUU | −21_1-1_wobble_G-U |
| | ACATATATAA | UAACAUAUAU | −18_1-1_wobble_U-G |
| | TCACTGATTGT | AAUCACUGAU | −17_1-1_wobble_G-U |
| | CTGGGAGGTG | UGUCUGGGAG | −14_1-1_wobble_U-G |
| | TGATTT | GUGUGAUUU | −5_6-6_internal_loop- |
| | (SEQ ID | (SEQ ID | symmetric_UCAGAG-AUAAUC |
| | NO: 187) | NO: 219) | 0_1-1_mismatch_A-C |
| | | | 8_1-1_wobble_G-U |
| | | | 21_1-1_wobble_G-U |
| | | | 24_1-1_wobble_U-G |
| | | | 29_1-1_wobble_U-G |
| | | | 34_1-1_wobble_G-U |
| | | | 37_1-1_wobble_U-G |
| | | | 39_1-1_wobble_G-U |
| | | | 44_6-6_internal_loop- |
| | | | symmetric_UUUUGU-UAAUCU |
| | | | 52_1-1_wobble_G-U |
| | | | 54_1-1_wobble_U-G |
| | | | 58_1-1_wobble_U-G |
| | | | 62_1-1_wobble_G-U |
| Broken | CTCTGCACTCA | CUCUGCACUCA | −27_1-1_wobble_U-G |
| | TCACTAATCTG | UCACUAAUCU | −22_1-1_wobble_U-G |
| | ATGCAGATCT | GAUGCAGAUC | −14_1-1_wobble_U-G |
| | TCTATAGGAT | UUCUAUAGGA | −9_2-2_bulge- |
| | CCACAGGGAG | UCCACAGGGA | symmetric_UC-UC |
| | GGGGCATTTT | GGGGGCAUUU | 0_1-1_mismatch_A-C |
| | AACATATCTCT | UAACAUAUCU | 37_1-1_wobble_U-G |
| | TCACTGATCAT | CUUCACUGAUC | 44_6-6_internal_loop- |
| | CCGGGAGGTG | AUCCGGGAGG | symmetric_UUUUGU-UAAUCU |
| | TAACTC | UGUAACUC | |
| | (SEQ ID | (SEQ ID | |
| | NO: 188) | NO: 220) | |
| P0 | CTCTGCACTCA | CUCUGCACUCA | −6_6-6_internal_loop- |
| | TCACACAAAA | UCACACAAAA | symmetric_UUCAGA-AGCUCC |
| | GATGCAAATC | GAUGCAAAUC | 0_1-1_mismatch_A-C |
| | TGTGGGCGGA | UGUGGGCGGA | 27_6-6_internal_loop- |
| | TCCACAGGGA | UCCACAGGGA | symmetric_UAUAGA-GUGGGC |
| | GGGGGCATTT | GGGGGCAUUU | |
| | TAACATATCA | UAACAUAUCA | |
| | GCTCCCTAATC | GCUCCCUAAUC | |
| | ATCCAGGAGA | AUCCAGGAGA | |
| | TGTAACTC | UGUAACUC | |
| | (SEQ ID | (SEQ ID | |
| | NO: 33) | NO: 40) | |
| P12 | CTCTGCACTCA | CUCUGCACUCA | −32_1-1_wobble_U-G |
| | TCACACAAAA | UCACACAAAA | −6_6-6_internal_loop- |
| | GATGCAAATC | GAUGCAAAUC | symmetric_UUCAGA-AAUAUU |
| | TAACCATGGA | UAACCAUGGA | 0_1-1_mismatch_A-C |
| | TCCACAGGGA | UCCACAGGGA | 27_6-6_internal_loop- |
| | GGGGGCATTT | GGGGGCAUUU | symmetric_UAUAGA-AACCAU |
| | TAACATATCA | UAACAUAUCA | |
| | ATATTCTAATC | AUAUUCUAAU | |
| | ATCCAGGAGA | CAUCCAGGAG | |
| | TGTAGCTC | AUGUAGCUC | |
| | (SEQ ID | (SEQ ID | |
| | NO: 189) | NO: 221) | |
| P14 | CTCTGCACTCA | CUCUGCACUCA | −35_1-1_wobble_G-U |
| | TCACACAAAA | UCACACAAAA | −32_1-1_wobble_U-G |
| | GATGCAAATC | GAUGCAAAUC | −15_1-1_wobble_U-G |
| | TAACCATGGA | UAACCAUGGA | −6_6-6_internal_loop- |
| | TCCACAGGGA | UCCACAGGGA | symmetric_UUCAGA-AAUAUU |
| | GGGGGCATTT | GGGGGCAUUU | 0_1-1_mismatch_A-C |
| | TAACATATCA | UAACAUAUCA | 27_6-6_internal_loop- |
| | ATATTCTAGTC | AUAUUCUAGU | symmetric_UAUAGA-AACCAU |
| | ATCCAGGAGA | CAUCCAGGAG | |
| | TGTAGCTT | AUGUAGCUU | |
| | (SEQ ID | (SEQ ID | |
| | NO: 190) | NO: 222) | |
| P16 | CTCTGCACTCA | CUCUGCACUCA | −35_1-1_wobble_G-U |
| | TCACACAAAA | UCACACAAAA | −32_1-1_wobble_U-G |
| | GATGCAAATC | GAUGCAAAUC | −15_1-1_wobble_U-G |
| | TAACCATGGA | UAACCAUGGA | −6_6-6_internal_loop- |
| | TCCGCAGGGA | UCCGCAGGGA | symmetric_UUCAGA-AAUAUU |
| | GGGGGTATTT | GGGGGUAUUU | 0_1-1_mismatch_A-C |
| | TAACATATCA | UAACAUAUCA | 8_1-1_wobble_G-U |
| | ATATTCTAGTC | AUAUUCUAGU | 20_1-1_wobble_U-G |
| | ATCCAGGAGA | CAUCCAGGAG | 27_6-6_internal_loop- |
| | TGTAGCTT | AUGUAGCUU | symmetric_UAUAGA-AACCAU |
| | (SEQ ID | (SEQ ID | |
| | NO: 191) | NO: 223) | |
| P18 | CTCTGTACTCA | CUCUGUACUCA | −35_1-1_wobble_G-U |
| | TCACACAAAA | UCACACAAAA | −32_1-1_wobble_U-G |
| | GATGCAGATC | GAUGCAGAUC | −15_1-1_wobble_U-G |
| | TAACCATGGA | UAACCAUGGA | −6_6-6_internal_loop- |
| | TCCGCAGGGA | UCCGCAGGGA | symmetric_UUCAGA-AAUAUU |
| | GGGGGTATTT | GGGGGUAUUU | 0_1-1_mismatch_A-C |
| | TAACATATCA | UAACAUAUCA | 8_1-1_wobble_G-U |
| | ATATTCTAGTC | AUAUUCUAGU | 20_1-1_wobble_U-G |
| | ATCCAGGAGA | CAUCCAGGAG | 27_6-6_internal_loop- |
| | TGTAGCTT | AUGUAGCUU | symmetric_UAUAGA-AACCAU |
| | (SEQ ID | (SEQ ID | 37_1-1_wobble_U-G |
| | NO: 192) | NO: 224) | 59_1-1_wobble_G-U |
| P20 | CTCTGTACTCA | CUCUGUACUCA | −35_1-1_wobble_G-U |
| | TCACACAAAA | UCACACAAAA | −32_1-1_wobble_U-G |
| | GATGCAGATC | GAUGCAGAUC | −27_1-1_wobble_U-G |
| | TAACCATGGA | UAACCAUGGA | −20_1-1_wobble_G-U |
| | TCCGCAGGGA | UCCGCAGGGA | −15_1-1_wobble_U-G |
| | GGGGGTATTT | GGGGGUAUUU | −6_6-6_internal_loop- |
| | TAACATATCA | UAACAUAUCA | symmetric_UUCAGA-AAUAUU |
| | ATATTCTAGTC | AUAUUCUAGU | 0_1-1_mismatch_A-C |
| | ATTCAGGAGG | CAUUCAGGAG | 8_1-1_wobble_G-U |
| | TGTAGCTT | GUGUAGCUU | 20_1-1_wobble_U-G |
| | (SEQ ID | (SEQ ID | 27_6-6_internal_loop- |
| | NO: 193) | NO: 225) | symmetric_UAUAGA-AACCAU |
| | | | 37_1-1_wobble_U-G |
| | | | 59_1-1_wobble_G-U |
| P22 | CTCTGTACTCA | CUCUGUACUCA | −35_1-1_wobble_G-U |
| | TCACACAAGA | UCACACAAGA | −32_1-1_wobble_U-G |
| | GATGCAGATC | GAUGCAGAUC | −27_1-1_wobble_U-G |
| | TAACCATGGA | UAACCAUGGA | −20_1-1_wobble_G-U |
| | TTCGCAGGGA | UUCGCAGGGA | −15_1-1_wobble_U-G |
| | GGGGGTATTT | GGGGGUAUUU | −6_6-6_internal_loop- |
| | TAACATATCA | UAACAUAUCA | symmetric_UUCAGA-AAUAUU |
| | ATATTCTAGTC | AUAUUCUAGU | 0_1-1_mismatch_A-C |
| | ATTCAGGAGG | CAUUCAGGAG | 8_1-1_wobble_G-U |
| | TGTAGCTT | GUGUAGCUU | 20_1-1_wobble_U-G |
| | (SEQ ID | (SEQ ID | 22_1-1_wobble_G-U |
| | NO: 194) | NO: 226) | 27_6-6_internal_loop- |
| | | | symmetric_UAUAGA-AACCAU |
| | | | 37_1-1_wobble_U-G |
| | | | 45_1-1_wobble_U-G |
| | | | 59_1-1_wobble_G-U |
| P24 | CTCTGTACTTA | CUCUGUACUU | −35_1-1_wobble_G-U |
| | TCACACAAGA | AUCACACAAG | −32_1-1_wobble_U-G |
| | GATGCAGATC | AGAUGCAGAU | −27_1-1_wobble_U-G |
| | TAACCATGGA | CUAACCAUGG | −20_1-1_wobble_G-U |
| | TTCGCAGGGA | AUUCGCAGGG | −18_1-1_wobble_U-G |
| | GGGGGTATTT | AGGGGGUAUU | −15_1-1_wobble_U-G |
| | TAACATATCA | UUAACAUAUC | −6_6-6_internal_loop- |
| | ATATTCTAGTC | AAUAUUCUAG | symmetric_UUCAGA-AAUAUU |
| | GTTCAGGAGG | UCGUUCAGGA | 0_1-1_mismatch_A-C |
| | TGTAGCTT | GGUGUAGCUU | 8_1-1_wobble_G-U |
| | (SEQ ID | (SEQ ID | 20_1-1_wobble_U-G |
| | NO: 195) | NO: 227) | 22_1-1_wobble_G-U |
| | | | 27_6-6_internal_loop- |
| | | | symmetric_UAUAGA-AACCAU |
| | | | 37_1-1_wobble_U-G |
| | | | 45_1-1_wobble_U-G |
| | | | 55_1-1_wobble_G-U |
| | | | 59_1-1_wobble_G-U |
| P26 | CTCTGTACTTA | CUCUGUACUU | −35_1-1_wobble_G-U |
| | TCACGCAAGA | AUCACGCAAG | −32_1-1_wobble_U-G |
| | GATGCAGATC | AGAUGCAGAU | −27_1-1_wobble_U-G |
| | TAACCATGGA | CUAACCAUGG | −20_1-1_wobble_G-U |
| | TTCGCAGGGA | AUUCGCAGGG | −18_1-1_wobble_U-G |
| | GGGGGTATTT | AGGGGGUAUU | −17_1-1_wobble_G-U |
| | TAACATATCA | UUAACAUAUC | −15_1-1_wobble_U-G |
| | ATATTCTAGTT | AAUAUUCUAG | −6_6-6_internal_loop- |
| | GTTCAGGAGG | UUGUUCAGGA | symmetric_UUCAGA-AAUAUU |
| | TGTAGCTT | GGUGUAGCUU | 0_1-1_mismatch_A-C |
| | (SEQ ID | (SEQ ID | 8_1-1_wobble_G-U |
| | NO: 196) | NO: 228) | 20_1-1_wobble_U-G |
| | | | 22_1-1_wobble_G-U |
| | | | 27_6-6_internal_loop- |
| | | | symmetric_UAUAGA-AACCAU |
| | | | 37_1-1_wobble_U-G |
| | | | 45_1-1_wobble_U-G |
| | | | 49_1-1_wobble_U-G |
| | | | 55_1-1_wobble_G-U |
| | | | 59_1-1_wobble_G-U |
| P28 | CTCTGTACTTA | CUCUGUACUU | −35_1-1_wobble_G-U |
| | TCACGCAAGA | AUCACGCAAG | −32_1-1_wobble_U-G |
| | GATGTAGATC | AGAUGUAGAU | −27_1-1_wobble_U-G |
| | TAACCATGGA | CUAACCAUGG | −20_1-1_wobble_G-U |
| | TTCGCAGGGA | AUUCGCAGGG | −18_1-1_wobble_U-G |
| | GGGGGTATTT | AGGGGGUAUU | −17_1-1_wobble_G-U |
| | TAACATATCA | UUAACAUAUC | −15_1-1_wobble_U-G |
| | ATATTCTGGTT | AAUAUUCUGG | −14_1-1_wobble_U-G |
| | GTTCAGGAGG | UUGUUCAGGA | −6_6-6_internal_loop- |
| | TGTAGCTT | GGUGUAGCUU | symmetric_UUCAGA-AAUAUU |
| | (SEQ ID | (SEQ ID | 0_1-1_mismatch_A-C |
| | NO: 197) | NO: 229) | 8_1-1_wobble_G-U |
| | | | 20_1-1_wobble_U-G |
| | | | 22_1-1_wobble_G-U |
| | | | 27_6-6_internal_loop- |
| | | | symmetric_UAUAGA-AACCAU |
| | | | 37_1-1_wobble_U-G |
| | | | 39_1-1_wobble_G-U |
| | | | 45_1-1_wobble_U-G |
| | | | 49_1-1_wobble_U-G |
| | | | 55_1-1_wobble_G-U |
| | | | 59_1-1_wobble_G-U |
| P30 | CTCTGTACTTA | CUCUGUACUU | −35_1-1_wobble_G-U |
| | TCACGCGAGA | AUCACGCGAG | −32_1-1_wobble_U-G |
| | GATGTAGATT | AGAUGUAGAU | −27_1-1_wobble_U-G |
| | TAACCATGGA | UUAACCAUGG | −20_1-1_wobble_G-U |
| | TTCGCAGGGA | AUUCGCAGGG | −18_1-1_wobble_U-G |
| | GGGGGTATTT | AGGGGGUAUU | −17_1-1_wobble_G-U |
| | TAACATATCA | UUAACAUAUC | −15_1-1_wobble_U-G |
| | ATATTCTGGTT | AAUAUUCUGG | −14_1-1_wobble_U-G |
| | GTTCAGGAGG | UUGUUCAGGA | −6_6-6_internal_loop- |
| | TGTAGCTT | GGUGUAGCUU | symmetric_UUCAGA-AAUAUU |
| | (SEQ ID | (SEQ ID | 0_1-1_mismatch_A-C |
| | NO: 198) | NO: 230) | 8_1-1_wobble_G-U |
| | | | 20_1-1_wobble_U-G |
| | | | 22_1-1_wobble_G-U |
| | | | 27_6-6_internal_loop- |
| | | | symmetric_UAUAGA-AACCAU |
| | | | 34_1-1_wobble_G-U |
| | | | 37_1-1_wobble_U-G |
| | | | 39_1-1_wobble_G-U |
| | | | 45_1-1_wobble_U-G |
| | | | 47_1-1_wobble_U-G |
| | | | 49_1-1_wobble_U-G |
| | | | 55_1-1_wobble_G-U |
| | | | 59_1-1_wobble_G-U |
| Broken | CTCTGTACTTA | CUCUGUACUU | −35_1-1_wobble_G-U |
| | TCACGCGAGA | AUCACGCGAG | −32_1-1_wobble_U-G |
| | GATGTAGATT | AGAUGUAGAU | −27_1-1_wobble_U-G |
| | TAACCATGGA | UUAACCAUGG | −20_1-1_wobble_G-U |
| | TTCGCAGGGA | AUUCGCAGGG | −18_1-1_wobble_U-G |
| | GGGGGTATTT | AGGGGGUAUU | −17_1-1_wobble_G-U |
| | TAACATATCTC | UUAACAUAUC | −15_1-1_wobble_U-G |
| | TGTTCTGGTTG | UCUGUUCUGG | −14_1-1_wobble_U-G |
| | TTCAGGAGGT | UUGUUCAGGA | −10_2-2_bulge- |
| | GTAGCTT | GGUGUAGCUU | symmetric_UU-UU |
| | (SEQ ID | (SEQ ID | 0_1-1_mismatch_A-C |
| | NO: 199) | NO: 231) | 8_1-1_wobble_G-U |
| | | | 20_1-1_wobble_U-G |
| | | | 22_1-1_wobble_G-U |
| | | | 27_6-6_internal_loop- |
| | | | symmetric_UAUAGA-AACCAU |
| | | | 34_1-1_wobble_G-U |
| | | | 37_1-1_wobble_U-G |
| | | | 39_1-1_wobble_G-U |
| | | | 45_1-1_wobble_U-G |
| | | | 47_1-1_wobble_U-G |
| | | | 49_1-1_wobble_U-G |
| | | | 55_1-1_wobble_G-U |
| | | | 59_1-1_wobble_G-U |
| P0 | CTCTGCACTCA | CUCUGCACUCA | −22_6-6_internal_loop- |
| | TCACACAAAA | UCACACAAAA | symmetric_UCUCCU-UACUCU |
| | GACCCCTCTCT | GACCCCUCUCU | −21_1-1_wobble_G-U |
| | TCTATAGGAT | UCUAUAGGAU | −14_1-1_wobble_U-G |
| | CCACAGGGAG | CCACAGGGAG | −13_1-0_bulge- |
| | GGGGCATTTT | GGGGCAUUUU | asymmetric_A- |
| | AACGACTCTG | AACGACUCUG | −7_1-3_bulge- |
| | TGTGAACGAT | UGUGAACGAU | asymmetric_G-GUG |
| | CATCTTACTCT | CAUCUUACUCU | −3->0_4-4_bulge- |
| | TGTAACTC | UGUAACUC | symmetric_UAUA-CGAC |
| | (SEQ ID | (SEQ ID | 36_6-6_internal_loop- |
| | NO: 79) | NO: 46) | symmetric_UUUGCA-CCCCUC |
| P12 | CTCTGCACTCA | CUCUGCACUCA | −31_1-1_wobble_U-G |
| | TCACATAAAA | UCACAUAAAA | −22_6-6_internal_loop- |
| | GAAGATTTTCT | GAAGAUUUUC | symmetric_UCUCCU-UUUCCC |
| | TCTATAGGATT | UUCUAUAGGA | −21_1-1_wobble_G-U |
| | CACAGGGAGG | UUCACAGGGA | −14_1-1_wobble_U-G |
| | GGGCATTTTA | GGGGGCAUUU | −13_1-0_bulge- |
| | ACGACTCTGT | UAACGACUCU | asymmetric_A- |
| | GTGAACGATC | GUGUGAACGA | −7_1-3_bulge- |
| | ATCTTTTCCCT | UCAUCUUUUCC | asymmetric_G-GUG |
| | GTGACTC | CUGUGACUC | −3->0_4-4_bulge- |
| | (SEQ ID | (SEQ ID | symmetric_UAUA-CGAC |
| | NO: 200) | NO: 232) | 22_1-1_wobble_G-U |
| | | | 36_4-4_bulge- |
| | | | symmetric_UUUG-AUUU |
| | | | 41_1-1_mismatch_A-A |
| | | | 48_1-1_wobble_G-U |
| P14 | CTCTGCACTCA | CUCUGCACUCA | −33_1-1_wobble_G-U |
| | TCACATAAGA | UCACAUAAGA | −31_1-1_wobble_U-G |
| | GAAGATTTTCT | GAAGAUUUUC | −22_6-6_internal_loop- |
| | TCTATAGGATT | UUCUAUAGGA | symmetric_UCUCCU-UUUCCC |
| | CACAGGGAGG | UUCACAGGGA | −21_1-1_wobble_G-U |
| | GGGCATTTTA | GGGGGCAUUU | −14_1-1_wobble_U-G |
| | ACGACTCTGT | UAACGACUCU | −13_1-0_bulge- |
| | GTGAACGATC | GUGUGAACGA | asymmetric_A- |
| | ATCTTTTCCCT | UCAUCUUUUCC | −7_1-3_bulge- |
| | GTGATTC | CUGUGAUUC | asymmetric_G-GUG |
| | (SEQ ID | (SEQ ID | −3->0_4-4_bulge- |
| | NO: 201) | NO: 233) | symmetric_UAUA-CGAC |
| | | | 22_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| P16 | CTCTGCACTTA | CUCUGCACUUA | −33_1-1_wobble_G-U |
| | TCACATAAGA | UCACAUAAGA | −31_1-1_wobble_U-G |
| | GAAGATTTTCT | GAAGAUUUUC | −22_6-6_internal_loop- |
| | TCTGTAGGATT | UUCUGUAGGA | symmetric_UCUCCU-UUUCCC |
| | CACAGGGAGG | UUCACAGGGA | −21_1-1_wobble_G-U |
| | GGGCATTTTA | GGGGGCAUUU | −14_1-1_wobble_U-G |
| | ACGACTCTGT | UAACGACUCU | −13_1-0_bulge- |
| | GTGAACGATC | GUGUGAACGA | asymmetric_A- |
| | ATCTTTTCCCT | UCAUCUUUUCC | −7_1-3_bulge- |
| | GTGATTC | CUGUGAUUC | asymmetric_G-GUG |
| | (SEQ ID | (SEQ ID | −3->0_4-4_bulge- |
| | NO: 202) | NO: 234) | symmetric_UAUA-CGAC |
| | | | 22_1-1_wobble_G-U |
| | | | 29_1-1_wobble_U-G |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 55_1-1_wobble_G-U |
| P18 | CTCTGCACTTA | CUCUGCACUUA | −33_1-1_wobble_G-U |
| | TCGCATAAGA | UCGCAUAAGA | −31_1-1_wobble_U-G |
| | GAAGATTTTCT | GAAGAUUUUC | −22_6-6_internal_loop- |
| | TTTGTAGGATT | UUUUGUAGGA | symmetric_UCUCCU-UUUCCC |
| | CACAGGGAGG | UUCACAGGGA | −21_1-1_wobble_G-U |
| | GGGCATTTTA | GGGGGCAUUU | −14_1-1_wobble_U-G |
| | ACGACTCTGT | UAACGACUCU | −13_1-0_bulge- |
| | GTGAACGATC | GUGUGAACGA | asymmetric_A- |
| | ATCTTTTCCCT | UCAUCUUUUCC | −7_1-3_bulge- |
| | GTGATTC | CUGUGAUUC | asymmetric_G-GUG |
| | (SEQ ID | (SEQ ID | −3->0_4-4_bulge- |
| | NO: 203) | NO: 235) | symmetric_UAUA-CGAC |
| | | | 22_1-1_wobble_G-U |
| | | | 29_1-1_wobble_U-G |
| | | | 31_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| P20 | CTCTGCACTTA | CUCUGCACUUA | −33_1-1_wobble_G-U |
| | TCGCATAAGA | UCGCAUAAGA | −31_1-1_wobble_U-G |
| | GAAGATTTTCT | GAAGAUUUUC | −22_6-6_internal_loop- |
| | TTTGTAGGGTT | UUUUGUAGGG | symmetric_UCUCCU-UUUCCC |
| | CGCAGGGAGG | UUCGCAGGGA | −21_1-1_wobble_G-U |
| | GGGCATTTTA | GGGGGCAUUU | −14_1-1_wobble_U-G |
| | ACGACTCTGT | UAACGACUCU | −13_1-0_bulge- |
| | GTGAACGATC | GUGUGAACGA | asymmetric_A- |
| | ATCTTTTCCCT | UCAUCUUUUCC | −7_1-3_bulge- |
| | GTGATTC | CUGUGAUUC | asymmetric_G-GUG |
| | (SEQ ID | (SEQ ID | −3->0_4-4_bulge- |
| | NO: 204) | NO: 236) | symmetric_UAUA-CGAC |
| | | | 20_1-1_wobble_U-G |
| | | | 22_1-1_wobble_G-U |
| | | | 24_1-1_wobble_U-G |
| | | | 29_1-1_wobble_U-G |
| | | | 31_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| P22 | CTCTGCGCTTA | CUCUGCGCUUA | −33_1-1_wobble_G-U |
| | TCGCATAAGA | UCGCAUAAGA | −31_1-1_wobble_U-G |
| | GAAGATTTTTT | GAAGAUUUUU | −22_6-6_internal_loop- |
| | TTTGTAGGGTT | UUUUGUAGGG | symmetric_UCUCCU-UUUCCC |
| | CGCAGGGAGG | UUCGCAGGGA | −21_1-1_wobble_G-U |
| | GGGCATTTTA | GGGGGCAUUU | −14_1-1_wobble_U-G |
| | ACGACTCTGT | UAACGACUCU | −13_1-0_bulge- |
| | GTGAACGATC | GUGUGAACGA | asymmetric_A- |
| | ATCTTTTCCCT | UCAUCUUUUCC | −7_1-3_bulge- |
| | GTGATTC | CUGUGAUUC | asymmetric_G-GUG |
| | (SEQ ID | (SEQ ID | −3->0_4-4_bulge- |
| | NO: 205) | NO: 237) | symmetric_UAUA-CGAC |
| | | | 20_1-1_wobble_U-G |
| | | | 22_1-1_wobble_G-U |
| | | | 24_1-1_wobble_U-G |
| | | | 29_1-1_wobble_U-G |
| | | | 31_1-1_wobble_G-U |
| | | | 34_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| | | | 58_1-1_wobble_U-G |
| P24 | CTTTGCGCTTA | CUUUGCGCUU | −33_1-1_wobble_G-U |
| | TCGCATAAGA | AUCGCAUAAG | −31_1-1_wobble_U-G |
| | GAAGATTTTTT | AGAAGAUUUU | −22_6-6_internal_loop- |
| | TTTGTAGGGTT | UUUUUGUAGG | symmetric_UCUCCU-UUUCCC |
| | CGCGGGGAGG | GUUCGCGGGG | −21_1-1_wobble_G-U |
| | GGGCATTTTA | AGGGGGCAUU | −14_1-1_wobble_U-G |
| | ACGACTCTGT | UUAACGACUC | −13_1-0_bulge- |
| | GTGAACGATC | UGUGUGAACG | asymmetric_A- |
| | ATCTTTTCCCT | AUCAUCUUUU | −7_1-3_bulge- |
| | GTGATTC | CCCUGUGAUUC | asymmetric_G-GUG |
| | (SEQ ID | (SEQ ID | −3->0_4-4_bulge- |
| | NO: 206) | NO: 238) | symmetric_UAUA-CGAC |
| | | | 18_1-1_wobble_U-G |
| | | | 20_1-1_wobble_U-G |
| | | | 22_1-1_wobble_G-U |
| | | | 24_1-1_wobble_U-G |
| | | | 29_1-1_wobble_U-G |
| | | | 31_1-1_wobble_G-U |
| | | | 34_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| | | | 58_1-1_wobble_U-G |
| | | | 62_1-1_wobble_G-U |
| P26 | TTTTGCGCTTA | UUUUGCGCUU | −35_1-1_wobble_G-U |
| | TCGCATAAGA | AUCGCAUAAG | −33_1-1_wobble_G-U |
| | GAAGATTTTTT | AGAAGAUUUU | −31_1-1_wobble_U-G |
| | TTTGTAGGGTT | UUUUUGUAGG | −22_6-6_internal_loop- |
| | CGCGGGGAGG | GUUCGCGGGG | symmetric_UCUCCU-UUUCCC |
| | GGGCATTTTA | AGGGGGCAUU | −21_1-1_wobble_G-U |
| | ACGACTCTGT | UUAACGACUC | −14_1-1_wobble_U-G |
| | GTGAACGATC | UGUGUGAACG | −13_1-0_bulge- |
| | ATCTTTTCCCT | AUCAUCUUUU | asymmetric_A- |
| | GTGATTT | CCCUGUGAUU | −7_1-3_bulge- |
| | (SEQ ID | U | asymmetric_G-GUG |
| | NO: 207) | (SEQ ID | −3->0_4-4_bulge- |
| | | NO: 239) | symmetric_UAUA-CGAC |
| | | | 18_1-1_wobble_U-G |
| | | | 20_1-1_wobble_U-G |
| | | | 22_1-1_wobble_G-U |
| | | | 24_1-1_wobble_U-G |
| | | | 29_1-1_wobble_U-G |
| | | | 31_1-1_wobble_G-U |
| | | | 34_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| | | | 58_1-1_wobble_U-G |
| | | | 62_1-1_wobble_G-U |
| | | | 64_1-1_wobble_G-U |
| P28 | TTTTGCGCTTA | UUUUGCGCUU | −35_1-1_wobble_G-U |
| | TCGCATAAGA | AUCGCAUAAG | −33_1-1_wobble_G-U |
| | GAAGATTTTTT | AGAAGAUUUU | −31_1-1_wobble_U-G |
| | TTTGTAGGGTT | UUUUUGUAGG | −22_6-6_internal_loop- |
| | CGCGGGGAGG | GUUCGCGGGG | symmetric_UCUCCU-UUUCCC |
| | GGGTATTTTA | AGGGGGUAUU | −21_1-1_wobble_G-U |
| | ACGACTCTGT | UUAACGACUC | −18_1-1_wobble_U-G |
| | GTGAACGATC | UGUGUGAACG | −14_1-1_wobble_U-G |
| | GTCTTTTCCCT | AUCGUCUUUU | −13_1-0_bulge- |
| | GTGATTT | CCCUGUGAUU | asymmetric_A- |
| | (SEQ ID | U | −7_1-3_bulge- |
| | NO: 208) | (SEQ ID | asymmetric_G-GUG |
| | | NO: 240) | −3->0_4-4_bulge- |
| | | | symmetric_UAUA-CGAC |
| | | | 8_1-1_wobble_G-U |
| | | | 18_1-1_wobble_U-G |
| | | | 20_1-1_wobble_U-G |
| | | | 22_1-1_wobble_G-U |
| | | | 24_1-1_wobble_U-G |
| | | | 29_1-1_wobble_U-G |
| | | | 31_1-1_wobble_G-U |
| | | | 34_1-1_wobble_G-U |
| | | | 36_4-4_bulge- |
| | | | symmetric_UUUG-AUUU |
| | | | 41_1-1_mismatch_A-A |
| | | | 45_1-1_wobble_U-G |
| | | | 48_1-1_wobble_G-U |
| | | | 51_1-1_wobble_U-G |
| | | | 55_1-1_wobble_G-U |
| | | | 58_1-1_wobble_U-G |
| | | | 62_1-1_wobble_G-U |
| | | | 64_1-1_wobble_G-U |
| Broken | CTCTGCACTCA | CUCUGCACUCA | −31_1-1_wobble_U-G |
| | TCACATAAAA | UCACAUAAAA | −26_1-1_mismatch_C-C |
| | GATGCTTTTCT | GAUGCUUUUC | −25_1-1_wobble_U-G |
| | TCTATAGGATT | UUCUAUAGGA | −21_1-1_wobble_G-U |
| | CACAGGGAGG | UUCACAGGGA | −14_1-1_wobble_U-G |
| | GGGCATTTTA | GGGGGCAUUU | −13_1-0_bulge- |
| | ACGACTCTGT | UAACGACUCU | asymmetric_A- |
| | GTGAACGATC | GUGUGAACGA | −7_1-3_bulge- |
| | ATCTAGGGCA | UCAUCUAGGG | asymmetric_G-GUG |
| | TGTGACTC | CAUGUGACUC | −3->0_4-4_bulge- |
| | (SEQ ID | (SEQ ID | symmetric_UAUA-CGAC |
| | NO: 209) | NO: 241) | 22_1-1_wobble_G-U |
| | | | 36_3-3_bulge- |
| | | | symmetric_UUU-UUU |
| | | | 48_1-1_wobble_G-U |

### Example 6

### Guide RNA Validation for Targeting DUX4

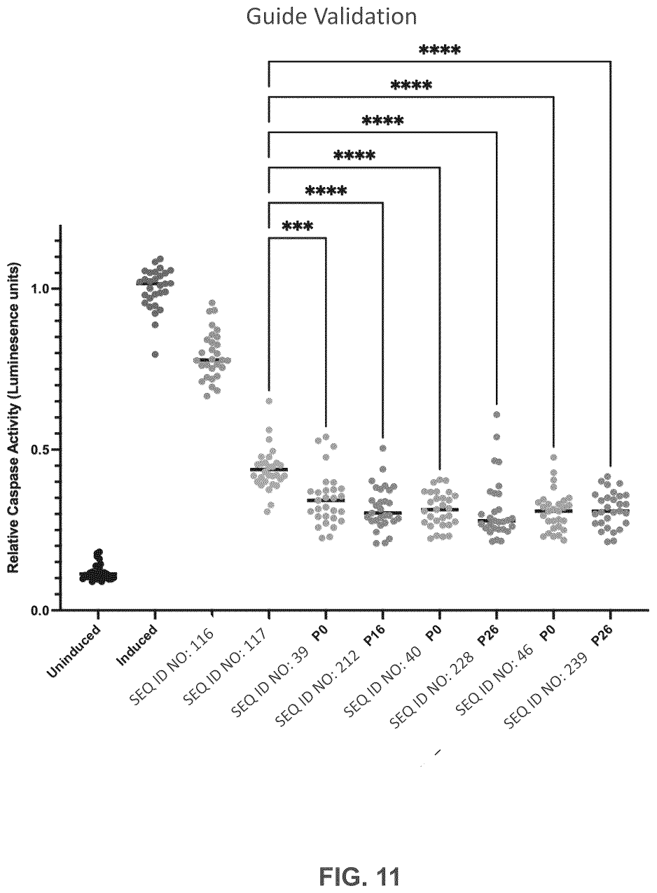

Guide RNAs and their respective progeny guides described in the examples above were tested in HEK293T cells integrated with a tetracycline (tet) inducible DUX4 expression system as described in Example 3. FIG. 11 shows the combined results for the reduction in caspase activity in the inducible DUX4 HEK293T cell line from 3 biologically independent experiments. The selected guide RNAs (SEQ ID NO: 39, 40, and 46) and their respective progeny guide RNAs (SEQ ID NO: 212, 228, and 239) were able to reduce caspase activity as compared to the guide RNAs (SEQ ID NO: 116 and 117). Please note, the SEQ ID NO: 116 control guide was less effective because of reduced transfection efficiency as compared to the selected guide RNAs and the progeny guide RNAs. These results show guide RNAs and respective progeny guide RNAs produced reduced caspase activity in DUX4 induced cells, indicating a reduction in induced DUX4 levels. Table 9 shows the relative caspase activity based on luminescence units from each guide RNA as compared to the untransfected induced cells.

TABLE 9
Caspase activity from transfected guide RNAs

| Guide RNA | Relative caspase activity to |
|---|---|
| SEQ ID NO | untransfected induced cells |
| 116 | 0.78 |
| 117 | 0.437 |
| 39 | 0.342 |
| 212 | 0.303 |
| 40 | 0.313 |
| 228 | 0.279 |
| 46 | 0.308 |
| 239 | 0.309 |

### Example 7

AAV Packaged gRNAs Containing Vector Genomes

Dual guide RNA vectors targeting the polyA signal sequence of DUX4 are packaged into AAVs. The dual guide RNA vectors comprise two guide RNAs, and one or more promoters and/or additional engineered guide RNA components, such as U7 and SmOPT sequences. To produce the AAV vectors, a producer cell line is transfected with: (1) DNA necessary for AAV replication and synthesis of an AAV capsid, (2) one or more helper constructs comprising the viral functions missing from the AAV vector, (3) a helper virus, and (4) a plasmid construct containing the genome of the AAV vector, which comprises the engineered guide RNA sequences. The guide RNA sequences for the dual guide constructions are shown in Table 10.

TABLE 10
Dual guide RNA vectors for AAV production

| Vector genome | | |
|---|---|---|
| Construct | | |
| Number | Guide RNA 1 | Guide RNA 2 |
| 1 | SEQ ID NO: 40 | SEQ ID NO: 228 |
| 2 | SEQ ID NO: 40 | SEQ ID NO: 46 |
| 3 | SEQ ID NO: 39 | SEQ ID NO: 212 |
| 4 | SEQ ID NO: 39 | SEQ ID NO: 46 |
| 5 | SEQ ID NO: 40 | SEQ ID NO: 39 |
| 6 | SEQ ID NO: 46 | SEQ ID NO: 239 |
| 7 | SEQ ID NO: 228 | SEQ ID NO: 239 |
| 8 | SEQ ID NO: 39 | SEQ ID NO: 239 |

### Example 8

### DUX4 Editing in Immortalized and Primary FSHD Patient Myotubes

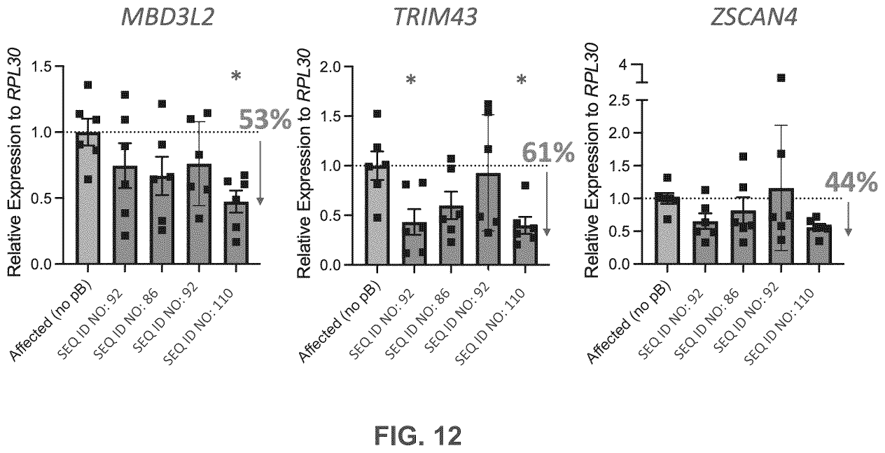

Immortalized affected FSHD cells were tested with transfected vectors expressing engineered guide RNAs for a reduction of DUX4 levels. The reduction in DUX4 levels was evaluated by examining the reduction in expression of genes downstream of DUX4. Immortalized affected FSHD myotubes were stably transfected with engineered guide RNAs targeting DUX4-FL mRNA. The target RNA sequence for DUX4-FL was: DNA—GATGATTAGTTCAGAGATATATTAAAATGCCCCCTCCCTGTGGATCCTATAGAAGAT TTGCATCTTTTGTGTGATGAGTGCAGAGATATGTCACAATATC (SEQ ID NO: 242) or RNA—GAUGAUUAGUUCAGAGAUAUAUUAAAAUGCCCCCUCCCUGUGGAUCCUAUAGAA GAUUUGCAUCUUUUGUGUGAUGAGUGCAGAGAUAUGUCACAAUAUC (SEQ ID NO: 243). The sequences of the engineered guide RNAs are shown in Table 11. FIG. 12 show affected FSHD myotubes demonstrated a decrease in DUX4-activated gene expression after stable transfection with engineered guide RNAs. The guide RNAs were transfected with different promoters and/or SmOPT variants. The results shown are from 3 biological replicates. There was a 53% decrease in MBD3L2 expression, a 61% decrease in TRIM43 expression, and a 44% decrease in ZSCAN4 expression. These results show engineered guide RNAs can successfully target DUX4 in immortalized FSHD cells and reduce expression of genes downstream of DUX4, such as MBD3L2, TRIM43 and ZSCAN4.

The guide RNAs from FIG. 12 are shown in Table 11.

TABLE 11
Guide RNA sequences for targeting the polyA
signal sequence of DUX4-FL

| Guide DNA sequence | Guide RNA sequence |
|---|---|
| GATATTGTGACATATCTCTGCACT | GAUAUUGUGACAUAUCUCUGCAC |
| CATCACACAAAAGATGCAAATCT | UCAUCACACAAAAGAUGCAAAUC |
| GTGGGCGGATCCACAGGGAGGGG | UGUGGGCGGAUCCACAGGGAGGG |
| GCATTTTAACATATCAGCTCCCTA | GGCAUUUUAACAUAUCAGCUCCC |
| ATCATC (SEQ ID NO: 56) | UAAUCAUC (SEQ ID NO: 92) |
| GATATTGTGACATATCTCTGCACT | GAUAUUGUGACAUAUCUCUGCAC |
| CATCACCTACTCGATGCAAATCTT | UCAUCACCUACUCGAUGCAAAUC |
| CTATAGGATCCACAGGGAGGGGG | UUCUAUAGGAUCCACAGGGAGGG |
| CATTTTAACATATGCAGCTACTAA | GGCAUUUUAACAUAUGCAGCUAC |
| TCATC (SEQ ID NO: 50) | UAAUCAUC (SEQ ID NO: 86) |
| GATATTGTGACATATCTCTGCACT | GAUAUUGUGACAUAUCUCUGCAC |
| CATCACACAAAAGATGCAAATCT | UCAUCACACAAAAGAUGCAAAUC |
| GTGGGCGGATCCACAGGGAGGGG | UGUGGGCGGAUCCACAGGGAGGG |
| GCATTTTAACATATCAGCTCCCTA | GGCAUUUUAACAUAUCAGCUCCC |
| ATCATC (SEQ ID NO: 56) | UAAUCAUC (SEQ ID NO: 92) |
| GATATTGTGACATATCTCTGCACT | GAUAUUGUGACAUAUCUCUGCAC |
| CATCACACTCCCTCTGCAAATCTT | UCAUCACACUCCCUCUGCAAAUC |
| CTATAGGATCCACAGGGAGGGGG | UUCUAUAGGAUCCACAGGGAGGG |
| CATTCTAATATATCTCTGACCCTC | GGCAUUCUAAUAUAUCUCUGACC |
| GCATC (SEQ ID NO: 74) | CUCGCAUC (SEQ ID NO: 110) |

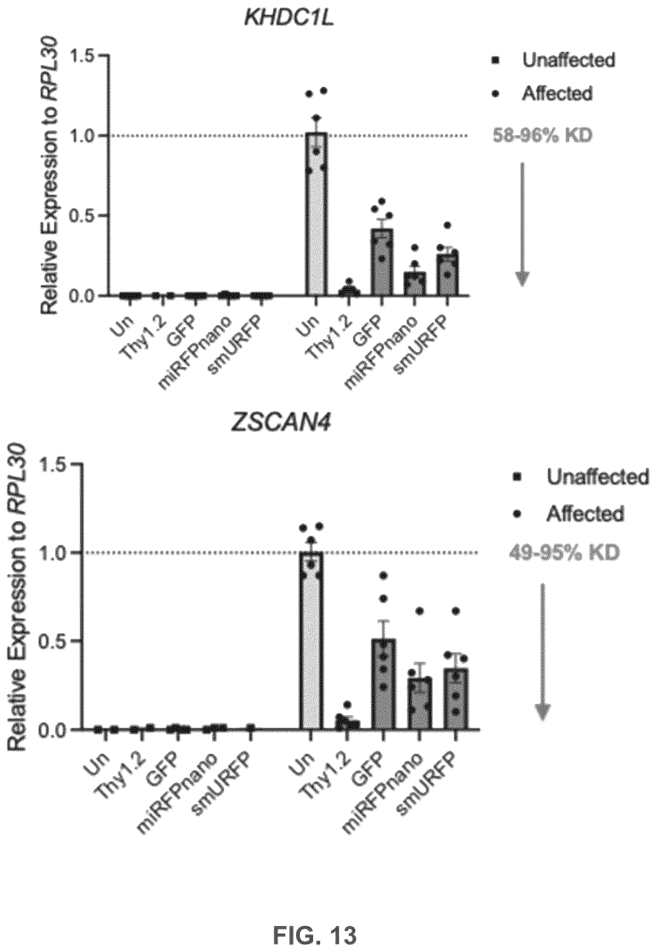

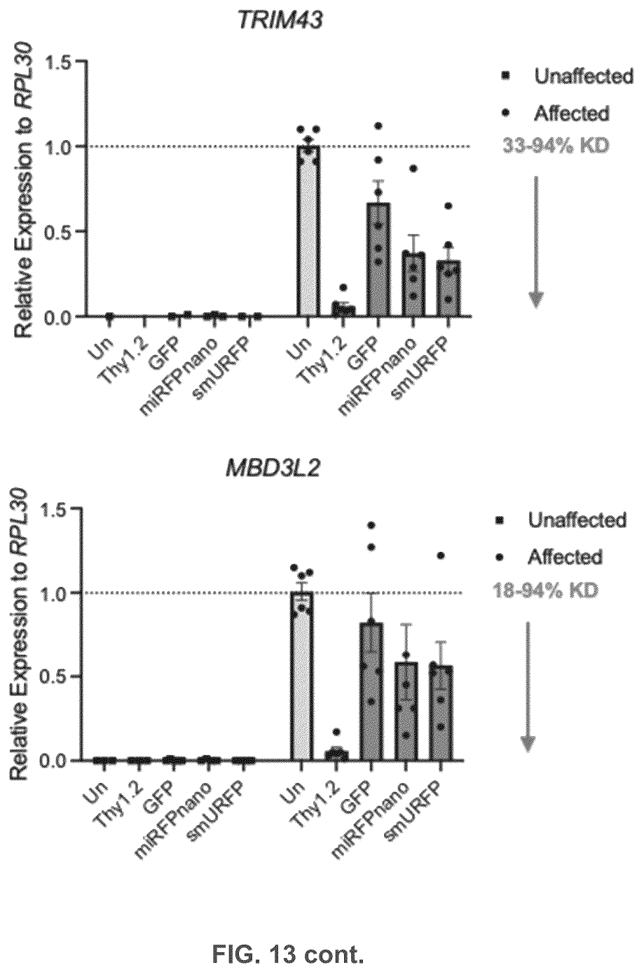

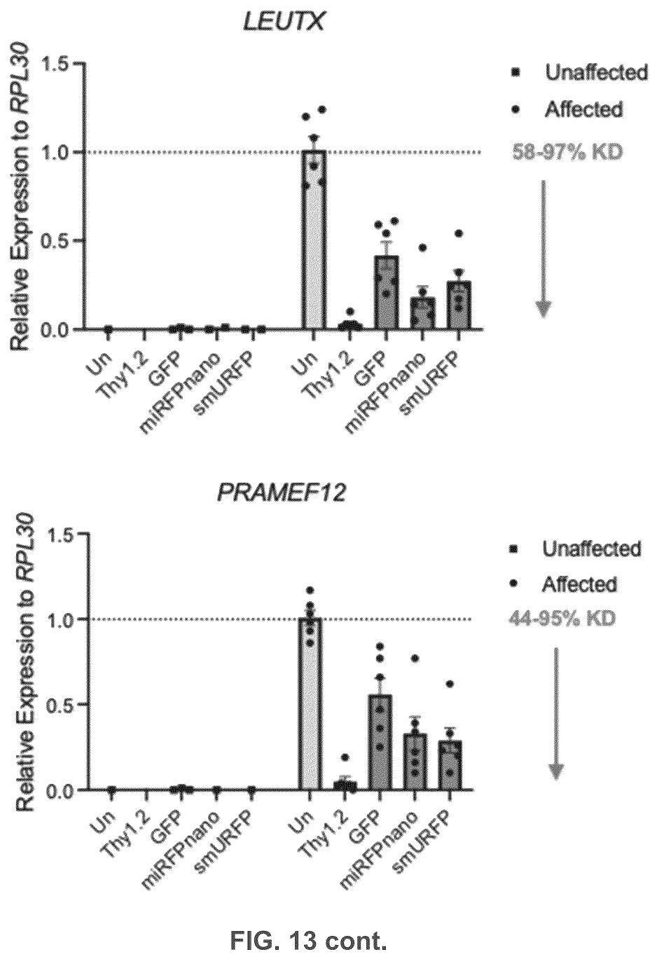

Following the stable transfection experiment, primary FSHD cells from unaffected and affected patients were transduced with a scAAV6 vector to determine if engineered guide RNAs could reduce DUX4 levels in primary FSHD cells by transduction. The reduction in DUX4 levels was evaluated by examining the reduction in expression of genes downstream of DUX4. Primary FSHD myotubes were transduced with scAAV6 vectors containing different transduction markers and a single copy of a guide RNA targeting DUX4. The target RNA sequence for DUX4 was: DNA—GAGTTACATCTCCTGGATGATTAGTTCAGAGATATATTAAAATGCCCCCTCCCTGTG GATCCTATAGAAGATTTGCATCTTTTGTGTGATGAGTGCAGAG (SEQ ID NO: 244) or RNA—GAGUUACAUCUCCUGGAUGAUUAGUUCAGAGAUAUAUUAAAAUGCCCCCUCCCUG UGGAUCCUAUAGAAGAUUUGCAUCUUUUGUGUGAUGAGUGCAGAG (SEQ ID NO: 245). FIG. 13 shows transduced affected FSHD myotubes demonstrated a decrease in DUX4-activated gene expression. The results shown are from 3 biological replicates and are compared to untransduced primary myotubes. There was a 58-96% decrease in KHDC1L expression, a 49-95% decrease in ZSCAN4 expression; a 34-94% decrease in TRIM43 expression, a 18-94% decrease in MBD3L2 expression, a 58-97% decrease in LEUTX expression, and a 44-95% decrease in PRAMEF12 expression. These results show engineered guide RNAs can successfully target DUX4 in primary cells and reduce expression of genes downstream of DUX4, such as KHDC1L, ZSCAN4, TRIM43, MBD3L2, LEUTX and PRAMEF12. The guide RNA from FIG. 13 is shown in Table 12.

TABLE 12
Guide RNA sequence for targeting the polyA
signal sequence of DUX4-FL

| | | Structural Features |
|---|---|---|
| | | of the guide-target |
| Guide DNA sequence | Guide RNA sequence | RNA scaffold |
| CTCTGCACTCATCACACAA | CUCUGCACUCAUCACAC | -6_6-6_internal_loop- |
| AAGATGCAAATCTGTGGGC | AAAAGAUGCAAAUCUGU | symmetric_UUCAGA- |
| GGATCCACAGGGAGGGGG | GGGCGGAUCCACAGGGA | AGCUCC |
| CATTTTAACATATCAGCTC | GGGGGCAUUUUAACAUA | 0_1-1_mismatch_A-C |
| CCTAATCATCCAGGAGATG | UCAGCUCCCUAAUCAUC | 27_6-6_internal_loop- |
| TAACTC (SEQ ID NO: 33) | CAGGAGAUGUAACUC | symmetric_UAUAGA- |
| | (SEQ ID NO: 40) | GUGGGC |

### Example 9

### AAV Transduction in FSHD Patient Myotubes

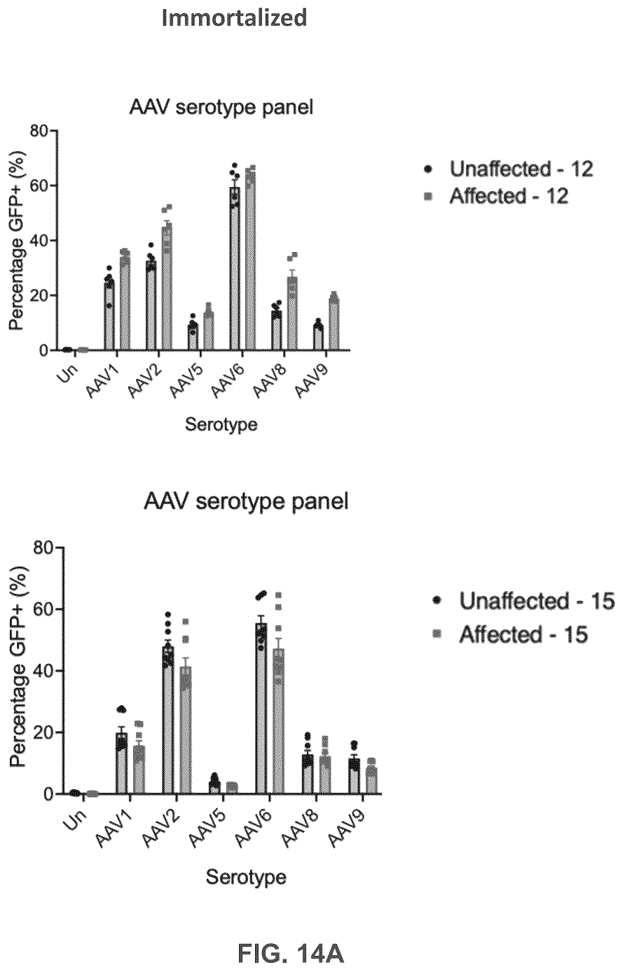

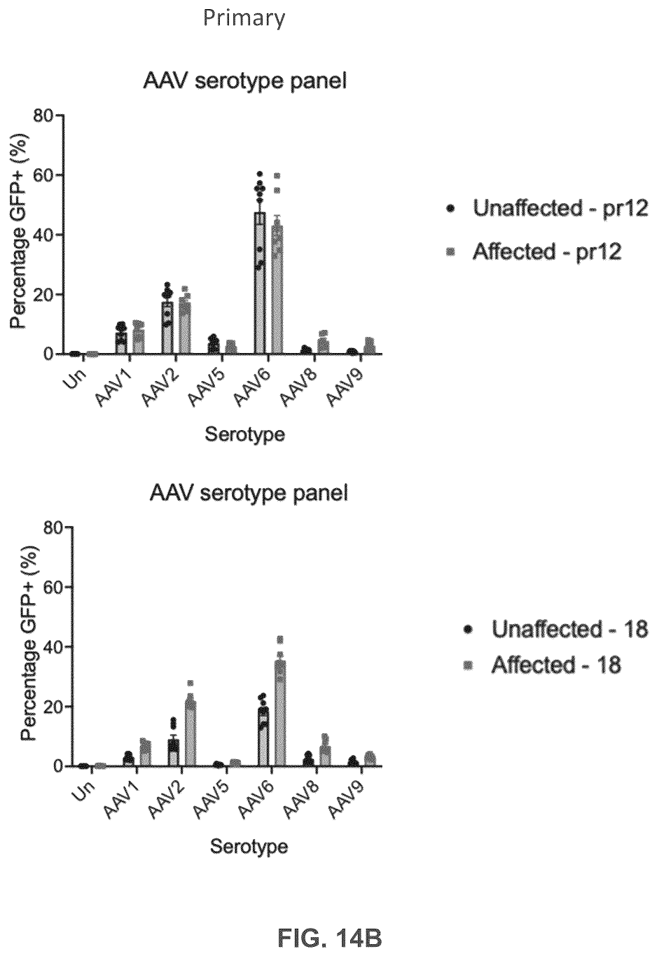

Transduction efficiencies by different AAV vectors were determined for immortalized and primary FSHD cells. The FSHD cells were transduced with scAAV1, scAAV2, scAAV5, scAAV6, scAAV8, and scAAV9, all carrying a GFP reporter. Cells were transduced with a 500,000 multiplicity of infection (MOI) and GFP expression was determined 4 days post-transfection via flow cytometry. FIG. 14A shows immortalized affected FSHD myotubes achieved over 40% transduction with scAAV6 (~50-60%), and scAAV2 (~40%). The immortalized cells tested were unaffected and affected (12Ubic or 12Abic) cells and unaffected and affected (15Vbic or 15Abic) cells. FIG. 14B shows primary affected FSHD myotubes achieved over 20% transduction with scAAV6 (~40%) and scAAV2 (~20%). The primary cells tested were unaffected and affected 12 (12Ubic or 12Abic) and unaffected and affected (18Ubic or 18Abic) cells. These results show scAAVs can successfully transduce immortalized and primary FSHD cells.

### Example 10

### AAV Transduction of Guide RNAs in a Nonhuman Primate

The testis and thymus are reported to have low-level expression of DUX4 in the nonhuman primate (NHP) model. The target DUX4 sequence in these tissues is determined and if the sequence is different than the human DUX4 sequence the double stranded RNA structure will be modeled with guide RNA candidates to attempt to predict cross-reactivity.

A nonhuman primate (NHP) is transduced with an AAV vector comprising a guide RNA targeting DUX4. The NHP model is used to establish in vivo biodistribution and off-target toxicology. The biodistribution is determined in tissues for the AAV (vg/dg). Guide RNA expression is also determined in tissues. Histopathology, exploratory toxicology, and off-target RNA editing are determined in the NHP model. After transduction, the tissues are collected and profiled by RNA sequencing, ddPCR, and histopathology to de-risk any on-target liabilities in the tissues.

### Example 11

### In-Cell Editing of the Polyadenylation Signal Sequence of DUX4

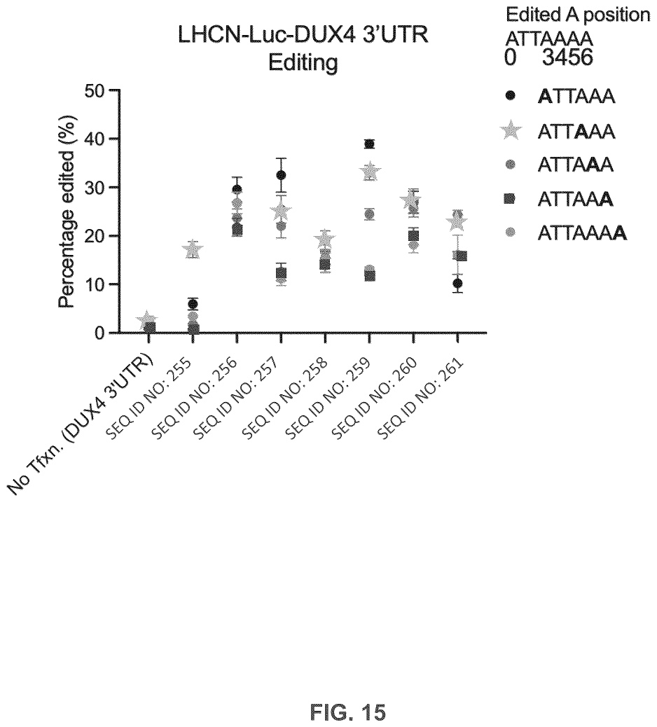

DUX4 targeting guide RNAs were tested for in-cell editing. LHCN muscle cells were engineered to stably express luciferase-DUX4-3′UTR. DUX4 targeting guide RNAs were transiently transfected into the LHCN muscle cells with stable expression of Luciferase-DUX4-3′UTR. The target RNA was SEQ ID NO: 1. Cells were harvested 48 hours post-transfection to identify the percent editing, which was determined by Sanger sequencing. As shown in FIG. 15, the polyadenylation sequence of non-canonical DUX4 transcripts was edited by all tested guide RNAs and all the guide RNAs facilitated editing of one or more adenosines. Editing of the polyA sequence was as high as about 40%. This data shows editing of multiple adenosines in the polyadenylation signal sequence of DUX4 is facilitated by the guide RNAs disclosed herein.

The guide RNAs from FIG. 15 are shown in Table 13.

TABLE 13
Guide RNA sequences for targeting the polyA signal sequence of DUX4

| | | Structural Features of the |
|---|---|---|
| Guide DNA sequence | Guide RNA sequence | guide-target RNA scaffold |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | 3_1-1_mismatch_A-C |
| CAAATCTTCTATAGACA | UGCAAAUCUUCUAUAGA | 20_6-6_internal_loop- |
| GGTCAGGGAGGGGGCAT | CAGGUCAGGGAGGGGGC | symmetric_UGGAUC-ACAGGU |
| TTCAATATATCTCTGAAC | AUUUCAAUAUAUCUCUG | 66_3-6_internal_loop- |
| TAATCATCGTGGAGAGA | AACUAAUCAUCGUGGAG | asymmetric_AAA-GACAUA |
| CCAT (SEQ ID NO: 248) | AGACCAU (SEQ ID NO: | 70_1-1_wobble_U-G |
| | 255) | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAACCCTCCC | ACUCAUCACACAACCCU | -9_6-6_internal_loop- |
| AAATCTTCTATAGGATCC | CCCAAAUCUUCUAUAGG | symmetric_UAGUUC-CGUGAU |
| ACAGGGAGGGGGCATTT | AUCCACAGGGAGGGGGC | 0_1-1_mismatch_A-C |
| TAACATATCTCTCGTGAT | AUUUUAACAUAUCUCUC | 40_6-6_internal_loop- |
| ATCATCGTGGAGAGACC | GUGAUAUCAUCGUGGAG | symmetric_CAUCUU-CCCUCC |
| AT (SEQ ID NO: 249) | AGACCAU (SEQ ID NO: | 66_3-6_internal_loop- |
| | 256) | asymmetric_AAA-GACAUA |
| | | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | -6_6-6_internal_loop- |
| CTCCCTATCTATAGGATC | UGCUCCCUAUCUAUAGG | symmetric_UUCAGA-CACCUC |
| CACAGGGAGGGGGCATT | AUCCACAGGGAGGGGGC | 0_1-1_mismatch_A-C |
| TTAACATATCCACCTCCT | AUUUUAACAUAUCCACC | 33_6-6_internal_loop- |
| AATCATCGTGGAGAGAC | UCCUAAUCAUCGUGGAG | symmetric_AGAUUU-UCCCUA |
| CAT (SEQ ID NO: 250) | AGACCAU (SEQ ID NO: | 66_3-6_internal_loop- |
| | 257) | asymmetric_AAA-GACAUA |
| | | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACCTGTCCTGC | ACUCAUCACACCUGUCC | -6_6-6_internal_loop- |
| AAATCTTCTATAGGATCC | UGCAAAUCUUCUAUAGG | symmetric_UUCAGA-AGCAUC |
| ACAGGGAGGGGGCATTT | AUCCACAGGGAGGGGGC | 3_1-1_mismatch_A-C |
| CAATATATCAGCATCCTA | AUUUCAAUAUAUCAGCA | 42_6-6_internal_loop- |
| ATCATCGTGGAGAGACC | UCCUAAUCAUCGUGGAG | symmetric_UCUUUU-CUGUCC |
| AT (SEQ ID NO: 251) | AGACCAU (SEQ ID NO: | 66_3-6_internal_loop- |
| | 258) | asymmetric_AAA-GACAUA |
| | | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | -5_6-6_internal_loop- |
| CCCTGGATCTATAGGATC | UGCCCUGGAUCUAUAGG | symmetric_UCAGAG-GAGAUC |
| CACAGGGAGGGGGCATT | AUCCACAGGGAGGGGGC | 33_6-6_internal_loop- |
| TTAATATATGAGATCACT | AUUUUAAUAUAUGAGA | symmetric_AGAUUU-CCUGGA |
| AATCATCGTGGAGAGAC | UCACUAAUCAUCGUGGA | 66_3-6_internal_loop- |
| CAT (SEQ ID NO: 252) | GAGACCAU (SEQ ID NO: | asymmetric_AAA-GACAUA |
| | 259) | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGACC | ACUCAUCACACAAAAGA | -3_6-6_internal_loop- |
| CCTCTCTTCTATAGGATC | CCCCUCUCUUCUAUAGG | symmetric_AGAGAU-UCAAAC |
| CACAGGGAGGGGGCATT | AUCCACAGGGAGGGGGC | 4_1-1_mismatch_A-C |
| CTAATATTCAAACGAAC | AUUCUAAUAUUCAAACG | 36_6-6_internal_loop- |
| TAATCATCGTGGAGAGA | AACUAAUCAUCGUGGAG | symmetric_UUUGCA-CCCCUC |
| CCAT (SEQ ID NO: 253) | AGACCAU (SEQ ID NO: | 66_3-6_internal_loop- |
| | 260) | asymmetric_AAA-GACAUA |
| | | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | -1_6-6_internal_loop- |
| CACAGGGGCTATAGGAT | UGCACAGGGGCUAUAGG | symmetric_AGAUAU-UCCCUG |
| CCACAGGGAGGGGGCAT | AUCCACAGGGAGGGGGC | 4_1-1_mismatch_A-C |
| TCTAATTCCCTGCTGAAC | AUUCUAAUUCCCUGCUG | 32_6-6_internal_loop- |
| TAATCATCGTGGAGAGA | AACUAAUCAUCGUGGAG | symmetric_AAGAUU-CAGGGG |
| CCAT (SEQ ID NO: 254) | AGACCAU (SEQ ID NO: | 66_3-6_internal_loop- |
| | 261) | asymmetric_AAA-GACAUA |
| | | 70_1-1_wobble_U-G |
| | | 72_1-1_wobble_G-U |
| | | 73_3-2_bulge-asymmetric_GUC- |
| | | UA |

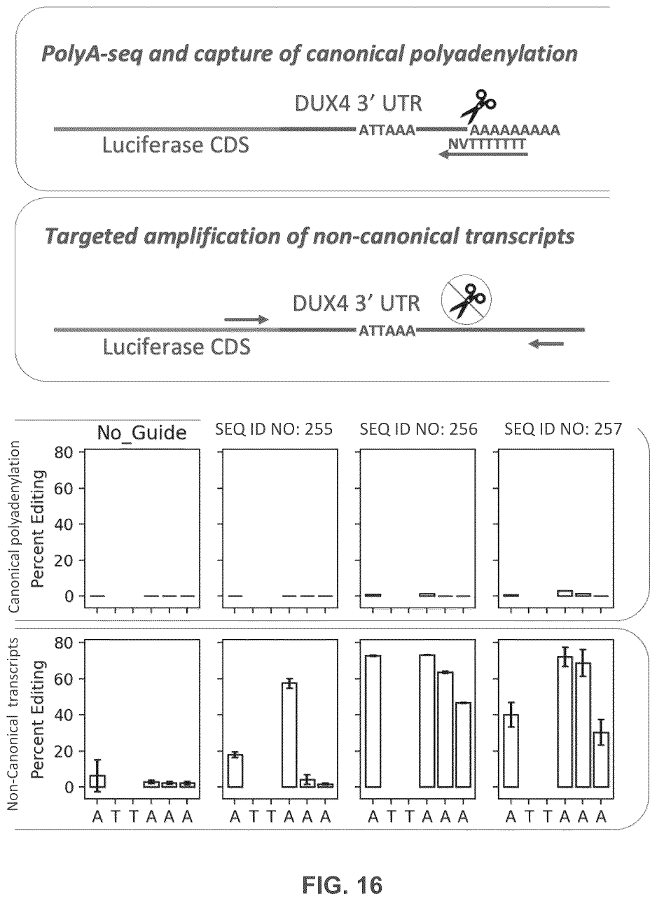

Further, cells were tested to determine editing in non-canonical DUX4 transcripts compared to transcripts with canonical polyadenylation and cleavage. HEK293 cells were engineered to stably express luciferase-DUX4-3′UTR. DUX4 guide RNAs were transiently transfected into the HEK293 cells with stable expression of Luciferase-DUX4-3′UTR. Cells were harvested 48 hours post-transfection and PolyA-seq was used to capture the canonical polyadenylation site. The target RNA was SEQ ID NO: 1. FIG. 16 shows editing of the polyadenylation sequence was found only in non-canonical transcripts, indicating DUX4 guide RNAs significantly impair cleavage and polyadenylation. Targeted amplification and Sanger sequencing of non-canonical minority transcripts showed extensive editing (e.g., up to about 70%) of the polyadenylation signal sequence in non-canonical transcripts while there was minimal to no editing in the canonical polyadenylation signal sequence. This data shows DUX4 polyadenylation sequence editing disrupts canonical cleavage and maturation.

The guide RNAs from FIG. 16 are shown in Table 14.

TABLE 14
Guide RNA sequences for targeting the polyA signal
sequence of DUX4

| | | Structural Features of the |
|---|---|---|
| Guide DNA sequence | Guide RNA sequence | guide-target RNA scaffold |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | 3_1-1_mismatch_A-C |
| CAAATCTTCTATAGACA | UGCAAAUCUUCUAUAGA | 20_6-6_internal_loop- |
| GGTCAGGGAGGGGGCAT | CAGGUCAGGGAGGGGGC | symmetric_UGGAUC-ACAGGU |
| TTCAATATATCTCTGAAC | AUUUCAAUAUAUCUCUG | 66_3-6_internal_loop- |
| TAATCATCGTGGAGAGA | AACUAAUCAUCGUGGAG | asymmetric_AAA-GACAUA |
| CCAT (SEQ ID NO: 248) | AGACCAU (SEQ ID NO: | 70_1-1_wobble_U-G |
| | 255) | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAACCCTCCC | ACUCAUCACACAACCCU | -9_6-6_internal_loop- |
| AAATCTTCTATAGGATCC | CCCAAAUCUUCUAUAGG | symmetric_UAGUUC-CGUGAU |
| ACAGGGAGGGGGCATTT | AUCCACAGGGAGGGGGC | 0_1-1_mismatch_A-C |
| TAACATATCTCTCGTGAT | AUUUUAACAUAUCUCUC | 40_6-6_internal_loop- |
| ATCATCGTGGAGAGACC | GUGAUAUCAUCGUGGAG | symmetric_CAUCUU-CCCUCC |
| AT (SEQ ID NO: 249) | AGACCAU (SEQ ID NO: | 66_3-6_internal_loop- |
| | 256) | asymmetric_AAA-GACAUA |
| | | 70_1-1_wobble_U-G |
| | | 72_1-1_wobble_G-U |
| | | 73_3-2_bulge-asymmetric_GUC- |
| | | UA |
| TAGGTCTCCACCGGATAT | UAGGUCUCCACCGGAUA | -21_2-2_bulge-symmetric_UG- |
| TGTGACATATCTCTGCAC | UUGUGACAUAUCUCUGC | GU |
| TCATCACACAAAAGATG | ACUCAUCACACAAAAGA | -6_6-6_internal_loop- |
| CTCCCTATCTATAGGATC | UGCUCCCUAUCUAUAGG | symmetric_UUCAGA-CACCUC |
| CACAGGGAGGGGGCATT | AUCCACAGGGAGGGGGC | 0_1-1_mismatch_A-C |
| TTAACATATCCACCTCCT | AUUUUAACAUAUCCACC | 33_6-6_internal_loop- |
| AATCATCGTGGAGAGAC | UCCUAAUCAUCGUGGAG | symmetric_AGAUUU-UCCCUA |
| CAT (SEQ ID NO: 250) | AGACCAU (SEQ ID NO: | 66 3-6_internal_loop- |
| | 257) | asymmetric_AAA-GACAUA |
| | | 70_1-1_wobble_U-G |
| | | 72_1-1_wobble_G-U |
| | | 73_3-2_bulge- |
| | | asymmetric_GUC-UA |

### Example 12

Gene Knockdown with DUX4-Targeted gRNA in a Mouse Model

A DUX4 inducible mouse model was used to test knockdown of DUX4-activated genes, as a proxy for DUX4-FL knockdown, by guide RNAs targeting DUX4 administered with an AAV6 vector. DUX4 transgene expression in the mice was induced by tamoxifen intraperitoneal injection 2 weeks prior to the harvest time point. The AAV6 vector comprising a polynucleotide encoding a DUX4 guide RNA (CTCTGCACTCATCACACAAAAGATGCAAATCTGTGGGCGGATCCACAGGGAGGGGG CATTTTAACATATCAGCTCCCTAATCATCCAGGAGATGTAACTC—SEQ ID NO: 33, encoding an RNA sequence of CUCUGCACUCAUCACACAAAAGAUGCAAAUCUGUGGGCGGAUCCACAGGGAGGGG GCAUUUUAACAUAUCAGCUCCCUAAUCAUCCAGGAGAUGUAACUC—SEQ ID NO: 40) and a RAB7A guide RNA was administered 4 weeks prior to the harvest time point. 5 female DUX4-inducible mice were included in each study arm (control and treatment). The treatment group received a bilateral intramuscular injection. The left tibialis anterior muscle received about 1E11 vg, and the right tibialis anterior muscle received PBS. The control group received an intramuscular injection of the vehicle control (PBS).

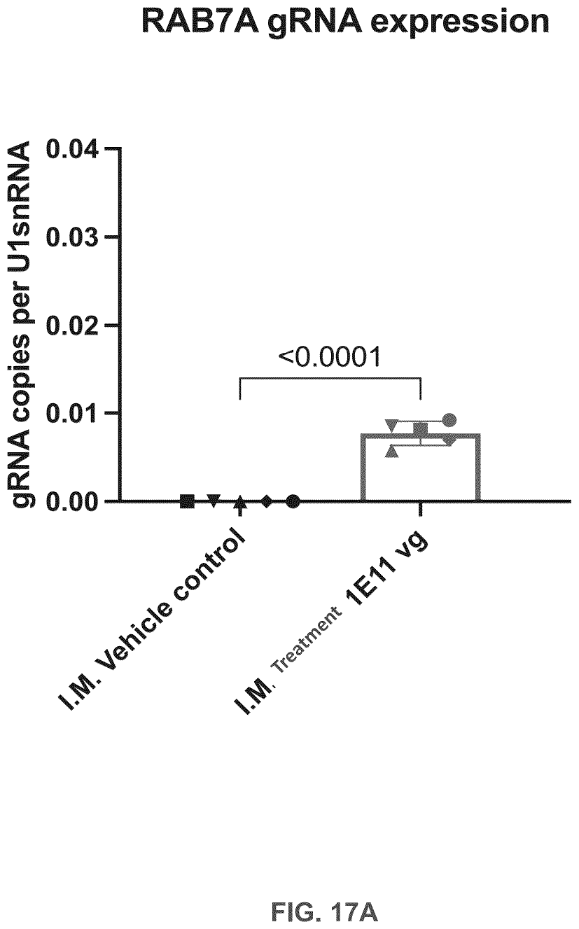

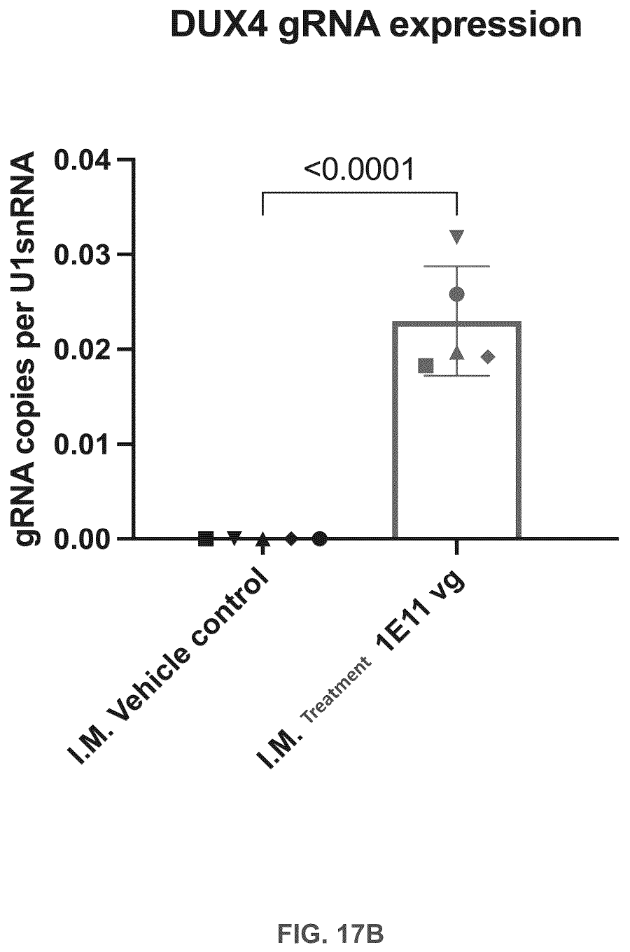

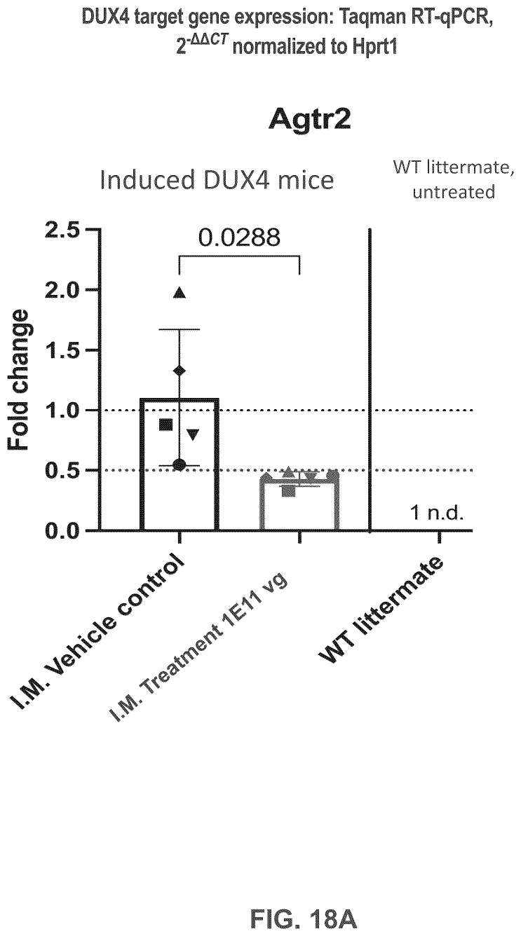

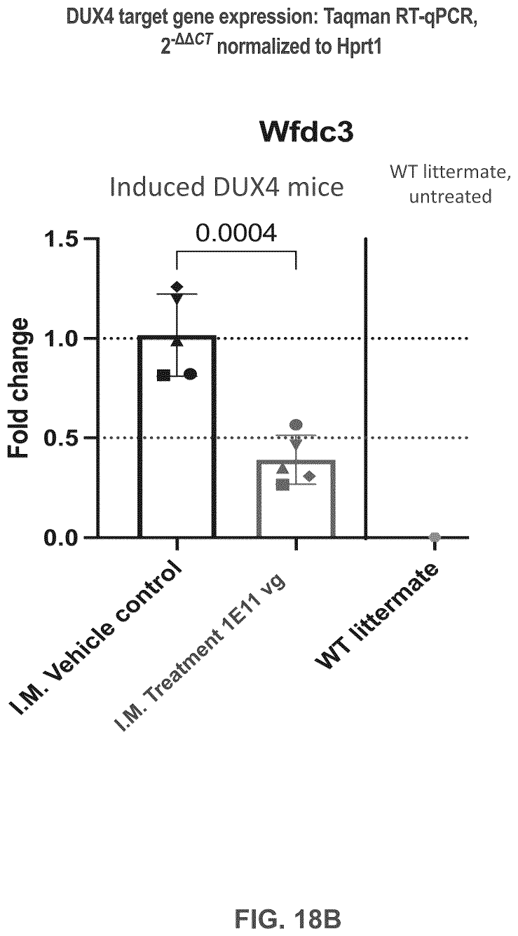

The AAV6 vector produced the DUX4 and RAB7A guide RNAs in the treatment mouse group, which is shown in FIG. 17A (RAB7A) and FIG. 17B (DUX4). The figures showed the RAB7A cassette produced less guide RNA than the DUX4 cassette. To determine knockdown, the Agtr2 and Wfdc3 DUX-4 activated genes were examined. Agtr2 and Wfdc3 were knocked down about 57% and 61%, respectively. The knockdown for Agtr2 is shown in FIG. 18A, and the knockdown for Wfdc3 is shown in FIG. 18B. These results show that targeting the DUX4 polyadenylation signal sequence with a polynucleotide encoding a DUX4 guide RNA ensues knockdown of expression in genes downstream of DUX4.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1-108. (canceled)

109. An engineered guide RNA capable of hybridizing to a DUX4 target sequence of a DUX4-FL mRNA, wherein the DUX4 target sequence comprises a polyA signal sequence, and wherein the engineered guide RNA has at least 90% sequence identity to SEQ ID NO: 39.

110. The engineered guide RNA of claim 109, wherein the engineered guide RNA upon hybridization to the target sequence, forms a guide-target RNA scaffold comprising at least three structural features, wherein at least one of the at least three structure features is a 6/6 symmetric internal loop.

111. The engineered guide RNA of claim 109, wherein the engineered guide comprises a length of (i) from 95 to 105 nucleotides, or (ii) about 100 nucleotides.

112. The engineered guide RNA of claim 109, wherein the engineered guide RNA comprises a sequence that is SEQ ID NO: 39.

113. The engineered guide RNA of claim 109, wherein the structural features comprise a mismatch and at least a 6/6 symmetric internal loop.

114. The engineered guide RNA of claim 109, wherein the structural features comprise a mismatch and at least two 6/6 symmetric internal loops.

115. The engineered guide RNA of claim 109, wherein the 6/6 symmetric internal loop is at position −5 and/or position 44 relative to a target adenosine at position 0.

116. The engineered guide RNA of claim 109, wherein the engineered guide RNA hybridizes to at least 80 bases of the DUX4 target sequence that has at least 80% sequence identity to
(SEQ ID NO: 1)
ACCUGGAUUAGAGUUACAUCUCCUGGAUGAUUAGUUCAGAGAUAUAUUAA
AAUGCCCCCUCCCUGUGGAUCCUAUAGAAGAUUUGCAUCUUUUGUGUGAU
GAGUGCAGAG.

117. The engineered guide RNA of claim 109, wherein the engineered guide RNA when hybridized to the DUX4 target sequence of the DUX4-FL mRNA facilitates at least 30% knockdown of a DUX4 protein encoded by the DUX4-FL mRNA.

118. The engineered guide RNA of claim 109, wherein the engineered guide RNA when hybridized to the DUX4 target sequence facilitates at least 30% knockdown of a gene downstream of DUX4 and wherein the gene downstream of DUX4 comprises SLC34A2, LEUTX, ZSCAN4, PRAMEF12, TRIM43, KHDC1L, Wfdc3, Agtr2, DEFB103, or MBD3L2.

119. The engineered guide RNA of claim 109, wherein:
the engineered guide RNA when hybridized to the DUX4 target sequence facilitates RNA editing by an RNA editing entity of one or more adenosines in the polyA signal sequence; and the RNA editing entity comprises a human ADAR1, or a human ADAR2.

120. A polynucleotide encoding the engineered guide RNA of claim 109, wherein the polynucleotide encoding the engineered guide RNA has a polynucleotide sequence with at least 90% sequence identity to SEQ ID NO: 32.

121. The polynucleotide of claim 120, wherein the polynucleotide encoding the engineered guide RNA comprises a polynucleotide sequence that is SEQ ID NO: 32.

122. An AAV vector comprising a polynucleotide encoding the engineered guide RNA of claim 109.

123. The AAV vector of claim 122, wherein the AAV vector is AAV1, AAV2, AAV3, AAV4, AAV5, AAV6, AAV7, AAV8, AAV9, AAV10, AAV11, or a derivative, a chimera, or a variant thereof.

124. The AAV vector of claim 122, wherein the AAV vector is a recombinant AAV (rAAV) vector, a hybrid AAV vector, a chimeric AAV vector, a self-complementary AAV (scAAV) vector, or any combination thereof.

125. An AAV vector comprising a polynucleotide an engineered guide RNA, wherein the polynucleotide encoding the engineered guide RNA has a polynucleotide sequence with at least 90% sequence identity to SEQ ID NO: 32.

126. An engineered guide RNA or a polynucleotide encoding the engineered guide RNA, comprising a polynucleotide sequence with at least 90% sequence identity to SEQ ID NO: 32 or SEQ ID NO: 39

127. An engineered guide RNA or a polynucleotide encoding the engineered guide RNA, wherein the engineered guide RNA hybridizes to at least 80 bases of a target RNA sequence with at least 80% sequence identity to ACCUGGAUUAGAGUUACAUCUCCUGGAUGAUUAGUUCAGAGAUAUAUUAAAA UGCCCCCUCCCUGUGGAUCCUAUAGAAGAUUUGCAUCUUUUGUGUGAUGAGU GCAGAG (SEQ ID NO: 1) and facilitates a protein knockdown or an mRNA knockdown.
